SEQUENCE LISTING (50 sequences)

SEQ ID NO: 1 - protein, 5 aa, Mus musculus
SYWMN

SEQ ID NO: 2 - protein, 17 aa, Mus musculus
QIYPGDGDTHYNGKFKG

SEQ ID NO: 3 - protein, 7 aa, Mus musculus
SNWVGSY

SEQ ID NO: 4 - protein, 16 aa, Mus musculus
KSSQSLLESDGKTYLN

SEQ ID NO: 5 - protein, 7 aa, Mus musculus
LVSRLDS

SEQ ID NO: 6 - protein, 9 aa, Mus musculus
WQGTHLPQT

SEQ ID NO: 7 - protein, 116 aa, Mus musculus
QVQLQQSGAELVRPGSSVKISCKSSGYAFSSYWMNWVKQRPGQGLEWIGQIYPGDGDTHYNGKFKGKATLTADKSSSTAYMQLSRLTSEDSAVYFCSSSNWVGSYWGQGTLVTVSA

SEQ ID NO: 8 - protein, 116 aa, synthetic construct
  REGION 1..116: note = Humanized 5H10 Heavy chain variable region VH1
QVQLVQSGAELKKPGSSVKISCKSSGYAFSSYWMNWVKQRPGQGLEWIGQIYPGDGDTHYNGKFKGKATLTADKSTSTAYMELSSLTSEDSAVYFCSSSNWVGSYWGQGTLVTVSS

SEQ ID NO: 9 - protein, 116 aa, synthetic construct
  REGION 1..116: note = Humanized 5H10 Heavy chain variable region VH2
QVQLVQSGAEVKKPGSSVKISCKSSGYAFSSYWMNWVKQRPGQGLEWIGQIYPGDGDTHYNGKFKGKATLTADKSTSTAYMELSSLRSEDTAVYFCSSSNWVGSYWGQGTLVTVSS

SEQ ID NO: 10 - protein, 116 aa, synthetic construct
  REGION 1..116: note = Humanized 5H10 Heavy chain variable region VH3
QVQLVQSGAEVKKPGSSVKVSCKSSGYAFSSYWMNWVRQRPGQGLEWIGQIYPGDGDTHYNGKFKGKATLTADKSTSTAYMELSSLRSEDTAVYFCSSSNWVGSYWGQGTLVTVSS

SEQ ID NO: 11 - protein, 116 aa, synthetic construct
  REGION 1..116: note = Humanized 5H10 Heavy chain variable region VH4
QVQLVQSGAEVKKPGSSVKVSCKSSGYAFSSYWMNWVRQRPGQGLEWIGQIYPGDGDTHYNGKFKGRVTITADKSTSTAYMELSSLRSEDTAVYFCSSSNWVGSYWGQGTLVTVSS

SEQ ID NO: 12 - protein, 116 aa, synthetic construct
  REGION 1..116: note = Humanized 5H10 Heavy chain variable region VH5
QVQLVQSGAEVKKPGSSVKVSCKSSGYAFSSYWMNWVRQRPGQGLEWIGQIYPGDGDTHYNGKFQGRVTITADKSTSTAYMELSSLRSEDTAVYYCSSSNWVGSYWGQGTLVTVSS

SEQ ID NO: 13 - protein, 112 aa, Mus musculus
DVVMTQTPLTLSVTIGQTASISCKSSQSLLESDGKTYLNWLSQRPGQSPKRLIYLVSRLDSGVPDRFTGSGSGTDFTLKISRVEAEDLGVYYCWQGTHLPQTFGGGTKLEIK

SEQ ID NO: 14 - protein, 112 aa, synthetic construct
  REGION 1..112: note = Humanized 5H10 Light chain variable region VK1
DVVMTQSPLTLSVTLGQPASISCKSSQSLLESDGKTYLNWLQQRPGQSPRRLIYLVSRLDSGVPDRFTGSGSGTDFTLKISRVEAEDVGVYYCWQGTHLPQTFGGGTKVEIK

SEQ ID NO: 15 - protein, 112 aa, synthetic construct
  REGION 1..112: note = Humanized 5H10 Light chain variable region VK2
DVVMTQSPLSLPVTLGQPASISCKSSQSLLESDGKTYLNWLQQRPGQSPRRLIYLVSRLDSGVPDRFTGSGSGTDFTLKISRVEAEDVGVYYCWQGTHLPQTFGGGTKVEIK

SEQ ID NO: 16 - protein, 112 aa, synthetic construct
  REGION 1..112: note = Humanized 5H10 Light chain variable region VK3
DVVMTQSPLSLPVTLGQPASISCKSSQSLLESDGKTYLNWLQQRPGQSPRRLIYLVSRLDSGVPDRFSGSGSGTDFTLKISRVEAEDVGVYYCWQGTHLPQTFGGGTKVEIK

SEQ ID NO: 17 - protein, 112 aa, synthetic construct
  REGION 1..112: note = Humanized 5H10 Light chain variable region VK4
DVVMTQSPLSLPVTLGQPASISCKSSQSLLESDGKTYLNWFQQRPGQSPRRLIYLVSRLDSGVPDRFSGSGSGTDFTLKISRVEAEDVGVYYCWQGTHLPQTFGGGTKVEIK

SEQ ID NO: 18 - DNA, 348 bp, Mus musculus
  source 1..348: mol_type = other DNA
caggttcagctgcagcagtctggggctgagctcgtgaggcctgggtcctcagtgaagatttcctgcaagtcttctggctatgcattcagtagctactggatgaactgggtgaagcagaggcctggacagggtcttgagtggattggacagatttatcctggagatggtgatactcactacaatggaaagttcaagggcaaagccacactgactgcagacaaatcctccagcacagcctacatgcagctcagcaggctaacatctgaggactctgcggtctatttctgttcaagttcaaactgggtcgggagttactggggccaagggactctggtcaccgtctcctca

SEQ ID NO: 19 - DNA, 348 bp, synthetic construct
  misc_feature 1..348: note = Humanized heavy chain variable region VH1
  source 1..348: mol_type = other DNA
caggtccagctggtgcaatctggggctgagctgaagaagcctgggtcctcggtgaagatttcctgcaagtcttctggatatgcattcagcagctattggatgaactgggtgaagcagaggcctggacaagggcttgagtggattggacagatctatcctggagatggtgatactcactacaatggaaagttcaagggcaaagccacgctgaccgcggacaaatccacgagcacagcctacatggagctgagcagcctgacatctgaggactctgccgtgtatttctgttcaagttcaaactgggtcgggagttactggggccaaggaaccctggtcaccgtctcctca

SEQ ID NO: 20 - DNA, 348 bp, synthetic construct
  misc_feature 1..348: note = Humanized heavy chain variable region VH2
  source 1..348: mol_type = other DNA
caggtccagctggtgcaatctggggctgaggtgaagaagcctgggtcctcggtgaagatttcctgcaagtcttctggatatgcattcagcagctattggatgaactgggtgaagcagaggcctggacaagggcttgagtggattggacagatctatcctggagatggtgatactcactacaatggaaagttcaagggcaaagccacgctgaccgcggacaaatccacgagcacagcctacatggagctgagcagcctgagatctgaggacacggccgtgtatttctgttcaagttcaaactgggtcgggagttactggggccaaggaaccctggtcaccgtctcctca

SEQ ID NO: 21 - DNA, 348 bp, synthetic construct
  misc_feature 1..348: note = Humanized heavy chain variable region VH3
  source 1..348: mol_type = other DNA
caggtccagctggtgcaatctggggctgaggtgaagaagcctgggtcctcggtgaaggtttcctgcaagtcttctggatatgcattcagcagctattggatgaactgggtgcgacagaggcctggacaagggcttgagtggattggacagatctatcctggagatggtgatactcactacaatggaaagttcaagggcaaagccacgctgaccgcggacaaatccacgagcacagcctacatggagctgagcagcctgagatctgaggacacggccgtgtatttctgttcaagttcaaactgggtcgggagttactggggccaaggaaccctggtcaccgtctcctca

SEQ ID NO: 22 - DNA, 348 bp, synthetic construct
  misc_feature 1..348: note = Humanized heavy chain variable region VH4
  source 1..348: mol_type = other DNA
caggtccagctggtgcaatctggggctgaggtgaagaagcctgggtcctcggtgaaggtttcctgcaagtcttctggatatgcattcagcagctattggatgaactgggtgcgacagaggcctggacaagggcttgagtggattggacagatctatcctggagatggtgatactcactacaatggaaagttcaagggcagagtcacgattaccgcggacaaatccacgagcacagcctacatggagctgagcagcctgagatctgaggacacggccgtgtatttctgttcaagttcaaactgggtcgggagttactggggccaaggaaccctggtcaccgtctcctca

SEQ ID NO: 23 - DNA, 348 bp, synthetic construct
  misc_feature 1..348: note = Humanized heavy chain variable region VH5
  source 1..348: mol_type = other DNA
caggtccagctggtgcaatctggggctgaggtgaagaagcctgggtcctcggtgaaggtttcctgcaagtcttctggatatgcattcagcagctattggatgaactgggtgcgacagaggcctggacaagggcttgagtggattggacagatctatcctggagatggtgatactcactacaatggaaagttccagggcagagtcacgattaccgcggacaaatccacgagcacagcctacatggagctgagcagcctgagatctgaggacacggccgtgtattactgttcaagttcaaactgggtcgggagttactggggccaaggaaccctggtcaccgtctcctca

SEQ ID NO: 24 - DNA, 336 bp, Mus musculus
  source 1..336: mol_type = other DNA
gatgttgtgatgacccagactccactcactttgtcggttaccattggacaaacagcctccatctcttgcaagtcaagtcagagcctcttagaaagtgatggaaagacatatttgaattggttgtcacagaggccaggccagtctccaaagcgcctaatctatctggtgtctagactggactctggagtccctgacaggttcactggcagtggatcagggacagatttcacactgaaaatcagcagagtggaggctgaggatttgggagtttattattgttggcaaggcacacatcttcctcagacgttcggtggaggcaccaagctggagatcaaa

SEQ ID NO: 25 - DNA, 336 bp, synthetic construct
  misc_feature 1..336: note = Humanized light chain variable region VK1
  source 1..336: mol_type = other DNA
gatgttgtgatgactcagtctccactcactctgtcggtcacccttggacagccggcctccatctcctgcaagtctagtcaaagcctcttagaaagtgatggaaagacctacttgaattggttgcagcagaggccaggccaatctccaaggcgcctaatttatctggtttctagactggactctggggtcccagacagattcactggcagtgggtcaggcactgatttcacactgaaaatcagcagggtggaggctgaggatgttggggtttattactgctggcaaggcacacatcttcctcagactttcggcggagggaccaaggtggagatcaaa

SEQ ID NO: 26 - DNA, 336 bp, synthetic construct
  misc_feature 1..336: note = Humanized light chain variable region VK2
  source 1..336: mol_type = other DNA
gatgttgtgatgactcagtctccactctccctgcccgtcacccttggacagccggcctccatctcctgcaagtctagtcaaagcctcttagaaagtgatggaaagacctacttgaattggttgcagcagaggccaggccaatctccaaggcgcctaatttatctggtttctagactggactctggggtcccagacagattcactggcagtgggtcaggcactgatttcacactgaaaatcagcagggtggaggctgaggatgttggggtttattactgctggcaaggcacacatcttcctcagactttcggcggagggaccaaggtggagatcaaa

SEQ ID NO: 27 - DNA, 336 bp, synthetic construct
  misc_feature 1..336: note = Humanized light chain variable region VK3
  source 1..336: mol_type = other DNA
gatgttgtgatgactcagtctccactctccctgcccgtcacccttggacagccggcctccatctcctgcaagtctagtcaaagcctcttagaaagtgatggaaagacctacttgaattggttgcagcagaggccaggccaatctccaaggcgcctaatttatctggtttctagactggactctggggtcccagacagattcagcggcagtgggtcaggcactgatttcacactgaaaatcagcagggtggaggctgaggatgttggggtttattactgctggcaaggcacacatcttcctcagactttcggcggagggaccaaggtggagatcaaa

SEQ ID NO: 28 - DNA, 336 bp, synthetic construct
  misc_feature 1..336: note = Humanized light chain variable region VK4
  source 1..336: mol_type = other DNA
gatgttgtgatgactcagtctccactctccctgcccgtcacccttggacagccggcctccatctcctgcaagtctagtcaaagcctcttagaaagtgatggaaagacctacttgaattggtttcagcagaggccaggccaatctccaaggcgcctaatttatctggtttctagactggactctggggtcccagacagattcagcggcagtgggtcaggcactgatttcacactgaaaatcagcagggtggaggctgaggatgttggggtttattactgctggcaaggcacacatcttcctcagactttcggcggagggaccaaggtggagatcaaa

SEQ ID NO: 29 - protein, 40 aa, Homo sapiens
PEKDSRVSVTKPFMLPPVAASSLRNDSSSSNRKAKNPPGD

SEQ ID NO: 30 - protein, 21 aa, Homo sapiens
ASSLRNDSSSSNRKAKNPPGD

SEQ ID NO: 31 - protein, 8 aa, Homo sapiens
SSNRKAKN

SEQ ID NO: 32 - protein, 14 aa, Homo sapiens
SSSSNRKAKNPPGD

SEQ ID NO: 33 - protein, 14 aa, Homo sapiens
ASSLRNDSSSSNRK

SEQ ID NO: 34 - protein, 29 aa, Homo sapiens
DSRVSVTKPFMLPPVAASSLRNDSSSSNR

SEQ ID NO: 35 - protein, 7 aa, Homo sapiens
SNRKAKN

SEQ ID NO: 36 - protein, 13 aa, Homo sapiens
ASSLRNDSSSSNR

SEQ ID NO: 37 - protein, 17 aa, synthetic construct
  REGION 1..17: note = VH5 CDR2
QIYPGDGDTHYNGKFQG

SEQ ID NO: 38 - protein, 443 aa, synthetic construct
  REGION 1..443: note = VH0 IgG4 heavy chain
QVQLQQSGAELVRPGSSVKISCKSSGYAFSSYWMNWVKQRPGQGLEWIGQIYPGDGDTHYNGKFKGKATLTADKSSSTAYMQLSRLTSEDSAVYFCSSSNWVGSYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 39 - protein, 219 aa, synthetic construct
  REGION 1..219: note = VK0 Kappa light chain
DVVMTQTPLTLSVTIGQTASISCKSSQSLLESDGKTYLNWLSQRPGQSPKRLIYLVSRLDSGVPDRFTGSGSGTDFTLKISRVEAEDLGVYYCWQGTHLPQTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 40 - protein, 327 aa, synthetic construct
  REGION 1..327: note = heavy chain constant region
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 41 - protein, 107 aa, Homo sapiens [sequence omitted]

SEQ ID NO: 42 - protein, 443 aa, synthetic construct
  REGION 1..443: note = 5H10 VH1 Heavy Chain
QVQLVQSGAELKKPGSSVKISCKSSGYAFSSYWMNWVKQRPGQGLEWIGQIYPGDGDTHYNGKFKGKATLTADKSTSTAYMELSSLTSEDSAVYFCSSSNWVGSYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 43 - protein, 443 aa, synthetic construct
  REGION 1..443: note = 5H10 VH2 Heavy Chain
QVQLVQSGAEVKKPGSSVKISCKSSGYAFSSYWMNWVKQRPGQGLEWIGQIYPGDGDTHYNGKFKGKATLTADKSTSTAYMELSSLRSEDTAVYFCSSSNWVGSYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 44 - protein, 443 aa, synthetic construct
  REGION 1..443: note = 5H10 VH3 Heavy Chain
QVQLVQSGAEVKKPGSSVKVSCKSSGYAFSSYWMNWVRQRPGQGLEWIGQIYPGDGDTHYNGKFKGKATLTADKSTSTAYMELSSLRSEDTAVYFCSSSNWVGSYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 443 aa, synthetic construct
  REGION 1..443: note = 5H10 VH4 Heavy Chain
QVQLVQSGAEVKKPGSSVKVSCKSSGYAFSSYWMNWVRQRPGQGLEWIGQIYPGDGDTHYNGKFKGRVTITADKSTSTAYMELSSLRSEDTAVYFCSSSNWVGSYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 46 - protein, 443 aa, synthetic construct
  REGION 1..443: note = 5H10 VH5 Heavy Chain
QVQLVQSGAEVKKPGSSVKVSCKSSGYAFSSYWMNWVRQRPGQGLEWIGQIYPGDGDTHYNGKFQGRVTITADKSTSTAYMELSSLRSEDTAVYYCSSSNWVGSYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 47 - protein, 219 aa, synthetic construct
  REGION 1..219: note = 5H10 VK1 Light Chain
DVVMTQSPLTLSVTLGQPASISCKSSQSLLESDGKTYLNWLQQRPGQSPRRLIYLVSRLDSGVPDRFTGSGSGTDFTLKISRVEAEDVGVYYCWQGTHLPQTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 219 aa, synthetic construct
  REGION 1..219: note = 5H10 VK2 Light Chain
DVVMTQSPLSLPVTLGQPASISCKSSQSLLESDGKTYLNWLQQRPGQSPRRLIYLVSRLDSGVPDRFTGSGSGTDFTLKISRVEAEDVGVYYCWQGTHLPQTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 219 aa, synthetic construct
  REGION 1..219: note = 5H10 VK3 Light Chain
DVVMTQSPLSLPVTLGQPASISCKSSQSLLESDGKTYLNWLQQRPGQSPRRLIYLVSRLDSGVPDRFSGSGSGTDFTLKISRVEAEDVGVYYCWQGTHLPQTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 219 aa, synthetic construct
  REGION 1..219: note = 5H10 VK4 Light Chain
DVVMTQSPLSLPVTLGQPASISCKSSQSLLESDGKTYLNWFQQRPGQSPRRLIYLVSRLDSGVPDRFSGSGSGTDFTLKISRVEAEDVGVYYCWQGTHLPQTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2026/0184776 A1
(43) Pub. Date: Jul. 2, 2026

(54) ANTI-STEM CELL FACTOR ANTIBODIES AND METHODS OF USE THEREOF

(71) Applicant: Insmed Incorporated, Bridgewater, NJ (US)

(72) Inventor: Martin PHILLIPS, Bryn Mawr, PA (US)

(73) Assignee: Insmed Incorporated, Bridgewater, NJ (US)

(21) Appl. No.: 19/367,478

(22) Filed: Oct. 23, 2025

Related U.S. Application Data
(60) Provisional application No. 63/711,558, filed on Oct. 24, 2024.

(51) Int. Cl. C07K 16/24; A61K 39/00; A61P 17/00

(52) U.S. Cl. CPC C07K 16/24; A61P 17/00; A61K 2039/505; A61K 2039/545; C07K 2317/24; C07K 2317/56; C07K 2317/92

(57) ABSTRACT

The disclosure relates to methods for treating atopic dermatitis with an antibody that binds to stem cell factor 248 (SCF248).

### CROSS-REFERENCE TO RELATED APPLICATIONS

This application claims priority to U.S. Application No. 63/711,558 filed on Oct. 24, 2024, the contents of which is incorporated by reference herein in its entirety for all purposes.

### REFERENCE TO AN ELECTRONIC SEQUENCE LISTING

The contents of the electronic sequence listing (OPSL_006_01US_SeqList_ST26.xml; Size: 65,524 bytes; and Date of Creation: Oct. 22, 2025) are herein incorporated by reference in its entirety.

### FIELD

The present invention relates to antibodies and antigen-binding fragments thereof that bind to Stem Cell Factor (SCF) and particular portions thereof, and to methods of using such antibodies and antigen-binding fragments.

### BACKGROUND

Inflammatory diseases are a major cause of morbidity and mortality worldwide. Some types of chronic inflammation can lead to fibrosis, which is the formation or development of excess fibrous connective tissue in an organ or tissue as a reparative or reactive process, as opposed to formation of fibrous tissue as a normal constituent of an organ or tissue. Chronic inflammation as well as fibrosis can affect nearly all tissues and organ systems, and fibrotic tissue remodeling can influence cancer metastasis and accelerate chronic graft rejection in transplant recipients.

Stem cell factor (SCF) and its receptor c-Kit are important factors of the perpetuation of chronic inflammation and in fibrotic diseases (El-Koraie, et al., Kidney Int. 60: 167(2001); Powell, et al., Am. J. Physiol. 289: G2 (2005); El Kossi, et al., Am. J. Kidney Dis. 41: 785(2003); Powell, et al., Am. J. Physiol. 277: C183 (1999) Ding et al J Pathol. 2013 June; 230(2):205-14., Berlin et al Lab Invest. 2006 June; 86(6):557-65, Rasky et al Am J Physiol Lung Cell Mol Physiol. 2020 Jan. 1; 318(1):L200-L211). c-Kit is a type III receptor-tyrosine kinase that is present in many cell types (Orr-Urtreger et al., Development 109: 911 (1990). Immune cells such as mast cells, eosinophils, and innate lymphoid cells 2 and 3 (ILC2 and ILC3) are all c-Kit+ cells that may drive the chronic inflammatory process, depending on the disease and organ involved. Upon initiation of an inflammatory response, various mediators, including SCF, activate c-Kit+ immune cells, which in turn produce cytokines that cause fibroblasts to become activated myofibroblasts. Myofibroblasts secrete extracellular matrix proteins, collagen, and fibronectin, resulting in fibrosis of tissue. Activated myofibroblasts, activated epithelia, endothelia, macrophages, eosinophils, mast cells, monocytes, and other cells also express SCF on the cell surface, which activates more c-Kit+ immune cells, resulting in more cytokine release and perpetuating the inflammation.

There is a need in the art for more efficient and more specific treatments for inflammatory diseases. The present disclosure addresses this and other needs.

### SUMMARY OF THE DISCLOSURE

Provided herein is a method for treating atopic dermatitis in a human patient with atopic dermatitis, comprising administering to the human patient a therapeutically effective dose of a full length anti-stem cell factor 248 (anti-SCF248) antibody, wherein the full length anti-SCF248 antibody comprises two heavy chains each having the amino acid sequence of SEQ ID NO: 42 and two light chains each having the amino acid sequence of SEQ ID NO: 49; wherein the therapeutically effective dose is from 10 mg to 600 mg regardless of the human patient's body weight, and wherein treating is reducing or eliminating itching or a skin rash in the human patient.

Provided herein is a method for treating atopic dermatitis in an human patient with atopic dermatitis, comprising administering to the patient a therapeutically effective dosage regimen of a full length anti-stem cell factor 248 (anti-SCF248) antibody, wherein the full length anti-SCF248 antibody comprises two heavy chains each having the amino acid sequence of SEQ ID NO:42 and two light chains each having the amino acid sequence of SEQ ID NO:49, wherein the method reduces or eliminates itching or a skin rash, the dosage regimen comprising: a) administering to the human patient a first dose of 10 mg to 600 mg of the full length anti-SCF248 antibody regardless of the patient's body weight; and b) administering to the human patient a second dose of 10 mg to 600 mg of the full length anti-SCF248 antibody regardless of the patient's body weight; and wherein the method results in the patient achieving at least a 50% reduction from baseline Eczema Activity Severity Index (EASI) scale score sixteen weeks after receiving the first dose.

Provided herein is a method for treating atopic dermatitis in an human patient with atopic dermatitis, comprising administering to the patient a therapeutically effective dosage regimen of a full length anti-stem cell factor 248 (anti-SCF248) antibody, wherein the full length anti-SCF248 antibody comprises two heavy chains each having the amino acid sequence of SEQ ID NO:42 and two light chains each having the amino acid sequence of SEQ ID NO:49, wherein the method reduces or eliminates itching or a skin rash, the dosage regimen comprising: a) administering to the human patient a first dose of 10 mg to 600 mg of the full length anti-SCF248 antibody regardless of the patient's body weight; and b) administering to the human patient a second dose of 10 mg to 600 mg of the full length anti-SCF248 antibody regardless of the patient's body weight; wherein the method results in the patient achieving at least a one point reduction from baseline validated Investigator's Global Assessment (vIGA) score for atopic dermatitis sixteen weeks after receiving the first dose.

In embodiments, provided herein is a method for eliminating itching or a skin rash in an human patient with atopic dermatitis, comprising administering to the human patient a therapeutically effective dose of a full length anti-stem cell factor 248 (anti-SCF248) antibody, wherein the full length anti-SCF248 antibody comprises two heavy chains each having the amino acid sequence of SEQ ID NO: 42 and two light chains each having the amino acid sequence of SEQ ID NO: 49; and wherein the therapeutically effective dose is from 10 mg to 600 mg regardless of the human patient's body weight.

In one aspect, the present disclosure provides antibodies and fragments thereof that specifically bind to stem cell factor (SCF). In some embodiments, the antibodies and fragments thereof specifically bind to the SCF isoform SCF248. In some embodiments, the antibodies and fragments thereof comprise heavy chain complementarity determining regions (CDRs), wherein heavy chain CDR1 CDR2, and CDR3 comprise SEQ ID NOs: 1, 2, and 3, respectively. In some embodiments, the antibodies and fragments thereof comprise light chain CDRs, wherein the light chain CDR1 CDR2, and CDR3 comprise SEQ ID NOs: 4, 5, and 6, respectively. In some embodiments, the antibodies and fragments thereof comprise heavy chain CDR1, CDR2, and CDR3 comprising SEQ ID NOs: 1, 37, and 3, respectively. In some embodiments, the antibodies and fragments thereof comprise a heavy chain variable region comprising at least 80%, at least 85%, at least 90%, at least 95%, or at least 99% identity to a sequence selected from the group consisting of SEQ ID NOs: 7, 8, 9, 10, 11, and 12. In some embodiments, the antibodies and fragments thereof comprise a light chain variable region comprising at least 80%, at least 85%, at least 90%, at least 95%, or at least 99% identity to a sequence selected from the group consisting of SEQ ID NOs: 13, 14, 15, 16, and 17. In some embodiments, the antibodies and fragments thereof comprise a heavy chain variable region comprising an amino acid sequence selected from the group consisting of SEQ ID NOs: 7, 8, 9, 10, 11, and 12, and a light chain variable region comprising an amino acid sequence selected from the group consisting of SEQ ID NOs: 13, 14, 15, 16, and 17.

In some embodiments, the antibody or fragment thereof comprises a heavy chain variable region amino acid sequence according to SEQ ID NO: 7 and a light chain variable region amino acid sequence according to SEQ ID NO: 16. In some embodiments, the antibody or fragment thereof of claim 1, wherein the antibody or fragment thereof comprises a heavy chain variable region amino acid sequence according to SEQ ID NO: 8 and a light chain variable region amino acid sequence according to SEQ ID NO: 16. In some embodiments, the antibody or fragment thereof comprises a heavy chain variable region amino acid sequence according to SEQ ID NO: 9 and a light chain variable region amino acid sequence according to SEQ ID NO: 16. In some embodiments, the antibody or fragment thereof comprises a heavy chain variable region amino acid sequence according to SEQ ID NO: 10 and a light chain variable region amino acid sequence according to SEQ ID NO: 16. In some embodiments, the antibody or fragment thereof comprises a heavy chain variable region amino acid sequence according to SEQ ID NO: 11 and a light chain variable region amino acid sequence according to SEQ ID NO: 16. In some embodiments, the antibody or fragment thereof comprises a heavy chain variable region amino acid sequence according to SEQ ID NO: 12 and a light chain variable region amino acid sequence according to SEQ ID NO: 16.

In some embodiments, the antibody or fragment thereof is humanized. In some embodiments, the antibody is a monoclonal antibody. In some embodiments, the antibody comprises a human IgG1 domain or a human IgG4 domain. In some embodiments, the antibody is an antigen binding fragment, wherein the fragment is selected from a Fab, F(ab′)2, Fab′, scFv, and single domain antibody (sdAb).

In some embodiments, the antibody or fragment thereof blocks the interaction between SCF (e.g. SCF248) and c-Kit. In some embodiments, the antibody specifically binds to SCF248. In some embodiments, the antibody does not bind to SCF220. In some embodiments, the antibody prevents the interaction of SCF248 and c-kit by causing the internalization of SCF, making it unavailable on the cell surface.

In one aspect, the present disclosure provides pharmaceutical compositions comprising the antibody or fragment thereof provided herein. In some embodiments, the pharmaceutical composition comprises a pharmaceutically acceptable carrier, diluent or excipient.

In some embodiments, the present disclosure provides isolated nucleic acid molecules encoding the antibody or fragment thereof provided herein. In some embodiments, the present disclosure provides an expression vector comprising the nucleic acid encoding the antibody or fragment thereof. In some embodiments, the present disclosure provides a recombinant host cell comprising the expression vector.

In one aspect, the present disclosure provides methods for making an antibody that specifically binds to stem cell factor isoform 248 (SCF248), the method comprising immunizing a host animal with a peptide comprising SEQ ID NO: 30 (ASSLRNDSSSSNRKAKNPPGD) or a fragment thereof, and obtaining an antibody from the immunized host animal. In some embodiments, the host animal is not a human. In some embodiments, the fragment of SEQ ID NO: 30 comprises at least 5, at least 6, at least 7, at least 8, at least 9, at least 10, at least 11, at least 12, at least 13, at least 14, at least 15, at least 16, at least 17, at least 18, at least 19, or 20 contiguous amino acids of SEQ ID NO: 30. In some embodiments, the fragment of SEQ ID NO: 30 comprises 5, 6, 7, 8, 9, 10, 11, 12, 13, 14, 15, 16, 17, 18, 19, or 20 contiguous amino acids of SEQ ID NO: 30. In some embodiments, the N-terminal amino acid of the fragment of SEQ ID NO: 30 is the alanine at position 1 at the N-terminus of SEQ ID NO: 30. In some embodiments, the method comprises immunizing the host animal with a peptide consisting of SEQ ID NO: 30. In some embodiments, the antibody from the immunized host animal is obtained from an immune cell isolated from the host animal. In some embodiments, the method further comprises generating a hybridoma using the immune cell. Thus, in some embodiments, the present disclosure provides hybridomas that produce monoclonal antibodies described herein.

In one aspect, the present disclosure provides an antibody or fragment thereof that specifically binds to SCF248, wherein the antibody or fragment thereof binds to an epitope comprising at least 8, at least 9, at least 10, at least 11, at least 12, or at least 13 contiguous amino acids of SEQ ID NO: 33, wherein the antibody inhibits the interaction of SCF248 with c-Kit. In further embodiments, the epitope comprises SEQ ID NO: 33 or SEQ ID NO: 36. In yet further embodiments, the epitope consists of SEQ ID NO: 33 or SEQ ID NO: 36.

In one aspect, the present disclosure provides compositions and methods for inhibiting the interaction between SCF and c-Kit. C-kit is expressed on immune cells, hematopoietic stem cells, and some structural cells. C-kit's ligand SCF248 can be upregulated on myofibroblasts, activated epithelia, endothelia, macrophages, eosinophils, mast cells, monocytes, and others. In some embodiments, the compositions and methods specifically inhibit the interaction between SCF248 and c-Kit. For example, in some embodiments, the compositions and methods specifically inhibit the interaction between SCF248 on myofibroblasts and c-Kit on immune cells. As another example, in some embodiments, the compositions and methods provided herein specifically inhibit the interaction between SCF248 on myofibroblasts, activated epithelia, endothelia, macrophages, eosinophils, mast cells, and/or monocytes; with c-Kit on immune cells and/or structural cells. In some embodiments, the methods comprise contacting SCF248 on myofibroblasts with an antibody or fragment thereof provided herein. In some embodiments, the antibody or fragment thereof provided herein blocks binding of SCF248 to c-Kit. In some embodiments, the blocking is via steric hindrance. In some embodiments, the antibody or fragment thereof provided herein internalizes SCF248.

In some embodiments, the present disclosure provides methods for inhibiting inflammation in a subject in need thereof, the method comprising administering to the subject an antibody or fragment thereof provided herein. In some embodiments, the present disclosure provides methods for inhibiting an inflammatory disease in a subject in need thereof, the method comprising administering to the subject an antibody or fragment thereof provided herein. In further embodiments, the inflammatory disease is a chronic inflammatory disease. In some embodiments, the present disclosure provides methods for treating inflammation and/or a chronic inflammatory disease in a subject in need thereof, the method comprising administering to the subject an antibody or fragment thereof provided herein.

In some embodiments, the present disclosure provides methods for inhibiting fibrosis in a subject in need thereof, the method comprising administering to the subject an antibody or fragment thereof provided herein. In some embodiments, the present disclosure provides methods for treating a fibrotic disease in a subject in need thereof, the method comprising administering to the subject an antibody or fragment thereof provided herein. In embodiments, the method further comprises administering one or more additional therapy and/or therapeutic agent.

In some embodiments, the inflammatory disease or fibrotic disease is selected from the group consisting of urticaria, atopic dermatitis, bullous pemphigoid, scleroderma, systemic sclerosis, non-alcoholic steatohepatitis (NASH), primary sclerosing cholangitis, liver cirrhosis, pulmonary fibrosis (e.g., idiopathic pulmonary fibrosis (IPF), scleroderma lung fibrosis), chronic obstructive pulmonary disease (COPD), acute respiratory distress syndrome (ARDS), cystic fibrosis, peribronchial fibrosis, hypersensitivity pneumonitis, asthma, bleomycin lung, endomyocardial fibrosis, fibromyalgia, eosinophilic esophagitis, radiation fibrosis, rheumatoid arthritis, and inflammatory bowel disease.

### BRIEF DESCRIPTION OF THE FIGURES

FIG. 1 provides a schematic overview of the tissue injury/inflammatory disease process.

FIG. 2 shows an exemplary mechanism of an anti-SCF248 antibody of the instant disclosure, 5H10.

FIG. 3 shows the isoforms of SCF, SCF220 and SCF248; and the monomeric cleaved extracellular domain, SCF165. SCF165 is released upon cleavage of SCF248 at its cleavage site within the Exon 6 region.

FIG. 4A is a set of histograms showing the binding of murine 5H10 antibody to control cells that do not expression SCF (left panel), cells that express SCF220 but not SCF248 (middle panel), and cells that express SCF248 but not SCF220 (right panel).

FIG. 4B shows the binding of murine 5H10 antibody to the 165 amino acid cleaved SCF extracellular domain (ECD) versus the complete 194 amino acid SCF ECD.

FIG. 5 shows the mean fluorescence intensity (MFI) as measured by flow cytometry after contacting cultured human IPF myofibroblasts with PHRODO™ red-labeled 2G8, 5H10, or control IgG antibodies.

FIG. 6 shows the activation of the P13K/AKT pathway and the MEK/ERK pathway of c-kit signaling after contacting eosinophils with an SCF248-expressing cells in the presence of 5H10 antibody or IgG control. 5H10 antibody significantly reduced activation of both pathways.

FIGS. 7A-7B shows binding by flow cytometry of murine 2G8 and murine 5H10 antibodies to early (FIG. 7A) and late (FIG. 7B) passage S1/S14 hSCF248 (ATCC® CRL2454™) cells.

FIGS. 8A-8B shows binding of murine 2G8 and murine 5H10 to S1/S14 hSCF220 (ATCC® CRL2453™) cells (FIG. 8A) and S1/S14 hSCF248 cells (FIG. 8B) at early passage and to hygromycin B treated cells.

FIGS. 9A-9B shows binding of 2G8 humanized variants at different antibody concentrations by flow cytometry to S1/S14 hSCF248 cells (FIG. 9A) and S1/S14 hSCF220 cells (FIG. 9B).

FIGS. 10A-10B shows binding of 5H10 humanized variants at different antibody concentrations by flow cytometry to S1/S14 hSCF248 cells (FIG. 10A) and S1/S14 hSCF220 cells (FIG. 10B).

FIGS. 11A-11D shows binding of 2G8 humanized variants (FIG. 11A) and 5H10 humanized variants (FIG. 11B, 11C, 11D) at different antibody concentrations by flow cytometry to S1/S14 hSCF248 cells. In FIG. 11C, the indicated VH is paired with VK3. In FIG. 11D, the 5H10 antibody shown is VH1/VK3.

FIGS. 12A-12D show the change in mRNA level of the CCL11 (FIG. 12A), Collagen 1A1 (FIG. 12B), fibronectin (FIG. 12C), or collagen 3 (FIG. 12D) after preincubation of human IPF myofibroblasts (Mfb) with a positive control (irrelevant antibody) or the antibody indicated under each bar in the figure. The murine parent antibody is indicated as “5H10” in the figure; humanized 5H10 antibodies VH1/VK3, VH2/VK3, VH3/VK3, VH4/VK3, and VH5/VK3 were also tested as shown. Antibody concentrations tested were 1 μg/mL or 10 μg/mL.

FIG. 13 shows the internalization of PHRODO™ red labeled murine 5H10 antibody, VH0/VK0 chimeric antibody, and humanized variants VH1/VK3 and VH2/VK3. Arrows point to exemplary cells exhibiting internalized antibody.

FIG. 14 shows the effect of 5H10 humanized variants on PBMC viability.

FIGS. 15A-15H shows the CD4+ T cell responses induced by the indicated 5H10 humanized variants in an EPISCREEN™ time course T cell proliferation assay. Exenatide and KLH, shown in FIGS. 15G and 15H, respectively, are positive controls.

FIG. 16 shows variance analysis (ANOVA) of an EPISCREEN™ time course T cell proliferation assay.

FIG. 17 shows lung histology of bleomycin control animals (left panel) and animals treated with bleomycin and 5H10 (20 mg/kg).

FIG. 18 shows decreases in lung hydroxyproline in animals treated with bleomycin and control IgG vs. animals treated with bleomycin and 5H10 (20 mg/kg; referred to in the figure as anti-SCF248).

FIG. 19 shows the body weight as a % of the weight measured at day 0, over time in naïve mice, mice treated with bleomycin to induce lung fibrosis, and control Ig antibody (Bleo+cIg), and mice treated with bleomycin and murine 5H10 antibody (referred to in the figure as anti-SCF248). Antibody was administered at the indicated timepoints.

FIG. 20 shows decreases in mRNA for inflammatory cytokine and markers of myofibroblast activation (TGFβ, CCL2, Co11a1, Fibronectin (fn), smooth muscle actin (acta2), and stem cell factor (kitlg)) in animals treated with bleomycin and control IgG vs. animals treated with bleomycin and 5H10 (20 mg/kg; referred to in the figure as anti-SCF248).

FIG. 21 shows decreases in lung mast cells, eosinophils and ILC2 lymphocytes in animals treated with bleomycin and control IgG vs. animals treated with bleomycin and 5H10 (20 mg/kg; referred to in the figure as anti-SCF248).

FIG. 22 shows that pulmonary function testing as measured by forced expiratory volume (left panel), forced expiratory flow (middle panel), and change in pulmonary pressure (right panel) were significantly improved in animals treated with bleomycin and 5H10 (20 mg/kg; referred to in the figure as anti-SCF248) compared to animals treated with bleomycin and control IgG.

FIG. 23 shows a schematic of a study design in an in vivo chronic allergic asthma model used to test the humanized antibodies.

FIGS. 24A-24E provide results of treatment with humanized 5H10 antibodies in the in vivo model of chronic allergic asthma. FIG. 24A shows that airway resistance as measured was significantly reduced in animals treated with VH1/VK3 compared to PBS control. IL-13 mRNA (FIG. 24B), Collagen 1 mRNA (FIG. 24C), and Collagen 3 mRNA (FIG. 24D) in lung tissues were also reduced in animals treated with VH1/VK3 compared to the chronic asthma (PBS) control. FIG. 24E shows that SCF248 mRNA expression was also reduced in animals treated with VH1/VK3 5H10 antibody compared to PBS control.

FIGS. 25A-25C show that antibody VH1/VK3 reduced mRNA levels of mucus protein Gob5 (FIG. 25A), IL-13 (FIG. 25B), and IL-5 (FIG. 25C), at concentrations of 1 mg/kg and 5 mg/kg in vivo.

FIGS. 26A-26B show single ascending dose (SAD) mouse pharmacokinetics (PK) for the murine 5H10 antibody administered at 0.3, 3, and 30 mg/kg. FIG. 26A shows the PK experimental data alone and FIG. 26B shows the PK data overlaid with model-based predictions at each dose using the fit values CL, V1, V2, and Q.

FIG. 27 shows a timeline of the dosing schedule for a study of 5H10 murine antibody evaluated in a bleomycin-induced lung fibrosis model.

FIG. 28 shows a model-based prediction of PK in a bleomycin-induced fibrosis model. Doses of 0.3, 3, and 20 mg/kg were administered at Days 8 and 12 of the study.

FIGS. 29A-29B shows the arithmetic mean and standard deviation for serum concentrations of OpSCF following single subcutaneous or intravenous doses of OpSCF via the linear (FIG. 29A) and semi-logarithmic scale (FIG. 29B) in a SAD study. (IV=intravenous; N=number of subjects; PK=pharmacokinetic; SC=subcutaneous; SD=standard deviation. For IV treatments, the postdose times presented are relative to the end of infusion. Actual times relative to the start of the infusion were used in PK analysis.)

FIGS. 30A-30F shows pharmacokinetics after a single dose of OpSCF, including AUCₜ (FIG. 30A), DAUCₜ (FIG. 30B), AUC_{inf} (FIG. 30C), DAUC_{inf} (FIG. 30C), Cₘₐₓ (FIG. 30E), and DCₘₐₓ (FIG. 30F). (CI=confidence interval.)

FIGS. 31A-31E show the arithmetic mean and standard deviation (SD) of serum concentrations at Day 1 (linear FIG. 31A, logarithmic FIG. 31B), Day 22 (linear FIG. 31C, logarithmic FIG. 31D), and overall (linear, FIG. 31E) in a multiple ascending dose (MAD) study. (IV=intravenous; N=number of subjects; PK=pharmacokinetic; SC=subcutaneous; SD=standard deviation. Dose levels are delivered via IV route on Day 1 and SC route on Days 8, 5 and 22. For IV treatments, the postdose times are relative to the end of the infusion. Actual times relative to the start of the infusion were used in PK analysis.)

FIG. 32 shows a study timeline of a Phase 2a randomized controlled trial evaluating the safety and efficacy of a monoclonal antibody OpSCF in adults with moderate to severe atopic dermatitis (AD).

FIG. 33 shows the distribution of subjects randomized to the OpSCF or placebo arm of a Phase 2a randomized clinical trial evaluating the safety and efficacy of OpSCF. (OLE, open label extension.)

FIGS. 34A-34C shows a comparison of vIGA-AD scores after 16 weeks among subjects administered OpSCF versus placebo. FIG. 34A shows the mean vIGA-AD scores over 16 weeks in the OpSCF arm versus the placebo arm. FIG. 34B shows the number of subjects achieving vIGA 0 or 1 and a ≥2 point reduction after 16 weeks in the OpSCF arm versus the placebo arm. FIG. 34C shows the percent of subjects achieving IGA scores of 0 or 1 after 16 weeks in the OpSCF group compared to the placebo and a dupilumab group. A comparison was also conducted between placebo adjusted dupilumab and OpSCF measures.

FIGS. 35A-35C shows a comparison of EASI scores after 16 weeks among subjects administered OpSCF versus placebo. FIG. 35A shows the percent of subjects with EASI scores of 90 among the placebo arm, the OpSCF arm, and the placebo-adjusted OpSCF response. FIG. 35B shows a comparison in the percentage of subjects with EASI 90 over 16 weeks in the OpSCF study arm versus placebo study arm. FIG. 35C shows a comparison of subjects that achieved EASI 90 after 16 weeks in the placebo, and OpSCF groups, compared to historical dupilumab results (labeled “SOLO1” and “SOLO2”; Simpson et al., N Engl J Med, 2016; 375:2335-48). A comparison was also conducted between placebo adjusted dupilumab and OpSCF measures.

FIGS. 36A-36B shows a comparison of the percent change (FIG. 36A) and absolute change (FIG. 36B) in body surface area (BSA) affected by AD over 16 weeks between the OpSCF and placebo study arms.

FIG. 37 shows a comparison of the vIGA-AD scores of the placebo versus OpSCF study arms between weeks 16 and 44 in the open label extension phase of the Phase 2a randomized controlled trial.

FIG. 38 shows a comparison of the mean EASI scores of the placebo versus OpSCF study arms between weeks 16 and 44 in the open label extension phase of the Phase 2a randomized controlled trial.

### DETAILED DESCRIPTION

Stem Cell Factor (SCF) is a key mediator of acute and chronic inflammation, fibrotic diseases, and tissue remodeling diseases. The interaction of SCF with c-Kit on immune cells initiates and perpetuates inflammation and fibrosis. The present disclosure provides compositions and methods for inhibiting the interaction of SCF with c-Kit. In one aspect, the present disclosure provides compositions and methods for preventing the inflammatory form of SCF, SCF248, from interacting with c-Kit and thus reduces and/or prevents activation of immune cells. Thus, the present disclosure provides methods for treating chronic inflammation and fibrotic and tissue remodeling diseases. In one aspect, the present disclosure provides compositions and methods for reducing the accumulation (e.g., proliferation and/or retention) of immune cells in an organ or tissue. For example, the disclosure provides compositions and methods that prevent SCF248 from interacting with c-Kit and thus reduces and/or prevents accumulation of immune cells in organs or tissues. In some embodiments, the disclosure provides compositions and methods for reducing and/or preventing the activation and/or accumulation in organs or tissues of mast cells, eosinophils, type 2 innate lymphoid (ILC2) cells, and type 3 innate lymphoid (ILC3) cells.

In particular, the present disclosure provides antibodies and fragments thereof that specifically bind to SCF and block or inhibit its interaction with c-Kit. In some embodiments, the antibodies and fragments provided herein bind to SCF and inhibit the activity of c-Kit and c-Kit+ cells. The disclosure also provides methods for generating antibodies and fragments thereof that specifically bind to SCF, as well as diagnostic and therapeutic methods of use thereof. In one aspect, the antibodies and fragments thereof provided herein specifically bind to the SCF isoform that drives inflammation, SCF248. Thus, the present disclosure provides specific, effective compositions and methods for inhibiting inflammation and fibrosis and treating chronic inflammatory diseases and fibrotic diseases.

### Definitions

As used herein, the term “antibody” refers to a binding protein having at least one antigen binding domain. The antibodies and fragments thereof of the present invention may be whole antibodies or any fragment thereof. Thus, the antibodies and fragments of the invention include monoclonal antibodies or fragments thereof and antibody variants or fragments thereof, as well as immunoconjugates. In some embodiments, the antibodies and fragments of the invention include two heavy chains and two light chains. Antigen binding fragments include Fab fragments, Fab′ fragments, F(ab′)2 fragments, bispecific Fab dimers (Fab2), trispecific Fab trimers (Fab3), Fv, single chain Fv proteins (“scFv”), bis-scFv, (scFv)2, minibodies, diabodies, triabodies, tetrabodies, disulfide stabilized Fv proteins (“dsFv”), single-domain antibodies (sdAb, nanobody), heavy-chain only antibodies (e.g., camelid VHH, camelid nanobody, shark Ig NAR), and portions of full length antibodies responsible for antigen binding. An isolated antibody or antigen binding fragment thereof is one which has been identified and separated and/or recovered from a component of its natural environment. Described herein are methods of using a humanized monoclonal antibody comprising two heavy chains that each have an amino acid sequence of SEQ ID NO: 42 and two light chains that each have an amino acid sequence of SEQ ID NO: 49. The aforementioned monoclonal antibody is referred to interchangeably as “humanized 5H10” and “OpSCF” throughout the present disclosure.

In some embodiments, the antibodies and antigen binding fragments thereof are isolated antibodies and fragments thereof, Thus, the present invention provides isolated antibodies and antigen binding fragments thereof, and nucleic acids encoding such antibodies and fragments, as well as compositions comprising such isolated antibodies, fragments, and nucleic acids. The term “isolated” refers to a compound of interest (e.g., an antibody or nucleic acid) that has been separated from its natural environment. The present invention further provides pharmaceutical compositions comprising the isolated antibodies or fragments thereof, or nucleic acids encoding such antibodies or fragments, and further comprising one or more pharmaceutically acceptable carrier. Pharmaceutically acceptable carriers include, for example, excipients, diluents, encapsulating materials, fillers, buffers, or other agents.

As used herein, the term “derived” when used to refer to a molecule or polypeptide relative to a reference antibody or other binding protein, means a molecule or polypeptide that is specific for, and capable of binding to, the same epitope as the reference antibody or other binding protein.

As used herein, the phrase “specific for” may mean that the antibody does not bind to the target due to only non-specific interactions, and this property can be determined by comparison to an isotype control or similar. Specific binding does not necessarily require, although it may include, exclusive binding to a single target. In embodiments, the antibodies provided herein specifically bind to SCF248, and do not bind SCF220.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

A “variant” of a polypeptide (e.g., an antigen binding protein, or an antibody) comprises an amino acid sequence wherein one or more amino acid residues are inserted into, deleted from and/or substituted into the amino acid sequence relative to another polypeptide sequence. Variants include antibodies and fragments thereof that have a recited percent identity to an antibody or fragment provided herein or to an antibody or fragment having a recited DNA or amino acid sequence.

The term “identity” refers to a relationship between the sequences of two or more polypeptide molecules or two or more nucleic acid molecules, as determined by aligning and comparing the sequences. “Percent identity,” “percent homology,” “sequence identity,” or “sequence homology” and the like mean the percent of identical residues between the amino acids or nucleotides in the compared molecules and is calculated based on the size of the smallest of the molecules being compared. For these calculations, gaps in alignments (if any) are preferably addressed by a particular mathematical model or computer program (i.e., an “algorithm”). Methods that can be used to calculate the identity of the aligned nucleic acids or polypeptides include those described in Computational Molecular Biology, (Lesk, A. M., ed.), 1988, New York: Oxford University Press; Biocomputing Informatics and Genome Projects, (Smith, D. W., ed.), 1993, New York: Academic Press; Computer Analysis of Sequence Data, Part I, (Griffin, A. M., and Griffin, H. G., eds.), 1994, New Jersey: Humana Press; von Heinje, G., 1987, Sequence Analysis in Molecular Biology, New York: Academic Press; Sequence Analysis Primer, (Gribskov, M. and Devereux, J., eds.), 1991, New York: M. Stockton Press; and Carillo et al., 1988, SIAM J. Applied Math. 48:1073. In calculating percent identity, the sequences being compared are typically aligned in a way that gives the largest match between the sequences.

The term “light chain” includes a full-length light chain and fragments thereof having sufficient variable region sequence to confer binding specificity. A full-length light chain includes a variable region domain and a constant region domain. The variable region domain of the light chain is at the amino-terminus of the polypeptide. Light chains include kappa chains and lambda chains.

The term “heavy chain” includes a full-length heavy chain and fragments thereof having sufficient variable region sequence to confer binding specificity. A full-length heavy chain includes a variable region domain, three constant region domains, C_{H}1, C_{H}2, and C_{H}3. The variable heavy domain is at the amino-terminus of the polypeptide, and the C_{H} domains are at the carboxyl-terminus, with the C_{H}3 being closest to the carboxy-terminus of the polypeptide. Heavy chains can be of any isotype, including IgG (including IgG1, IgG2, IgG3 and IgG4 subtypes), IgA (including IgAQ1 and IgA2 subtypes), IgM and IgE. The term “isotype” refers to the antibody class encoded by the heavy chain constant region genes. In some embodiments, the antibodies provided herein have an IgG4 heavy chain, or an IgG4 heavy chain comprising certain amino acid mutations. For example, in some embodiments, the IgG4 comprises a mutation at position 228 (EU numbering scheme, Kabat et al. Sequence of proteins of immunologic interest, 5th ed Bethesda, MD, NIH 1991) to inhibit Fab arm exchange. For example, in some embodiments, the IgG4 heavy chain is an IgG4 S228P heavy chain. In some embodiments, the heavy chain comprises one or more amino acid mutations that reduce binding to Fc receptors, and thereby reduce or eliminate effector function of the antibody. For example, the heavy chain may comprise mutations at one or more of positions 233, 234, 235, 236, 237, 265, 309, 331, and 409 (EU numbering).

The term “variable region” or “variable domain” refers to a portion of the light and/or heavy chains of an antibody, typically including approximately the amino-terminal 120 to 130 amino acids in the heavy chain and about 100 to 110 amino terminal amino acids in the light chain. In certain embodiments, variable regions of different antibodies differ extensively in amino acid sequence even among antibodies of the same species. The variable region of an antibody typically determines specificity of a particular antibody for its target. The term “target,” as used herein, refers to a molecule or a portion of a molecule capable of being bound by an antigen binding protein. In certain embodiments, a target can have one or more epitopes. In certain embodiments, a target is an antigen. The use of “antigen” in the phrase “antigen binding protein” simply denotes that the protein sequence that comprises the antigen can be bound by an antibody. In this context, it does not require that the protein be foreign or that it be capable of inducing an immune response.

The term “epitope” includes any determinant capable being bound by an antigen binding protein, such as an antibody or to a T-cell receptor. An epitope is a region of an antigen that is bound by an antigen binding protein that targets that antigen, and when the antigen is a protein, includes specific amino acids that directly contact the antigen binding protein. Most often, epitopes reside on proteins, but in some instances can reside on other kinds of molecules, such as nucleic acids. Epitope determinants can include chemically active surface groupings of molecules such as amino acids, sugar side chains, phosphoryl or sulfonyl groups, and can have specific three dimensional structural characteristics, and/or specific charge characteristics. Generally, antibodies specific for a particular target antigen will preferentially recognize an epitope on the target antigen in a complex mixture of proteins and/or macromolecules. Antibody epitopes may be linear or conformational. In embodiments, the epitope provided herein is a linear epitope.

The use of the singular includes the plural unless specifically stated otherwise. The word “a” or “an” means “at least one” unless specifically stated otherwise. The use of “or” means “and/or” unless stated otherwise. The meaning of the phrase “at least one” is equivalent to the meaning of the phrase “one or more.” Furthermore, the use of the term “including,” as well as other forms, such as “includes” and “included,” is not limiting. Also, terms such as “element” or “component” encompass both elements or components comprising one unit and elements or components comprising more than one unit unless specifically stated otherwise. As used herein, the term “about” refers to an amount more or less than the stated parameter value, for example plus or minus five or ten percent of the object that “about” modifies, or as one of skill in the art would recognize from the context (e.g., approximately 50% of the interval between values). The term “about” also includes the value referenced.

### Stem Cell Factor

In humans, there are at least two forms of SCF, which have different structures and activities. SCF220 functions in several homeostatic functions, including hematopoiesis and spermatogenesis and is found in bone marrow, testis, and other tissues and organs. SCF220 is slowly cleavable and sometimes called “membrane SCF.” In contrast, SCF248 is rapidly cleavable and comprises a cleavage site in exon 6, located between the N-terminal c-kit binding domain and the transmembrane domain. SCF248 may be referred to as “soluble SCF”. Exon 6 is excluded from SCF220 via alternative splicing, and SCF220 thus lacks this cleavage site. A monomeric, extracellular domain (SCF165) is the cleavage product and serves as a biomarker in plasma for chronic inflammatory diseases. Plasma may also contain detectable levels of SCF extracellular domain that comes from SCF220, but the majority of detectable extracellular domain is expected to be SCF165. SCF248 is the isoform found on myofibroblasts, activated epithelial cells, and other cells, which activates immune cells during inflammation and contributes to perpetuation of fibrosis. More specifically, SCF248 binds to c-Kit on immune cells, initiating production of cytokines that activate fibroblasts to become myofibroblasts, which secrete extracellular matrix proteins, collagen, and fibronectin. The activated myofibroblasts as well as activated epithelia, endothelia, macrophages, eosinophils, mast cells, monocytes, and other cells also express SCF on the cell surface, activating more c-Kit+immune cells, resulting in further cytokine release and immune activation and fibrotic responses.

The antibodies and antigen-binding fragments thereof disclosed herein are specific for SCF. In some embodiments, the antibodies and fragments thereof are specific for human SCF. In some embodiments, the antibodies and fragments thereof are specific for SCF248. In some embodiments, the antibodies bind SCF248 and do not bind other isoforms of SCF. In some embodiments, the antibodies bind SCF248 and do not bind to SCF220. In some embodiments, the present disclosure provides methods for making an antibody or fragment thereof that is specific for SCF248. Exemplary antibodies and fragments that are specific for SCF248, as well as methods for making and using the antibodies and fragments, are provided in the present disclosure. In some embodiments, the antibodies and fragments thereof provided herein breaks the positive feedback loop between SCF248 expressed on various cell types and cKit+ immune cells, by binding to SCF248 and blocking the interaction between SCF248 and c-Kit.

### Antibodies and Fragments

The present disclosure provides antibodies, including monoclonal antibodies, and fragments thereof. The antibody fragments provided herein that are specific for SCF (e.g., SCF248) are sometimes referred to herein as antigen-binding fragments, meaning that they comprise the portion of the parent antibody that is capable of binding the target antigen (SCF, e.g., SCF248). “Antibody fragment,” “antigen binding fragment” and the like are used interchangeably herein. Examples of antibody fragments include Fab fragments, Fab′ fragments, F (ab)′ fragments, Fv fragments, isolated CDR regions, bispecific Fab dimers (Fab2), trispecific Fab trimers (Fab3), single chain Fv proteins (“scFv”), bis-scFv, (scFv) 2, minibodies, diabodies, triabodies, tetrabodies, disulfide stabilized Fv proteins (“dsFv”), single-domain antibodies (sdAb, nanobody), heavy-chain only antibodies (e.g., camelid VHH, camelid nanobody, shark Ig NAR), and portions of full length antibodies responsible for antigen binding.

A “Fab fragment” comprises one light chain and the C_{H}1 and variable regions of one heavy chain. The heavy chain of a Fab molecule cannot form a disulfide bond with another heavy chain molecule. A “Fab′ fragment” comprises one light chain and a portion of one heavy chain that contains the VH domain and the C_{H}1 domain and also the region between the C_{H}1 and C_{H}2 domains, such that an interchain disulfide bond can be formed between the two heavy chains of two Fab′ fragments to form an F(ab′)₂ molecule. A “F(ab′)₂ fragment” contains two light chains and two heavy chains containing a portion of the constant region between the C_{H}1 and C_{H}2 domains, such that an interchain disulfide bond is formed between the two heavy chains. A F(ab′)₂ fragment thus is composed of two Fab′ fragments that are held together by a disulfide bond between the two heavy chains. A “Fv fragment” comprises the variable regions from both the heavy and light chains, but lacks the constant regions. “scFvs” are Fv molecules in which the heavy and light chain variable regions have been connected by a flexible linker to form a single polypeptide chain, which forms an antigen binding region.

In some aspects, the antibodies and fragments thereof provided herein are defined by their complementary determining regions (CDRs). CDRs are part of the variable chains in antibodies; each of the light and heavy chain variable regions comprises three CDRs, CDR1, CDR2, and CDR3. The CDRs of an antibody determine antigen specificity. In certain embodiments, definitive delineation of a CDR and identification of residues comprising the binding site of an antibody is accomplished by solving the structure of the antibody and/or solving the structure of the antibody-ligand complex. In certain embodiments, that can be accomplished by any of a variety of techniques known to those skilled in the art, such as X-ray crystallography. In certain embodiments, various methods of analysis can be employed to identify or approximate the CDR regions. Examples of such methods include, but are not limited to, the Kabat definition, the Chothia definition, the AbM definition and the contact definition.

The Kabat definition is a standard for numbering the residues in an antibody and is typically used to identify CDR regions. See, e.g., Johnson & Wu, Nucleic Acids Res., 28:214-8 (2000). The Chothia definition is similar to the Kabat definition, but the Chothia definition takes into account positions of certain structural loop regions. See, e.g., Chothia et al., J. Mol. Biol., 196:901-17 (1986); Chothia et al., Nature, 342:877-83 (1989). The AbM definition uses an integrated suite of computer programs produced by Oxford Molecular Group that model antibody structure. See, e.g., Martin et al., Proc Natl Acad Sci (USA), 86:9268-9272 (1989); “AbM™, A Computer Program for Modeling Variable Regions of Antibodies,” Oxford, UK; Oxford Molecular, Ltd. The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl., 3:194-198 (1999). The contact definition is based on an analysis of the available complex crystal structures. See, e.g., MacCallum et al., J. Mol. Biol., 5:732-45 (1996).

Antibodies and fragments thereof may also include recombinant polypeptides, fusion proteins, and bi-specific antibodies. The anti-SCF antibodies and fragments thereof disclosed herein may be of an IgG1, IgG2, IgG3, or IgG4 isotype. In one embodiment, the anti-SCF antibodies and fragments thereof disclosed herein are of an IgG1 or an IgG4 isotype. The anti-SCF antibodies and fragments thereof of the present invention may be derived from any species including, but not limited to, mouse, rat, rabbit, primate, llama, camel, goat, shark, chicken, and human. The SCF antibodies and fragments thereof may be chimeric, humanized, or fully human antibodies. In one embodiment, the anti-SCF antibodies are murine antibodies. In another embodiment, the anti-SCF antibodies are chimeric antibodies. In a further embodiment, the chimeric antibodies are mouse-human chimeric antibodies. In another embodiment, the antibodies are derived from mice and are humanized.

A “chimeric antibody” is an antibody having at least a portion of the heavy chain variable region and at least a portion of the light chain variable region derived from one species; and at least a portion of a constant region derived from another species. For example, in one embodiment, a chimeric antibody may comprise murine variable regions and a human constant region.

A “humanized antibody” is an antibody containing complementarity determining regions (CDRs) that are derived from a non-human antibody; and framework regions as well as constant regions that are derived from a human antibody. For example, the anti-SCF antibodies provided herein may comprise CDRs derived from one or more murine antibodies and human framework and constant regions. Thus, in one embodiment, the humanized antibody provided herein binds to the same epitope on SCF as the murine antibody from which the antibody's CDRs are derived.

In some embodiments, the antibodies and fragments thereof provided herein comprise a heavy and light chain, each of which comprises three CDRs. The amino acid sequences of exemplary heavy chain CDR1, CDR2, and CDR3 (HCDR1, HCDR2, and HCDR3, respectively) and light chain CDR1, CDR2, and CDR3 (LCDR1, LCDR2, and LCDR3, respectively) are provided below in Table 1. Table 1 also provides the amino acid sequences of exemplary heavy and light chain variable regions. In some embodiments, the present disclosure provides antibodies referred to herein as “5H10” and “2G8”. The heavy chain variable regions of humanized 5H10 or 2G8 are referred to herein as VH1, VH2, VH3, VH4, and VH5. 5H10 VH0 is the variable heavy chain of the murine parent antibody generated via the methods described herein. VH1, VH2, VH3, VH4, and VH5 are each humanized heavy chain variable regions derived from 5H10 VH0 or 2G8 VH0. The 5H10 antibody comprises a kappa light chain. The murine parent antibody variable light chain is referred to herein as 5H10 VK0. VK1, VK2, VK3, and VK4 are each humanized light chain variable regions derived from VK0. The 2G8 antibody comprises a lambda light chain. The murine parent antibody variable light chain is referred to herein as 2G8 VL0. VL1, VL2, VL3, and VL4 are each humanized light chain variable regions derived from VL0.

TABLE 1
Exemplary anti-SCF antibody sequences

| SEQ | | |
|---|---|---|
| ID | | |
| NO | Description | |
| 1 | 5H10 Heavy | SYWMN |
| | chain CDR1 | |
| 2 | 5H10 Heavy | QIYPGDGDTHYNGKFKG |
| | chain CDR2 | |
| 3 | 5H10 Heavy | SNWVGSY |
| | chain CDR3 | |
| 4 | 5H10 Light | KSSQSLLESDGKTYLN |
| | chain CDR1 | |
| 5 | 5H10 Light | LVSRLDS |
| | chain CDR2 | |
| 6 | 5H10 Light | WQGTHLPQT |
| | chain CDR3 | |
| 7 | 5H10 Heavy | QVQLQQSGAELVRPGSSVKISCKSSGYAFSSYWMNWVKQRPGQG |
| | chain variable | LEWIGQIYPGDGDTHYNGKFKGKATLTADKSSSTAYMQLSRLTSE |
| | region VH0 | DSAVYFCSSSNWVGSYWGQGTLVTVSA |
| | (murine parent) | |
| 8 | 5H10 Heavy | QVQLVQSGAELKKPGSSVKISCKSSGYAFSSYWMNWVKQRPGQG |
| | chain variable | LEWIGQIYPGDGDTHYNGKFKGKATLTADKSTSTAYMELSSLTSE |
| | region VH1 | DSAVYFCSSSNWVGSYWGQGTLVTVSS |
| | (humanized) | |
| 9 | 5H10 Heavy | QVQLVQSGAEVKKPGSSVKISCKSSGYAFSSYWMNWVKQRPGQG |
| | chain variable | LEWIGQIYPGDGDTHYNGKFKGKATLTADKSTSTAYMELSSLRSE |
| | region VH2 | DTAVYFCSSSNWVGSYWGQGTLVTVSS |
| | (humanized) | |
| 10 | 5H10 Heavy | QVQLVQSGAEVKKPGSSVKVSCKSSGYAFSSYWMNWVRQRPGQ |
| | chain variable | GLEWIGQIYPGDGDTHYNGKFKGKATLTADKSTSTAYMELSSLRS |
| | region VH3 | EDTAVYFCSSSNWVGSYWGQGTLVTVSS |
| | (humanized) | |
| 11 | 5H10 Heavy | QVQLVQSGAEVKKPGSSVKVSCKSSGYAFSSYWMNWVRQRPGQ |
| | chain variable | GLEWIGQIYPGDGDTHYNGKFKGRVTITADKSTSTAYMELSSLRSE |
| | region VH4 | DTAVYFCSSSNWVGSYWGQGTLVTVSS |
| | (humanized) | |
| 12 | 5H10 Heavy | QVQLVQSGAEVKKPGSSVKVSCKSSGYAFSSYWMNWVRQRPGQ |
| | chain variable | GLEWIGQIYPGDGDTHYNGKFQGRVTITADKSTSTAYMELSSLRSE |
| | region VH5 | DTAVYYCSSSNWVGSYWGQGTLVTVSS |
| | (humanized) | |
| 13 | 5H10 Light | DVVMTQTPLTLSVTIGQTASISCKSSQSLLESDGKTYLNWLSQRPG |
| | chain variable | QSPKRLIYLVSRLDSGVPDRFTGSGSGTDFTLKISRVEAEDLGVYY |
| | region VK0 | CWQGTHLPQTFGGGTKLEIK |
| | (murine parent) | |
| 14 | 5H10 Light | DVVMTQSPLTLSVTLGQPASISCKSSQSLLESDGKTYLNWLQQRPG |
| | chain variable | QSPRRLIYLVSRLDSGVPDRFTGSGSGTDFTLKISRVEAEDVGVYY |
| | region VK1 | CWQGTHLPQTFGGGTKVEIK |
| | (humanized) | |
| 15 | 5H10 Light | DVVMTQSPLSLPVTLGQPASISCKSSQSLLESDGKTYLNWLQQRPG |
| | chain variable | QSPRRLIYLVSRLDSGVPDRFTGSGSGTDFTLKISRVEAEDVGVYY |
| | region VK2 | CWQGTHLPQTFGGGTKVEIK |
| | (humanized) | |
| 16 | 5H10 Light | DVVMTQSPLSLPVTLGQPASISCKSSQSLLESDGKTYLNWLQQRPG |
| | chain variable | QSPRRLIYLVSRLDSGVPDRFSGSGSGTDFTLKISRVEAEDVGVYY |
| | region VK3 | CWQGTHLPQTFGGGTKVEIK |
| | (humanized) | |
| 17 | 5H10 Light | DVVMTQSPLSLPVTLGQPASISCKSSQSLLESDGKTYLNWFQQRPG |
| | chain variable | QSPRRLIYLVSRLDSGVPDRFSGSGSGTDFTLKISRVEAEDVGVYY |
| | region VK4 | CWQGTHLPQTFGGGTKVEIK |
| | (humanized) | |
| 37 | 5H10 VH5 | QIYPGDGDTHYNGKFQG |
| | CDR2 | |

The skilled person will understand that the variable heavy and variable light chains may be independently selected, or mixed and matched, from the antibodies provided herein. Thus, in some embodiments, the antibodies and fragments thereof provided herein comprise heavy and light chain combinations selected from the group consisting of VH0/VK0, VH0/VK1, VH0/VK2, VH0/VK3, VH0/VK4, VH1/VK0, VH1/VK1, VH1/VK2, VH1/VK3, VH1/VK4, VH2/VK0, VH2/VK1, VH2/VK2, VH2/VK3, VH2/VK4, VH3/VK0, VH3/VK1, VH3/VK2, VH3/VK3, VH3/VK4, VH4/VK0, VH4/VK1, VH4/VK2, VH4/VK3, VH4/VK4, VH5/VK0, VH5/VK1, VH5/VK2, VH5/VK3, and VH5/VK4.

In some embodiments, the present disclosure provides antibodies or fragments comprising amino acid sequences having at least 80%, at least 85%, at least 90%, at least 95%, or at least 99% homology to an amino acid sequence selected from the group consisting of SEQ ID NOs: 7-12. In some embodiments, the present disclosure provides antibodies or fragments thereof comprising a heavy chain variable region according to a sequence selected from the group consisting of SEQ ID NOs: 7-12. In some embodiments, the present disclosure provides antibodies or fragments comprising amino acid sequences having at least 80%, at least 85%, at least 90%, at least 95%, or at least 99% homology to an amino acid sequence selected from the group consisting of SEQ ID NOs: 7-11, wherein the antibody or fragment comprises a heavy chain CDR1, CDR2, and CDR3 identical to SEQ ID NOs: 1, 2, and 3, respectively. In some embodiments, the present disclosure provides antibodies or fragments comprising amino acid sequences having at least 80%, at least 85%, at least 90%, at least 95%, or at least 99% homology to an amino acid sequence of SEQ ID NO: 12, wherein the antibody or fragment comprises a heavy chain CDR1, CDR2, and CDR3 identical to SEQ ID NOs: 1, 37, and 3, respectively.

In some embodiments, the present disclosure provides antibodies or fragments comprising amino acid sequences having at least 80%, at least 85%, at least 90%, at least 95%, or at least 99% homology to an amino acid sequence selected from the group consisting of SEQ ID NOs: 13-17. In some embodiments, the present disclosure provides antibodies or fragments thereof comprising a light chain variable region according to a sequence selected from the group consisting of SEQ ID NOs: 13-17. In some embodiments, the present disclosure provides antibodies or fragments comprising amino acid sequences having at least 80%, at least 85%, at least 90%, at least 95%, or at least 99% homology to an amino acid sequence selected from the group consisting of SEQ ID NOs: 13-17, wherein the antibody or fragment comprises a light chain CDR1, CDR2, and CDR3 identical to SEQ ID NOs: 4, 5, and 6, respectively.

In some embodiments, the present disclosure provides antibodies or fragments comprising amino acid sequences having at least 80%, at least 85%, at least 90%, at least 95%, or at least 99% homology to: SEQ ID NO: 7 and SEQ ID NO: 13; SEQ ID NO: 7 and SEQ ID NO: 14; SEQ ID NO: 7 and SEQ ID NO: 15; SEQ ID NO: 7 and SEQ ID NO: 16; SEQ ID NO: 7 and SEQ ID NO: 17; SEQ ID NO: 7 and SEQ ID NO: 13; SEQ ID NO: 7 and SEQ ID NO: 14; SEQ ID NO: 7 and SEQ ID NO: 15; SEQ ID NO: 7 and SEQ ID NO: 16; SEQ ID NO: 7 and SEQ ID NO: 17; SEQ ID NO: 8 and SEQ ID NO: 13; SEQ ID NO: 8 and SEQ ID NO: 14; SEQ ID NO: 8 and SEQ ID NO: 15; SEQ ID NO: 8 and SEQ ID NO: 16; SEQ ID NO: 8 and SEQ ID NO: 17; SEQ ID NO: 9 and SEQ ID NO: 13; SEQ ID NO: 9 and SEQ ID NO: 14; SEQ ID NO: 9 and SEQ ID NO: 15; SEQ ID NO: 9 and SEQ ID NO: 16; SEQ ID NO: 9 and SEQ ID NO: 17; SEQ ID NO: 10 and SEQ ID NO: 13; SEQ ID NO: 10 and SEQ ID NO: 14; SEQ ID NO: 10 and SEQ ID NO: 15; SEQ ID NO: 10 and SEQ ID NO: 16; SEQ ID NO: 10 and SEQ ID NO: 17; SEQ ID NO: 11 and SEQ ID NO: 13; SEQ ID NO: 11 and SEQ ID NO: 14; SEQ ID NO: 11 and SEQ ID NO: 15; SEQ ID NO: 11 and SEQ ID NO: 16; SEQ ID NO: 11 and SEQ ID NO: 17; SEQ ID NO: 12 and SEQ ID NO: 13; SEQ ID NO: 12 and SEQ ID NO: 14; SEQ ID NO: 12 and SEQ ID NO: 15; SEQ ID NO: 12 and SEQ ID NO: 16; or SEQ ID NO: 12 and SEQ ID NO: 17.

In particular embodiments, the antibodies and fragments thereof comprise heavy and light chain combinations selected from the group consisting of VH1/VK1, VH1/VK2, VH1/VK3, VH2/VK1, VH2/VK2, VH2/VK3, VH3/VK1, VH3/VK2, VH3/VK3, VH4/VK1, VH4/VK2, VH4/VK3, VH5/VK1, VH5/VK2, and VH5/VK3. In some embodiments, the antibodies comprise an amino acid sequence having at least 80%, at least 85%, at least 90%, at least 95%, or at least 99% sequence identity to SEQ ID NO: 8 or SEQ ID NO: 9; and an amino acid sequence having at least 80%, at least 85%, at least 90%, at least 95%, or at least 99% sequence identity to SEQ ID NO: 16. In some embodiments, the antibody, or fragment thereof, comprises an amino acid sequence having at least 80%, at least 85%, at least 90%, at least 95%, or at least 99% sequence identity to SEQ ID NO: 8 or SEQ ID NO: 9; and an amino acid sequence having at least 80%, at least 85%, at least 90%, at least 95%, or at least 99% sequence identity to SEQ ID NO: 16; wherein the antibody or fragment comprises a heavy chain CDR1, CDR2, and CDR3 identical to SEQ ID NOs: 1, 2, and 3, respectively, and a light chain CDR1, CDR2, and CDR3 identical to SEQ ID NOs: 4, 5, and 6, respectively. In some embodiments, the antibody, or fragment thereof, comprises an amino acid sequence having at least 95% or at least 99% sequence identity to SEQ ID NO: 8 or SEQ ID NO: 9; and an amino acid sequence having at least 95% or at least 99% sequence identity to SEQ ID NO: 16; wherein the antibody or fragment comprises a heavy chain CDR1, CDR2, and CDR3 identical to SEQ ID NOs: 1, 2, and 3, respectively, and a light chain CDR1, CDR2, and CDR3 identical to SEQ ID NOs: 4, 5, and 6, respectively. The antibody or fragment thereof may specifically bind to SCF248 but may not bind to SCF220. In some embodiments, the antibodies comprise a heavy chain variable region according to SEQ ID NO: 8 and a light chain variable region according to SEQ ID NO: 16. In some embodiments, the antibodies comprise a heavy chain variable region according to SEQ ID NO: 9 and a light chain variable region according to SEQ ID NO: 16.

In some embodiments, the antibodies and fragments provided herein comprise a heavy chain variable region amino acid sequence according to SEQ ID NO: 7, 8, 9, 10, 11, or 12, or a variant thereof; and/or comprise a light chain variable region amino acid sequence according to SEQ ID NO: 13, 14, 15, 16, or 17, or a variant thereof. Variants may comprise 1, 2, 3, 4, 5, 6, 7, 8, 9, or 10 amino acid substitutions or deletions, or a combination thereof. In some embodiments, the amino acid substitutions are conservative substitutions. The anti-SCF antibodies disclosed herein having one or more amino acid substitution, insertion, deletion, or combination thereof in the CDR or variable light or heavy chain region retain the biological activity of the corresponding anti-SCF antibody that does not have an amino acid substitution, insertion, or deletion relative to the sequences provided herein. Thus, the variant anti-SCF antibodies provided herein retain specific binding to SCF248. The terms percent homology, sequence identity, sequence homology, and the like are used interchangeably herein and refer to the number of identical amino acid sequences shared by two reference sequences, divided by the total number of amino acid positions, multiplied by 100.

In some embodiments, the present invention provides antibodies that bind to the same epitope as any one of the exemplary antibodies disclosed herein. Thus, in some embodiments, the present invention provides antibodies that compete for binding to SCF with the exemplary antibodies provided herein. For example, in some embodiments, the present disclosure provides antibodies that specifically bind to a region of the amino acid sequence provided herein as SEQ ID NO: 29. In some embodiments, antibodies provided herein specifically bind to an epitope comprising the amino acid sequence of SEQ ID NO: 33 (ASSLRNDSSSSNRK) or SEQ ID NO: 36 ASSLRNDSSSSNR). In some embodiments, the present disclosure provides antibodies that specifically bind to an epitope consisting of an amino acid sequence according to SEQ ID NO: 33 or SEQ ID NO: 36. In some embodiments, the present disclosure provides antibodies that specifically bind to an epitope comprising at least 5, at least 6, at least 7, at least 8, at least 9, or at least 10 contiguous amino acids of SEQ ID NO: 33.

In some embodiments, the antibodies and fragments thereof provided herein comprise the HCDR1, HCDR2, HCDR3, LCDR1, LCDR2, and/or LCDR3 of the heavy and light chain variable regions provided herein, or variants thereof. Thus, in some embodiments, the antibodies and fragments thereof provided herein include antibodies wherein the HCDRs are the HCDRs of SEQ ID NO: 7, 8, 9, 10, 11, or 12; and/or wherein the LCDRs are the LCDRs of SEQ ID NOs: 13, 14, 15, 16, or 17. For example, in some embodiments, the antibodies and fragments thereof comprise amino acids 31-35, 50-65, and 95-102 of any one of the heavy chain variable regions provided herein, as defined by the Kabat numbering scheme. In some embodiments, the antibodies and fragments thereof comprise amino acids 24-34, 50-56, and 89-97 of any one of the light chain variable regions provided herein, as defined by the Kabat numbering scheme.

Exemplary humanized antibodies are provided herein. Additional anti-SCF antibodies comprising the heavy and light chain CDRs provided herein, or variants thereof, may be generated using any human framework sequence, and are also encompassed in the present invention. In one embodiment, framework sequences suitable for use in the present invention include those framework sequences that are structurally similar to the framework sequences provided herein. Further modifications in the framework regions may be made to improve the properties of the antibodies provided herein. Such further framework modifications may include chemical modifications; point mutations to reduce immunogenicity or remove T cell epitopes; or back mutation to the residue in the original germline sequence.

In some embodiments, such framework modifications include those corresponding to the mutations exemplified herein, including backmutations to the germline sequence. For example, in one embodiment, one or more amino acids in the human framework regions of the VH and/or VL of the humanized antibodies provided herein are back mutated to the corresponding amino acid in the parent murine antibody. The present invention also encompasses humanized antibodies that bind to SCF (e.g., SCF248) and comprise framework modifications corresponding to the exemplary modifications described herein with respect to any suitable framework sequence, as well as other framework modifications that otherwise improve the properties of the antibodies. In other embodiments, the antibodies provided herein comprise one or more mutations to improve stability, improve solubility, alter glycosylation, and/or reduce immunogenicity, such as, for example, by targeted amino acid changes that reduce deamidation or oxidation, reduce isomerization, optimize the hydrophobic core and/or charge cluster residues, remove hydrophobic surface residues, optimize residues involved in the interface between the variable heavy and variable light chains, and/or modify the isoelectric point.

The anti-SCF antibodies and fragments thereof provided herein may further comprise Fc region modifications to alter effector functions. Fc modifications may be amino acid insertions, deletions, or substitutions, or may be chemical modifications. For example, Fc region modifications may be made to increase or decrease complement binding, to increase or decrease antibody-dependent cellular cytotoxicity, or to increase or decrease the half-life of the antibody. Some Fc modifications increase or decrease the affinity of the antibody for an Fcγ receptor such as FcγRI, FcγRII, FcγRIII, or FcRn. Various Fc modifications have been described in the art, for example, in Shields et al., J Biol. Chem 276; 6591 (2001); Tai et al. Blood 119; 2074 (2012); Spiekermann et al. J Exp. Med 196; 303 (2002); Moore et al. mAbs 2:2; 181 (2010); Medzihradsky Methods in Molecular Biology 446; 293 (2008); Mannan et al. Drug Metabolism and Disposition 35; 86 (2007); and Idusogie et al. J Immunol 164; 4178 (2000). In some embodiments, Fc region glycosylation patters are altered. In other embodiments, the Fc region is modified by pegylation (e.g., by reacting the antibody or fragment thereof with polyethylene glycol (PEG). Exemplary Fc modifications include modifications at one or more amino acid position selected from the group consisting of 228, 233, 234, 235, 236, 241, 248, 265, 297, 309, 331, and 409 (Kabat numbering; Kabat et al., Sequences of Immunological Interest, Fifth Edition, National Institute of Health, Bethesda, Md. (1991)). In embodiments, the antibody has modifications to reduce or abolish effector function. In embodiments, the antibody is an IgG1 antibody having one or more Fc modification selected from the group consisting of E233P, L234V, L234A, L235V, L235A, G236 (deleted), D265A, D270A, N297A and N297Q. In embodiments, the antibody is an IgG4 antibody having one or more Fc modification selected from the group consisting of S228P, E233P, F234A, F234V, L235A, L235V, S241P, L248E, D265A, D265T, L309L, and R409K. In embodiments, the anti-SCF antibodies provided herein comprise a S241P mutation and an L248E mutation.

In embodiments, the present disclosure provides antibodies provided herein that comprise a human IgG4 constant region according to SEQ ID NOs: 40 and 41. In embodiments, the present disclosure provides antibodies comprising at least about 80%, at least about 85%, at least about 90%, at least about 95%, or about 99% sequence identity to SEQ ID NO: 40, 41, 42, 43, 44, 45, 46, 47, 48, 49, or 50. In embodiments, the present disclosure provides antibodies comprising a heavy chain according to SEQ ID NO: 40 and a light chain according to SEQ ID NO: 41. In embodiments, the present disclosure provides antibodies comprising a heavy chain according to SEQ ID NO: 42, 43, 44, 45, or 46 and a light chain according to SEQ ID NO: 47, 48, 49, or 50. In embodiments, the present disclosure provides an antibody comprising a heavy chain according to SEQ ID NO: 42 and a light chain according to SEQ ID NO: 49. In embodiments, the present disclosure provides an antibody comprising a heavy chain according to SEQ ID NO: 43 and a light chain according to SEQ ID NO: 49. In embodiments, the present disclosure provides an antibody comprising a heavy chain according to SEQ ID NO: 44 and a light chain according to SEQ ID NO: 49. In embodiments, the present disclosure provides an antibody comprising a heavy chain according to SEQ ID NO: 45 and a light chain according to SEQ ID NO: 49. In embodiments, the present disclosure provides an antibody comprising a heavy chain according to SEQ ID NO: 46 and a light chain according to SEQ ID NO: 49.

In some embodiments, the present disclosure provides methods for making antibodies that specifically bind to SCF248. The SCF248 isoform of SCF include exon 6, which comprises a cleavage site between two alanine residues (amino acids 16 and 17 of SEQ ID NO: 34, which provides the amino acid sequence of exon 6). Previous anti-SCF antibodies were generated by immunizing mice with a peptide spanning exon 6 and part of Exon 7 (see, e.g., U.S. Pat. No. 8,911,729, which is hereby incorporated by reference in its entirety for all purposes). Since SCF220 is associated with homeostatic activities, any cross-reactivity with SCF220 would be detrimental as it would result in various off-target effects in subjects. Advantageously, the antibodies provided in the present disclosure bind to SCF248 with very high specificity. In some embodiments, the antibodies provided herein are specific for SCF248 and do not bind to SCF220. Thus, the antibodies provided herein are capable of specifically inhibiting the interaction between SCF248 and c-Kit that induces and perpetuates chronic inflammatory responses and fibrosis. Moreover, the antibodies provided herein are capable of specifically inducing the internalization of SCF and thereby reducing the interaction between SCF248 and c-Kit. Accordingly, in some embodiments the present disclosure provides antibodies that are specific for SCF248 and are safe and effective in various inflammatory and fibrotic diseases discussed herein and known in the art.

For preparation of monoclonal antibodies, any technique that provides for the production of antibody molecules by continuous cell lines in culture may be used (see e.g., Harlow and Lane, Antibodies: A Laboratory Manual, Cold Spring Harbor Laboratory Press, Cold Spring Harbor, N.Y.). These include, but are not limited to, the hybridoma technique originally developed by Kohler and Milstein and the trioma technique, the human B-cell hybridoma technique (See, e.g., Kozbor et al., Immunol. Today, 4:72 (1983)), and the EBV-hybridoma technique to produce human monoclonal antibodies (Cole et al., in Monoclonal Antibodies and Cancer Therapy, Alan R. Liss, Inc., pp. 77-96 (1985)). Alternatively, the antibodies may be made by recombinant DNA methods. In some embodiments, antibodies in accordance with the present disclosure may be made by isolating monoclonal antibodies from phage display libraries using the techniques described, for example, in Clackson et al., Nature 352:624-28 (1991) and Marks et al., J. Mol. Biol. 222(3):581-97 (1991). In some embodiments, the antibodies are fully human antibodies constructed by combining Fv clone variable domain sequence(s) selected from human-derived phage display or yeast display libraries with known human constant domain sequence(s).

In some embodiments provided herein, the antibodies are prepared from a hybridoma. Using the hybridoma method, a mouse, hamster, or other appropriate host animal, is immunized by injecting an immunizing peptide to elicit the production by lymphocytes of antibodies that will specifically bind to an immunizing antigen. Alternatively, lymphocytes can be immunized in vitro. Following immunization, the lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen as determined by immunoprecipitation, immunoblotting, or by an in vitro binding assay such as radioimmunoassay (RIA) or enzyme-linked immunosorbent assay (ELISA) can then be propagated in vitro (e.g., in culture) using standard methods (Goding, Monoclonal Antibodies: Principles and Practice, Academic Press, 1986) or in vivo as ascites tumors in an animal. The monoclonal antibodies can then be purified from the culture medium or ascites fluid as described for polyclonal antibodies above.

In some embodiments, the antibodies provided herein are generated using the murine hybridoma system. Hybridoma production in the mouse is a well-established procedure. Immunization protocols and techniques for isolation of immunized splenocytes for fusion are known in the art. Fusion partners (e.g., murine myeloma cells) and fusion procedures are also known. Embodiments of the technology herein provide antibodies (e.g., monoclonal antibodies) produced from a hybridoma prepared by immunizing mice with a peptide that is a portion or fragment of the SCF protein.

In some embodiments, the antibodies specific for SCF248 provided herein are generated by immunizing mice with a peptide having an amino acid sequence that is largely or exclusively within exon 6. For example, the immunizing peptide comprises any stretch of 5 or more amino acids within SEQ ID NO: 34. As another example, the immunizing peptide comprises any stretch of 5 or more amino acids beginning at amino acid position 20 of SEQ ID NO: 29. As another example, the immunizing peptide comprises a stretch of 5 or more amino acids beginning at amino acid position 20 of SEQ ID NO: 29 and ending at any one of positions 25 to 38 of SEQ ID NO: 29. Thus, in some embodiments, the immunizing peptide comprises the amino acid sequence of exon 6 after the cleavage site, and is either fully contained within exon 6 or comprises only 1, 2, 3, 4, or 5 amino acids of exon 7. In some embodiments, the immunizing peptide comprises or consists of SEQ ID NO: 30. In some embodiments, the immunizing peptide comprises any of the peptides provided herein or conservative variants thereof. Conservative variants may comprise 1, 2, 3, 4, or 5 amino acid substitutions or deletions, or a combination thereof. As provided above, in some embodiments, the antibodies generated using the immunizing peptides provided herein have an epitope that falls entirely or largely within exon 6. By “largely within” it is meant that at least 75%, at least 80%, at least 85%, at least 90%, or at least 95% of the peptide falls within exon 6. In some embodiments, the epitope begins at the cleavage site of exon 6 (i.e., between the alanines at amino acid positions 19 and 20 of SEQ ID NO: 29 and extends to the end of exon 6. In some embodiments, the epitope begins at the cleavage site of exon 6 and extends to the 1^{st}, 2^{nd}, 3^{rd}, 4^{th}, or 5^{th} n-terminal amino acid of the transmembrane domain. In some embodiments, the epitope comprises or consists of SEQ ID NO: 33. In some embodiments, the antibody referred to herein as 5H10 (including the murine, chimeric, and humanized 5H10 antibodies) binds to an epitope of SCF comprising or consisting of SEQ ID NO: 33.

In some embodiments, the methods provided herein were used to generate antibodies referred to herein as 5H10. In some embodiments, the antibody “5H10” is also referred to herein as “OpSCF.” Antibody 5H10 advantageously binds SCF248 with high specificity and does not bind SCF220. The amino acid sequences of the murine parent antibody 5H10, as well as humanized variants thereof, are provided herein (see, Table 1).

In one embodiment, the present invention provides bispecific or multispecific antibodies specific for SCF and at least one other antigen or epitope. The anti-SCF antibodies and fragments thereof provided herein may be tested for binding to SCF using the binding assays provided herein, or any other binding assay known in the art.

Unless otherwise stated, the practice of the present invention employs conventional molecular biology, cell biology, biochemistry, and immunology techniques that are well known in the art and described, for example, in Methods in Molecular Biology, Humana Press; Molecular Cloning: A Laboratory Manual, second edition (Sambrook et al., 1989), Current Protocols in Immunology (J. E. Coligan et al., eds., 1991); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Phage display: a laboratory manual (C. Barbas III et al, Cold Spring Harbor Laboratory Press, 2001); and Using antibodies: a laboratory manual (E. Harlow and D. Lane (Cold Spring Harbor Laboratory Press, 1999).

### Methods of Treatment

In one aspect, the present disclosure provides methods for treating and/or preventing any disease or condition associated with immune cell migration, activation, and/or proliferation via interaction of SCF248 with c-Kit on immune cells. Thus, in some embodiments, the present disclosure provides methods for inhibiting or preventing activation of immune cells; as well as reducing or preventing the accumulation of immune cells within organs or tissues, thereby treating or preventing various diseases and disorders that involve inflammation. In some embodiments, the immune cells are selected from the group consisting of mast cells, innate lymphoid cells (ILCs, such as ILC2 or ILC3 cells), and eosinophils.

As used herein, the terms “treatment” or “treating” refers to both therapeutic treatment and prophylactic or preventive measures. Subjects in need of treatment include those subjects that already have the disease or condition, as well as those that may develop the disease or condition and in whom the object is to prevent, delay, or diminish the disease or condition. In embodiments, treatments for atopic dermatitis (“AD”) are included herein. In embodiments, treating AD comprises reducing or eliminating a symptom associated with AD. Non-limiting examples of AD symptoms include pruritus, dry skin, eczematous lesions, lichenification, redness on skin, irritation in skin, a rash that oozes, weeps clear fluid, or bleeds, and thickened or hardened skin. In embodiments, treating atopic dermatitis comprises eliminating or reducing a skin rash or itching in a patient with atopic dermatitis. In embodiments, treating atopic dermatitis comprises reducing the severity of AD lesions. In embodiments, reducing the severity of AD lesions includes reducing erythema, induration/papulation, lichenification, and oozing/crusting. In embodiments, treating AD comprises reducing the body surface area (BSA) that is affected by AD. In embodiments, treating AD encompasses reducing or eliminating eczema.

As used herein, the term “subject” denotes a mammal, such as a rodent, a feline, a canine, and a primate. Preferably, a subject according to the invention is a human. As used herein, the term “patient” denotes a human subject. In embodiments, the subject is a human patient. In embodiments, the human patient has atopic dermatitis.

The term “therapeutically effective amount,” as used herein, refers to the amount of a compound or composition that is necessary to provide a therapeutic and/or preventative benefit to the subject or human patient.

In one aspect the present invention provides methods for treating a subject or human patient for an inflammatory disease, a fibrotic disease, and/or a tissue remodeling disease. In some embodiments, the inflammatory disease is a chronic inflammatory disease.

Chronic inflammatory, fibrotic, and tissue remodeling diseases include diseases of the lung, kidney, liver, heart, skin, connective tissue, and other tissues. Exemplary inflammatory, fibrotic or tissue remodeling diseases include, without limitation, pulmonary fibrosis (e.g., idiopathic pulmonary fibrosis (IPF), scleroderma lung fibrosis, scleroderma-related interstitial lung disease (SSc-ILD), pulmonary fibrosis associated with a lung infection or pneumonia, pulmonary fibrosis associated with systemic lupus erythematosus and/or rheumatoid arthritis, sarcoidosis), chronic obstructive pulmonary disease (COPD), acute respiratory distress syndrome (ARDS), cystic fibrosis, peribronchial fibrosis, bleomycin lung, hypersensitivity pneumonitis, asthma, fibrothorax, mediastinal fibrosis, chronic rhinosinusitis, urticaria (e.g., chronic spontaneous urticaria), atopic dermatitis, dermatomyositis, nodular subepidermal fibrosis, scleroderma, keloid, renal fibrosis, chronic kidney disease, glomerulonephritis, chronic renal allograft rejection, nephropathy (e.g., IgA nephropathy, focal segmental glomerulosclerosis, rapidly progressive glomerulonephritis, crescentic glomerulonephritis, lupus nephritis, hypertensive nephropathy, or diabetic nephropathy), non-alcoholic steatohepatitis (NASH), liver cirrhosis, hepatic fibrosis, primary sclerosing cholangitis, primary biliary cirrhosis, fibromyalgia, gingival fibrosis, radiation-induced fibrosis, eosinophilic esophagitis, arthrofibrosis, and atrial fibrosis, endomyocardial fibrosis, parenchymal fibrosis, fibrous histocytoma, or glial scarring.

In some embodiments, the antibodies and fragments thereof disclosed herein may be administered to the subject by at least one route selected from parenteral, subcutaneous, intramuscular, intravenous, intrarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intratympanic, intrauterine, intravesical, intravitreal, bolus, subconjunctival, oral, vaginal, rectal, buccal, sublingual, intranasal, intratumoral, and transdermal.

In embodiments, the antibodies and fragments thereof disclosed herein may be administered to a subject in need thereof in combination with one or more additional therapy. The one or more additional therapy may be a procedure such as a surgical procedure, or may be a therapeutic agent, such as an agent designed to mitigate or reduce symptoms of a disease or disorder associated with fibrosis and/or inflammation.

The present invention is further illustrated by reference to the following Examples. However, it should be noted that these Examples, like the embodiments described above, are illustrative and are not to be construed as restricting the scope of the invention in any way.

### Methods for Treating Atopic Dermatitis

Provided herein are methods for treating atopic dermatitis (“AD”), which is used interchangeably herein with “eczema.” The methods described herein for treating AD comprise administering a full length humanized anti-stem cell factor 248 (anti-SCF248) antibody comprising two heavy chains, each having an amino acid sequence of SEQ ID NO: 42, and two light chains, each having an amino acid sequence of SEQ ID NO: 49. This anti-SCF248 antibody is referred to herein as “OpSCF” or “humanized 5H10.”

Atopic dermatitis (AD), a form of eczema, is a chronic inflammatory skin disease. AD typically presents symptoms including, but not limited to, pruritus, dry skin, eczematous lesions, lichenification, redness, irritation, rashes that may ooze, weep clear fluid or bleed, and thickening and hardening of affected skin. In some embodiments, treating according to the methods described herein reduces or eliminates one or more symptoms associated with AD. In embodiments, the efficacy of treating atopic dermatitis treatments is assessed using one or more clinical endpoints selected from the group consisting of: Eczema Area and Severity Index (EASI), validated Investigational Global Assessment for atopic dermatitis (AD) (vIGA-AD), Peak Pruritus Numerical Rating Scale (PP-NRS), percent body surface area (BSA) affected by AD, Atopic Dermatitis Control Tool (ADCT) score, Patient Oriented Eczema Measure (POEM) score, and Dermatology Life Quality Index (DLQI) score. Each of these endpoints is described herein in detail.

In embodiments, provided herein is a method for treating AD in a human patient with atopic dermatitis, comprising administering to the human patient a therapeutically effective dose of a full length anti-stem cell factor 248 (anti-SCF248) antibody, wherein the full length anti-SCF248 antibody comprises two heavy chains each having the amino acid sequence of SEQ ID NO: 42 and two light chains each having the amino acid sequence of SEQ ID NO: 49; wherein the therapeutically effective dose is from 10 mg to 600 mg regardless of the human patient's body weight, and wherein treating is reducing or eliminating itching or a skin rash in the human patient.

Provided herein is a method for treating AD in a human patient with atopic dermatitis, comprising administering to the patient a therapeutically effective dosage regimen of a full length anti-stem cell factor 248 (anti-SCF248) antibody, wherein the full length anti-SCF248 antibody comprises two heavy chains each having the amino acid sequence of SEQ ID NO:42 and two light chains each having the amino acid sequence of SEQ ID NO:49, wherein the method reduces or eliminates itching or a skin rash, the dosage regimen comprising: a) administering to the human patient a first dose of 10 mg to 600 mg of the full length anti-SCF248 antibody regardless of the patient's body weight; and b) administering to the human patient a second dose of 10 mg to 600 mg of the full length anti-SCF248 antibody regardless of the patient's body weight; and wherein the method results in the patient achieving at least a 50% reduction from baseline Eczema Activity Severity Index (EASI) scale score sixteen weeks after receiving the first dose.

Provided herein is a method for treating AD in a human patient with atopic dermatitis, comprising administering to the patient a therapeutically effective dosage regimen of a full length anti-stem cell factor 248 (anti-SCF248) antibody, wherein the full length anti-SCF248 antibody comprises two heavy chains each having the amino acid sequence of SEQ ID NO:42 and two light chains each having the amino acid sequence of SEQ ID NO:49, wherein the method reduces or eliminates itching or a skin rash, the dosage regimen comprising: a) administering to the human patient a first dose of 10 mg to 600 mg of the full length anti-SCF248 antibody regardless of the patient's body weight; and b) administering to the human patient a second dose of 10 mg to 600 mg of the full length anti-SCF248 antibody regardless of the patient's body weight; wherein the method results in the patient achieving at least a one point reduction from baseline validated Investigator's Global Assessment (vIGA) score for AD sixteen weeks after receiving the first dose.

In embodiments, provided herein is a method for eliminating itching or a skin rash in an human patient with AD, comprising administering to the human patient a therapeutically effective dose of a full length anti-stem cell factor 248 (anti-SCF248) antibody, wherein the full length anti-SCF248 antibody comprises two heavy chains each having the amino acid sequence of SEQ ID NO: 42 and two light chains each having the amino acid sequence of SEQ ID NO: 49; and wherein the therapeutically effective dose is from 10 mg to 600 mg regardless of the human patient's body weight.

### Therapeutically Effective Doses for Treating AD

In embodiments, a therapeutically effective dose for treating AD ranges from 10 mg to 600 mg. In embodiments, a therapeutically effective dose for treating AD is 10 mg, 20 mg, 30 mg, 40 mg, 50 mg, 60 mg, 70 mg, 80 mg, 90 mg, 100 mg, 110 mg, 120 mg, 130 mg, 140 mg, 150 mg, 160 mg, 170 mg, 180 mg, 190 mg, 200 mg, 210 mg, 220 mg, 230 mg, 240 mg, 250 mg, 260 mg, 270 mg, 280 mg, 290 mg, 300 mg, 310 mg, 320 mg, 330 mg, 340 mg, 350 mg, 360 mg, 370 mg, 380 mg, 390 mg, 400 mg, 410 mg, 420 mg, 430 mg, 440 mg, 450 mg, 460 mg, 470 mg, 480 mg, 490 mg, 500 mg, 510 mg, 520 mg, 530 mg, 540 mg, 550 mg, 560 mg, 570 mg, 580 mg, 590 mg, or 600 mg. In embodiments, a therapeutically effective dose for treating AD is 10 mg, 30 mg, 100 mg, 300 mg, and 600 mg.

### Therapeutically Effective Dosage Regimens for Treating AD

In embodiments, the methods described herein comprise administering a single dose of OpSCF. In embodiments, the methods provided herein comprise administering multiple doses of OpSCF. In embodiments, the methods comprise administering from 1 to 50 doses of OpSCF, including 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 doses, including all ranges and values therebetween, of OpSCF.

In embodiments, the methods provided herein comprise administering a first dose and a second dose of OpSCF. In embodiments, the first dose and second dose are different.

In embodiments, the methods comprise administering the first dose from one to 50 times. In embodiments, the first dose is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 times, including all ranges therebetween. In embodiments, the first dose is administered over a 1 week, 2 week, 3 weeks, 4 weeks, 5 weeks, 6 week, 7 week, 8 week, 9 week, 10 week, 11 week, 12 week, 13 week, 14 week, 15 week, 16 week, 17 week, 18 week, 19 week, or 20 week period. In embodiments, the first dose is administered every week, every two weeks, every three weeks, every four weeks, every five weeks, every six weeks, every seven weeks, every eight weeks, every nine weeks, or every ten weeks. In embodiments, the first dose is administered every two weeks. In embodiments, the first dose is administered every four weeks.

In embodiments, the methods comprise administering the second dose from one to 50 times. In embodiments, the second dose is administered 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 times, including all ranges therebetween. In embodiments, the second dose is administered over a 1 week, 2 week, 3 weeks, 4 weeks, 5 week, 6 week, 7 week, 8 week, 9 week, 10 week, 11 week, 12 week, 13 week, 14 week, 15 week, 16 week, 17 week, 18 week, 19 week, 20 week, 21 week, 22 week, 23 week, 24 week, 25 week, 26 week, 27 week, 28 week, 29 week, 30 week, 31 week, 32 week, 33 week, 34 week, 35 week, 36 week, 37 week, 38 week, 39 week, 40 week, 41 week, 42 week, 43 week, 44 week, 45 week, 46 week, 47 week, 48 week, 49 week, 50 week, 51 week, or 52 week period. In embodiments, the second dose is administered every week, every two weeks, every three weeks, every four weeks, every five weeks, every six weeks, every seven weeks, every eight weeks, every nine weeks, or every ten weeks. In embodiments, the second dose is administered every four weeks.

In embodiments, the methods comprise administering 600 mg of OpSCF every two weeks for 16 weeks. In embodiments, the methods comprise administering 600 mg of OpSCF every four weeks for up to 36 weeks. In embodiments, the methods comprise administering 600 mg of OpSCF every two weeks for 16 weeks and then subsequently administering 600 mg of OpSCF every four weeks for up to 36 weeks. In embodiments, OpSCF is administered subcutaneously.

In embodiments, the methods comprise administering 600 mg of OpSCF every two weeks for 14 weeks. In embodiments, the methods comprise administering 600 mg of OpSCF every four weeks for up to 36 weeks. In embodiments, the methods comprise administering 600 mg of OpSCF every two weeks for 14 weeks and then subsequently administering 600 mg of OpSCF every four weeks for up to 36 weeks. In embodiments, OpSCF is administered subcutaneously.

### Pharmacokinetics

Provided herein are pharmacokinetic parameters associated with a therapeutically effective dose. In embodiments, the pharmacokinetic parameters are provided by a therapeutically effective subcutaneous dose, a therapeutically effective intravenous dose, or a combination thereof. In embodiments, the pharmacokinetic parameters described herein are provided by a single therapeutic dose. In embodiments, the pharmacokinetic parameters are provided by multiple therapeutic doses. In embodiments, the pharmacokinetic parameters described herein are provided by a single therapeutic dose comprising 10 mg, 30 mg, 100 mg, 300 mg, or 600 mg. In embodiments, the pharmacokinetic parameters are provided by a single subcutaneous therapeutic dose comprising 10 mg, 30 mg, 100 mg, 300 mg, or 600 mg. In embodiments, the pharmacokinetic parameters described herein are provided by a single intravenous therapeutic dose comprising 100 mg 600 mg. In embodiments, the pharmacokinetic parameters described herein are provided by a single intravenous therapeutic dose comprising 10 mg, 30 mg or 300 mg. In embodiments, the pharmacokinetic parameters described herein are provided by multiples doses comprising 30 mg, 100 mg, and 300 mg.

Exemplary pharmacokinetic parameters are described in Table 2 below.

TABLE 2
Exemplary pharmacokinetic parameters

| Parameter | Description |
|---|---|
| AUC_{inf} | area under the serum concentration-time curve |
| | from time 0 to infinity |
| AUCₜ | area under the serum concentration-time curve |
| | from time 0 to the last measurable concentration |
| AUC_{0-168 hours} | area under the serum concentration-time curve |
| (also referred to | over a dosing interval |
| herein as AUCₜₐᵤ) | |
| CL or CL/F | clearance |
| Cₘₐₓ | maximum serum concentration |
| Cₘᵢₙ | minimum serum concentration |
| DAUCₜ | AUCₜ normalized by dose administered |
| DAUC_{inf} | AUC_{inf} normalized by dose administered |
| t_{1/2} | terminal half-life |
| Tₗₐₛₜ | time of the last quantifiable concentration |
| Tₘₐₓ | time to Cₘₐₓ |
| V or V/F | volume of distribution |
| Vₛₛ | volume of distribution at steady state |
| % AUCₑₓₜᵣₐₚ | % AUCextrap percentage of area under the |
| | concentration-time curve due to extrapolation from |
| | the last quantifiable concentration to infinity |

### Cₘₐₓ

In embodiments, administering a therapeutically effective dose of OpSCF provides a maximum blood plasma concentration (Cₘₐₓ) ranging from about 0.05 ng/ml to about 400,000 ng/mL, including about 0.05 ng/mL, about 0.5 ng/mL, about 1 ng/mL, about 500 ng/ml, about 1,000 ng/mL, about 5,000 ng/mL, about 10,000 ng/mL, about 15,000 ng/ml, about 20,000 ng/ml, about 25,000 ng/mL, about 30,000 ng/mL, about 35,000 ng/mL, about 40,000 ng/mL, about 45,000 ng/mL, about 50,000 ng/mL, about 55,000 ng/mL, about 60,000 ng/mL, about 65,000 ng/ml, about 70,000 ng/mL, about 75,000 ng/mL, about 80,000 ng/mL, about 85,000 ng/mL, about 90,000 ng/mL, about 95,000 ng/mL, about 100,000 ng/mL, about 105,000 ng/mL, about 110,000 ng/mL, about 115,000 ng/mL, about 120,000 ng/ml, about 125,000 ng/ml, about 130,000 ng/ml, about 135,000 ng/ml, about 140,000 ng/mL, about 145,000 ng/ml, about 150,000 ng/mL, about 155,000 ng/mL, about 160,000 ng/mL, about 165,000 ng/ml, about 170,000 ng/ml, about 175,000 ng/mL, about 180,000 ng/mL, about 185,000 ng/mL, about 190,000 ng/ml, about 195,000 ng/mL, about 200,000 ng/mL, about 205,000 ng/mL, about 210,000 ng/ml, about 215,000 ng/mL, about 220,000 ng/mL, about 225,000 ng/mL, about 230,000 ng/ml, about 235,000 ng/mL, about 240,000 ng/mL, about 245,000 ng/mL, about 250,000 ng/ml, about 255,000 ng/mL, about 260,000 ng/mL, about 265,000 ng/ml, about 270,000 ng/ml, about 275,000 ng/mL, about 280,000 ng/mL, about 285,000 ng/mL, about 290,000 ng/ml, about 295,000 ng/mL, about 300,000 ng/mL, about 305,000 ng/mL, about 310,000 ng/ml, about 315,000 ng/mL, about 320,000 ng/ml, about 325,000 ng/mL, about 330,000 ng/ml, about 335,000 ng/mL, about 340,000 ng/mL, about 345,000 ng/mL, about 350,000 ng/ml, about 355,000 ng/mL, about 360,000 ng/mL, about 365,000 ng/mL, about 370,000 ng/ml, about 375,000 ng/ml, about 380,000 ng/mL, about 385,000 ng/mL, about 390,000 ng/ml, about 395,000 ng/mL, and about 400,000 ng/mL, including all values and ranges therebetween.

In embodiments, administering a therapeutically effective dose of OpSCF provides a maximum blood plasma concentration (Cₘₐₓ) ranging from about 0.05 ng/ml to about 400,000 ng/mL, about 1,000 ng/mL to about 400,000 ng/mL, about 10,000 ng/ml to about 400,000 mg/mL, about 50,000 ng/mL to about 400,000 ng/mL, about 100,000 ng/mL to about 400,000 ng/ml, about 150,000 ng/mL to about 400,000 ng/mL, about 200,000 ng/ml to about 400,000 ng/ml, about 250,000 ng/mL to about 4000,000 ng/mL, about 300,000 ng/mL to about 400,000 ng/ml, about 350,000 ng/mL to about 400,000 ng/mL, about 0.05 ng/mL to about 300,000 ng/mL, about 1,000 ng/mL to about 300,000 ng/mL, about 10,000 ng/mL to about 300,000 mg/mL, about 50,000 ng/mL to about 300,000 ng/mL, about 100,000 ng/mL to about 300,000 ng/mL, about 150,000 ng/mL to about 300,000 ng/mL, about 200,000 ng/ml to about 300,000 ng/mL, about 250,000 ng/mL to about 300,000 ng/mL, about 0.05 ng/mL to about 200,000 ng/mL, about 1,000 ng/mL to about 200,000 ng/mL, about 10,000 ng/mL to about 200,000 mg/mL, about 50,000 ng/mL to about 200,000 ng/mL, about 100,000 ng/mL to about 200,000 ng/mL, about 150,000 ng/ml to about 200,000 ng/mL, about 0.05 ng/mL to about 100,000 ng/mL, about 1,000 ng/mL to about 100,000 mg/mL, about 10,000 ng/ml to about 100,000 ng/ml, about 50,000 ng/mL to about 100,000 ng/mL, about 0.05 ng/mL to about 50,000 ng/mL, about 1,000 ng/mL to about 50,000 mg/mL, about 10,000 ng/mL to about 50,000 ng/mL, about 0.05 ng/ml to about 10,000 ng/ml, about 1,000 ng/mL to about 10,000 ng/mL, or about 0.05 ng/ml to about 1,000 ng/ml, including any ranges or values therebetween.

In embodiments, administering a therapeutically effective dose of OpSCF provides a maximum blood plasma concentration (Cₘₐₓ) ranging from about 800 ng/mL to about 300,000 ng/ml (e.g., 854 ng/ml, 3580 ng/mL, 9900 ng/ml, 30000 ng/mL, 82300 ng/mL, 42700 ng/ml, or 2888000 ng/mL, including any values or ranges therebetween.)

In embodiments, administering a therapeutically effective dose of OpSCF (i.e., 10 mg of OpSCF) subcutaneously provides a maximum blood plasma concentration (Cₘₐₓ) ranging from about 683 ng/mL to about 1,068 ng/ml, about 733 ng/mL to about 1,068 ng/ml, about 783 ng/ml to about 1,068 ng/mL, about 823 ng/ml to about 1,068 ng/mL, about 873 ng/mL to about 1,068 ng/mL, about 923 ng/mL to about 1,068 ng/mL, about 973 ng/mL to about 1,068 ng/mL, 1,023 to about 1,068 ng/ml, about 683 ng/ml to about 1,023 ng/mL, about 733 ng/ml to about 1,023 ng/mL, about 783 ng/mL to about 1,023 ng/ml, about 823 ng/ml to about 1,023 ng/ml, about 873 ng/ml to about 1,023 ng/ml, about 923 ng/ml to about 1,023 ng/ml, about 973 ng/ml to about 1,023 ng/ml, about 683 ng/ml to about 973 ng/mL, about 733 ng/mL to about 973 ng/ml, about 783 ng/mL to about 973 ng/mL, about 823 ng/ml to about 973 ng/mL, about 873 ng/ml to about 973 ng/mL, about 923 ng/ml to about 973 ng/mL, about 683 ng/mL to about 923 ng/ml, about 733 ng/ml to about 923 ng/ml, about 783 ng/mL to about 923 ng/ml, about 823 ng/mL to about 923 ng/mL, about 873 ng/ml to about 923 ng/ml, about 683 ng/ml to about 873 ng/ml, about 733 ng/ml to about 873 ng/ml, about 783 ng/mL to about 873 ng/ml, about 823 ng/ml to about 873 ng/mL, about 683 ng/mL to about 823 ng/mL, about 733 ng/mL to about 823 ng/ml, about 783 ng/mL to about 823 ng/mL, about 683 ng/ml to about 783 ng/ml, about 733 ng/ml to about 783 ng/ml, or about 683 ng/ml to about 733 ng/ml, including any values or ranges therebetween. In embodiments, administering a therapeutically effective dose of OpSCF (i.e., 10 mg of OpSCF) subcutaneously provides a maximum blood plasma concentration (Cₘₐₓ) ranging from about 810 ng/mL to about 898 ng/mL, about 830 ng/ml to about 898 ng/mL, about 850 ng/ml to about 898 ng/mL, about 870 ng/ml to about 898 ng/mL, about 890 ng/ml to about 898 ng/mL, about 810 ng/ml to about 870 ng/mL, about 830 ng/mL to about 870 ng/mL, about 850 ng/ml to about 870 ng/mL, about 810 ng/mL to about 850 ng/ml, about 830 ng/mL to about 850 ng/ml, about 810 ng/ml to about 830 ng/ml, including any ranges or values therein. In embodiments, a 10 mg subcutaneous dose of the OpSCF provides a maximum blood plasma concentration (Cₘₐₓ) within the range of 80% to 125% of 854±43.8 ng/ml OpSCF. In embodiments, the therapeutically effective dose of the OpSCF provides a maximum blood plasma concentration (Cₘₐₓ) of about 854±43.8 ng/ml OpSCF.

In embodiments, administering a therapeutically effective dose of OpSCF (i.e., 30 mg of OpSCF) subcutaneously provides a maximum blood plasma concentration (Cₘₐₓ) ranging from about 2,864 ng/mL to about 4,475, about 3,064 ng/ml to about 4,475 ng/ml, about 3,264 ng/ml to about 4,475 ng/mL, about 3,464 ng/mL to about 4,475 ng/ml, about 3,664 ng/mL to about 4,475 ng/ml, about 3,864 ng/ml to about 4,475 ng/mL, 4,064 ng/mL to about 4,475 ng/mL, 4,264 ng/ml to about 4,475 ng/ml, about 2,864 ng/mL to about 4,264, about 3,064 ng/mL to about 4,264 ng/mL, about 3,264 ng/mL to about 4,264 ng/ml, about 3,464 ng/ml to about 4,264 ng/ml, about 3,664 ng/ml to about 4,264 ng/mL, about 3,864 ng/ml to about 4,264 ng/ml, 4,064 ng/ml to about 4,264 ng/ml, about 2,864 ng/ml to about 4,064, about 3,064 ng/ml to about 4,064 ng/ml, about 3,264 ng/mL to about 4,064 ng/ml, about 3,464 ng/mL to about 4,064 ng/ml, about 3,664 ng/ml to about 4,064 ng/ml, about 3,864 ng/mL to about 4,064 ng/ml, about 2,864 ng/mL to about 3,864, about 3,064 ng/mL to about 3,864 ng/ml, about 3,264 ng/mL to about 3,864 ng/ml, about 3,464 ng/ml to about 3,864 ng/mL, about 3,664 ng/mL to about 3,864 ng/mL, about 2,864 ng/ml to about 3,664, about 3,064 ng/mL to about 3,664 ng/mL, about 3,264 ng/mL to about 3,664 ng/ml, about 3,464 ng/mL to about 3,664 ng/ml, about 2,864 ng/ml to about 3,464, about 3,064 ng/ml to about 3,464 ng/mL, about 3,264 ng/mL to about 3,464 ng/ml, about 2,864 ng/ml to about 3,264, about 3,064 ng/mL to about 3,264 ng/ml, or about 2,864 ng/ml to about 3,064, including any ranges or values therebetween. In embodiments, administering a therapeutically effective dose of OpSCF (i.e., 30 mg of OpSCF) subcutaneously provides a maximum blood plasma concentration (Cₘₐₓ) ranging from about 3,538 ng/mL to about 3,622 ng/ml, about 3,558 ng/ml to about 3,622 ng/ml, about 3,578 ng/mL to about 3,622 ng/ml, about 3,598 ng/mL to about 3,622 ng/mL, about 3,618 ng/mL to about 3,622 ng/mL, about 3,538 ng/ml to about 3,598 ng/mL, about 3,558 ng/mL to about 3,598 ng/mL, about 3,578 ng/mL to about 3,598 ng/mL, about 3,538 ng/mL to about 3,578 ng/mL, about 3,558 ng/ml to about 3,578 ng/ml, or about 3,538 ng/mL to about 3,558 ng/ml of OpSCF, including any values or rangers therebetween. In embodiments, a 30 mg subcutaneous dose of OpSCF provides a maximum blood plasma concentration (Cₘₐₓ) within the range of 80% to 125% of 3,580±41.8 ng/mL. In embodiments, a 30 mg subcutaneous dose of OpSCF provides a maximum blood plasma concentration (Cₘₐₓ) of about 3,580±41.8 ng/ml OpSCF.

In embodiments, administering a therapeutically effective dose of OpSCF (i.e., 100 mg of OpSCF) subcutaneously provides a maximum blood plasma concentration (Cₘₐₓ) ranging from about 7,920 ng/mL to about 12,375 ng/ml, about 8,420 ng/ml to about 12,375 ng/mL, about 8,920 ng/ml to about 12,375 ng/mL, about 9,420 ng/mL to about 12,375 ng/mL, about 9,920 ng/ml to about 12,375 ng/mL, about 10,420 ng/mL to about 12,375 ng/mL, about 10,920 ng/mL to about 12,375 ng/ml, about 11,420 ng/mL to about 12,375 ng/mL, about 11,920 ng/ml to about 12,375 ng/mL, about 7,920 ng/mL to about 11,920 ng/mL, about 8,420 ng/mL to about 11,920 ng/ml, about 8,920 ng/mL to about 11,920 ng/mL, about 9,420 ng/ml to about 11,920 ng/mL, about 9,920 ng/ml to about 11,920 ng/mL, about 10,420 ng/mL to about 11,920 ng/mL, about 10,920 ng/ml to about 11,920 ng/mL, about 11,420 ng/mL to about 11,920 ng/mL, about 7,920 ng/ml to about 11,420 ng/mL, about 8,420 ng/ml to about 11,420 ng/mL, about 8,920 ng/mL to about 11,420 ng/mL, about 9,420 ng/ml to about 11,420 ng/mL, about 9,920 ng/mL to about 11,420 ng/mL, about 10,420 ng/mL to about 11,420 ng/mL, about 10,920 ng/mL to about 11,420 ng/mL, about 7,920 ng/ml to about 10,920 ng/ml, about 8,420 ng/ml to about 10,920 ng/ml, about 8,920 ng/mL to about 10,920 ng/mL, about 9,420 ng/ml to about 10,920 ng/mL, about 9,920 ng/ml to about 10,920 ng/mL, about 10,420 ng/mL to about 10,920 ng/mL, about 7,920 ng/ml to about 10,420 ng/mL, about 8,420 ng/ml to about 10,420 ng/mL, about 8,920 ng/mL to about 10,420 ng/mL, about 9,420 ng/mL to about 10,420 ng/mL, about 9,920 ng/ml to about 10,420 ng/ml, about 7,920 ng/ml to about 9,920 ng/mL, about 8,420 ng/mL to about 9,920 ng/ml, about 8,920 ng/mL to about 9,920 ng/mL, about 9,420 ng/mL to about 9,920 ng/mL, about 9,920 ng/mL to about 9,920 ng/mL, about 7,920 ng/ml to about 9,420 ng/mL, about 8,420 ng/mL to about 9,420 ng/mL, about 8,920 ng/mL to about 9,420 ng/mL, about 7,920 ng/ml to about 8,920 ng/ml, about 8,420 ng/mL to about 8,920 ng/mL, about 7,920 ng/ml to about 8,420 ng/ml of OpSCF, including any ranges or values therebetween. In embodiments, administering a therapeutically effective dose of OpSCF (i.e., 100 mg of OpSCF) subcutaneously provides a maximum blood plasma concentration (Cₘₐₓ) ranging from about 9,866 ng/ml to about 9,933 ng/mL, about 9,886 ng/ml to about 9,933 ng/mL, about 9,906 ng/mL to about 9,933 ng/mL, about 9,926 ng/ml to about 9,933 ng/mL, about 9,866 ng/mL to about 9,906 ng/ml, about 9,886 ng/mL to about 9,906 ng/ml, or about 9,866 ng/mL to about 9,886 ng/mL of OpSCF, including any values or ranges therebetween. In embodiments, a 100 mg subcutaneous dose of the OpSCF provides a maximum blood plasma concentration (Cₘₐₓ) within the range of 80% to 125% of 9,900±33.2 ng/ml of OpSCF. In embodiments, a 100 mg subcutaneous dose of OpSCF provides a maximum blood plasma concentration (Cₘₐₓ) of about 9,900±33.2 ng/ml of OpSCF.

In embodiments, administering a therapeutically effective dose of OpSCF (i.e., 300 mg of OpSCF) subcutaneously provides a maximum blood plasma concentration (Cₘₐₓ) ranging from about 24,000 ng/mL to about 37,500 ng/mL, about 26,000 ng/ml to about 37,500 ng/mL, about 28,000 ng/mL to about 37,500 ng/mL, about 30,000 ng/mL to about 37,500 ng/mL, about 32,000 ng/mL to about 37,500 ng/mL, about 34,000 ng/mL to about 37,500 ng/ml, about 36,000 ng/ml to about 37,500 ng/mL, about 24,000 ng/mL to about 36,000 ng/mL, about 26,000 ng/ml to about 36,000 ng/mL, about 28,000 ng/mL to about 36,000 ng/mL, about 30,000 ng/mL to about 36,000 ng/mL, about 32,000 ng/ml to about 36,000 ng/mL, about 34,000 ng/ml to about 36,000 ng/ml, about 24,000 ng/mL to about 34,000 ng/mL, about 26,000 ng/mL to about 34,000 ng/ml, about 28,000 ng/mL to about 34,000 ng/mL, about 30,000 ng/mL to about 34,000 ng/ml, about 32,000 ng/mL to about 34,000 ng/mL, about 24,000 ng/mL to about 32,000 ng/ml, about 26,000 ng/ml to about 32,000 ng/mL, about 28,000 ng/ml to about 32,000 ng/mL, about 30,000 ng/mL to about 32,000 ng/mL, about 24,000 ng/mL to about 30,000 ng/mL, about 26,000 ng/ml to about 30,000 ng/mL, about 28,000 ng/mL to about 30,000 ng/mL, about 24,000 ng/ml to about 28,000 ng/ml, about 26,000 ng/mL to about 28,000 ng/mL, or about 24,000 ng/ml to about 26,000 ng/ml, including any values or ranges therebetween. In embodiments, administering a therapeutically effective dose of OpSCF (i.e., 300 mg of OpSCF) subcutaneously provides a maximum blood plasma concentration (Cₘₐₓ) ranging from about 29,924 ng/mL to about 30,076 ng/ml, about 29,944 ng/ml to about 30,076 ng/mL, about 29,964 ng/mL to about 30,076 ng/ml, about 29,984 ng/mL to about 30,076 ng/mL, about 30,004 ng/mL to about 30,076 ng/mL, about 30,024 ng/ml to about 30,076 ng/mL, about 30,044 ng/mL to about 30,076 ng/mL, about 30,064 ng/ml to about 30,076 ng/mL, about 29,924 ng/mL to about 30,044 ng/mL, about 29,944 ng/mL to about 30,044 ng/mL, about 29,964 ng/ml to about 30,044 ng/mL, about 29,984 ng/mL to about 30,044 ng/ml, about 30,004 ng/mL to about 30,044 ng/mL, about 30,024 ng/mL to about 30,044 ng/mL, about 29,924 ng/ml to about 30,024 ng/ml, about 29,944 ng/mL to about 30,024 ng/mL, about 29,964 ng/mL to about 30,024 ng/mL, about 29,984 ng/mL to about 30,024 ng/ml, about 30,004 ng/ml to about 30,024 ng/mL, about 29,924 ng/mL to about 30,004 ng/mL, about 29,944 ng/mL to about 30,004 ng/mL, about 29,964 ng/ml to about 30,004 ng/mL, about 29,984 ng/ml to about 30,004 ng/mL, about 29,924 ng/ml to about 29,984 ng/mL, about 29,944 ng/ml to about 29,984 ng/ml, about 29,964 ng/mL to about 29,984 ng/mL, or about 29,924 ng/ml to about 29,964 ng/ml of OpSCF, including an values or ranges therebetween. In embodiments, a 300 mg subcutaneous dose of OpSCF provides a maximum blood plasma concentration (Cₘₐₓ) within the range of 80% to 125% of 30,000±75.8 ng/mL OpSCF. In embodiments, a 300 mg subcutaneous dose of OpSCF provides a maximum blood plasma concentration (Cₘₐₓ) of about 30,000±75.8 ng/mL.

In embodiments, administering a therapeutically effective dose of OpSCF (i.e., 600 mg of OpSCF) subcutaneously provides a maximum blood plasma concentration (Cₘₐₓ) ranging from about 65,840 ng/mL to about 102,875 ng/mL, about 70,840 ng/mL to about 102,875 ng/ml, about 75,840 ng/mL to about 102,875 ng/mL, about 80,840 ng/mL to about 102,875 ng/mL, about 85,840 ng/mL to about 102,875 ng/mL, about 90,840 ng/ml to about 102,875 ng/ml, about 95,840 ng/ml to about 102,875 ng/mL, about 65,840 ng/ml to about 95,840 ng/mL, about 70,840 ng/mL to about 95,840 ng/mL, about 75,840 ng/ml to about 95,840 ng/mL, about 80,840 ng/ml to about 95,840 ng/mL, about 85,840 ng/ml to about 95,840 ng/mL, about 90,840 ng/mL to about 95,840 ng/ml, about 65,840 ng/mL to about 90,840 ng/mL, about 70,840 ng/mL to about 90,840 ng/ml, about 75,840 ng/mL to about 90,840 ng/mL, about 80,840 ng/mL to about 90,840 ng/ml, about 85,840 ng/ml to about 90,840 ng/mL, about 65,840 ng/ml to about 85,840 ng/mL, about 70,840 ng/ml to about 85,840 ng/mL, about 75,840 ng/ml to about 85,840 ng/ml, about 80,840 ng/mL to about 85,840 ng/mL, about 65,840 ng/mL to about 80,840 ng/mL, about 70,840 ng/ml to about 80,840 ng/mL, about 75,840 ng/mL to about 80,840 ng/mL, about 65,840 ng/mL to about 75,840 ng/ml, about 70,840 ng/ml to about 75,840 ng/mL, about 65,840 ng/mL to about 70,840 ng/ml of OpSCF, including any values or ranges therebetween. In embodiments, administering a therapeutically effective dose of OpSCF (i.e., 600 mg of OpSCF) subcutaneously provides a maximum blood plasma concentration (Cₘₐₓ) ranging from about 82,251 ng/mL to about 82,349 ng/mL, about 82,271 ng/mL to about 82,349 ng/mL, about 82,291 ng/mL to about 82,349 ng/ml, about 83,311 ng/ml to about 82,349 ng/ml, about 83,331 ng/mL to about 82,349 ng/ml, about 82,251 ng/mL to about 83,331 ng/mL, about 82,271 ng/ml to about 83,331 ng/mL, about 82,291 ng/ml to about 83,331 ng/mL, about 83,311 ng/ml to about 83,331 ng/ml, 82,251 ng/mL to about 83,311 ng/mL, about 82,271 ng/mL to about 83,311 ng/ml, about 82,291 ng/mL to about 83,311 ng/mL, 82,251 ng/mL to about 83,291 ng/mL, about 82,271 ng/ml to about 83,291 ng/mL, about, 82,251 ng/ml to about 83,271 ng/ml of OpSCF, including any values or ranged therebetween. In embodiments, a 600 mg subcutaneous dose of OpSCF provides a maximum blood plasma concentration (Cₘₐₓ) within the range of 80% to 125% of 82,300±48.1 ng/ml of OpSCF. In embodiments, a 600 mg subcutaneous dose of OpSCF provides a maximum blood plasma concentration (Cₘₐₓ) of about 82,300±48.1 ng/ml OpSCF.

In embodiments, administering a therapeutically effective dose of OpSCF (i.e., 100 mg of OpSCF) intravenously provides a maximum blood plasma concentration (Cₘₐₓ) ranging from about 34,160 ng/ml to about 53,375 ng/mL, about 39,160 ng/mL to about 53,375 ng/ml, about 44,160 ng/mL to about 53,375 ng/mL, about 49,160 ng/mL to about 53,375 ng/mL, about 34,160 ng/mL to about 49,160 ng/mL, about 39,160 ng/mL to about 49,160 ng/ml, about 44,160 ng/ml to about 49,160 ng/mL, about 34,160 ng/mL to about 44,160 ng/ml, about 39,160 ng/ml to about 44,160 ng/mL, or about 34,160 ng/mL to about 39,160 of OpSCF, including any ranges and values therebetween. In embodiments, administering a therapeutically effective dose of OpSCF (i.e., 100 mg of OpSCF) intravenously provides a maximum blood plasma concentration (Cₘₐₓ) ranging from about 42,684 ng/mL to about 42,716 ng/mL, about 42,689 ng/mL to about 42,716 ng/ml, about 42,694 ng/mL to about 42,716 ng/mL, about 42,699 ng/mL to about 42,716 ng/ml, about 42,704 ng/mL to about 42,716 ng/mL, about 42,709 ng/mL to about 42,716 ng/ml, about 42,684 ng/ml to about 42,709 ng/mL, about 42,689 ng/ml to about 42,709 ng/mL, about 42,694 ng/ml to about 42,709 ng/mL, about 42,699 ng/ml to about 42,709 ng/mL, about 42,704 ng/ml to about 42,709 ng/ml, about 42,684 ng/mL to about 42,704 ng/mL, about 42,689 ng/mL to about 42,704 ng/ml, about 42,694 ng/mL to about 42,704 ng/mL, about 42,699 ng/mL to about 42,704 ng/mL, about 42,684 ng/mL to about 42,699 ng/mL, about 42,689 ng/mL to about 42,699 ng/ml, about 42,694 ng/mL to about 42,699 ng/mL, about 42,684 ng/ml to about 42,694 ng/mL, about 42,689 ng/ml to about 42,694 ng/mL, or about 42,684 ng/mL to about 42,689 ng/mL of OpSCF, including any values or ranged therebetween. In embodiments, a 100 mg intravenous dose of OpSCF provides a maximum blood plasma concentration (Cₘₐₓ) within the range of 80% to 125% of 42,700±15.8 ng/ml of OpSCF. In embodiments, a 100 mg intravenous dose of OpSCF provides a maximum blood plasma concentration (Cₘₐₓ) of about 42,700±15.8 ng/ml of OpSCF.

In embodiments, administering a therapeutically effective dose of OpSCF (i.e., 600 mg of OpSCF) intravenously provides a maximum blood plasma concentration (Cₘₐₓ) ranging from about 230,400 ng/mL to about 360,000 ng/mL, about 240,400 ng/ml to about 360,000 ng/ml, about 250,400 ng/mL to about 360,000 ng/mL, about 250,400 ng/ml to about 360,000 ng/ml, about 260,400 ng/mL to about 360,000 ng/mL, about 270,400 ng/ml to about 360,000 ng/ml, about 280,400 ng/mL to about 360,000 ng/mL, about 290,400 ng/ml to about 360,000 ng/ml, about 300,400 ng/mL to about 360,000 ng/mL, about 310,400 ng/ml to about 360,000 ng/mL, about 320,400 ng/mL to about 360,000 ng/mL, about 330,400 ng/mL to about 360,000 ng/ml, about 340,400 ng/mL to about 360,000 ng/mL, about 350,400 ng/mL to about 360,000 ng/ml, 230,400 ng/mL to about 340,400 ng/mL, about 240,400 ng/ml to about 340,400 ng/mL, about 250,400 ng/ml to about 340,400 ng/mL, about 250,400 ng/mL to about 340,400 ng/ml, about 260,400 ng/mL to about 340,400 ng/mL, about 270,400 ng/ml to about 340,400 ng/ml, about 280,400 ng/mL to about 340,400 ng/mL, about 290,400 ng/ml to about 340,400 ng/ml, about 300,400 ng/mL to about 340,400 ng/mL, about 310,400 ng/mL to about 340,400 ng/ml, about 320,400 ng/mL to about 340,400 ng/mL, about 330,400 ng/mL to about 340,400 ng/ml, 230,400 ng/mL to about 320,400 ng/mL, about 240,400 ng/mL to about 320,400 ng/mL, about 250,400 ng/mL to about 320,400 ng/mL, about 250,400 ng/mL to about 320,400 ng/ml, about 260,400 ng/mL to about 320,400 ng/mL, about 270,400 ng/mL to about 320,400 ng/mL, about 280,400 ng/ml to about 320,400 ng/mL, about 290,400 ng/mL to about 320,400 ng/ml, about 300,400 ng/mL to about 320,400 ng/mL, about 310,400 ng/mL to about 320,400 ng/mL, 230,400 ng/mL to about 300,400 ng/mL, about 240,400 ng/mL to about 300,400 ng/mL, about 250,400 ng/ml to about 300,400 ng/ml, about 250,400 ng/mL to about 300,400 ng/ml, about 260,400 ng/ml to about 300,400 ng/mL, about 270,400 ng/ml to about 300,400 ng/ml, about 280,400 ng/ml to about 300,400 ng/mL, about 290,400 ng/mL to about 300,400 ng/mL, 230,400 ng/ml to about 280,400 ng/mL, about 240,400 ng/mL to about 280,400 ng/mL, about 250,400 ng/ml to about 280,400 ng/mL, about 250,400 ng/mL to about 280,400 ng/mL, about 260,400 ng/ml to about 280,400 ng/mL, about 270,400 ng/ml to about 280,400 ng/mL, 230,400 ng/ml to about 260,400 ng/mL, about 240,400 ng/mL to about 260,400 ng/mL, about 250,400 ng/mL to about 260,400 ng/mL, about 250,400 ng/mL to about 260,400 ng/ml, about 260,400 ng/ml to about 260,400 ng/mL, about 270,400 ng/mL to about 260,400 ng/mL, about 230,400 ng/ml to about 240,400 ng/ml of OpSCF, including any ranges and values therebetween. In embodiments, administering a therapeutically effective dose of OpSCF (i.e., 600 mg of OpSCF) intravenously provides a maximum blood plasma concentration (Cₘₐₓ) ranging from about 287,961 ng/mL to about 288,039 ng/mL, about 287,971 ng/mL to about 288,039 ng/mL, about 287,981 ng/mL to about 288,039 ng/mL, about 287,991 ng/mL to about 288,039 ng/mL, about 288,001 ng/mL to about 288,039 ng/mL, about 288,011 ng/ml to about 288,039 ng/mL, about 288,021 ng/ml to about 288,039 ng/mL, about 288,031 ng/mL to about 288,039 ng/ml, 287,961 ng/mL to about 288,021 ng/ml, about 287,971 ng/mL to about 288,021 ng/ml, about 287,981 ng/mL to about 288,021 ng/ml, about 287,991 ng/mL to about 288,021 ng/mL, about 288,001 ng/mL to about 288,021 ng/ml, about 288,011 ng/ml to about 288,021 ng/mL, 287,961 ng/ml to about 288,001 ng/mL, about 287,971 ng/ml to about 288,001 ng/mL, about 287,981 ng/mL to about 288,001 ng/mL, about 287,991 ng/mL to about 288,001 ng/mL, 287,961 ng/mL to about 287,981 ng/mL, about 287,971 ng/ml to about 287,981 ng/mL, or about 287,961 ng/ml to about 287,971 ng/ml of OpSCF, including any values or ranges therebetween. In embodiments, a 600 mg intravenous dose of OpSCF provides a maximum blood plasma concentration (Cₘₐₓ) within the range of 80% to 125% of 288,000±38.6 ng/mL OpSCF. In embodiments, a 600 mg intravenous dose of OpSCF provides a maximum blood plasma concentration (Cₘₐₓ) of about 288,000±38.6 ng/ml OpSCF.

In embodiments, a 30 mg intravenous dose of OpSCF provides a Cₘₐₓ within the range of 80% to 125% of 11700±22.2 ng/mL of OpSCF. In embodiments, a 30 mg intravenous dose of OpSCF provides a Cₘₐₓ of about 11700±22.2 ng/ml of OpSCF.

In embodiments, a 100 mg intravenous dose of OpSCF provides a Cₘₐₓ within the range of 80% to 125% of 37400±17.9 ng/ml of OpSCF. In embodiments, a 100 mg intravenous dose of OpSCF provides a Cₘₐₓ of about 37400±17.9 ng/ml of OpSCF.

In embodiments, a 300 mg intravenous dose of OpSCF provides a Cₘₐₓ within the range of 80% to 125% of 108000±28.8 ng/mL of OpSCF. In embodiments, a 300 mg intravenous dose of OpSCF provides a Cₘₐₓ of about 108000±28.8 h* ng/ml of OpSCF.

In embodiments, administering multiple 30 mg doses of OpSCF provides a Cₘₐₓ within the range of 80% to 125% of 9370±34.6 ng/ml of OpSCF. In embodiments, administering multiple 30 mg doses of OpSCF provides a Cₘₐₓ of about 9370±34.6 ng/ml of OpSCF.

In embodiments, administering multiple 100 mg doses of OpSCF provides a Cₘₐₓ within the range of 80% to 125% of 39800±27.6 ng/ml of OpSCF. In embodiments, administering multiple 100 mg doses of OpSCF provides a Cₘₐₓ of about 39800±27.6 ng/ml of OpSCF.

In embodiments, administering multiple 300 mg doses of OpSCF provides a Cₘₐₓ within the range of 80% to 125% of 120000±32.3 ng/ml of OpSCF. In embodiments, administering multiple 300 mg doses of OpSCF provides a Cₘₐₓ of about 120000±32.3 ng/mL of OpSCF.

In embodiments, Cₘₐₓ is expressed by DCₘₐₓ that is normalized by the dose administered. The value “DCₘₐₓ” refers to Cₘₐₓ divided by the dose.

In embodiments, administering multiple 30 mg doses of OpSCF provides a Cₘᵢₙ within the range of 80% to 125% of 6030±29.2 ng/ml of OpSCF. In embodiments, administering multiple 30 mg doses of OpSCF provides a Cₘᵢₙ of about 6030±29.2 ng/ml of OpSCF.

In embodiments, administering multiple 100 mg doses of OpSCF provides a Cₘᵢₙ within the range of 80% to 125% of 28300±26.7 ng/mL of OpSCF. In embodiments, administering multiple 100 mg doses of OpSCF provides a Cₘᵢₙ of about 28300±26.7 ng/ml of OpSCF.

In embodiments, administering multiple 300 mg doses of OpSCF provides a Cₘᵢₙ within the range of 80% to 125% of 63300±77 ng/ml of OpSCF. In embodiments, administering multiple 300 mg doses of the OpSCF provides a Cₘᵢₙ of about 63300±77 ng/ml of OpSCF.

In embodiments, Cₘᵢₙ is expressed by DCₘᵢₙ that is normalized by the dose administered. The value “DCₘᵢₙ” refers to Cₘᵢₙ divided by the dose.

AUCₜ (AUC from Time 0 to the Last Measurable Concentration)

In embodiments, administering a therapeutically effective dose of OpSCF provides an AUCₜ level ranging from about 0.05 h*ng/ml to about 100000000 h*ng/m, including about 0.05 h*ng/mL, about 0.5 h*ng/mL, about 1000000 h*ng/ml, about 2000000 h*ng/mL, about 3000000 h*ng/mL, about 4000000 h*ng/ml, about 5000000 h*ng/mL, about 6000000 h*ng/ml, about 7000000 h*ng/mL, about 8000000 h*ng/mL, about 9000000 h*ng/mL, about 10000000 h*ng/ml, about 11000000 h*ng/mL, about 12000000 h*ng/ml, about 13000000 h*ng/mL, about 14000000 h*ng/mL, about 15000000 h*ng/ml, about 16000000 h*ng/ml, about 17000000 h*ng/ml, about 18000000 h*ng/mL, about 19000000 h*ng/ml, about 20000000 h*ng/ml, about 21000000 h*ng/mL, about 22000000 h*ng/mL, about 23000000 h*ng/mL, about 24000000 h*ng/ml, about 25000000 h*ng/ml, about 26000000 h*ng/ml, about 27000000 h*ng/ml, about 28000000 h*ng/mL, about 29000000 h*ng/mL, about 30000000 h*ng/mL, about 31000000 h*ng/mL, about 32000000 h*ng/mL, about 33000000 h*ng/ml, about 34000000 h*ng/ml, about 35000000 h*ng/mL, about 36000000 h*ng/mL, about 37000000 h*ng/mL, about 38000000 h*ng/mL, about 39000000 h*ng/mL, about 40000000 h*ng/ml, about 41000000 h*ng/ml, about 42000000 h*ng/ml, about 43000000 h*ng/mL, about 44000000 h*ng/ml, about 45000000 h*ng/ml, about 46000000 h*ng/mL, about 47000000 h*ng/ml, about 48000000 h*ng/mL, about 49000000 h*ng/ml, about 50000000 h*ng/mL, about 51000000 h*ng/mL, about 52000000 h*ng/ml, about 53000000 h*ng/ml, about 54000000 h*ng/mL, about 55000000 h*ng/ml, about 56000000 h*ng/mL, about 57000000 h*ng/mL, about 58000000 h*ng/mL, about 59000000 h*ng/ml, about 60000000 h*ng/ml, about 61000000 h*ng/ml, about 62000000 h*ng/ml, about 63000000 h*ng/mL, about 64000000 h*ng/mL, about 65000000 h*ng/mL, about 66000000 h*ng/ml, about 67000000 h*ng/mL, about 68000000 h*ng/mL, about 69000000 h*ng/ml, about 70000000 h*ng/ml, about 71000000 h*ng/mL, about 72000000 h*ng/mL, about 73000000 h*ng/mL, about 74000000 h*ng/mL, about 75000000 h*ng/ml about, 76000000 h*ng/ml, about 77000000 h*ng/mL, about 78000000 h*ng/mL, about 79000000 h*ng/ml, about 80000000 h*ng/ml, about 81000000 h*ng/ml, about 82000000 h*ng/mL, about 83000000 h*ng/ml, about 84000000 h*ng/mL, about 85000000 h*ng/mL, about 86000000 h*ng/ml, about 87000000 h*ng/ml, about 88000000 h*ng/ml, about 89000000 h*ng/ml, about 90000000 h*ng/ml, about 91000000 h*ng/mL, about 92000000 h*ng/ml, about 92300000 h*ng/mL, about 93300000 h*ng/m, about 94300000 h*ng/m, about 95300000 h*ng/m, about 96300000 h*ng/m, about 97300000 h*ng/m, about 98300000 h*ng/m, about 99300000 h*ng/m, or about 100000000 h*ng/m, or including any values or ranges therebetween.

In embodiments, administering a therapeutically effective dose of OpSCF provides an AUCₜ level ranging from about 0.05 h*ng/ml to about 100,000,000 h*ng/mL, about 500,000 h*ng/ml to about 100,000,000 h*ng/ml, about 1,000,000 h*ng/mL to about 100,000,000 h*ng/ml, about 10,000,000 h*ng/ml to about 100,000,000 h*ng/mL, about 20,000,000 h*ng/ml to about 100,000,000 h*ng/mL, about 30,000,000 h*ng/mL to about 100,000,000 h*ng/ml, about 40,000,000 h*ng/mL to about 100,000,000 h*ng/mL, about 50,000,000 h*ng/ml to about 100,000,000 h*ng/mL, about 60,000,000 h*ng/ml to about 100,000,000 h*ng/ml, about 70,000,000 h*ng/ml to about 100,000,000 h*ng/mL, about 80,000,000 h*ng/ml to about 100,000,000 h*ng/mL, about 90,000,000 h*ng/ml to about 100,000,000 h*ng/ml, about 0.05 h*ng/mL to about 92,300,000 h*ng/mL, about 500,000 h*ng/mL to about 92,300,000 h*ng/ml, about 1,000,000 h*ng/ml to about 92,300,000 h*ng/mL, about 10,000,000 h*ng/ml to about 92,300,000 h*ng/mL, about 20,000,000 h*ng/mL to about 92,300,000 h*ng/mL, about 30,000,000 h*ng/mL to about 92,300,000 h*ng/mL, about 40,000,000 h*ng/mL to about 92,300,000 h*ng/ml, about 50,000,000 h*ng/mL to about 92,300,000 h*ng/mL, about 60,000,000 h*ng/ml to about 92,300,000 h*ng/mL, about 70,000,000 h*ng/mL to about 92,300,000 h*ng/mL, about 80,000,000 h*ng/mL to about 92,300,000 h*ng/mL, about 90,000,000 h*ng/mL to about 92,300,000 h*ng/ml, 0.05 h*ng/mL to about 80,000,000 h*ng/mL, about 500,000 h*ng/ml to about 80,000,000 h*ng/mL, about 1,000,000 h*ng/mL to about 80,000,000 h*ng/mL, about 10,000,000 h*ng/ml to about 80,000,000 h*ng/mL, about 20,000,000 h*ng/mL to about 80,000,000 h*ng/ml, about 30,000,000 h*ng/mL to about 80,000,000 h*ng/ml, about 40,000,000 h*ng/ml to about 80,000,000 h*ng/mL, about 50,000,000 h*ng/mL to about 80,000,000 h*ng/mL, about 60,000,000 h*ng/ml to about 80,000,000 h*ng/mL, about 70,000,000 h*ng/mL to about 80,000,000 h*ng/ml, 0.05 h*ng/ml to about 70,000,000 h*ng/mL, about 500,000 h*ng/ml to about 70,000,000 h*ng/mL, about 1,000,000 h*ng/mL to about 70,000,000 h*ng/mL, about 10,000,000 h*ng/ml to about 70,000,000 h*ng/mL, about 20,000,000 h*ng/mL to about 70,000,000 h*ng/ml, about 30,000,000 h*ng/mL to about 70,000,000 h*ng/mL, about 40,000,000 h*ng/ml to about 70,000,000 h*ng/mL, about 50,000,000 h*ng/mL to about 70,000,000 h*ng/mL, about 60,000,000 h*ng/ml to about 70,000,000 h*ng/mL, 0.05 h*ng/ml to about 60,000,000 h*ng/ml, about 500,000 h*ng/mL to about 60,000,000 h*ng/mL, about 1,000,000 h*ng/mL to about 60,000,000 h*ng/mL, about 10,000,000 h*ng/mL to about 60,000,000 h*ng/mL, about 20,000,000 h*ng/ml to about 60,000,000 h*ng/mL, about 30,000,000 h*ng/mL to about 60,000,000 h*ng/ml, about 40,000,000 h*ng/mL to about 60,000,000 h*ng/ml, about 50,000,000 h*ng/ml to about 60,000,000 h*ng/mL, 0.05 h*ng/mL to about 50,000,000 h*ng/mL, about 500,000 h*ng/ml to about 50,000,000 h*ng/mL, about 1,000,000 h*ng/mL to about 50,000,000 h*ng/mL, about 10,000,000 h*ng/mL to about 50,000,000 h*ng/ml, about 20,000,000 h*ng/ml to about 50,000,000 h*ng/mL, about 30,000,000 h*ng/mL to about 50,000,000 h*ng/mL, about 40,000,000 h*ng/ml to about 50,000,000 h*ng/mL, 0.05 h*ng/mL to about 40,000,000 h*ng/mL, about 500,000 h*ng/ml to about 40,000,000 h*ng/mL, about 1,000,000 h*ng/mL to about 40,000,000 h*ng/mL, about 10,000,000 h*ng/mL to about 40,000,000 h*ng/mL, about 20,000,000 h*ng/ml to about 40,000,000 h*ng/mL, about 30,000,000 h*ng/mL to about 40,000,000 h*ng/mL, 0.05 h*ng/mL to about 30,000,000 h*ng/mL, about 500,000 h*ng/ml to about 30,000,000 h*ng/mL, about 1,000,000 h*ng/ml to about 30,000,000 h*ng/ml, about 10,000,000 h*ng/mL to about 30,000,000 h*ng/mL, about 20,000,000 h*ng/mL to about 30,000,000 h*ng/ml, 0.05 h*ng/ml to about 20,000,000 h*ng/ml, about 500,000 h*ng/mL to about 20,000,000 h*ng/mL, about 1,000,000 h*ng/ml to about 20,000,000 h*ng/ml, about 10,000,000 h*ng/mL to about 20,000,000 h*ng/mL, 0.05 h*ng/ml to about 10,000,000 h*ng/ml, about 500,000 h*ng/ml to about 10,000,000 h*ng/mL, about 1,000,000 h*ng/mL to about 10,000,000 h*ng/ml, 0.05 h*ng/mL to about 1,000,000 h*ng/mL, about 500,000 h*ng/mL to about 1,000,000 h*ng/ml, or 0.05 h*ng/mL to, about 500,000 h*ng/mL, including any values or ranges therebetween.

In embodiments, administering a therapeutically effective dose of OpSCF provides an AUCₜ level ranging from about 555,000 h*ng/mL to about 92,300,000 h*ng/ml (e.g., 555,000, 2240000, 6570000, 2400000, 49600000, 11300000 or 92300000 h*ng/ml, including any values or ranges therebetween.)

In embodiments, administering a therapeutically effective dose of OpSCF (i.e., 10 mg of OpSCF) subcutaneously provides an AUCₜ level ranging from about 444,000 h*ng/mL to about 693,750 h*ng/ml (e.g., about 444000 h*ng/mL, 454000 h*ng/ml, 464000 h*ng/ml, 474000 h*ng/mL, 484000 h*ng/mL, 494000 h*ng/mL, 504000 h*ng/ml, 514000 h*ng/ml, 524000 h*ng/ml, 534000 h*ng/mL, 544000 h*ng/ml, 554000 h*ng/ml, 564000 h*ng/mL, 574000 h*ng/mL, 584000 h*ng/mL, 594000 h*ng/mL, 604000 h*ng/ml, 614000 h*ng/mL, 624000 h*ng/mL, 634000 h*ng/ml, 644000 h*ng/mL, 654000 h*ng/mL, 664000 h*ng/ml, 674000 h*ng/ml, 684000 h*ng/ml, or 693,750 h*ng/mL, including any ranges or values therebetween). In embodiments, administering a therapeutically effective dose of OpSCF (i.e., 10 mg of OpSCF) subcutaneously provides an AUCₜ level ranging from about 554,964 h*ng/mL to about 555,036 h*ng/ml (e.g., about 554964 h*ng/mL, 554969 h*ng/ml, 554974 h*ng/ml, 554979 h*ng/mL, 554984 h*ng/ml, 554989 h*ng/ml, 554994 h*ng/ml, 554999 h*ng/mL, 555004 h*ng/mL, 555009 h*ng/ml, 555014 h*ng/ml, 555019 h*ng/ml, 555024 h*ng/mL, 555029 h*ng/mL, or 555034 h*ng/ml, including any values or ranges therebetween). In embodiments, a 10 mg subcutaneous dose of OpSCF provides an AUCₜ within the range of 80% to 125% of 555000±35.9 h*ng/mL L. In embodiments, a 10 mg subcutaneous dose of OpSCF provides an AUCₜ of about 555000±35.9 h*ng/mL.

In embodiments, administering a therapeutically effective dose of OpSCF (i.e., 30 mg of OpSCF) subcutaneously provides an AUCₜ level ranging from about 1,792,000 h*ng/ml to about 2,800,000 h*ng/mL (e.g., about 1792000 h*ng/mL, 1802000 h*ng/ml, 1812000 h*ng/ml, 1822000 h*ng/mL, 1832000 h*ng/mL, 1842000 h*ng/mL, 1852000 h*ng/ml, 1862000 h*ng/ml, 1872000 h*ng/mL, 1882000 h*ng/mL, 1892000 h*ng/mL, 1902000 h*ng/ml, 1912000 h*ng/mL, 1922000 h*ng/mL, 1932000 h*ng/mL, 1942000 h*ng/mL, 1952000 h*ng/mL, 1962000 h*ng/mL, 1972000 h*ng/mL, 1982000 h*ng/mL, 1992000 h*ng/mL, 2002000 h*ng/mL, 2012000 h*ng/ml, 2022000 h*ng/mL, 2032000 h*ng/mL, 2042000 h*ng/mL, 2052000 h*ng/ml, 2062000 h*ng/ml, 2072000 h*ng/mL, 2082000 h*ng/mL, 2092000 h*ng/mL, 2102000 h*ng/mL, 2112000 h*ng/ml, 2122000 h*ng/mL, 2132000 h*ng/mL, 2142000 h*ng/ml, 2152000 h*ng/ml, 2162000 h*ng/ml, 2172000 h*ng/ml, 2182000 h*ng/mL, 2192000 h*ng/mL, 2202000 h*ng/ml, 2212000 h*ng/mL, 2222000 h*ng/mL, 2232000 h*ng/mL, 2242000 h*ng/mL, 2252000 h*ng/mL, 2262000 h*ng/ml, 2272000 h*ng/mL, 2282000 h*ng/mL, 2292000 h*ng/mL, 2302000 h*ng/mL, 2312000 h*ng/ml, 2322000 h*ng/mL, 2332000 h*ng/mL, 2342000 h*ng/mL, 2352000 h*ng/mL, 2362000 h*ng/ml, 2372000 h*ng/mL, 2382000 h*ng/mL, 2392000 h*ng/mL, 2402000 h*ng/ml, 2412000 h*ng/ml, 2422000 h*ng/mL, 2432000 h*ng/mL, 2442000 h*ng/mL, 2452000 h*ng/ml, 2462000 h*ng/mL, 2472000 h*ng/mL, 2482000 h*ng/mL, 2492000 h*ng/ml, 2502000 h*ng/mL, 2512000 h*ng/ml, 2522000 h*ng/mL, 2532000 h*ng/ml, 2542000 h*ng/mL, 2552000 h*ng/mL, 2562000 h*ng/ml, 2572000 h*ng/ml, 2582000 h*ng/mL, 2592000 h*ng/mL, 2602000 h*ng/ml, 2612000 h*ng/ml, 2622000 h*ng/mL, 2632000 h*ng/mL, 2642000 h*ng/mL, 2652000 h*ng/mL, 2662000 h*ng/ml, 2672000 h*ng/mL, 2682000 h*ng/ml, 2692000 h*ng/mL, 2702000 h*ng/mL, 2712000 h*ng/ml, 2722000 h*ng/ml, 2732000 h*ng/ml, 2742000 h*ng/ml, 2752000 h*ng/ml, 2762000 h*ng/mL, 2772000 h*ng/ml, 2782000 h*ng/ml, 2792000 h*ng/mL, or 2800000 h*ng/mL including any ranges or values therebetween). In embodiments, administering a therapeutically effective dose of OpSCF (i.e., 30 mg of OpSCF) subcutaneously provides an AUCₜ level ranging from about 2,239,977 h*ng/ml to about 2,240,024 h*ng/ml (e.g., about 2239977 h*ng/ml, 2239982 h*ng/mL, 2239987 h*ng/mL, 2239992 h*ng/mL, 2239997 h*ng/ml, 2240002 h*ng/mL, 2240007 h*ng/mL, 2240012 h*ng/ml, 2240017 h*ng/mL, 2240022 h*ng/mL or 2,240,024 h*ng/ml, including any values or ranges therebetween). In embodiments, a 30 mg subcutaneous dose of OpSCF provides an AUCₜ within the range of 80% to 125% of 2240000±23.4 h*ng/mL L. In embodiments, a 30 mg subcutaneous dose of OpSCF provides an AUCₜ of about 2240000±23.4 h*ng/mL.

In embodiments, administering a therapeutically effective dose of OpSCF (i.e., 100 mg of OpSCF) subcutaneously provides an AUCₜ level ranging from about 5,256,000 h*ng/ml to about 8,212,500 h*ng/mL (e.g., about 5256000 h*ng/ml, 5306000 h*ng/ml, 5356000 h*ng/ml, 5406000 h*ng/mL, 5456000 h*ng/mL, 5506000 h*ng/mL, 5556000 h*ng/mL, 5606000 h*ng/mL, 5656000 h*ng/mL, 5706000 h*ng/mL, 5756000 h*ng/mL, 5806000 h*ng/mL, 5856000 h*ng/ml, 5906000 h*ng/ml, 5956000 h*ng/mL, 6006000 h*ng/mL, 6056000 h*ng/mL, 6106000 h*ng/mL, 6156000 h*ng/mL, 6206000 h*ng/mL, 6256000 h*ng/mL, 6306000 h*ng/ml, 6356000 h*ng/ml, 6406000 h*ng/mL, 6456000 h*ng/mL, 6506000 h*ng/ml, 6556000 h*ng/mL, 6606000 h*ng/mL, 6656000 h*ng/mL, 6706000 h*ng/mL, 6756000 h*ng/mL, 6806000 h*ng/mL, 6856000 h*ng/mL, 6906000 h*ng/mL, 6956000 h*ng/mL, 7006000 h*ng/mL, 7056000 h*ng/mL, 7106000 h*ng/mL, 7156000 h*ng/mL, 7206000 h*ng/mL, 7256000 h*ng/mL, 7306000 h*ng/mL, 7356000 h*ng/ml, 7406000 h*ng/mL, 7456000 h*ng/mL, 7506000 h*ng/ml, 7556000 h*ng/mL, 7606000 h*ng/ml, 7656000 h*ng/ml, 7706000 h*ng/mL, 7756000 h*ng/ml, 7806000 h*ng/ml, or 8,212,500 h*ng/ml, including any values or ranges therebetween). In embodiments, a 100 mg subcutaneous dose of OpSCF provides an AUCₜ within the range of 80% to 125% of 6,570,000±26.3 h*ng/ml L. In embodiments, a 100 mg subcutaneous dose of OpSCF provides an AUCₜ of about 6,570,000±26.3 h*ng/mL.

In embodiments, administering a therapeutically effective dose of OpSCF (i.e., 300 mg of OpSCF) subcutaneously provides an AUCₜ ranging from about 1,920,000 h*ng/ml to about 3,000,000 h*ng/ml (e.g., 1920000 h*ng/mL, 1940000 h*ng/mL, 1990000 h*ng/ml, 2040000 h*ng/ml, 2090000 h*ng/mL, 2140000 h*ng/mL, 2190000 h*ng/mL, 2240000 h*ng/ml, 2290000 h*ng/mL, 2340000 h*ng/mL, 2390000 h*ng/ml, 2440000 h*ng/mL, 2490000 h*ng/mL, 2540000 h*ng/mL, 2590000 h*ng/mL, 2640000 h*ng/mL, 2690000 h*ng/ml, 2740000 h*ng/ml, 2790000 h*ng/ml, 2840000 h*ng/ml, 2890000 h*ng/mL, 2940000 h*ng/ml, 2990000 h*ng/ml, or 3,000,000 h*ng/mL, including any values or rangers therebetween). In embodiments, a 300 mg subcutaneous dose of OpSCF provides an AUCₜ within the range of 80% to 125% of 2,400,000±40.7 h*ng/mL L. In embodiments, a 300 mg subcutaneous dose of OpSCF provides an AUCₜ of about 2,400,000±40.7 h*ng/mL.

In embodiments, administering a therapeutically effective dose of OpSCF (i.e., 600 mg of OpSCF) subcutaneously provides an AUCₜ ranging from about 39,680,000 h*ng/ml to about 62,000,000 h*ng/ml (e.g., about 39680000 h*ng/mL, 40180000 h*ng/mL, 40680000 h*ng/ml, 41180000 h*ng/ml, 41680000 h*ng/ml, 42180000 h*ng/ml, 42680000 h*ng/ml, 43180000 h*ng/ml, 43680000 h*ng/mL, 44180000 h*ng/mL, 44680000 h*ng/ml, 45180000 h*ng/ml, 45680000 h*ng/ml, 46180000 h*ng/ml, 46680000 h*ng/ml, 47180000 h*ng/mL, 47680000 h*ng/mL, 48180000 h*ng/mL, 48680000 h*ng/ml, 49180000 h*ng/ml, 49680000 h*ng/ml, 50180000 h*ng/ml, 50680000 h*ng/ml, 51180000 h*ng/mL, 51680000 h*ng/ml, 52180000 h*ng/mL, 52680000 h*ng/mL, 53180000 h*ng/mL, 53680000 h*ng/mL, 54180000 h*ng/ml, 54680000 h*ng/ml, 55180000 h*ng/mL, 55680000 h*ng/ml, 56180000 h*ng/mL, 56680000 h*ng/ml, 57180000 h*ng/mL, 57680000 h*ng/ml, 58180000 h*ng/ml, 58680000 h*ng/mL, 59180000 h*ng/ml, 59680000 h*ng/mL, 60180000 h*ng/mL, 60680000 h*ng/ml, 61180000 h*ng/ml, 61680000 h*ng/mL, or 62000000 h*ng/ml, including any values or rangers therebetween). In embodiments, a 600 mg subcutaneous dose of OpSCF provides an AUCₜ within the range of 80% to 125% of 49,600,000±32.5 h*ng/mL L. In embodiments, a 600 mg subcutaneous dose of OpSCF provides an AUCₜ of about 49,600,000±32.5 h*ng/mL.

In embodiments, administering a therapeutically effective dose of OpSCF (i.e., 100 mg of OpSCF) intravenously provides an AUCₜ ranging from about 9,040,000 h*ng/mL to about 14,125,000 h*ng/ml (e.g., about 9040000 h*ng/ml, 9090000 h*ng/ml, 9140000 h*ng/ml, 9190000 h*ng/mL, 9240000 h*ng/mL, 9290000 h*ng/mL, 9340000 h*ng/ml, 9390000 h*ng/ml, 9440000 h*ng/mL, 9490000 h*ng/mL, 9540000 h*ng/mL, 9590000 h*ng/ml, 9640000 h*ng/ml, 9690000 h*ng/mL, 9740000 h*ng/mL, 9790000 h*ng/mL, 9840000 h*ng/mL, 9890000 h*ng/ml, 9940000 h*ng/mL, 9990000 h*ng/mL, 10040000 h*ng/ml, 10090000 h*ng/mL, 10140000 h*ng/mL, 10190000 h*ng/mL, 10240000 h*ng/mL, 10290000 h*ng/mL, 10340000 h*ng/ml, 10390000 h*ng/mL, 10440000 h*ng/ml, 10490000 h*ng/mL, 10540000 h*ng/ml, 10590000 h*ng/ml, 10640000 h*ng/mL, 10690000 h*ng/mL, 10740000 h*ng/ml, 10790000 h*ng/ml, 10840000 h*ng/mL, 10890000 h*ng/mL, 10940000 h*ng/ml, 10990000 h*ng/mL, 11040000 h*ng/mL, 11090000 h*ng/mL, 11140000 h*ng/mL, 11190000 h*ng/ml, 11240000 h*ng/ml, 11290000 h*ng/ml, 11340000 h*ng/ml, 11390000 h*ng/ml, 11440000 h*ng/ml, 11490000 h*ng/mL, 11540000 h*ng/ml, 11590000 h*ng/ml, 11640000 h*ng/ml, 11690000 h*ng/ml, 11740000 h*ng/ml, 11790000 h*ng/ml, 11840000 h*ng/ml, 11890000 h*ng/ml, 11940000 h*ng/mL, 11990000 h*ng/mL, 12040000 h*ng/mL, 12090000 h*ng/ml, 12140000 h*ng/ml, 12190000 h*ng/ml, 12240000 h*ng/ml, 12290000 h*ng/ml, 12340000 h*ng/ml, 12390000 h*ng/mL, 12440000 h*ng/mL, 12490000 h*ng/mL, 12540000 h*ng/ml, 12590000 h*ng/ml, 12640000 h*ng/ml, 12690000 h*ng/mL, 12740000 h*ng/ml, 12790000 h*ng/ml, 12840000 h*ng/mL, 12890000 h*ng/mL, 12940000 h*ng/mL, 12990000 h*ng/ml, 13040000 h*ng/ml, 13090000 h*ng/ml, 13140000 h*ng/ml, 13190000 h*ng/ml, 13240000 h*ng/mL, 13290000 h*ng/ml, 13340000 h*ng/ml, 13390000 h*ng/mL, 13440000 h*ng/mL, 13490000 h*ng/ml, 13540000 h*ng/ml, 13590000 h*ng/ml, 13640000 h*ng/ml, 13690000 h*ng/ml, 13740000 h*ng/mL, 13790000 h*ng/mL, 13840000 h*ng/mL, 13890000 h*ng/ml, 13940000 h*ng/ml, 13990000 h*ng/mL, 14040000 h*ng/mL, 14090000 h*ng/mL, or 14,125,000 h*ng/mL, including any values or ranges therebetween). In embodiments, a 100 mg intravenous dose of OpSCF provides an AUCₜ within the range of 80% to 125% of 11300000±19.9 h*ng/mL L. In embodiments, a 100 mg intravenous dose of OpSCF provides an AUCₜ of about 11300000±19.9 h*ng/mL.

In embodiments, administering a therapeutically effective dose of OpSCF (i.e., 600 mg of OpSCF) intravenously provides an AUCₜ ranging from about 73,840,000 h*ng/ml to about 115,375,000 h*ng/ml (e.g., 73840000 h*ng/ml, 74340000 h*ng/ml, 74840000 h*ng/ml, 75340000 h*ng/mL, 75840000 h*ng/mL, 76340000 h*ng/ml, 76840000 h*ng/mL, 77340000 h*ng/ml, 77840000 h*ng/ml, 78340000 h*ng/ml, 78840000 h*ng/ml, 79340000 h*ng/ml, 79840000 h*ng/mL, 80340000 h*ng/mL, 80840000 h*ng/ml, 81340000 h*ng/ml, 81840000 h*ng/mL, 82340000 h*ng/ml, 82840000 h*ng/mL, 83340000 h*ng/mL, 83840000 h*ng/ml, 84340000 h*ng/ml, 84840000 h*ng/ml, 85340000 h*ng/ml, 85840000 h*ng/ml, 86340000 h*ng/ml, 86840000 h*ng/ml, 87340000 h*ng/mL, 87840000 h*ng/mL, 88340000 h*ng/ml, 88840000 h*ng/mL, 89340000 h*ng/mL, 89840000 h*ng/mL, 90340000 h*ng/ml, 90840000 h*ng/ml, 91340000 h*ng/mL, 91840000 h*ng/mL, 92340000 h*ng/ml, 92840000 h*ng/ml, 93340000 h*ng/mL, 93840000 h*ng/mL, 94340000 h*ng/ml, 94840000 h*ng/mL, 95340000 h*ng/ml, 95840000 h*ng/mL, 96340000 h*ng/mL, 96840000 h*ng/mL, 97340000 h*ng/ml, 97840000 h*ng/mL, 98340000 h*ng/ml, 98840000 h*ng/mL, 99340000 h*ng/ml, 99840000 h*ng/mL, 100340000 h*ng/mL, 100840000 h*ng/mL, 101340000 h*ng/ml, 101840000 h*ng/ml, 102340000 h*ng/ml, 102840000 h*ng/ml, 103340000 h*ng/ml, 103840000 h*ng/ml, 104340000 h*ng/ml, 104840000 h*ng/mL, 105340000 h*ng/ml, 105840000 h*ng/ml, 106340000 h*ng/mL, 106840000 h*ng/mL, 107340000 h*ng/ml, 107840000 h*ng/ml, 108340000 h*ng/ml, 108840000 h*ng/ml, 109340000 h*ng/mL, 109840000 h*ng/mL, 110340000 h*ng/mL, 110840000 h*ng/mL, 111340000 h*ng/ml, 111840000 h*ng/ml, 112340000 h*ng/ml, 112840000 h*ng/ml, 113340000 h*ng/ml, 113840000 h*ng/mL, 114340000 h*ng/mL, 114840000 h*ng/ml, 115340000 h*ng/ml, or 115375000 h*ng/ml, including any values or rangers therebetween. In embodiments, a 600 mg intravenous dose of OpSCF provides an AUCₜ within the range of 80% to 125% of 92300000±25.0 h*ng/ml L. In embodiments, a 600 mg intravenous dose of OpSCF provides an AUCₜ of about 92300000±25.0 h*ng/ml.

In embodiments, AUCₜ is expressed by DAUCₜ that is normalized by the dose administered. The value “DAUCₜ” refers to AUCₜ divided by the dose.

AUC_{0-168 hours}

In embodiments, a 30 mg intravenous dose of the OpSCF provides a AUC_{0-168 hours} within the range of 80% to 125% of 1100000±15.5 h*ng/mL. In embodiments, a 30 mg multiple dose of the OpSCF provides an AUC_{0-168 hours} of about 1100000±15.5 h*ng/ml.

In embodiments, a 100 mg intravenous dose of the OpSCF provides a AUC_{0-168 hours} within the range of 80% to 125% of 4200000±17.7 h*ng/mL. In embodiments, a 100 mg multiple dose of the OpSCF provides an AUC_{0-168 hours} of about 4200000±17.7 h*ng/ml.

In embodiments, a 300 mg intravenous dose of the OpSCF provides a AUC_{0-168 hours} within the range of 80% to 125% of 10800000±23.0 h*ng/mL. In embodiments, a 300 mg multiple dose of the OpSCF provides an AUC_{0-168 hours} of about 10800000±23.0 h*ng/mL.

In embodiments, administering multiple 30 mg doses of the OpSCF provides an AUC_{0-168 hours} within the range of 80% to 125% of 1350000±30.1 h*ng/mL. In embodiments, administering multiple 30 mg doses of OpSCF provides an AUC_{0-168 hours} of about 1350000±30.1 h*ng/mL.

In embodiments, administering multiple 100 mg doses of OpSCF provides an AUC_{0-168 hours} within the range of 80% to 125% of 6080000±27.1 h*ng/mL. In embodiments, administering multiple 100 mg doses of OpSCF provides an AUC_{0-168 hours} of about 6080000±27.1 h*ng/ml.

In embodiments, administering multiple 300 mg doses of OpSCF provides an AUC_{0-168 hours} within the range of 80% to 125% of 17200000±35.6 h*ng/ml. In embodiments, administering multiple 300 mg doses of OpSCF provides an AUC_{0-168 hours} of about 17200000±35.6 h*ng/mL.

In embodiments, AUC_{0-168 hours} is expressed by DAUC_{0-168 hours} that is normalized by the dose administered. The value “DAUC_{0-168 hours}” refers to AUC_{0-168 hours} divided by the dose.

AUC_{inf} (AUC from Time 0 Extrapolated to Infinity)

In embodiments, administering a therapeutically effective dose of OpSCF provides an AUC_{inf} ranging from about 0.05 h*ng/mL to about 100000000 h*ng/m, including about 0.05 h*ng/mL, about 0.5 h*ng/ml, about 1000000 h*ng/mL, about 2000000 h*ng/mL, about 3000000 h*ng/ml, about 4000000 h*ng/mL, about 5000000 h*ng/ml, about 6000000 h*ng/ml, about 7000000 h*ng/mL, about 8000000 h*ng/ml, about 9000000 h*ng/mL, about 10000000 h*ng/ml, about 11000000 h*ng/mL, about 12000000 h*ng/mL, about 13000000 h*ng/ml, about 14000000 h*ng/ml, about 15000000 h*ng/ml, about 16000000 h*ng/ml, about 17000000 h*ng/ml, about 18000000 h*ng/ml, about 19000000 h*ng/mL, about 20000000 h*ng/ml, about 21000000 h*ng/mL, about 22000000 h*ng/ml, about 23000000 h*ng/mL, about 24000000 h*ng/ml, about 25000000 h*ng/ml, about 26000000 h*ng/mL, about 27000000 h*ng/ml, about 28000000 h*ng/mL, about 29000000 h*ng/mL, about 30000000 h*ng/ml, about 31000000 h*ng/mL, about 32000000 h*ng/ml, about 33000000 h*ng/mL, about 34000000 h*ng/ml, about 35000000 h*ng/mL, about 36000000 h*ng/mL, about 37000000 h*ng/mL, about 38000000 h*ng/ml, about 39000000 h*ng/mL, about 40000000 h*ng/mL, about 41000000 h*ng/ml, about 42000000 h*ng/mL, about 43000000 h*ng/mL, about 44000000 h*ng/mL, about 45000000 h*ng/ml, about 46000000 h*ng/ml, about 47000000 h*ng/ml, about 48000000 h*ng/ml, about 49000000 h*ng/mL, about 50000000 h*ng/mL, about 51000000 h*ng/ml, about 52000000 h*ng/mL, about 53000000 h*ng/ml, about 54000000 h*ng/ml, about 55000000 h*ng/ml, about 56000000 h*ng/ml, about 57000000 h*ng/mL, about 58000000 h*ng/ml, about 59000000 h*ng/mL, about 60000000 h*ng/mL, about 61000000 h*ng/mL, about 62000000 h*ng/ml, about 63000000 h*ng/mL, about 64000000 h*ng/ml, about 65000000 h*ng/mL, about 66000000 h*ng/mL, about 67000000 h*ng/mL, about 68000000 h*ng/mL, about 69000000 h*ng/ml, about 70000000 h*ng/mL, about 71000000 h*ng/ml, about 72000000 h*ng/mL, about 73000000 h*ng/mL, about 74000000 h*ng/ml, about 75000000 h*ng/ml about, 76000000 h*ng/ml, about 77000000 h*ng/mL, about 78000000 h*ng/ml, about 79000000 h*ng/ml, about 80000000 h*ng/mL, about 81000000 h*ng/ml, about 82000000 h*ng/ml, about 83000000 h*ng/ml, about 84000000 h*ng/mL, about 85000000 h*ng/mL, about 86000000 h*ng/ml, about 87000000 h*ng/ml, about 88000000 h*ng/mL, about 89000000 h*ng/mL, about 90000000 h*ng/ml, about 91000000 h*ng/mL, about 92000000 h*ng/ml, about 92300000 h*ng/mL, about 93300000 h*ng/m, about 94300000 h*ng/m, about 95300000 h*ng/m, about 96300000 h*ng/m, about 97300000 h*ng/m, about 98300000 h*ng/m, about 99300000 h*ng/m, or about 100000000 h*ng/m, including any values or ranges therebetween.

In embodiments, administering a therapeutically effective dose of OpSCF provides an AUC_{inf} ranging from about 0.05 h*ng/mL to about 100,000,000 h*ng/mL, about 500,000 h*ng/ml to about 100,000,000 h*ng/mL, about 1,000,000 h*ng/mL to about 100,000,000 h*ng/ml, about 10,000,000 h*ng/ml to about 100,000,000 h*ng/ml, about 20,000,000 h*ng/ml to about 100,000,000 h*ng/mL, about 30,000,000 h*ng/mL to about 100,000,000 h*ng/ml, about 40,000,000 h*ng/mL to about 100,000,000 h*ng/ml, about 50,000,000 h*ng/ml to about 100,000,000 h*ng/mL, about 60,000,000 h*ng/mL to about 100,000,000 h*ng/ml, about 70,000,000 h*ng/mL to about 100,000,000 h*ng/mL, about 80,000,000 h*ng/ml to about 100,000,000 h*ng/mL, about 90,000,000 h*ng/mL to about 100,000,000 h*ng/ml, 0.05 h*ng/mL to about 93,300,000 h*ng/ml, about 500,000 h*ng/mL to about 93,300,000 h*ng/ml, about 1,000,000 h*ng/mL to about 93,300,000 h*ng/ml, about 10,000,000 h*ng/mL to about 93,300,000 h*ng/mL, about 20,000,000 h*ng/mL to about 93,300,000 h*ng/mL, about 30,000,000 h*ng/ml to about 93,300,000 h*ng/mL, about 40,000,000 h*ng/mL to about 93,300,000 h*ng/mL, about 50,000,000 h*ng/mL to about 93,300,000 h*ng/mL, about 60,000,000 h*ng/mL to about 93,300,000 h*ng/mL, about 70,000,000 h*ng/mL to about 93,300,000 h*ng/mL, about 80,000,000 h*ng/ml to about 93,300,000 h*ng/mL, about 90,000,000 h*ng/mL to about 93,300,000 h*ng/ml, about 92,300,000 h*ng/ml to about 93,300,000 h*ng/ml, about 0.05 h*ng/mL to about 92,300,000 h*ng/mL, about 500,000 h*ng/mL to about 92,300,000 h*ng/mL, about 1,000,000 h*ng/mL to about 92,300,000 h*ng/mL, about 10,000,000 h*ng/mL to about 92,300,000 h*ng/mL, about 20,000,000 h*ng/ml to about 92,300,000 h*ng/mL, about 30,000,000 h*ng/mL to about 92,300,000 h*ng/mL, about 40,000,000 h*ng/mL to about 92,300,000 h*ng/mL, about 50,000,000 h*ng/mL to about 92,300,000 h*ng/mL, about 60,000,000 h*ng/mL to about 92,300,000 h*ng/mL, about 70,000,000 h*ng/mL to about 92,300,000 h*ng/mL, about 80,000,000 h*ng/ml to about 92,300,000 h*ng/mL, about 90,000,000 h*ng/mL to about 92,300,000 h*ng/mL, 0.05 h*ng/mL to about 80,000,000 h*ng/mL, about 500,000 h*ng/mL to about 80,000,000 h*ng/ml, about 1,000,000 h*ng/ml to about 80,000,000 h*ng/mL, about 10,000,000 h*ng/mL to about 80,000,000 h*ng/mL, about 20,000,000 h*ng/mL to about 80,000,000 h*ng/mL, about 30,000,000 h*ng/mL to about 80,000,000 h*ng/mL, about 40,000,000 h*ng/mL to about 80,000,000 h*ng/mL, about 50,000,000 h*ng/ml to about 80,000,000 h*ng/mL, about 60,000,000 h*ng/mL to about 80,000,000 h*ng/mL, about 70,000,000 h*ng/mL to about 80,000,000 h*ng/ml, 0.05 h*ng/ml to about 70,000,000 h*ng/ml, about 500,000 h*ng/mL to about 70,000,000 h*ng/ml, about 1,000,000 h*ng/ml to about 70,000,000 h*ng/ml, about 10,000,000 h*ng/mL to about 70,000,000 h*ng/mL, about 20,000,000 h*ng/mL to about 70,000,000 h*ng/mL, about 30,000,000 h*ng/mL to about 70,000,000 h*ng/mL, about 40,000,000 h*ng/mL to about 70,000,000 h*ng/ml, about 50,000,000 h*ng/mL to about 70,000,000 h*ng/mL, about 60,000,000 h*ng/ml to about 70,000,000 h*ng/mL, 0.05 h*ng/mL to about 60,000,000 h*ng/mL, about 500,000 h*ng/mL to about 60,000,000 h*ng/mL, about 1,000,000 h*ng/ml to about 60,000,000 h*ng/mL, about 10,000,000 h*ng/mL to about 60,000,000 h*ng/ml, about 20,000,000 h*ng/ml to about 60,000,000 h*ng/mL, about 30,000,000 h*ng/mL to about 60,000,000 h*ng/mL, about 40,000,000 h*ng/mL to about 60,000,000 h*ng/mL, about 50,000,000 h*ng/mL to about 60,000,000 h*ng/ml, 0.05 h*ng/mL to about 50,000,000 h*ng/mL, about 500,000 h*ng/mL to about 50,000,000 h*ng/mL, about 1,000,000 h*ng/mL to about 50,000,000 h*ng/mL, about 10,000,000 h*ng/ml to about 50,000,000 h*ng/mL, about 20,000,000 h*ng/mL to about 50,000,000 h*ng/ml, about 30,000,000 h*ng/mL to about 50,000,000 h*ng/ml, about 40,000,000 h*ng/ml to about 50,000,000 h*ng/mL, 0.05 h*ng/mL to about 40,000,000 h*ng/mL, about 500,000 h*ng/mL to about 40,000,000 h*ng/mL, about 1,000,000 h*ng/ml to about 40,000,000 h*ng/mL, about 10,000,000 h*ng/mL to about 40,000,000 h*ng/mL, about 20,000,000 h*ng/ml to about 40,000,000 h*ng/mL, about 30,000,000 h*ng/mL to about 40,000,000 h*ng/mL, 0.05 h*ng/mL to about 30,000,000 h*ng/mL, about 500,000 h*ng/mL to about 30,000,000 h*ng/ml, about 1,000,000 h*ng/mL to about 30,000,000 h*ng/mL, about 10,000,000 h*ng/ml to about 30,000,000 h*ng/mL, about 20,000,000 h*ng/ml to about 30,000,000 h*ng/mL, 0.05 h*ng/mL to about 20,000,000 h*ng/mL, about 500,000 h*ng/ml to about 20,000,000 h*ng/mL, about 1,000,000 h*ng/mL to about 20,000,000 h*ng/mL, about 10,000,000 h*ng/ml to about 20,000,000 h*ng/mL, 0.05 h*ng/mL to about 10,000,000 h*ng/mL, about 500,000 h*ng/ml to about 10,000,000 h*ng/mL, about 1,000,000 h*ng/mL to about 10,000,000 h*ng/ml, 0.05 h*ng/ml to about 1,000,000 h*ng/mL, about 500,000 h*ng/mL to about 1,000,000 h*ng/ml, or 0.05 h*ng/mL to, about 500,000 h*ng/mL, including any values or ranges therebetween.

In embodiments, administering a therapeutically effective dose of OpSCF provides an AUC_{inf} ranging from about 615000 h*ng/mL to about 93300000 h*ng/ml (e.g., about 615000, 2290000, 6660000, 24800000, 50200000, 11400000 or 93300000 h*ng/ml, including any values or ranges therebetween).

In embodiments, administering a therapeutically effective dose of OpSCF (i.e., 10 mg of OpSCF) subcutaneously provides an AUC_{inf} ranging from about 492,000 h*ng/ml to about 768750 h*ng/ml (e.g., about 492000 h*ng/mL, 497000 h*ng/mL, 502000 h*ng/mL, 507000 h*ng/ml, 512000 h*ng/ml, 517000 h*ng/ml, 522000 h*ng/ml, 527000 h*ng/ml, 532000 h*ng/ml, 537000 h*ng/ml, 542000 h*ng/ml, 547000 h*ng/ml, 552000 h*ng/ml, 557000 h*ng/ml, 562000 h*ng/mL, 567000 h*ng/ml, 572000 h*ng/ml, 577000 h*ng/mL, 582000 h*ng/mL, 587000 h*ng/ml, 592000 h*ng/ml, 597000 h*ng/ml, 602000 h*ng/ml, 607000 h*ng/ml, 612000 h*ng/ml, 617000 h*ng/mL, 622000 h*ng/ml, 627000 h*ng/ml, 632000 h*ng/mL, 637000 h*ng/mL, 642000 h*ng/ml, 647000 h*ng/mL, 652000 h*ng/ml, 657000 h*ng/ml, 662000 h*ng/mL, 667000 h*ng/mL, 672000 h*ng/ml, 677000 h*ng/ml, 682000 h*ng/mL, 687000 h*ng/ml, 692000 h*ng/ml, 697000 h*ng/mL, 702000 h*ng/ml, 707000 h*ng/mL, 712000 h*ng/ml, 717000 h*ng/ml, 722000 h*ng/ml, 727000 h*ng/ml, 732000 h*ng/mL, 737000 h*ng/mL, 742000 h*ng/ml, 747000 h*ng/ml, 752000 h*ng/ml, 757000 h*ng/mL, 762000 h*ng/mL, 767000 h*ng/mL, or 768750 h*ng/ml, including any values or ranges therebetween). In embodiments, a 10 mg subcutaneous dose of OpSCF provides a AUC_{inf} within the range of 80% to 125% of 615000±30.8 h*ng/mL L. In embodiments, a 10 mg subcutaneous dose of OpSCF provides a AUC_{inf} of about 615000±30.8 h*ng/mL.

In embodiments, administering a therapeutically effective dose of OpSCF (i.e., 30 mg of OpSCF) subcutaneously provides an AUC_{inf} ranging from about 1842000 h*ng/ml to about 2862500 h*ng/ml (e.g., about 1842000 h*ng/ml, 1852000 h*ng/ml, 1862000 h*ng/mL, 1872000 h*ng/mL, 1882000 h*ng/mL, 1892000 h*ng/mL, 1902000 h*ng/mL, 1912000 h*ng/ml, 1922000 h*ng/mL, 1932000 h*ng/mL, 1942000 h*ng/mL, 1952000 h*ng/ml, 1962000 h*ng/ml, 1972000 h*ng/mL, 1982000 h*ng/mL, 1992000 h*ng/mL, 2002000 h*ng/mL, 2012000 h*ng/ml, 2022000 h*ng/mL, 2032000 h*ng/mL, 2042000 h*ng/mL, 2052000 h*ng/mL, 2062000 h*ng/ml, 2072000 h*ng/mL, 2082000 h*ng/mL, 2092000 h*ng/mL, 2102000 h*ng/ml, 2112000 h*ng/ml, 2122000 h*ng/mL, 2132000 h*ng/mL, 2142000 h*ng/mL, 2152000 h*ng/mL, 2162000 h*ng/mL, 2172000 h*ng/mL, 2182000 h*ng/mL, 2192000 h*ng/mL, 2202000 h*ng/mL, 2212000 h*ng/ml, 2222000 h*ng/ml, 2232000 h*ng/mL, 2242000 h*ng/mL, 2252000 h*ng/ml, 2262000 h*ng/ml, 2272000 h*ng/mL, 2282000 h*ng/mL, 2292000 h*ng/ml, 2302000 h*ng/ml, 2312000 h*ng/ml, 2322000 h*ng/mL, 2332000 h*ng/mL, 2342000 h*ng/mL, 2352000 h*ng/ml, 2362000 h*ng/ml, 2372000 h*ng/mL, 2382000 h*ng/mL, 2392000 h*ng/mL, 2402000 h*ng/mL, 2412000 h*ng/ml, 2422000 h*ng/mL, 2432000 h*ng/mL, 2442000 h*ng/mL, 2452000 h*ng/ml, 2462000 h*ng/ml, 2472000 h*ng/mL, 2482000 h*ng/mL, 2492000 h*ng/ml, 2502000 h*ng/mL, 2512000 h*ng/ml, 2522000 h*ng/mL, 2532000 h*ng/mL, 2542000 h*ng/ml, 2552000 h*ng/mL, 2562000 h*ng/mL, 2572000 h*ng/mL, 2582000 h*ng/mL, 2592000 h*ng/mL, 2602000 h*ng/mL, 2612000 h*ng/ml, 2622000 h*ng/ml, 2632000 h*ng/mL, 2642000 h*ng/mL, 2652000 h*ng/mL, 2662000 h*ng/ml, 2672000 h*ng/mL, 2682000 h*ng/mL, 2692000 h*ng/mL, 2702000 h*ng/ml, 2712000 h*ng/mL, 2722000 h*ng/mL, 2732000 h*ng/mL, 2742000 h*ng/mL, 2752000 h*ng/mL, 2762000 h*ng/ml, 2772000 h*ng/mL, 2782000 h*ng/mL, 2792000 h*ng/mL, 2802000 h*ng/ml, 2812000 h*ng/ml, 2822000 h*ng/mL, 2832000 h*ng/mL, 2842000 h*ng/mL, 2852000 h*ng/ml, 2862000 h*ng/ml, or 2862500 h*ng/mL, including any values or ranges therebetween). In embodiments, a 30 mg subcutaneous dose of OpSCF provides a AUC_{inf} within the range of 80% to 125% of 2290000±23.0 h*ng/mL L. In embodiments, a 30 mg subcutaneous dose of OpSCF provides a AUC_{inf} of about 2290000±23.0 h*ng/mL.

In embodiments, administering a therapeutically effective dose of OpSCF (i.e., 100 mg of OpSCF) subcutaneously provides an AUC_{inf} ranging from about 5,328,000 h*ng/ml to about 8,325,000 h*ng/ml (e.g., about 5328000 h*ng/ml, 5378000 h*ng/mL, 5428000 h*ng/ml, 5478000 h*ng/mL, 5528000 h*ng/mL, 5578000 h*ng/mL, 5628000 h*ng/mL, 5678000 h*ng/ml, 5728000 h*ng/mL, 5778000 h*ng/mL, 5828000 h*ng/ml, 5878000 h*ng/mL, 5928000 h*ng/ml, 5978000 h*ng/mL, 6028000 h*ng/mL, 6078000 h*ng/mL, 6128000 h*ng/mL, 6178000 h*ng/ml, 6228000 h*ng/mL, 6278000 h*ng/mL, 6328000 h*ng/mL, 6378000 h*ng/mL, 6428000 h*ng/mL, 6478000 h*ng/mL, 6528000 h*ng/mL, 6578000 h*ng/mL, 6628000 h*ng/mL, 6678000 h*ng/ml, 6728000 h*ng/mL, 6778000 h*ng/mL, 6828000 h*ng/mL, 6878000 h*ng/mL, 6928000 h*ng/mL, 6978000 h*ng/mL, 7028000 h*ng/mL, 7078000 h*ng/mL, 7128000 h*ng/mL, 7178000 h*ng/mL, 7228000 h*ng/mL, 7278000 h*ng/mL, 7328000 h*ng/mL, 7378000 h*ng/mL, 7428000 h*ng/ml, 7478000 h*ng/mL, 7528000 h*ng/mL, 7578000 h*ng/mL, 7628000 h*ng/ml, 7678000 h*ng/ml, 7728000 h*ng/mL, 7778000 h*ng/mL, 7828000 h*ng/mL, 7878000 h*ng/mL, 7928000 h*ng/mL, 7978000 h*ng/mL, 8028000 h*ng/mL, 8078000 h*ng/mL, 8128000 h*ng/mL, 8178000 h*ng/ml, 8228000 h*ng/ml, 8278000 h*ng/ml, or 8325000 h*ng/ml, including any values or ranges therebetween). In embodiments, a 100 mg subcutaneous dose of OpSCF provides an AUC_{inf} within the range of 80% to 125% of 6660000±26.5 h*ng/mL L. In embodiments, a 100 mg subcutaneous dose of OpSCF provides an AUC_{inf} of about 6660000±26.5 h*ng/mL.

In embodiments, administering a therapeutically effective dose of OpSCF (i.e., 300 mg of OpSCF) subcutaneously provides an AUC_{inf} ranging from about 19840000 h*ng/mL to about 31000000 h*ng/mL (e.g., about 19840000 h*ng/ml, 19940000 h*ng/ml, 20040000 h*ng/ml, 20140000 h*ng/ml, 20240000 h*ng/ml, 20340000 h*ng/mL, 20440000 h*ng/ml, 20540000 h*ng/mL, 20640000 h*ng/mL, 20740000 h*ng/ml, 20840000 h*ng/mL, 20940000 h*ng/ml, 21040000 h*ng/ml, 21140000 h*ng/ml, 21240000 h*ng/ml, 21340000 h*ng/ml, 21440000 h*ng/ml, 21540000 h*ng/ml, 21640000 h*ng/ml, 21740000 h*ng/ml, 21840000 h*ng/ml, 21940000 h*ng/ml, 22040000 h*ng/mL, 22140000 h*ng/ml, 22240000 h*ng/ml, 22340000 h*ng/mL, 22440000 h*ng/ml, 22540000 h*ng/mL, 22640000 h*ng/mL, 22740000 h*ng/ml, 22840000 h*ng/mL, 22940000 h*ng/ml, 23040000 h*ng/mL, 23140000 h*ng/ml, 23240000 h*ng/mL, 23340000 h*ng/mL, 23440000 h*ng/ml, 23540000 h*ng/ml, 23640000 h*ng/ml, 23740000 h*ng/ml, 23840000 h*ng/ml, 23940000 h*ng/ml, 24040000 h*ng/ml, 24140000 h*ng/ml, 24240000 h*ng/mL, 24340000 h*ng/mL, 24440000 h*ng/ml, 24540000 h*ng/ml, 24640000 h*ng/ml, 24740000 h*ng/mL, 24840000 h*ng/mL, 24940000 h*ng/mL, 25040000 h*ng/mL, 25140000 h*ng/mL, 25240000 h*ng/ml, 25340000 h*ng/mL, 25440000 h*ng/ml, 25540000 h*ng/ml, 25640000 h*ng/mL, 25740000 h*ng/ml, 25840000 h*ng/ml, 25940000 h*ng/ml, 26040000 h*ng/ml, 26140000 h*ng/ml, 26240000 h*ng/mL, 26340000 h*ng/mL, 26440000 h*ng/ml, 26540000 h*ng/ml, 26640000 h*ng/ml, 26740000 h*ng/ml, 26840000 h*ng/ml, 26940000 h*ng/ml, 27040000 h*ng/ml, 27140000 h*ng/ml, 27240000 h*ng/mL, 27340000 h*ng/ml, 27440000 h*ng/mL, 27540000 h*ng/mL, 27640000 h*ng/mL, 27740000 h*ng/mL, 27840000 h*ng/mL, 27940000 h*ng/ml, 28040000 h*ng/mL, 28140000 h*ng/ml, 28240000 h*ng/ml, 28340000 h*ng/ml, 28440000 h*ng/ml, 28540000 h*ng/ml, 28640000 h*ng/ml, 28740000 h*ng/mL, 28840000 h*ng/mL, 28940000 h*ng/ml, 29040000 h*ng/ml, 29140000 h*ng/mL, 29240000 h*ng/ml, 29340000 h*ng/ml, 29440000 h*ng/ml, 29540000 h*ng/mL, 29640000 h*ng/ml, 29740000 h*ng/ml, 29840000 h*ng/ml, 29940000 h*ng/ml, 30040000 h*ng/ml, 30140000 h*ng/ml, 30240000 h*ng/mL, 30340000 h*ng/ml, 30440000 h*ng/ml, 30540000 h*ng/ml, 30640000 h*ng/ml, 30740000 h*ng/ml, 30840000 h*ng/ml, 30940000 h*ng/ml, or 31000000 h*ng/mL, including any values or ranges therebetween). In embodiments, a 300 mg subcutaneous dose of OpSCF provides an AUC_{inf} within the range of 80% to 125% of 24800000±39.3 h*ng/mL L. In embodiments, a 300 mg subcutaneous dose of OpSCF provides an AUC_{inf} of about 24800000±39.3 h*ng/mL.

In embodiments, administering a therapeutically effective dose of OpSCF (i.e., 600 mg of OpSCF) subcutaneously provides an AUC_{inf} ranging from about 40160000 h*ng/mL to about 62750000 h*ng/ml (e.g., about 40160000 h*ng/mL, 40660000 h*ng/ml, 41160000 h*ng/ml, 41660000 h*ng/ml, 42160000 h*ng/mL, 42660000 h*ng/ml, 43160000 h*ng/ml, 43660000 h*ng/mL, 44160000 h*ng/mL, 44660000 h*ng/mL, 45160000 h*ng/ml, 45660000 h*ng/ml, 46160000 h*ng/ml, 46660000 h*ng/ml, 47160000 h*ng/mL, 47660000 h*ng/ml, 48160000 h*ng/mL, 48660000 h*ng/mL, 49160000 h*ng/mL, 49660000 h*ng/ml, 50160000 h*ng/mL, 50660000 h*ng/ml, 51160000 h*ng/ml, 51660000 h*ng/ml, 52160000 h*ng/ml, 52660000 h*ng/ml, 53160000 h*ng/ml, 53660000 h*ng/mL, 54160000 h*ng/ml, 54660000 h*ng/ml, 55160000 h*ng/mL, 55660000 h*ng/ml, 56160000 h*ng/mL, 56660000 h*ng/ml, 57160000 h*ng/ml, 57660000 h*ng/ml, 58160000 h*ng/ml, 58660000 h*ng/ml, 59160000 h*ng/ml, 59660000 h*ng/mL, 60160000 h*ng/ml, 60660000 h*ng/mL, 61160000 h*ng/ml, 61660000 h*ng/ml, 62160000 h*ng/mL, 62660000 h*ng/mL, or 62750000 h*ng/ml, including any values or ranges therebetween). In embodiments, a 600 mg subcutaneous dose of OpSCF provides an AUC_{inf} within the range of 80% to 125% of 50200000±32.9 h*ng/mL L. In embodiments, a 600 mg subcutaneous dose of OpSCF provides an AUC_{inf} of about 50200000±32.9 h*ng/ml.

In embodiments, administering a therapeutically effective dose of OpSCF (i.e., 100 mg of OpSCF) intravenously provides an AUC_{inf} level ranging from about 9120000 h*ng/mL to about 14250000 h*ng/ml (e.g., about 9120000 h*ng/mL, 9220000 h*ng/ml, 9320000 h*ng/ml, 9420000 h*ng/mL, 9520000 h*ng/mL, 9620000 h*ng/mL, 9720000 h*ng/mL, 9820000 h*ng/mL, 9920000 h*ng/ml, 10020000 h*ng/ml, 10120000 h*ng/ml, 10220000 h*ng/mL, 10320000 h*ng/mL, 10420000 h*ng/ml, 10520000 h*ng/ml, 10620000 h*ng/ml, 10720000 h*ng/ml, 10820000 h*ng/mL, 10920000 h*ng/ml, 11020000 h*ng/ml, 11120000 h*ng/ml, 11220000 h*ng/mL, 11320000 h*ng/ml, 11420000 h*ng/mL, 11520000 h*ng/mL, 11620000 h*ng/ml, 11720000 h*ng/ml, 11820000 h*ng/mL, 11920000 h*ng/mL, 12020000 h*ng/mL, 12120000 h*ng/mL, 12220000 h*ng/ml, 12320000 h*ng/mL, 12420000 h*ng/mL, 12520000 h*ng/ml, 12620000 h*ng/ml, 12720000 h*ng/mL, 12820000 h*ng/mL, 12920000 h*ng/ml, 13020000 h*ng/mL, 13120000 h*ng/mL, 13220000 h*ng/ml, 13320000 h*ng/ml, 13420000 h*ng/mL, 13520000 h*ng/ml, 13620000 h*ng/ml, 13720000 h*ng/ml, 13820000 h*ng/ml, 13920000 h*ng/mL, 14020000 h*ng/mL, 14120000 h*ng/mL, 14220000 h*ng/mL, or 14250000 h*ng/ml, including any values or ranges therebetween). In embodiments, a 100 mg intravenous dose of OpSCF provides an AUC_{inf} within the range of 80% to 125% of 11400000±20.2 h*ng/mL L. In embodiments, a 100 mg intravenous dose of OpSCF provides an AUC_{inf} of about 11400000±20.2 h*ng/mL.

In embodiments, administering a therapeutically effective dose of OpSCF (i.e., 600 mg of OpSCF) intravenously provides an AUC_{inf} ranging from about 74640000 h*ng/mL to about 116625000 h*ng/ml (e.g., about 74640000 h*ng/ml, 75140000 h*ng/mL, 75640000 h*ng/ml, 76140000 h*ng/ml, 76640000 h*ng/ml, 77140000 h*ng/ml, 77640000 h*ng/ml, 78140000 h*ng/ml, 78640000 h*ng/ml, 79140000 h*ng/ml, 79640000 h*ng/ml, 80140000 h*ng/ml, 80640000 h*ng/ml, 81140000 h*ng/mL, 81640000 h*ng/ml, 82140000 h*ng/ml, 82640000 h*ng/ml, 83140000 h*ng/mL, 83640000 h*ng/ml, 84140000 h*ng/mL, 84640000 h*ng/ml, 85140000 h*ng/ml, 85640000 h*ng/ml, 86140000 h*ng/mL, 86640000 h*ng/ml, 87140000 h*ng/mL, 87640000 h*ng/ml, 88140000 h*ng/ml, 88640000 h*ng/mL, 89140000 h*ng/ml, 89640000 h*ng/mL, 90140000 h*ng/mL, 90640000 h*ng/mL, 91140000 h*ng/ml, 91640000 h*ng/ml, 92140000 h*ng/ml, 92640000 h*ng/mL, 93140000 h*ng/mL, 93640000 h*ng/ml, 94140000 h*ng/ml, 94640000 h*ng/ml, 95140000 h*ng/mL, 95640000 h*ng/ml, 96140000 h*ng/ml, 96640000 h*ng/mL, 97140000 h*ng/ml, 97640000 h*ng/ml, 98140000 h*ng/ml, 98640000 h*ng/ml, 99140000 h*ng/mL, 99640000 h*ng/mL, 100140000 h*ng/ml, 100640000 h*ng/ml, 101140000 h*ng/ml, 101640000 h*ng/ml, 102140000 h*ng/ml, 102640000 h*ng/ml, 103140000 h*ng/mL, 103640000 h*ng/ml, 104140000 h*ng/ml, 104640000 h*ng/ml, 105140000 h*ng/mL, 105640000 h*ng/ml, 106140000 h*ng/ml, 106640000 h*ng/ml, 107140000 h*ng/ml, 107640000 h*ng/ml, 108140000 h*ng/ml, 108640000 h*ng/ml, 109140000 h*ng/ml, 109640000 h*ng/mL, 110140000 h*ng/ml, 110640000 h*ng/ml, 111140000 h*ng/ml, 111640000 h*ng/ml, 112140000 h*ng/ml, 112640000 h*ng/ml, 113140000 h*ng/mL, 113640000 h*ng/mL, 114140000 h*ng/ml, 114640000 h*ng/mL, 115140000 h*ng/ml, 115640000 h*ng/ml, 116140000 h*ng/ml, or 116625000 h*ng/mL, including any values or ranges therebetween). In embodiments, a 600 mg intravenous dose of OpSCF provides an AUC_{inf} within the range of 80% to 125% of 93300000±25.8 h*ng/ml L. In embodiments, an 600 mg intravenous dose of OpSCF provides a AUC_{inf} of about 93300000±25.8 h*ng/mL.

In embodiments, AUC_{inf} is expressed by DAUC_{inf} that is normalized by the dose administered. The value “DAUC_{inf}” refers to AUC_{inf} divided by the dose.

### % AUCₑₓₜᵣₐₚ

In embodiments, administering a therapeutically effective dose of OpSCF provides an % AUCₑₓₜᵣₐₚ ranging from about 0.05% to about 20%, including about 0.05%, about 0.5%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, or about 20%, including any values or ranges therebetween. In embodiments, administering a therapeutically effective dose of OpSCF provides an % AUCₑₓₜᵣₐₚ ranging from about 0.05% to about 20%, about 2% to about 20%, about 4% to about 20%, about 6% to about 20%, about 8% to about 20%, about 10% to about 20%, about 12% to about 20%, about 14% to about 20%, about 16% to about 20%, about 18% to about 20%, about 0.05% to about 15%, about 2% to about 15%, about 4% to about 15%, about 6% to about 15%, about 8% to about 15%, about 10% to about 15%, about 12% to about 15%, about 14% to about 15%, about 0.05% to about 10%, about 2% to about 10%, about 4% to about 10%, about 6% to about 10%, about 8% to about 10%, about 0.05% to about 5%, about 2% to about 5%, about 4% to about 5%, or about 0.05% to about 1%, including any values or ranges therebetween.

In embodiments, administering a therapeutically effective dose of OpSCF provides an % AUCₑₓₜᵣₐₚ level ranging from about 0.895% to about 11.1% (e.g., about 0.895%, 1.12%, 1.43%, 1.59%, 1.81%, 1.96%, 11.1%, including any values or ranges therebetween).

In embodiments, administering a therapeutically effective dose of OpSCF (i.e., 10 mg of OpSCF) subcutaneously provides a % AUCₑₓₜᵣₐₚ level ranging from about 8% to about 14% (e.g., about 8%, 9%, 10%, 11%, 12%, 13%, 14%, including any values or ranges therebetween. In embodiments, a 10 mg subcutaneous dose of OpSCF provides a % AUCₑₓₜᵣₐₚ within the range of 80% to 125% of 11.1±98.3%. In embodiments, a 10 mg subcutaneous dose of OpSCF provides a % AUCₑₓₜᵣₐₚ of about 11.1±98.3%.

In embodiments, administering a therapeutically effective dose of OpSCF (i.e., 30 mg of OpSCF) subcutaneously provides a % AUCₑₓₜᵣₐₚ level ranging from about 1.4% to about 2.3% (e.g., about 1.4%, 1.5%, 1.6%, 1.7%, 1.8%, 1.9%, 2.0%, 2.1%, 2.2%, or 2.3%, including any values or ranges therebetween. In embodiments, a 30 mg subcutaneous dose of OpSCF provides a % AUCₑₓₜᵣₐₚ within the range of 80% to 125% of 1.81±27.9%. In embodiments, a 30 mg subcutaneous dose of OpSCF provides a % AUCₑₓₜᵣₐₚ of about 1.81±27.9%.

In embodiments, administering a therapeutically effective dose of OpSCF (i.e., 100 mg of OpSCF) subcutaneously provides an % AUCₑₓₜᵣₐₚ level ranging from about 0.8% to about 1.4% (e.g., about 0.8%, 0.9%, 1.0%, 1.1%, 1.2%, 1.3%, or 1.4%, including any values or ranges therebetween). In embodiments, a 100 mg subcutaneous dose of OpSCF provides a % AUCₑₓₜᵣₐₚ within the range of 80% to 125% of 1.12±73.7%. In embodiments, a 100 mg subcutaneous dose of OpSCF provides a % AUCₑₓₜᵣₐₚ of about 1.12±73.7%.

In embodiments, administering a therapeutically effective dose of OpSCF (i.e., 300 mg of OpSCF) subcutaneously provides a % AUCₑₓₜᵣₐₚ level ranging from about 1.5% to about 2.5% (e.g., about 1.5%, 1.6%, 1.7%, 1.8%, 1.9%, 2.0%, 2.1%, 2.2%, 2.3%, 2.4% or 2.5%, including any values or ranges therebetween). In embodiments, a 300 mg subcutaneous dose of OpSCF provides a % AUCₑₓₜᵣₐₚ within the range of 80% to 125% of 1.96±311.5%. In embodiments, a 300 mg subcutaneous dose of OpSCF provides a % AUCₑₓₜᵣₐₚ of about 1.96±311.5%.

In embodiments, administering a therapeutically effective dose of OpSCF (i.e., 600 mg of OpSCF) subcutaneously provides an % AUCₑₓₜᵣₐₚ level ranging from about 0.7% to about 1.2% (e.g., about 0.7%, 0.8%, 0.9%, 1.0%, 1.1%, or 1.2%, including any values or ranges therebetween). In embodiments, a 300 mg subcutaneous dose of OpSCF provides a % AUCₑₓₜᵣₐₚ within the range of 80% to 125% of 0.895±117.8%. In embodiments, a 600 mg subcutaneous dose of OpSCF provides a % AUCₑₓₜᵣₐₚ of about 0.895±117.8%.

In embodiments, administering a therapeutically effective dose of OpSCF (i.e., 100 mg of OpSCF) intravenously provides an % AUCₑₓₜᵣₐₚ level ranging from about 1.2% to about 2.0% (e.g., about 1.2%, 1.3%, 1.4%, 1.5%, 1.6%, 1.7%, 1.8%, 1.9% or 2.0%, including any values or ranges therebetween). In embodiments, a 100 mg intravenous dose of OpSCF provides a % AUCₑₓₜᵣₐₚ within the range of 80% to 125% of 1.59±235.7%. In embodiments, a 100 mg intravenous dose of OpSCF provides a % AUCₑₓₜᵣₐₚ of about 1.59±235.7%.

In embodiments, administering a therapeutically effective dose of OpSCF (i.e., 600 mg of OpSCF) intravenously provides an % AUCₑₓₜᵣₐₚ level ranging from about 1.2% to about 2.0% (e.g., about 1.2%, 1.3%, 1.4%, 1.5%, 1.6%, 1.7%, 1.8%, 1.9% or 2.0%, including any values or ranges therebetween). In embodiments, a 600 mg intravenous dose of OpSCF provides a % AUCₑₓₜᵣₐₚ within the range of 80% to 125% of 1.43±296.3%. In embodiments, a 600 mg intravenous dose of OpSCF provides a % AUCₑₓₜᵣₐₚ of about 1.43±296.3%.

### Tₘₐₓ (h)

In embodiments, administering a therapeutically effective dose of OpSCF provides a time to maximum blood plasma concentration (Tₘₐₓ) ranging from about 0.05 hours to about 200 hours, including about 0.05 hours, about 0.5 hours, about 1 hours, about 2 hours, about 3 hours, about 4 hours, about 5 hours, about 6 hours, about 7 hours, about 8 hours, about 9 hours, about 10 hours, about 11 hours, about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 25 hours, about 26 hours, about 27 hours, about 28 hours, about 29 hours, about 30 hours, about 31 hours, about 32 hours, about 33 hours, about 34 hours, about 35 hours, about 36 hours, about 37 hours, about 38 hours, about 39 hours, about 40 hours, about 41 hours, about 42 hours, about 43 hours, about 44 hours, about 45 hours, about 46 hours, about 47 hours, about 48 hours, about 49 hours, about 50 hours, about 51 hours, about 52 hours, about 53 hours, about 54 hours, about 55 hours, about 56 hours, about 57 hours, about 58 hours, about 59 hours, about 60 hours, about 61 hours, about 62 hours, about 63 hours, about 64 hours, about 65 hours, about 66 hours, about 67 hours, about 68 hours, about 69 hours, about 70 hours, about 71 hours, about 72 hours, about 73 hours, about 74 hours, about 75 hours, about 76 hours, about 77 hours, about 78 hours, about 79 hours, about 80 hours, about 81 hours, about 82 hours, about 83 hours, about 84 hours, about 85 hours, about 86 hours, about 87 hours, about 88 hours, about 89 hours, about 90 hours, about 91 hours, about 92 hours, about 93 hours, about 94 hours, about 95 hours, about 96 hours, about 97 hours, about 98 hours, about 99 hours, about 100 hours, about 101 hours, about 102 hours, about 103 hours, about 104 hours, about 105 hours, about 106 hours, about 107 hours, about 108 hours, about 109 hours, about 110 hours, about 111 hours, about 112 hours, about 113 hours, about 114 hours, about 115 hours, about 116 hours, about 117 hours, about 118 hours, about 119 hours, about 120 hours, about 121 hours, about 122 hours, about 123 hours, about 124 hours, about 125 hours, about 126 hours, about 127 hours, about 128 hours, about 129 hours, about 130 hours, about 131 hours, about 132 hours, about 133 hours, about 134 hours, about 135 hours, about 136 hours, about 137 hours, about 138 hours, about 139 hours, about 140 hours, about 141 hours, about 142 hours, about 143 hours, about 144 hours, about 145 hours, about 146 hours, about 147 hours, about 148 hours, about 149 hours, about 150 hours, about 151 hours, about 152 hours, about 153 hours, about 154 hours, about 155 hours, about 156 hours, about 157 hours, about 158 hours, about 159 hours, about 160 hours, about 161 hours, about 162 hours, about 163 hours, about 164 hours, about 165 hours, about 166 hours, about 167 hours, about 168 hours, about 169 hours, about 170 hours, about 171 hours, about 172 hours, about 173 hours, about 174 hours, about 175 hours, about 176 hours, about 177 hours, about 178 hours, about 179 hours, about 180 hours, about 181 hours, about 182 hours, about 183 hours, about 184 hours, about 185 hours, about 186 hours, about 187 hours, about 188 hours, about 189 hours, about 190 hours, about 191 hours, about 192 hours, about 193 hours, about 194 hours, about 195 hours, about 196 hours, about 197 hours, about 198 hours, about 199 hours, or about 200 hours, including any values or ranges therebetween.

In embodiments, administering a therapeutically effective dose of OpSCF provides a time to maximum blood plasma concentration (Tₘₐₓ) 0.05 hours to about 200 hours, about 25 hours to about 200 hours, about 50 hours to about 200 hours, about 75 hours to about 200 hours, about 100 hours to about 200 hours, about 125 hours to about 200 hours, about 150 hours to about 200 hours, about 175 hours to about 200 hours, about 0.05 hours to about 175 hours, about 25 hours to about 175 hours, about 50 hours to about 175 hours, about 75 hours to about 175 hours, about 100 hours to about 175 hours, about 125 hours to about 175 hours, about 150 hours to about 175 hours, about 0.05 hours to about 150 hours, about 25 hours to about 150 hours, about 50 hours to about 150 hours, about 75 hours to about 150 hours, about 100 hours to about 150 hours, about 125 hours to about 150 hours, about 0.05 hours to about 150 hours, about 25 hours to about 150 hours, about 50 hours to about 150 hours, about 75 hours to about 150 hours, about 100 hours to about 150 hours, about 125 hours to about 150 hours, about 0.05 hours to about 125 hours, about 25 hours to about 125 hours, about 50 hours to about 125 hours, about 75 hours to about 125 hours, about 100 hours to about 125 hours, about 0.05 hours to about 100 hours, about 25 hours to about 100 hours, about 50 hours to about 100 hours, about 75 hours to about 100 hours, about 0.05 hours to about 75 hours, about 25 hours to about 75 hours, about 50 hours to about 75 hours, about 0.05 hours to about 50 hours, about 25 hours to about 50 hours, or about 0.05 hours to about 25 hours, including any values or ranges therebetween.

In embodiments, administering a therapeutically effective dose of OpSCF provides a time to maximum blood plasma concentration (Tₘₐₓ) ranging from about 4 hours to about 156 hours (e.g., about 4 hours, 108 hours, 132 hours or 156 hours, including any values or ranges therebetween).

In embodiments, administering a therapeutically effective dose of OpSCF (i.e., 10 mg of OpSCF) subcutaneously provides a time to maximum blood plasma concentration (Tₘₐₓ) ranging from 86 hours to about 135 hours (e.g., 86 hours, 87 hours, 88 hours, 89 hours, 90 hours, 91 hours, 92 hours, 93 hours, 94 hours, 95 hours, 96 hours, 97 hours, 98 hours, 99 hours, 100 hours, 101 hours, 102 hours, 103 hours, 104 hours, 105 hours, 106 hours, 107 hours, 108 hours, 109 hours, 110 hours, 111 hours, 112 hours, 113 hours, 114 hours, 115 hours, 116 hours, 117 hours, 118 hours, 119 hours, 120 hours, 121 hours, 122 hours, 123 hours, 124 hours, 125 hours, 126 hours, 127 hours, 128 hours, 129 hours, 130 hours, 131 hours, 132 hours, 133 hours, 134 hours, 135 hours, including any values or ranges therebetween). In embodiments, a 10 mg subcutaneous dose of OpSCF provides a Tₘₐₓ within the range of 80% to 125% of 108 hours. In embodiments, a 10 mg subcutaneous dose of OpSCF provides a Tₘₐₓ of about 108 hours.

In embodiments, administering a therapeutically effective dose of OpSCF (i.e., 30 mg of OpSCF) subcutaneously provides a time to maximum blood plasma concentration (Tₘₐₓ) ranging from 105 hours to about 165 hours (e.g., 105 hours, 106 hours, 107 hours, 108 hours, 109 hours, 110 hours, 111 hours, 112 hours, 113 hours, 114 hours, 115 hours, 116 hours, 117 hours, 118 hours, 119 hours, 120 hours, 121 hours, 122 hours, 123 hours, 124 hours, 125 hours, 126 hours, 127 hours, 128 hours, 129 hours, 130 hours, 131 hours, 132 hours, 133 hours, 134 hours, 135 hours, 136 hours, 137 hours, 138 hours, 139 hours, 140 hours, 141 hours, 142 hours, 143 hours, 144 hours, 145 hours, 146 hours, 147 hours, 148 hours, 149 hours, 150 hours, 151 hours, 152 hours, 153 hours, 154 hours, 155 hours, 156 hours, 157 hours, 158 hours, 159 hours, 160 hours, 161 hours, 162 hours, 163 hours, 164 hours, 165 hours, including any values or ranges therebetween). In embodiments, a 30 mg subcutaneous dose of OpSCF provides a Tₘₐₓ within the range of 80% to 125% of 132 hours. In embodiments, a 30 mg subcutaneous dose of OpSCF provides a Tₘₐₓ of about 132 hours.

In embodiments, administering a therapeutically effective dose of OpSCF (i.e., 100 mg of OpSCF) subcutaneously provides a time to maximum blood plasma concentration (Tₘₐₓ) ranging from 124 hours to about 195 hours (e.g., 124 hours, 125 hours, 126 hours, 127 hours, 128 hours, 129 hours, 130 hours, 131 hours, 132 hours, 133 hours, 134 hours, 135 hours, 136 hours, 137 hours, 138 hours, 139 hours, 140 hours, 141 hours, 142 hours, 143 hours, 144 hours, 145 hours, 146 hours, 147 hours, 148 hours, 149 hours, 150 hours, 151 hours, 152 hours, 153 hours, 154 hours, 155 hours, 156 hours, 157 hours, 158 hours, 159 hours, 160 hours, 161 hours, 162 hours, 163 hours, 164 hours, 165 hours, 166 hours, 167 hours, 168 hours, 169 hours, 170 hours, 171 hours, 172 hours, 173 hours, 174 hours, 175 hours, 176 hours, 177 hours, 178 hours, 179 hours, 180 hours, 181 hours, 182 hours, 183 hours, 184 hours, 185 hours, 186 hours, 187 hours, 188 hours, 189 hours, 190 hours, 191 hours, 192 hours, 193 hours, 194 hours, or 195 hours, including any values or ranges therebetween). In embodiments, a 100 mg subcutaneous dose of OpSCF provides a Tₘₐₓ within the range of 80% to 125% of 156 hours. In embodiments, a 100 mg subcutaneous dose of OpSCF provides a Tₘₐₓ of about 156 hours.

In embodiments, administering a therapeutically effective dose of OpSCF (i.e., 300 mg of OpSCF) subcutaneously provides a time to maximum blood plasma concentration (Tₘₐₓ) ranging from 124 hours to about 195 hours (e.g., 124 hours, 125 hours, 126 hours, 127 hours, 128 hours, 129 hours, 130 hours, 131 hours, 132 hours, 133 hours, 134 hours, 135 hours, 136 hours, 137 hours, 138 hours, 139 hours, 140 hours, 141 hours, 142 hours, 143 hours, 144 hours, 145 hours, 146 hours, 147 hours, 148 hours, 149 hours, 150 hours, 151 hours, 152 hours, 153 hours, 154 hours, 155 hours, 156 hours, 157 hours, 158 hours, 159 hours, 160 hours, 161 hours, 162 hours, 163 hours, 164 hours, 165 hours, 166 hours, 167 hours, 168 hours, 169 hours, 170 hours, 171 hours, 172 hours, 173 hours, 174 hours, 175 hours, 176 hours, 177 hours, 178 hours, 179 hours, 180 hours, 181 hours, 182 hours, 183 hours, 184 hours, 185 hours, 186 hours, 187 hours, 188 hours, 189 hours, 190 hours, 191 hours, 192 hours, 193 hours, 194 hours, or 195 hours, including any values or ranges therebetween). In embodiments, a 300 mg subcutaneous dose of OpSCF provides a Tₘₐₓ within the range of 80% to 125% of 156 hours. In embodiments, a 300 mg subcutaneous dose of OpSCF provides a Tₘₐₓ of about 156 hours.

In embodiments, administering a therapeutically effective dose of OpSCF (i.e., 600 mg of OpSCF) subcutaneously provides a time to maximum blood plasma concentration (Tₘₐₓ) ranging from 105 hours to about 165 hours (e.g., 105 hours, 106 hours, 107 hours, 108 hours, 109 hours, 110 hours, 111 hours, 112 hours, 113 hours, 114 hours, 115 hours, 116 hours, 117 hours, 118 hours, 119 hours, 120 hours, 121 hours, 122 hours, 123 hours, 124 hours, 125 hours, 126 hours, 127 hours, 128 hours, 129 hours, 130 hours, 131 hours, 132 hours, 133 hours, 134 hours, 135 hours, 136 hours, 137 hours, 138 hours, 139 hours, 140 hours, 141 hours, 142 hours, 143 hours, 144 hours, 145 hours, 146 hours, 147 hours, 148 hours, 149 hours, 150 hours, 151 hours, 152 hours, 153 hours, 154 hours, 155 hours, 156 hours, 157 hours, 158 hours, 159 hours, 160 hours, 161 hours, 162 hours, 163 hours, 164 hours, 165 hours, including any values or ranges therebetween). In embodiments, a 600 mg subcutaneous dose of OpSCF provides a Tₘₐₓ within the range of 80% to 125% of 132 hours. In embodiments, a 600 mg subcutaneous dose of OpSCF provides a Tₘₐₓ of about 132 hours.

In embodiments, administering a therapeutically effective dose of OpSCF (i.e., 100 mg of OpSCF) intravenously provides a time to maximum blood plasma concentration (Tₘₐₓ) ranging from about 3.2 hours to about 5 hours (e.g., 3.2 hours, 3.3 hours, 3.4 hours, 3.5 hours, 3.6 hours, 3.7 hours, 3.8 hours, 3.9 hours, 4.0 hours, 4.1 hours, 4.2 hours, 4.3 hours, 4.4 hours, 4.5 hours, 4.6 hours, 4.7 hours, 4.8 hours, 4.9 hours or 5 hours, including any values or ranges therebetween). In embodiments, a 100 mg intravenous dose of OpSCF provides a Tₘₐₓ within the range of 80% to 125% of 4 hours. In embodiments, a 100 mg intravenous dose of OpSCF provides a Tₘₐₓ of about 4 hours.

In embodiments, administering a therapeutically effective dose of OpSCF (i.e., 600 mg of OpSCF) intravenously provides a time to maximum blood plasma concentration (Tₘₐₓ) ranging from about 3.2 hours to about 5 hours (e.g., 3.2 hours, 3.3 hours, 3.4 hours, 3.5 hours, 3.6 hours, 3.7 hours, 3.8 hours, 3.9 hours, 4.0 hours, 4.1 hours, 4.2 hours, 4.3 hours, 4.4 hours, 4.5 hours, 4.6 hours, 4.7 hours, 4.8 hours, 4.9 hours or 5 hours, including any values or ranges therein). In embodiments, a 600 mg intravenous dose of OpSCF provides a Tₘₐₓ within the range of 80% to 125% of 4 hours. In embodiments, a 600 mg intravenous dose of OpSCF provides a Tₘₐₓ of about 4 hours.

In embodiments, a 30 mg intravenous dose of OpSCF provides a Tₘₐₓ within the range of 80% to 125% of 4 hours. In embodiments, a 30 mg intravenous dose of OpSCF provides a Tₘₐₓ of about 4 hours.

In embodiments, a 100 mg intravenous dose of OpSCF provides a Tₘₐₓ within the range of 80% to 125% of 14 hours. In embodiments, a 100 mg intravenous dose of OpSCF provides a Tₘₐₓ of about 14 hours.

In embodiments, a 300 mg intravenous dose of OpSCF provides a Tₘₐₓ within the range of 80% to 125% of 4 hours. In embodiments, a 300 mg intravenous dose of OpSCF provides a Tₘₐₓ of about 4 hours.

In embodiments, administering multiple 30 mg doses of OpSCF provides a Tₘₐₓ within the range of 80% to 125% of 120 hours. In embodiments, administering multiple 30 mg doses of OpSCF provides a Tₘₐₓ of about 120 hours.

In embodiments, administering multiple 100 mg doses of OpSCF provides a Tₘₐₓ within the range of 80% to 125% of 60 hours. In embodiments, administering multiple 100 mg doses of OpSCF provides a Tₘₐₓ of about 60 hours.

In embodiments, administering multiple 300 mg doses of OpSCF provides a Tₘₐₓ within the range of 80% to 125% of 108 hours. In embodiments, administering multiple 300 mg doses of OpSCF provides a Tₘₐₓ of about 108 hours.

### Tₗₐₛₜ

In embodiments, administering a therapeutically effective dose of OpSCF provides a time of the last quantifiable concentration (Tₗₐₛₜ) ranging from about 0.05 hours to about 4,000 hours, including about 0.05 hours, about 0.5 hours, about 1 hours, about 100 hours, about 200 hours, about 300 hours, about 400 hours, about 500 hours, about 600 hours, about 700 hours, about 800 hours, about 900 hours, about 1000 hours, about 1100 hours, about 1200 hours, about 1300 hours, about 1400 hours, about 1500 hours, about 1600 hours, about 1700 hours, about 1800 hours, about 1900 hours, about 2000 hours, about 2100 hours, about 2200 hours, about 2300 hours, about 2400 hours, about 2500 hours, about 2600 hours, about 2700 hours, about 2800 hours, about 2900 hours, about 3000 hours, about 3100 hours, about 3200 hours, about 3300 hours, about 3400 hours, about 3500 hours, about 3600 hours, about 3700 hours, about 3800 hours, about 3900 hours, about 4000 hours, including any ranges or values therebetween.

In embodiments, administering a therapeutically effective dose of OpSCF provides a time of the last quantifiable concentration (Tₗₐₛₜ) ranging from about 0.5 hours to about 4,000 hour, about 250 hours to about 4,000 hours, about 500 hours to about 4,000 hours, about 725 hours to about 4,000 hours, about 1,000 hours to about 4,000 hours, about 1,250 hours to about 4,000 hours, about 1,500 hours to about 4,000 hours, about 1,750 hours to about 4,000 hours, about 2,000 hours to about 4,000 hours, about 2,250 hours to about 4,000 hours, about 2,500 hours to about 4,000 hours, about 2,750 hours to about 4,000 hours, about 3,000 hours to about 4,000 hours, about 3,250 hours to about 4,000 hours, about 3,500 hours to about 4,000 hours, about 3,750 hours to about 4,000 hours, about 0.5 hours to about 3,500 hour, about 250 hours to about 3,500 hours, about 500 hours to about 3,500 hours, about 725 hours to about 3,500 hours, about 1,000 hours to about 3,500 hours, about 1,250 hours to about 3,500 hours, about 1,500 hours to about 3,500 hours, about 1,750 hours to about 3,500 hours, about 2,000 hours to about 3,500 hours, about 2,250 hours to about 3,500 hours, about 2,500 hours to about 3,500 hours, about 2,750 hours to about 3,500 hours, about 3,000 hours to about 3,500 hours, about 3,250 hours to about 3,500 hours, about 0.5 hours to about 3,000 hour, about 250 hours to about 3,000 hours, about 500 hours to about 3,000 hours, about 725 hours to about 3,000 hours, about 1,000 hours to about 3,000 hours, about 1,250 hours to about 3,000 hours, about 1,500 hours to about 3,000 hours, about 1,750 hours to about 3,000 hours, about 2,000 hours to about 3,000 hours, about 2,250 hours to about 3,000 hours, about 2,500 hours to about 3,000 hours, about 2,750 hours to about 3,000 hours, about 0.5 hours to about 2,500 hour, about 250 hours to about 2,500 hours, about 500 hours to about 2,500 hours, about 725 hours to about 2,500 hours, about 1,000 hours to about 2,500 hours, about 1,250 hours to about 2,500 hours, about 1,500 hours to about 2,500 hours, about 1,750 hours to about 2,500 hours, about 2,000 hours to about 2,500 hours, about 2,250 hours to about 2,500 hours, about 0.5 hours to about 2,000 hour, about 250 hours to about 2,000 hours, about 500 hours to about 2,000 hours, about 725 hours to about 2,000 hours, about 1,000 hours to about 2,000 hours, about 1,250 hours to about 2,000 hours, about 1,500 hours to about 2,000 hours, about 1,750 hours to about 2,000 hours, about 0.5 hours to about 1,500 hour, about 250 hours to about 1,500 hours, about 500 hours to about 1,500 hours, about 725 hours to about 1,500 hours, about 1,000 hours to about 1,500 hours, about 1,250 hours to about 1,500 hours, about 0.5 hours to about 1,000 hour, about 250 hours to about 1,000 hours, about 500 hours to about 1,000 hours, about 725 hours to about 1,000 hours, about 0.5 hours to about 500 hour, or about 250 hours to about 500 hours, including any values or ranges therebetween.

In embodiments, the therapeutically effective steady state plasma Tₗₐₛₜ provided by OpSCF of the present disclosure range from about 2020 hours to about 2520 hours (e.g., about 2020 hours, 2450 hours, or 2520 hours, including any values or ranges therebetween).

In embodiments, administering a therapeutically effective dose of OpSCF (i.e., 10 mg of OpSCF) subcutaneously provides a time of the last quantifiable concentration (Tₗₐₛₜ) ranging from about 1616 hours to about 2525 hours (e.g., about 1616 hours, 1636 hours, 1656 hours, 1676 hours, 1696 hours, 1716 hours, 1736 hours, 1756 hours, 1776 hours, 1796 hours, 1816 hours, 1836 hours, 1856 hours, 1876 hours, 1896 hours, 1916 hours, 1936 hours, 1956 hours, 1976 hours, 1996 hours, 2016 hours, 2036 hours, 2056 hours, 2076 hours, 2096 hours, 2116 hours, 2136 hours, 2156 hours, 2176 hours, 2196 hours, 2216 hours, 2236 hours, 2256 hours, 2276 hours, 2296 hours, 2316 hours, 2336 hours, 2356 hours, 2376 hours, 2396 hours, 2416 hours, 2436 hours, 2456 hours, 2476 hours, 2496 hours, 2516 hours, 2525 hours, including values or ranges therebetween). In embodiments, a 10 mg subcutaneous dose of OpSCF provides a Tₗₐₛₜ within the range of 80% to 125% of 2020 hours. In embodiments, a 10 mg subcutaneous dose of OpSCF provides a Tₗₐₛₜ of about 2020 hours.

In embodiments, administering a therapeutically effective dose of OpSCF (i.e., 30 mg of OpSCF) subcutaneously provides a time of the last quantifiable concentration (Tₗₐₛₜ) ranging from about 3150 hours (e.g., about 2016 hours, 2036 hours, 2056 hours, 2076 hours, 2096 hours, 2116 hours, 2136 hours, 2156 hours, 2176 hours, 2196 hours, 2216 hours, 2236 hours, 2256 hours, 2276 hours, 2296 hours, 2316 hours, 2336 hours, 2356 hours, 2376 hours, 2396 hours, 2416 hours, 2436 hours, 2456 hours, 2476 hours, 2496 hours, 2516 hours, 2536 hours, 2556 hours, 2576 hours, 2596 hours, 2616 hours, 2636 hours, 2656 hours, 2676 hours, 2696 hours, 2716 hours, 2736 hours, 2756 hours, 2776 hours, 2796 hours, 2816 hours, 2836 hours, 2856 hours, 2876 hours, 2896 hours, 2916 hours, 2936 hours, 2956 hours, 2976 hours, 2996 hours, 3016 hours, 3036 hours, 3056 hours, 3076 hours, 3096 hours, 3116 hours, 3136 hours, or 3150 hours, including values or ranges therebetween). In embodiments, a 30 mg subcutaneous dose of OpSCF provides a Tₗₐₛₜ within the range of 80% to 125% of 2520 hours. In embodiments, a 30 mg subcutaneous dose of OpSCF provides a Tₗₐₛₜ of about 2520 hours.

In embodiments, administering a therapeutically effective dose of OpSCF (i.e., 100 mg of OpSCF) subcutaneously provides a time of the last quantifiable concentration (Tₗₐₛₜ) ranging from about 2016 hours to about 3150 hours (e.g., about 2016 hours, 2036 hours, 2056 hours, 2076 hours, 2096 hours, 2116 hours, 2136 hours, 2156 hours, 2176 hours, 2196 hours, 2216 hours, 2236 hours, 2256 hours, 2276 hours, 2296 hours, 2316 hours, 2336 hours, 2356 hours, 2376 hours, 2396 hours, 2416 hours, 2436 hours, 2456 hours, 2476 hours, 2496 hours, 2516 hours, 2536 hours, 2556 hours, 2576 hours, 2596 hours, 2616 hours, 2636 hours, 2656 hours, 2676 hours, 2696 hours, 2716 hours, 2736 hours, 2756 hours, 2776 hours, 2796 hours, 2816 hours, 2836 hours, 2856 hours, 2876 hours, 2896 hours, 2916 hours, 2936 hours, 2956 hours, 2976 hours, 2996 hours, 3016 hours, 3036 hours, 3056 hours, 3076 hours, 3096 hours, 3116 hours, 3136 hours, or 3150 hours, including values or ranges therebetween). In embodiments, a 100 mg subcutaneous dose of OpSCF provides a Tₗₐₛₜ within the range of 80% to 125% of 2520 hours. In embodiments, a 100 mg subcutaneous dose of OpSCF provides a Tₗₐₛₜ of about 2520 hours.

In embodiments, administering a therapeutically effective dose of OpSCF (i.e., 300 mg of OpSCF) subcutaneously provides a time of the last quantifiable concentration (Tₗₐₛₜ) ranging from about 2016 hours to about 3150 hours (e.g., about 2016 hours, 2036 hours, 2056 hours, 2076 hours, 2096 hours, 2116 hours, 2136 hours, 2156 hours, 2176 hours, 2196 hours, 2216 hours, 2236 hours, 2256 hours, 2276 hours, 2296 hours, 2316 hours, 2336 hours, 2356 hours, 2376 hours, 2396 hours, 2416 hours, 2436 hours, 2456 hours, 2476 hours, 2496 hours, 2516 hours, 2536 hours, 2556 hours, 2576 hours, 2596 hours, 2616 hours, 2636 hours, 2656 hours, 2676 hours, 2696 hours, 2716 hours, 2736 hours, 2756 hours, 2776 hours, 2796 hours, 2816 hours, 2836 hours, 2856 hours, 2876 hours, 2896 hours, 2916 hours, 2936 hours, 2956 hours, 2976 hours, 2996 hours, 3016 hours, 3036 hours, 3056 hours, 3076 hours, 3096 hours, 3116 hours, 3136 hours, or 3150 hours, including values or ranges therebetween). In embodiments, a 300 mg subcutaneous dose of OpSCF provides a Tₗₐₛₜ within the range of 80% to 125% of 2520 hours. In embodiments, a 300 mg subcutaneous dose of OpSCF provides a Tₗₐₛₜ of about 2520 hours.

In embodiments, administering a therapeutically effective dose of OpSCF (i.e., 600 mg of OpSCF) subcutaneously provides a time of the last quantifiable concentration (Tₗₐₛₜ) ranging from about 1960 hours to about 3063 hours (e.g., about 1960 hours, 1980 hours, 2000 hours, 2020 hours, 2040 hours, 2060 hours, 2080 hours, 2100 hours, 2120 hours, 2140 hours, 2160 hours, 2180 hours, 2200 hours, 2220 hours, 2240 hours, 2260 hours, 2280 hours, 2300 hours, 2320 hours, 2340 hours, 2360 hours, 2380 hours, 2400 hours, 2420 hours, 2440 hours, 2460 hours, 2480 hours, 2500 hours, 2520 hours, 2540 hours, 2560 hours, 2580 hours, 2600 hours, 2620 hours, 2640 hours, 2660 hours, 2680 hours, 2700 hours, 2720 hours, 2740 hours, 2760 hours, 2780 hours, 2800 hours, 2820 hours, 2840 hours, 2860 hours, 2880 hours, 2900 hours, 2920 hours, 2940 hours, 2960 hours, 2980 hours, 3000 hours, 3020 hours, 3040 hours, 3060 hours, or 3063 hours, including values or ranges therebetween). In embodiments, a 600 mg subcutaneous dose of OpSCF provides a Tₗₐₛₜ within the range of 80% to 125% of 2450 hours. In embodiments, a 600 mg subcutaneous dose of OpSCF provides a Tₗₐₛₜ of about 2450 hours.

In embodiments, administering a therapeutically effective dose of OpSCF (i.e., 100 mg of OpSCF) intravenously provides a time of the last quantifiable concentration (Tₗₐₛₜ) ranging from about 2016 hours to about 3150 hours (e.g., about 2016 hours, 2036 hours, 2056 hours, 2076 hours, 2096 hours, 2116 hours, 2136 hours, 2156 hours, 2176 hours, 2196 hours, 2216 hours, 2236 hours, 2256 hours, 2276 hours, 2296 hours, 2316 hours, 2336 hours, 2356 hours, 2376 hours, 2396 hours, 2416 hours, 2436 hours, 2456 hours, 2476 hours, 2496 hours, 2516 hours, 2536 hours, 2556 hours, 2576 hours, 2596 hours, 2616 hours, 2636 hours, 2656 hours, 2676 hours, 2696 hours, 2716 hours, 2736 hours, 2756 hours, 2776 hours, 2796 hours, 2816 hours, 2836 hours, 2856 hours, 2876 hours, 2896 hours, 2916 hours, 2936 hours, 2956 hours, 2976 hours, 2996 hours, 3016 hours, 3036 hours, 3056 hours, 3076 hours, 3096 hours, 3116 hours, 3136 hours, or 3150 hours, including values or ranges therebetween). In embodiments, a 100 mg intravenous dose of OpSCF provides a Tₗₐₛₜ within the range of 80% to 125% of 2520 hours. In embodiments, a 100 mg intravenous dose of OpSCF provides a Tₗₐₛₜ of about 2520 hours.

In embodiments, administering a therapeutically effective dose of OpSCF (i.e., 600 mg of OpSCF) intravenously provides a time of the last quantifiable concentration (Tₗₐₛₜ) ranging from about 2016 hours to about 3150 hours (e.g., about 2016 hours, 2036 hours, 2056 hours, 2076 hours, 2096 hours, 2116 hours, 2136 hours, 2156 hours, 2176 hours, 2196 hours, 2216 hours, 2236 hours, 2256 hours, 2276 hours, 2296 hours, 2316 hours, 2336 hours, 2356 hours, 2376 hours, 2396 hours, 2416 hours, 2436 hours, 2456 hours, 2476 hours, 2496 hours, 2516 hours, 2536 hours, 2556 hours, 2576 hours, 2596 hours, 2616 hours, 2636 hours, 2656 hours, 2676 hours, 2696 hours, 2716 hours, 2736 hours, 2756 hours, 2776 hours, 2796 hours, 2816 hours, 2836 hours, 2856 hours, 2876 hours, 2896 hours, 2916 hours, 2936 hours, 2956 hours, 2976 hours, 2996 hours, 3016 hours, 3036 hours, 3056 hours, 3076 hours, 3096 hours, 3116 hours, 3136 hours, or 3150 hours, including values or ranges therebetween). In embodiments, a 600 mg intravenous dose of OpSCF provides a Tₗₐₛₜ within the range of 80% to 125% of 2520 hours. In embodiments, a 600 mg intravenous dose of OpSCF provides a Tₗₐₛₜ of about 2520 hours.

In embodiments, a 30 mg intravenous dose of OpSCF provides a Tₗₐₛₜ within the range of 80% to 125% of 168 hours. In embodiments, a 30 mg intravenous dose of OpSCF provides a Tₗₐₛₜ of about 168 hours.

In embodiments, a 100 mg intravenous dose of the OpSCF provides a Tₗₐₛₜ within the range of 80% to 125% of 168 hours. In embodiments, a 100 mg intravenous dose of OpSCF provides a Tₗₐₛₜ of about 168 hours.

In embodiments, a 300 mg intravenous dose of the OpSCF provides a Tₗₐₛₜ within the range of 80% to 125% of 168 hours. In embodiments, a 300 mg intravenous dose of OpSCF provides a Tₗₐₛₜ of about 168 hours.

In embodiments, administering multiple 30 mg doses of OpSCF provides a Tₗₐₛₜ within the range of 80% to 125% of 2520 hours. In embodiments, administering multiple 30 mg doses of OpSCF provides a Tₗₐₛₜ of about 2520 hours.

In embodiments, administering multiple 100 mg doses of OpSCF provides a Tₗₐₛₜ within the range of 80% to 125% of 2520 hours. In embodiments, administering multiple 100 mg doses of OpSCF provides a Tₗₐₛₜ of about 2520 hours.

In embodiments, administering multiple 300 mg doses of OpSCF provides a Tₗₐₛₜ within the range of 80% to 125% of 2520 hours. In embodiments, administering multiple 300 mg doses of OpSCF provides a Tₗₐₛₜ of about 2520 hours.

t_{1/2}

In embodiments, the terminal half life (t_{1/2}) of OpSCF ranges from about 0.05 hours to about 500 hours, 0.5 hours to about 500 hours, including about 0.5 hours, about 1 hours, about 10 hours, about 20 hours, about 30 hours, about 40 hours, about 50 hours, about 60 hours, about 70 hours, about 80 hours, about 90 hours, about 100 hours, about 110 hours, about 120 hours, about 130 hours, about 140 hours, about 150 hours, about 160 hours, about 170 hours, about 180 hours, about 190 hours, about 200 hours, about 210 hours, about 220 hours, about 230 hours, about 240 hours, about 250 hours, about 260 hours, about 270 hours, about 280 hours, about 290 hours, about 300 hours, about 310 hours, about 320 hours, about 330 hours, about 340 hours, about 350 hours, about 360 hours, about 370 hours, about 380 hours, about 390 hours, about 400 hours, about 410 hours, about 420 hours, about 430 hours, about 440 hours, about 450 hours, about 460 hours, about 470 hours, about 480 hours, about 490 hours, or about 500 hours, including any values or ranges therebetween.

### CL/F

In embodiments, the clearance (CL/F) of OpSCF ranges from about 0.0100 L/h to about 0.0200 L/h, including about 0.0100 L/h, about 0.0110 L/h, about 0.0120 L/h, about 0.0130 L/h, about 0.0140 L/h, about 0.0140 L/h, about 0.0150 L/h, about 0.0160 L/h, about 0.0170 L/h, about 0.0180 L/h, about 0.0190 L/h, or about 0.0200 L/h, including any values or ranges therebetween.

### V/F

In embodiments, the volume of distribution (V/F) of OpSCF ranges from about 0.05 L to about 20 L, including about 0.5 L, about 1 L, about 2 L, about 3 L, about 4 L, about 5 L, about 6 L, about 7 L, about 8 L, about 9 L, about 10 L, about 11 L, about 12 L, about 13 L, about 14 L, about 15 L, about 16 L, about 17 L, about 18 L, about 19 L, or about 20 L, including any values or ranges therebetween.

### Vₛₛ

In embodiments, the volume of distribution at steady state (Vₛₛ) of OpSCF ranges from about 0.05 L to about 10 L, including about 0.05 L, about 1 L, about 2 L, about 3 L, about 4 L, about 5 L, about 6 L, about 7 L, about 8 L, about 9 L or about 10 L, including any values or ranges therebetween.

### Serum Concentration

In embodiments, administering OpSCF according to the methods described herein provides a serum concentration of OpSCF ranging from about 0.05 mg/L to about 200 mg/L, including about 0.05 mg/L, about 0.5 mg/L, about 1 mg/L, about 2 mg/L, about 3 mg/L, about 4 mg/L, about 5 mg/L, about 6 mg/L, about 7 mg/L, about 8 mg/L, about 9 mg/L, about 10 mg/L, about 11 mg/L, about 12 mg/L, about 13 mg/L, about 14 mg/L, about 15 mg/L, about 16 mg/L, about 17 mg/L, about 18 mg/L, about 19 mg/L, about 20 mg/L, about 21 mg/L, about 22 mg/L, about 23 mg/L, about 24 mg/L, about 25 mg/L, about 26 mg/L, about 27 mg/L, about 28 mg/L, about 29 mg/L, about 30 mg/L, about 31 mg/L, about 32 mg/L, about 33 mg/L, about 34 mg/L, about 35 mg/L, about 36 mg/L, about 37 mg/L, about 38 mg/L, about 39 mg/L, about 40 mg/L, about 41 mg/L, about 42 mg/L, about 43 mg/L, about 44 mg/L, about 45 mg/L, about 46 mg/L, about 47 mg/L, about 48 mg/L, about 49 mg/L, about 50 mg/L, about 51 mg/L, about 52 mg/L, about 53 mg/L, about 54 mg/L, about 55 mg/L, about 56 mg/L, about 57 mg/L, about 58 mg/L, about 59 mg/L, about 60 mg/L, about 61 mg/L, about 62 mg/L, about 63 mg/L, about 64 mg/L, about 65 mg/L, about 66 mg/L, about 67 mg/L, about 68 mg/L, about 69 mg/L, about 70 mg/L, about 71 mg/L, about 72 mg/L, about 73 mg/L, about 74 mg/L, about 75 mg/L, about 76 mg/L, about 77 mg/L, about 78 mg/L, about 79 mg/L, about 80 mg/L, about 81 mg/L, about 82 mg/L, about 83 mg/L, about 84 mg/L, about 85 mg/L, about 86 mg/L, about 87 mg/L, about 88 mg/L, about 89 mg/L, about 90 mg/L, about 91 mg/L, about 92 mg/L, about 93 mg/L, about 94 mg/L, about 95 mg/L, about 96 mg/L, about 97 mg/L, about 98 mg/L, about 99 mg/L, about 100 mg/L, about 101 mg/L, about 102 mg/L, about 103 mg/L, about 104 mg/L, about 105 mg/L, about 106 mg/L, about 107 mg/L, about 108 mg/L, about 109 mg/L, about 110 mg/L, about 111 mg/L, about 112 mg/L, about 113 mg/L, about 114 mg/L, about 115 mg/L, about 116 mg/L, about 117 mg/L, about 118 mg/L, about 119 mg/L, about 120 mg/L, about 121 mg/L, about 122 mg/L, about 123 mg/L, about 124 mg/L, about 125 mg/L, about 126 mg/L, about 127 mg/L, about 128 mg/L, about 129 mg/L, about 130 mg/L, about 131 mg/L, about 132 mg/L, about 133 mg/L, about 134 mg/L, about 135 mg/L, about 136 mg/L, about 137 mg/L, about 138 mg/L, about 139 mg/L, about 140 mg/L, about 141 mg/L, about 142 mg/L, about 143 mg/L, about 144 mg/L, about 145 mg/L, about 146 mg/L, about 147 mg/L, about 148 mg/L, about 149 mg/L, about 150 mg/L, about 151 mg/L, about 152 mg/L, about 153 mg/L, about 154 mg/L, about 155 mg/L, about 156 mg/L, about 157 mg/L, about 158 mg/L, about 159 mg/L, about 160 mg/L, about 161 mg/L, about 162 mg/L, about 163 mg/L, about 164 mg/L, about 165 mg/L, about 166 mg/L, about 167 mg/L, about 168 mg/L, about 169 mg/L, about 170 mg/L, about 171 mg/L, about 172 mg/L, about 173 mg/L, about 174 mg/L, about 175 mg/L, about 176 mg/L, about 177 mg/L, about 178 mg/L, about 179 mg/L, about 180 mg/L, about 181 mg/L, about 182 mg/L, about 183 mg/L, about 184 mg/L, about 185 mg/L, about 186 mg/L, about 187 mg/L, about 188 mg/L, about 189 mg/L, about 190 mg/L, about 191 mg/L, about 192 mg/L, about 193 mg/L, about 194 mg/L, about 195 mg/L, about 196 mg/L, about 197 mg/L, about 198 mg/L, about 199 mg/L, about 200 mg/L, including any values or ranges therebetween.

In embodiments, administering OpSCF according to the methods described herein provides a serum concentration of OpSCF ranging from about 0.05 mg/L to about 200 mg/L, about 20 mg/L to about 200 mg/L, about 40 mg/L to about 200 mg/L, about 60 mg/L to about 200 mg/L, about 80 mg/L to about 200 mg/L, about 100 mg/L to about 200 mg/L, about 120 mg/L to about 200 mg/L, about 140 mg/L to about 200 mg/L, about 160 mg/L to about 200 mg/L, about 180 mg/L to about 200 mg/L, about 0.05 mg/L to about 180 mg/L, about 20 mg/L to about 180 mg/L, about 40 mg/L to about 180 mg/L, about 60 mg/L to about 180 mg/L, about 80 mg/L to about 180 mg/L, about 100 mg/L to about 180 mg/L, about 120 mg/L to about 180 mg/L, about 140 mg/L to about 180 mg/L, about 160 mg/L to about 180 mg/L, about 0.05 mg/L to about 160 mg/L, about 20 mg/L to about 160 mg/L, about 40 mg/L to about 160 mg/L, about 60 mg/L to about 160 mg/L, about 80 mg/L to about 160 mg/L, about 100 mg/L to about 160 mg/L, about 120 mg/L to about 160 mg/L, about 140 mg/L to about 160 mg/L, about 0.05 mg/L to about 140 mg/L, about 20 mg/L to about 140 mg/L, about 40 mg/L to about 140 mg/L, about 60 mg/L to about 140 mg/L, about 80 mg/L to about 140 mg/L, about 100 mg/L to about 140 mg/L, about 120 mg/L to about 140 mg/L, about 0.05 mg/L to about 120 mg/L, about 20 mg/L to about 120 mg/L, about 40 mg/L to about 120 mg/L, about 60 mg/L to about 120 mg/L, about 80 mg/L to about 120 mg/L, about 100 mg/L to about 120 mg/L, about 0.05 mg/L to about 100 mg/L, about 20 mg/L to about 100 mg/L, about 40 mg/L to about 100 mg/L, about 60 mg/L to about 100 mg/L, about 80 mg/L to about 100 mg/L, about 0.05 mg/L to about 80 mg/L, about 20 mg/L to about 80 mg/L, about 40 mg/L to about 80 mg/L, about 60 mg/L to about 80 mg/L, about 0.05 mg/L to about 60 mg/L, about 20 mg/L to about 60 mg/L, about 40 mg/L to about 60 mg/L, about 0.05 mg/L to about 40 mg/L, about 20 mg/L to about 40 mg/L, or about 0.05 mg/L to about 20 mg/L, including any ranges or values therebetween.

In embodiments, administering OpSCF according to the methods described herein provides a serum concentration of OpSCF ranging from about 40.2 mg/L to about 114 mg/L, including any ranges or values therebetween.

In embodiments, administering OpSCF according to the methods described herein provides a serum concentration within the range of 80% to 12% of 40.2±14.3 mg/L of OpSCF. In embodiments, administering OpSCF according to the methods described herein provides a serum concentration of about 40.2±14.3 mg/L of OpSCF.

In embodiments, administering OpSCF according to the methods described herein provides a serum concentration within the range of 80% to 12% of 63.6±26.9 mg/L of OpSCF. In embodiments, administering OpSCF according to the methods described herein provides a serum concentration of about 63.6±26.9 mg/L of OpSCF.

In embodiments, administering OpSCF according to the methods described herein provides a serum concentration within the range of 80% to 12% of 72±32.1 mg/L of OpSCF. In embodiments, administering OpSCF according to the methods described herein provides a serum concentration of about 72±32.1 mg/L of OpSCF.

In embodiments, administering OpSCF according to the methods described herein provides a serum concentration within the range of 80% to 12% of 79±36.5 mg/L of OpSCF. In embodiments, administering OpSCF according to the methods described herein provides a serum concentration of about 79±36.5 mg/L of OpSCF.

In embodiments, administering OpSCF according to the methods described herein provides a serum concentration within the range of 80% to 12% of 85.7±46.4 mg/L of OpSCF. In embodiments, administering OpSCF according to the methods described herein provides a serum concentration of about 85.7±46.4 mg/L of OpSCF.

In embodiments, administering OpSCF according to the methods described herein provides a serum concentration within the range of 80% to 12% of 82.4±39.9 mg/L of OpSCF. In embodiments, administering OpSCF according to the methods described herein provides a serum concentration of about 82.4±39.9 mg/L of OpSCF.

In embodiments, administering OpSCF according to the methods described herein provides a serum concentration within the range of 80% to 12% of 114±56.9 mg/L of OpSCF. In embodiments, administering OpSCF according to the methods described herein provides a serum concentration of about 114±56.9 mg/L of OpSCF.

In embodiments, administering OpSCF according to the methods described herein provides a serum concentration within the range of 80% to 12% of 111±53.1 mg/L of OpSCF. In embodiments, administering OpSCF according to the methods described herein provides a serum concentration of about 111±53.1 mg/L of OpSCF.

In embodiments, administering OpSCF according to the methods described herein provides a serum concentration within the range of 80% to 12% of 80.6±34.5 mg/L of OpSCF. In embodiments, administering OpSCF according to the methods described herein provides a serum concentration of about 80.6±34.5 mg/L of OpSCF.

### Clinical Endpoints

The efficacy of the methods described herein for treating atopic dermatitis is assessed using one or more of Eczema Area and Severity Index (EASI), validated Investigational Global Assessment for atopic dermatitis (AD) (vIGA-AD), Peak Pruritus Numerical Rating Scale (PP-NRS), percent body surface area (BSA) affected by AD, Atopic Dermatitis Control Tool (ADCT) score, Patient Oriented Eczema Measure (POEM) score, and Dermatology Life Quality Index (DLQI) score.

### Eczema Area and Severity Index (EASI)

In embodiments, administering OpSCF according to the methods described herein reduces an Eczema Area and Severity Index (EASI) score of an adult human patient. EASI scores range from 0 to 72 and are a validated measure of activity of atopic dermatitis, where a score of 0 indicates clear or no eczema, 0.1 to 1.0 indicates almost clear, 1.1 to 7 indicates mild disease, 7.1 to 21 indicates moderate disease, 21.1 to 50 indicates severe disease, and greater than 51 indicates very severe disease. An exemplary EASI calculation is described in Hanifin J al. The Eczema Area and Severity Index-A Practical Guide. Dermatitis. 2022 May-Jun. 1; 33(3):187-192. This reference is incorporated by reference herein in its entirety for all purposes. Briefly, to calculate the EASI scores the body can be divided into four regions: head and neck, upper extremities, trunk, and lower extremities. The EASI score assesses the severity of four characteristics of atopic dermatitis (AD): erythema, edema/papulation, excoriation, and lichenification. The severity of each characteristic is scored on a scale of 0 to 3, with 0 being absent and 3 being severe. The percentage of body surface area (BSA) for each region is assigned, and each subtotal score is multiplied by that percentage.

In embodiments, a patient exhibits at least 50%, at least 55%, at least 60%, at least 65%, at least 70%, at least 75%, at least 80%, at least 85%, at least 90%, at least 95%, or 100% reduction in an EASI score after treating according to the methods described herein compared to an EASI score before treating. In embodiments, the patient exhibits at least a 70%, 75%, 80%, 85%, or 90% reduction in an EASI scale score after treating according to the methods described herein compared to an EASI scale score before treating.

vIGA-AD

In embodiments, administering OpSCF according to the methods described herein reduces a validated Investigational Global Assessment for atopic dermatitis (AD) (vIGA-AD). The vIGA-AD scale is a clinician rated scale to assess the overall severity of AD lesions at a given time point. The vIGA-AD score ranges from 0 to 4, including 0 (clear), 1 (almost clear), 2 (mild), 3 (moderate), and 4 (severe) based on four clinical features of AD lesions: erythema, induration/papulation, lichenification and oozing/crusting. An exemplary method of determining a vIGA-AD can be found in Simpson E L, et al. The Validated Investigator Global Assessment for Atopic Dermatitis (vIGA-AD™): a clinical outcome measure for the severity of atopic dermatitis. Br J Dermatol. 2022 October; 187 (4): 531-538. This reference is incorporated by reference herein in its entirety.

In embodiments, a patient exhibits at least a one point, two point, three point, or four point reduction in a vIGA-AD score after treating according to the methods described herein compared to a vIGA-AD score before treating. In embodiments, after administering OpSCF according to the methods described herein, the patient exhibits a vIGA-AD score of 0 or 1.

### PP-NRS

In embodiments, administering OpSCF according to the methods described herein reduces a patient's score on the Peak Pruritus Numerical Rating Scale (PP-NRS). PP-NRS is a validated scale of subject-reported itch that is designed to measure peak pruritus, or ‘worst’ itch, over the previous 24 hours based on the following question: ‘On a scale of 0 to 10, with 0 being “no itch” and 10 being “worst itch imaginable”. In embodiments, a patient exhibits at least a one point, two point, three point, four point, five point, 6 point, 7 point, 8 point, 9 point or 10 point reduction in a PP-NRS score for AD after treating according to the methods described herein compared to a PP-NRS score for AD before treating. In embodiments, after administering OpSCF according to the methods described herein the patient exhibits a PP-NRS score for AD of 0 to 5.

### BSA

In embodiments, administering OpSCF according to the methods described herein reduces the body surface area (BSA) affected by AD. In embodiments, a patient exhibits at least 50%, at least 55%, at least 60%, at least 65%, at least 70%, at least 75%, at least 80%, at least 85%, at least 90%, at least 95%, or 100% reduction in an AD related BSA percentage after treating according to the methods described herein compared to an AD related BSA percentage before treating. In embodiments, the patient exhibits at least a 70%, 75%, 80%, 85%, or 90% reduction in an AD related BSA percentage after treating according to the methods described herein compared to an AD related BSA percentage before treating.

### Atopic Dermatitis Control Tool

In embodiments, administering OpSCF according to the methods described herein reduces an Atopic Dermatitis Control Tool (ADCT) score. The ADCT includes size questions that evaluate criteria of AD over the past week: overall symptom severity, days with intense itching, bother intensity, sleep problems, impact on daily activities, and impact on mood or emotions. ADCT scores range from 0 to 24, with scores of 0-4 based on increased severity for each of the six questions.

In embodiments, a patient exhibits at least 50%, at least 55%, at least 60%, at least 65%, at least 70%, at least 75%, at least 80%, at least 85%, at least 90%, at least 95%, or 100% reduction in an ADCT score after treating according to the methods described herein compared to an ADCT score before treating. In embodiments, the patient exhibits at least a 70%, 75%, 80%, 85%, or 90% reduction in an ADCT score after according to the methods described herein compared to an ADCT score before treating.

### Patient Oriented Eczema Measure

In embodiments, administering OpSCF according to the methods described herein reduces a Patient Oriented Eczema Measure (POEM) score. The POEM assessment includes a questionnaire with 7 items that patients complete to evaluate their eczema symptoms over the past week, with each item having a score ranging from 0 (not at all) to 4 (very much). The total score ranges from 0 to 28, with a higher score indicating more severe symptoms.

In embodiments, a patient exhibits at least 50%, at least 55%, at least 60%, at least 65%, at least 70%, at least 75%, at least 80%, at least 85%, at least 90%, at least 95%, or 100% reduction in a POEM score after treating according to the methods described herein compared to a POEM score before treating. In embodiments, the patient exhibits at least a 70%, 75%, 80%, 85%, or 90% reduction in a POEM score after treating according to the methods described herein compared to a POEM score before treating.

### Dermatology Life Quality Index

In embodiments, administering OpSCF according to the methods described herein reduces a Dermatology Life Quality Index (DLQI) score. The DLQI assessment includes a questionnaire with 10 items to be completed by patients that cover various topics (e.g., symptoms, daily activity, work/school, personal relationship and treatment.) Each question is given a score from 0 to 3, with a higher score indicating a greater negative impact on quality of life. The total score ranges from 0 to 30, with a higher score indicating a larger effect of AD on quality of life.

In embodiments, a patient exhibits at least 50%, at least 55%, at least 60%, at least 65%, at least 70%, at least 75%, at least 80%, at least 85%, at least 90%, at least 95%, or 100% reduction in a DLQI score after treating according to the methods described herein compared to an DLQI score before treating. In embodiments, the patient exhibits at least a 70%, 75%, 80%, 85%, or 90% reduction in a DLQI score after treating according to the methods described herein compared to an DLQI score before treating.

### EXAMPLES

The following examples are given for the purpose of illustrating various embodiments of the disclosure and are not meant to limit the present disclosure in any fashion. Changes therein and other uses which are encompassed within the spirit of the disclosure, as defined by the scope of the claims, will be recognized by those skilled in the art.

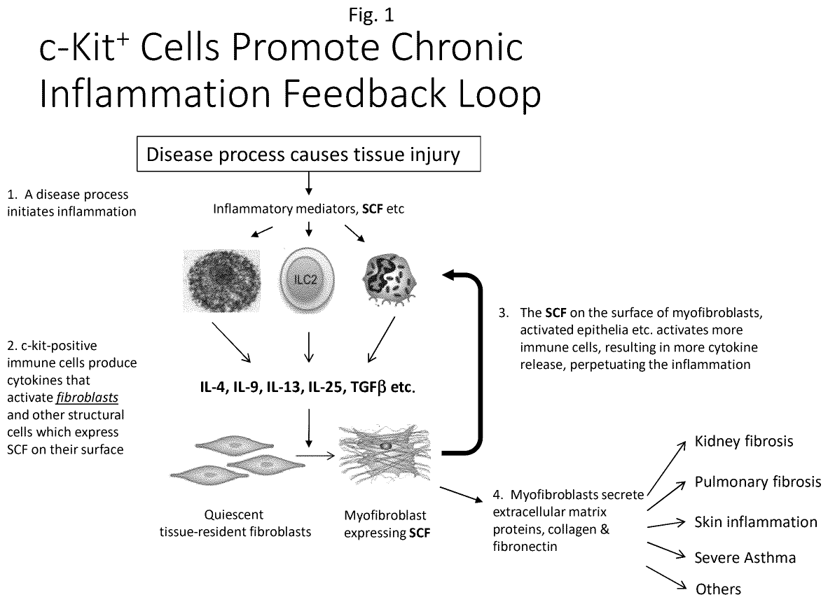

An overview of the tissue injury/disease process is summarized in FIG. 1. A disease process initiates inflammation. c-Kit+ immune cells produce cytokines that cause fibroblasts to change into activated myofibroblasts which express SCF248 on their surface. The expression of SCF248 on the surface of myofibroblasts and other cells activates more immune cells, resulting in cytokine release of IL-4, IL-9, IL-13, IL-25, TGFβ, and other cytokines, perpetuating inflammation. Myofibroblasts secrete extracellular matrix proteins, collagen, and fibronectin, leading to fibrosis and remodeling diseases such as pulmonary fibrosis, skin fibrosis, severe asthma, and other diseases.

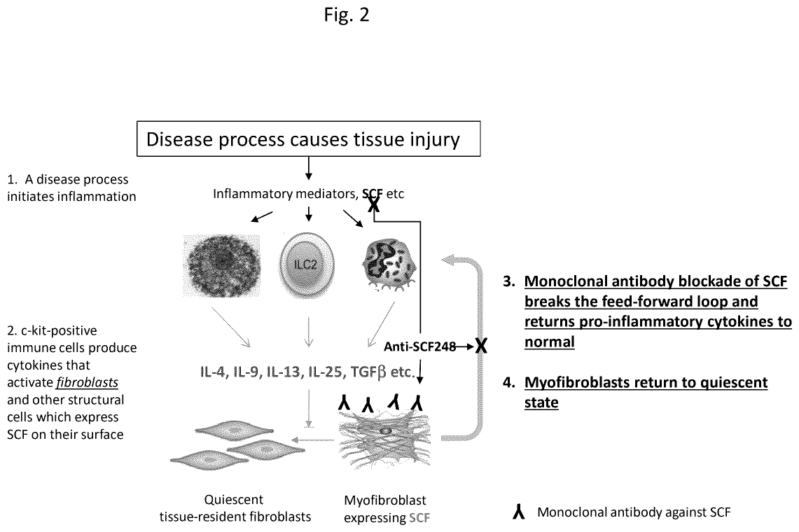

An exemplary mechanism of an antibody of the instant disclosure which targets SCF248 (said antibody referred to herein as OpSCF and/or as 5H10) is summarized in FIG. 2.

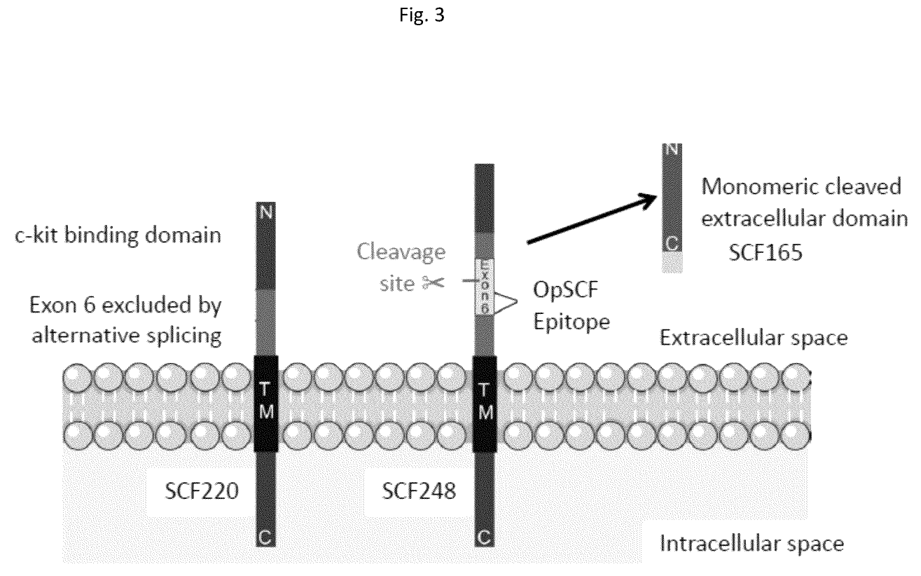

As provided above, SCF has two isoforms which result from alternative splicing: SCF248 and SCF220. SCF248 and SCF220 differ by exon 6. SCF220 is associated with homeostatic functions, and SCF248 is associated with inflammation and fibrosis. SCF248 activates immune cells during inflammation and is sometimes called “soluble SCF.” SCF248 is expressed on various cell types including myofibroblasts, activated epithelia, endothelia, macrophages, eosinophils, mast cells, and monocytes (FIG. 3). The SCF248 isoform results in cleavage of monomeric cleaved extracellular domain, called SCF165. The amino acid sequence of exon 6 is provided herein as SEQ ID NO: 34.

### Example 1: Production of Anti-SCF mAbs Utilizing Hybridoma Technology

A peptide comprising ASSLRNDSSSSNRKAKNPPGD (SEQ ID NO: 30) was used to generate antibodies that bind to SCF248. The immunization peptide comprised a portion of exon 6, i.e. the SCF248 isoform of stem cell factor. In particular, the immunization peptide comprised a portion of exon 6 that begins after a cleavage site as defined herein. Mice were immunized with a peptide according to SEQ ID NO: 30 with a standard protocol. The determination of high titer serum antibodies indicated the appropriate immunization and fusion hybridomas were made. Culture supernatants were analyzed from individual clones for SCF-specific antibodies and chosen based upon specificity. Hybridomas producing specific monoclonal antibodies against the peptide were propagated and the monoclonal with the highest titer was subsequently tested in biologically relevant cultures. Antibody 5H10 had high specificity for SCF248 and no cross-reactivity with SCF220. No other monoclonal antibodies produced by the hybridomas had high specificity for SCF248 without cross-reactivity with SCF220. Thus, 5H10 was selected for further characterization, development, and chimerization and subsequent humanization.

### Example 2: 5H10 Binding to SCF248 Complete Extracellular Domain

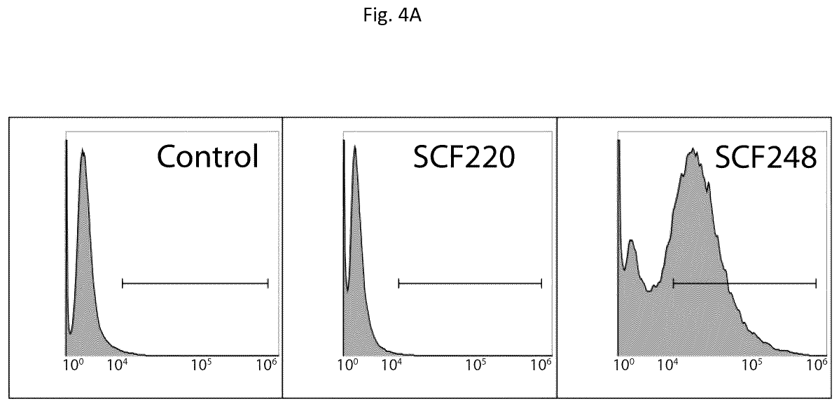

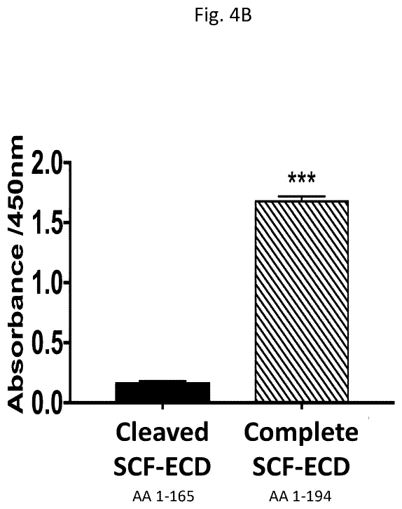

The murine 5H10 antibody obtained as described in Example 1 was directly conjugated with a fluorescent marker and the labeled antibody was incubated with S1/S14 hSCF248 cells, which express SCF248; S1/S14 hSCF220 cells, which express SCF220; or control cells that do not express SCF. Binding of the labeled antibody to the cells was assessed by flow cytometry. The specificity of 5H10 for SCF248 and lack of crossreactivity with SCF220 is shown in FIG. 4A.

Binding of the murine 5H10 antibody to the cleaved extracellular domain (ECD) containing only amino acids 1-165 of SCF, vs the complete ECD containing amino acids 1-194 of SCF, was assessed by an ELISA method. The antibody bound to the complete SCF ECD but not to the cleaved SCF ECD (FIG. 4B), demonstrating that the antibody is specific for the complete extracellular domain and does not bind to the monomeric cleaved ECD that circulates in blood.

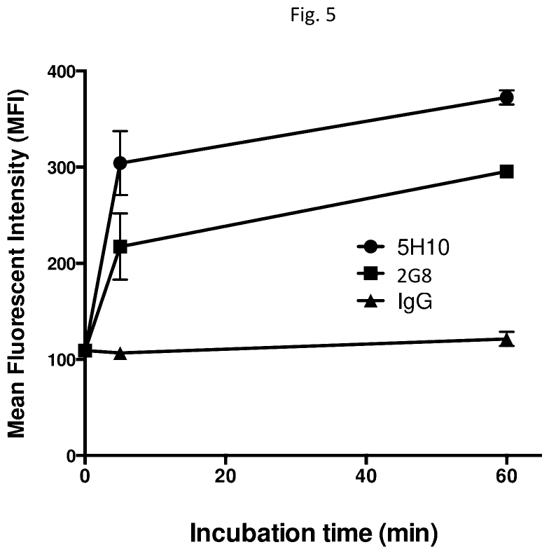

To assess the ability of 2G8 and 5H10 antibodies to internalize SCF248 on myofibroblasts, antibodies were labeled with PHRODO™ red, which is colorless at neutral pH and fluoresces red at the low pH within an endosome. Labeled antibodies were incubated with cultured human IPF myofibroblasts for 45 minutes and red fluorescence was visualized by microscopy. As shown in FIG. 5, the dye-labeled antibodies, but not control IgG, were rapidly internalized. 5H10 was internalized more rapidly and resulted in higher fluorescence compared to 2G8.

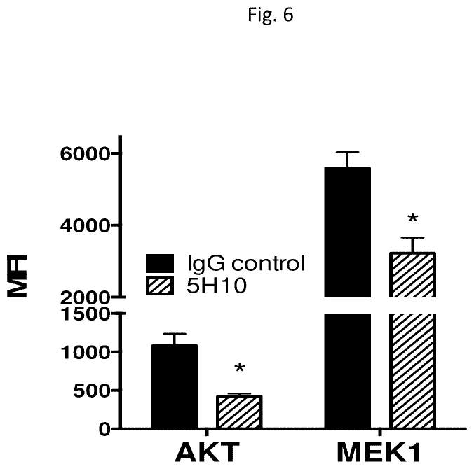

SCF triggers c-kit to signal by two distinct pathways: the MEK/ERK pathway and the P13K/AKT pathway. A study was conducted to determine whether the murine 5H10 antibody inhibits intracellular signaling in c-kit positive cells in either or both of these pathways. Eosinophils were incubated with SCF248-expressing cell lines, in the presence of either 5H10 or IgG control, and phospho-protein expression was measured with a BIO-RAD® BIO-PLEX® assay system. 5H10 significantly decreased the phospho-MEK and phosphor-AKT levels, indicating that the antibody significantly reduced c-kit mediated intracellular signaling (FIG. 6).

Taken together, the results of these studies indicated that antibody 5H10 binds specifically to and internalizes SCF248, and does not cross-react with the SCF220 isoform or the cleaved ECD. Moreover, 5H10 significantly inhibits the intracellular signaling pathways in c-kit positive cells that perpetuate inflammation.

### Example 3: Humanization of Murine Antibody 5H10

Chimeric antibodies derived from 5H10 were produced by subcloning the variable domains of the heavy and light chains into a vector with a human IgG4 backbone. Chimeric antibodies were expressed and purified using standard protocols. 2G8 is a previously developed antibody that binds to SCF248 and SCF220, and contains a lambda light chain. The chimeric heavy and light chains of 2G8 were named VH0 and VL0, respectively. 5H10, the SCF248-specific antibody provided herein, contains a kappa light chain. The chimeric heavy and light chains of 5H10 were named VH0 and VK0, respectively.

The chimeric antibodies were humanized. Humanized heavy chains retained the same complementarity-determining regions (CDRs) but more “human-like” framework regions, and several humanized variants of each of 2G8 and 5H10 variable heavy chains, referred to herein as VH1, VH2, VH3, VH4, and VH5, were generated. Humanized kappa light chain variants of 5H10, referred to herein as VK1, VK2, VK3, and VK4, were also generated. Humanized lambda light chains of 2G8 were named VL1, VL2, VL3, and VL4. The 2G8 and 5H10 combinations of chimeric and humanized light chains and heavy chains tested are shown in Table 3 and Table 4, respectively. As shown in Table 3, certain heavy and light chain combinations of the 5H10 antibody variants resulted in high binding to hSCF248. Binding data used to determine the binding score are provided below in Example 5.

TABLE 3
Binding score for 2G8 chimeric and humanized
clones to Sl/Sl4 hSCF248 cells

| 2G8 mAb | Binding Score |
|---|---|
| VH0/VL0 | high |
| VH1/VL1 | Moderate high |
| VH1/VL3 | moderate |
| VH1/VL4 | Moderate |
| VH2/VL1 | Moderate |
| VH2/VL4 | Moderate |
| VH3/VL1 | Moderate |
| VH3/VL4 | Moderate |
| VH4/VL1 | Moderate |
| VH4/VL2 | Moderate |
| VH5/VL1 | Moderate high |
| VH5/VL4 | Moderate |

TABLE 4
Binding score for 5H10 chimeric and humanized
clones to Sl/Sl4 hSCF248 cells

| 5H10 mAb | Binding Score |
|---|---|
| VH0/VK0 | high |
| VH1/VK1 | High |
| VH1/VK2 | High |
| VH1/VK3 | High |
| VH1/VK4 | No binding |
| VH2/VK2 | Moderate high |
| VH2/VK3 | high |
| VH3/VK2 | Moderate |
| VH3/VK3 | Moderate |
| VH4/VK2 | Moderate low |
| VH4/VK3 | Moderate low |
| VH5/VK2 | Low |
| VH5/VK3 | low |

Binding affinity was also assessed using a surface plasmon resonance (BIACORE™) analysis. The surface plasmon resonance (BIACORE™) data showed that the affinity for immobilized SCF248 peptide antigen of all humanized 5H10 antibodies having the VK1, VK2, or VK3 light chain was very similar to the binding affinity of the parental murine 5H10 using this assay. Humanized 5H10 antibodies having a VK4 light chain did not bind to the peptide.

TABLE 5
Surface Plasmon Resonance (BIACORE ™) data

| | VK0 | VK1 | VK2 | VK3 | VK4 |
|---|---|---|---|---|---|
| VH0 | 1.00 | 0.91 | | | |
| VH1 | 0.92 | 0.99 | 0.97 | 0.94 | — |
| VH2 | | 0.98 | 0.99 | 0.94 | — |
| VH3 | | 1.15 | 1.16 | 1.10 | — |
| VH3 | | 1.27 | 1.32 | 0.85 | — |
| VH5 | | 1.07 | 0.93 | 1.09 | — |

### Example 4: Evaluation of Anti-SCF Chimeric Antibody Binding by Flow Cytometry

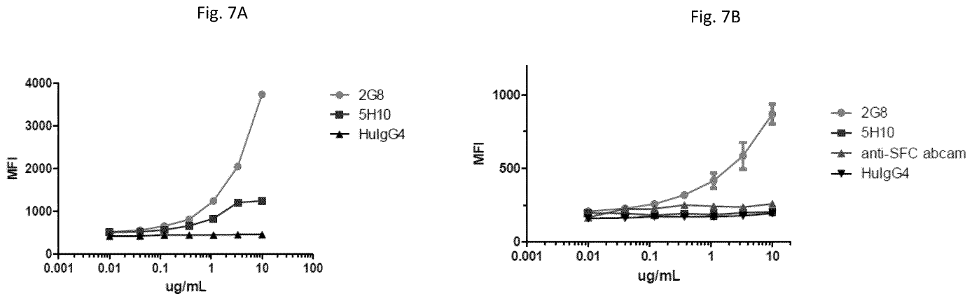

S1/S14 hSCF248 cells, a transfected cell line that expresses SCF248, were utilized to test the binding of the chimeric antibodies 2G8 and 5H10. An anti-SCF antibody was used as a positive control for SCF binding. A human IgG4 antibody was used as a negative isotype control antibody. S1/S14 hSCF248 cells at early passage (P3) were compared to cells at later passage (P5). As expected, the negative control (human IgG4 antibody) did not bind to either cell population. 5H10 bound to cells at early passage (FIG. 7A), but was not detected at later passage (FIG. 7B), due to loss of expression of SCF248 over multiple passages. Similarly, the maximum mean fluorescence intensity (MFI) detected with 10 μg/mL of 2G8 was reduced approximately four times at P5 compared to P3.

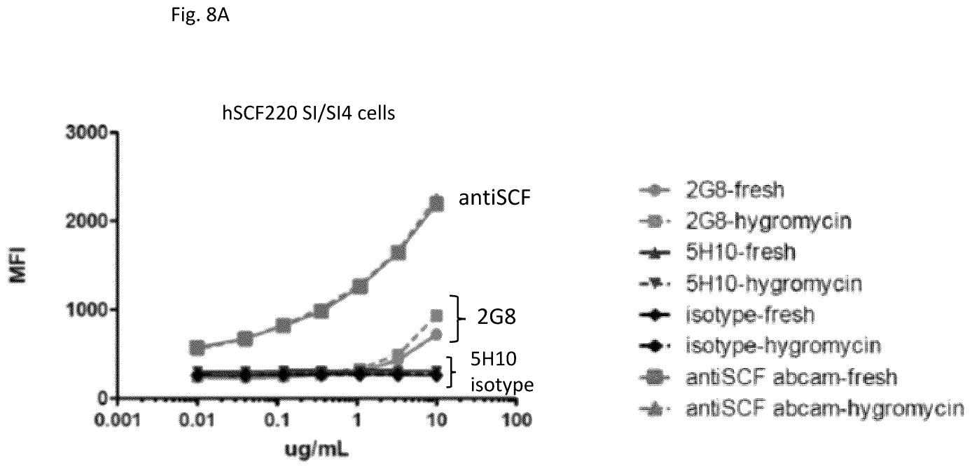

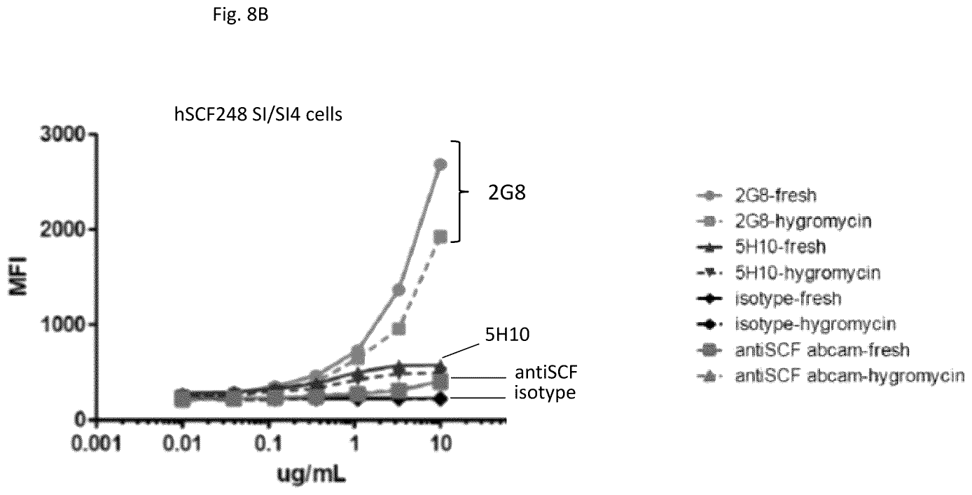

Similar observations were seen with hygromycin B-treated cells. Hygromycin selection was used to enrich the fraction of S1/S14 cells expressing the relevant human SCF sequence. The binding of the two chimeric anti-SCF mAbs 2G8 and 5H10 and humanized anti-SCF antibodies were evaluated via flow cytometry. S1/S14 hSCF248 cells and S1/S14 hSCF220 cells, which are SCF248+ and SCF220+, respectively were utilized to test the binding and specificity of the chimeric antibodies 2G8 and 5H10. An anti-SCF antibody was used as a positive control for SCF binding. A human IgG4 antibody was used as a negative isotype control antibody. Hygromycin B treated cells were compared to early passage (P3) cells. The maximum mean fluorescent intensities (MFIs) and antibody dose response curves were similar to early passage (P3) cells (FIG. 8A, FIG. 8B).

### Example 5: Binding Assessment of 2G8 and 5H10 Humanized mAbs by Flow Cytometry

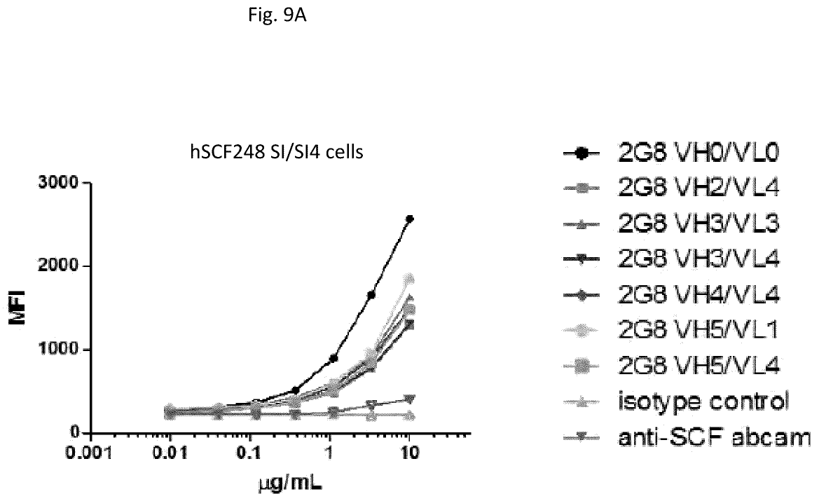

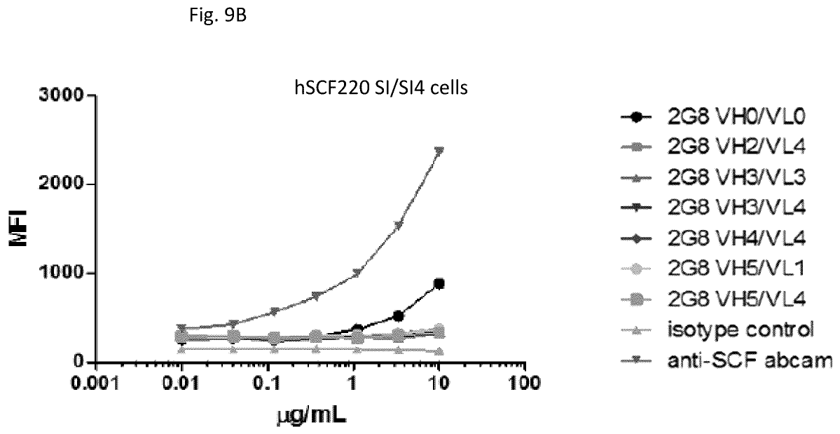

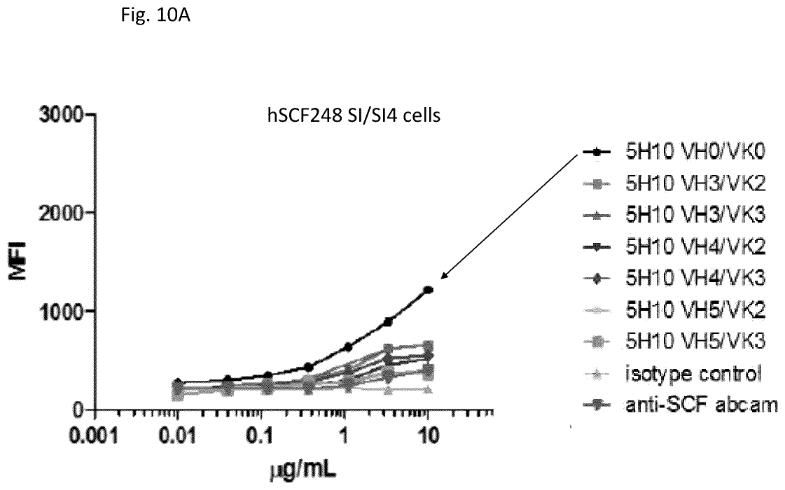

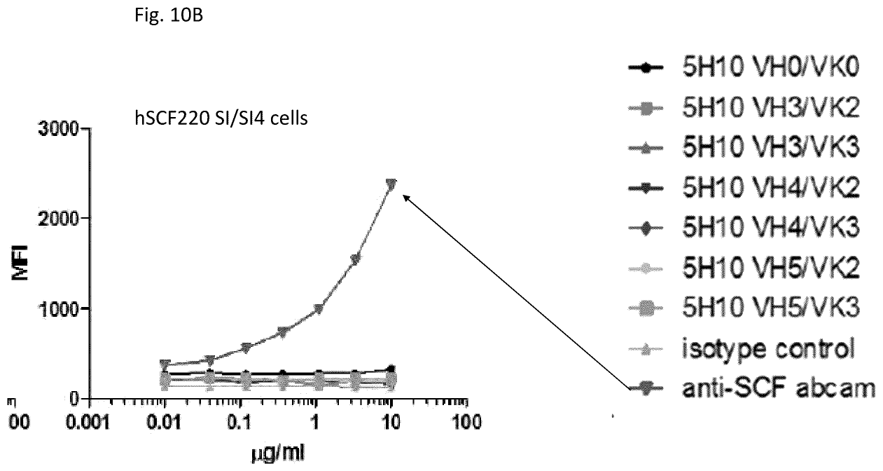

S1/S14 hSCF248 cells and S1/S14 hSCF220 cells, which are SCF248+ and SCF220+, respectively were utilized to test the binding and specificity of the chimeric antibodies 2G8 and 5H10, and humanized variants thereof. In both sets of experiments, a human IgG4 antibody was used as a negative isotype control antibody. The isotype control did not bind to S1/S14 hSCF248 cells or S1/S14 hSCF220 cells (FIGS. 9A, 9B, 10A, and 10B). The commercially available anti-SCF antibody (Abcam, Cat #EP665Y/ab52603) was found to bind S1/S14 hSCF220 cells and weakly to S1/S14 hSCF248 cells (only at a 1:25 dilution) (FIGS. 9A, 9B, 10A, and 10B). 2G8 VH0/VL0 (chimeric 2G8) exhibited stronger binding than its humanized clones. However, 2G8 VH0/VL0 also showed binding to S1/S14 hSCF220 cells at antibody concentrations of 3.3 and 10 μg/mL (FIG. 9A, FIG. 9B). 5H10 VH0/VK0 showed higher binding compared to humanized clones 5H10 VH3/VK2, 5H10 VH3/VK3, 5H10 VH4/VK2, 5H10 VH4/VK3, 5H10 VH5/VK2, and 5H10 VH5/VK3 (FIG. 10A). No binding of 5H10 or any humanized variants thereof to S1/S14 hSCF220 cells was observed (FIG. 10B). Differences among the indicated 5H10 humanized variants in terms of 50% maximal binding (BC₅₀) in this study are shown in Table 6. The binding of the 5H10 clones reached saturation at 3.3 μg/mL on S1/S14 hSCF248 cells. (FIG. 10A).

TABLE 6
BC₅₀ (μg/mL) values for binding of chimeric and humanized
5H10 variants, isotype control, and commercially available
anti-SCF antibody to Sl/Sl4 hSCF248 cells

| Sample ID | BC₅₀ (μg/mL) |
|---|---|
| 5H10 VH0/VK0 | 5.47 |
| 5H10 VH3/VK2 | 1.36 |
| 5H10 VH3/VK3 | 0.90 |
| 5H10 VH4/VK2 | 1.91 |
| 5H10 VH4/VK3 | 1.18 |
| 5H10 VH5/VK2 | 2.12 |
| 5H10 VH5/VK3 | 0.67 |
| Isotype control | n/a |
| Anti-SCF Abcam | 3.02 |

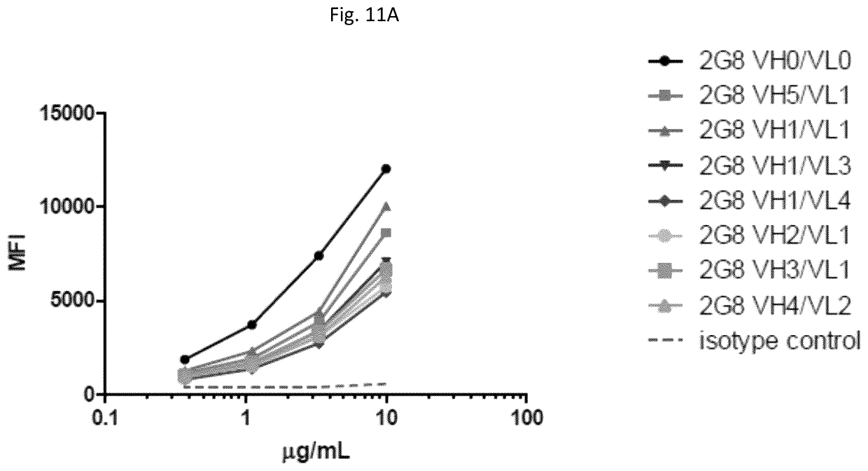

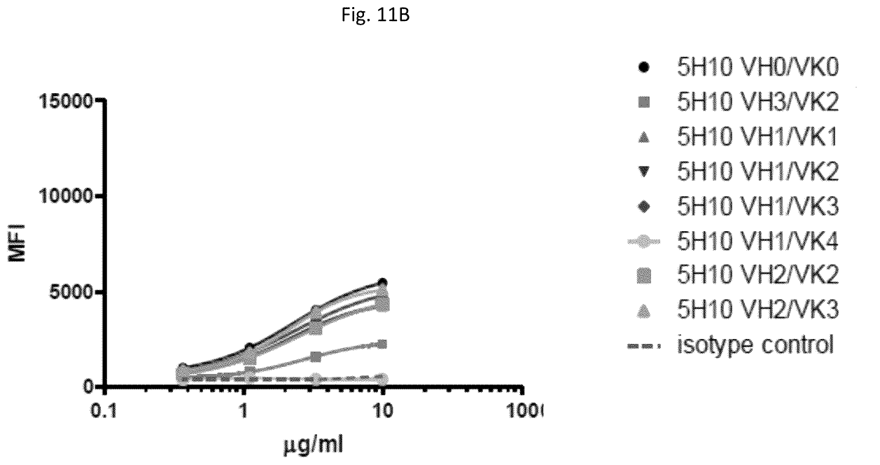

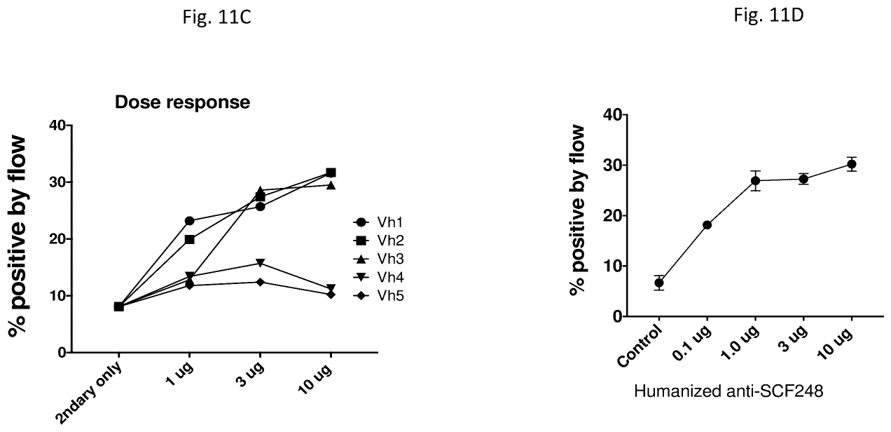

S1/S14 hSCF248 cells were utilized to test the binding of additional humanized variants of 5H10 and 2G8. 2G8 VH0/VL0 (chimeric) showed stronger cell binding than the humanized variants (FIG. 11A). The binding profiles of the humanized 5H10 clones 5H10 VH1/VK1, 5H10 VH1/VK2, 5H10 VH1/VK3, 5H10 VH2/VK2, 5H10 VH2/VK3 were comparable to that of 5H10 VH0/VK0 (FIG. 11B). In agreement with surface plasmon resonance (BIACORE™) data, presented above, the humanized variant 5H10 VH1/VK4 lost target binding (FIG. 11B). The isotype control did not bind to the S1/S14 hSCF248 cells. Based on the data presented in these studies, the humanized 2G8 and 5H10 mAbs were assigned a binding score to S1/S14 hSCF248 cells, which is presented in Tables 3 and 4, above.

In a separate experiment, 5H10 clones VH1/VK3, VH2/VK3, VH3/VK3, VH4/VK3, and VH5/VK3 were assessed by flow cytometry for binding to the SCF248-expressing cell line. As shown in FIGS. 11C and 11D, VH1/VK3 and VH2/VK3 exhibited high binding, maximized at 1 μg/mL. The negative control was secondary antibody only. No binding was observed with the control SCF220-expressing cell line (not shown).

### Example 6: In Vitro Blockade of the Interaction of SCF and c-Kit

The humanized 5H10 antibodies were tested for their capacity to inhibit the SCF-c-kit interaction and the inflammation feed-forward loop in vitro. Cultured human IPF myofibroblasts (Mfb), which express surface SCF248, were overlaid with LAD2 mast cells, an SCF-responsive cell line. Absent any other intervention, the Mfb stimulate the LAD2 cells, which produce cytokines to stimulate Mfb to produce additional cytokines and extracellular matrix proteins. In this assay, the readout for inflammation and the feed-forward loop is mRNA for CCL11, collagens 1 and 3, and fibronectin.

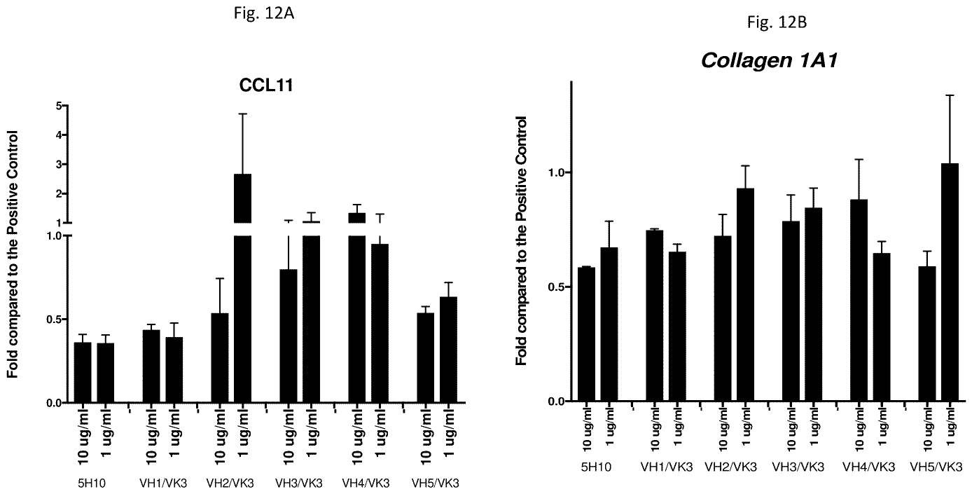

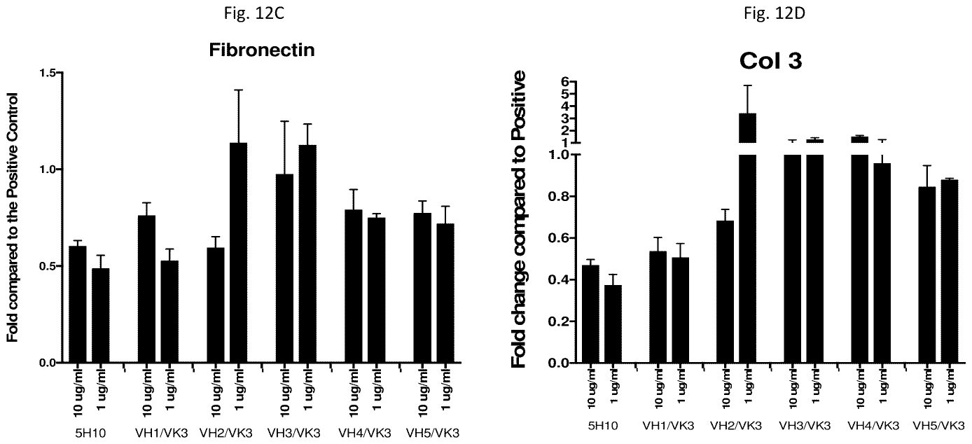

Murine 5H10 and humanized (VH1/VK3, VH2/VK3, VH3/VK3, VH4/VK3, and VH5/VK3) 5H10 antibodies were pre-incubated with Mfb at concentrations of 1 μg/mL and 10 μg/mL to assess their capacity to inhibit the feed-forward loop. Results are shown in FIGS. 12A-12D. The humanized VH1/VK3 antibody consistently demonstrated inhibition of the SCF-c-kit interaction, even at the lower concentration.

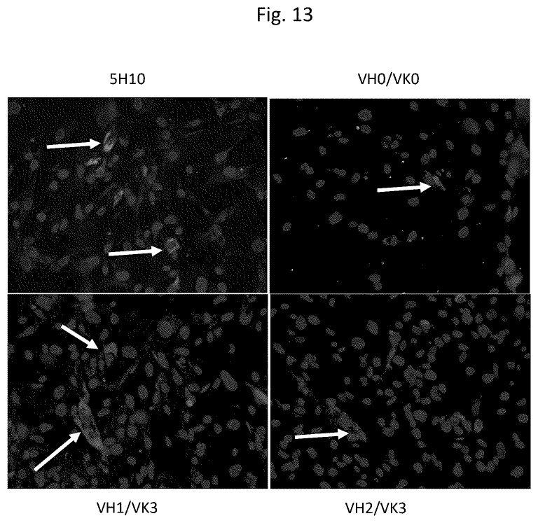

To assess the ability of humanized antibodies (5H10 VH1/VK3 and VH2/VK3) to internalize SCF248 on myofibroblasts, antibodies were labeled with PHRODO™ red dye, which is colorless at neutral pH and fluoresces red at the low pH within an endosome. Labeled antibodies were incubated with cultured human IPF myofibroblasts for 45 minutes and red fluorescence was visualized by microscopy. As shown in FIG. 13, like the murine parent 5H10 antibody and the chimeric antibody (VH0/VK0), the humanized antibodies were rapidly internalized.

### Example 7: Immunogenicity of Chimeric Antibody 5H10 and Humanized Lead Candidates

The immunogenic potential of the five humanized antibodies 5H10 VH1/VK3, 5H10 VH2/VK3, 5H10 VH3/VK3, 5H10 VH4/VK3, and 5H10 VH5/VK3 was compared to the chimeric antibody 5H10 VH0/VK0.

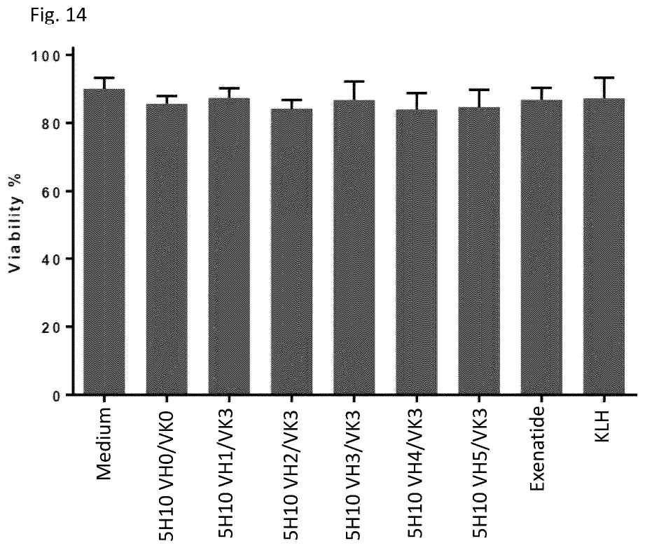

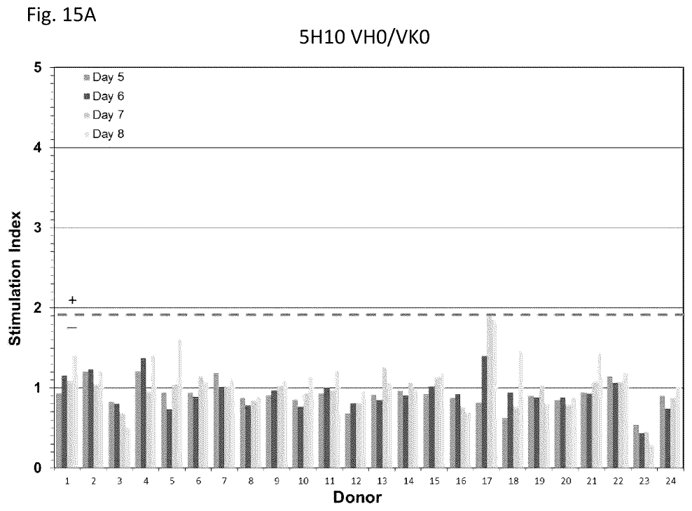

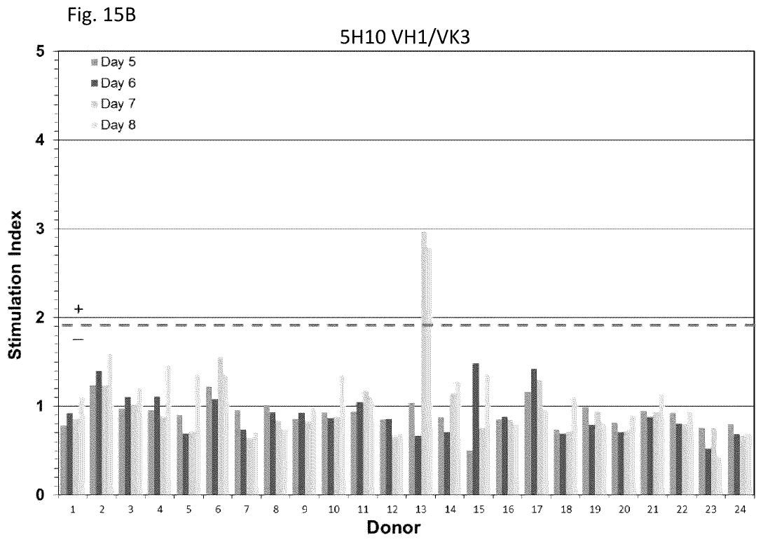

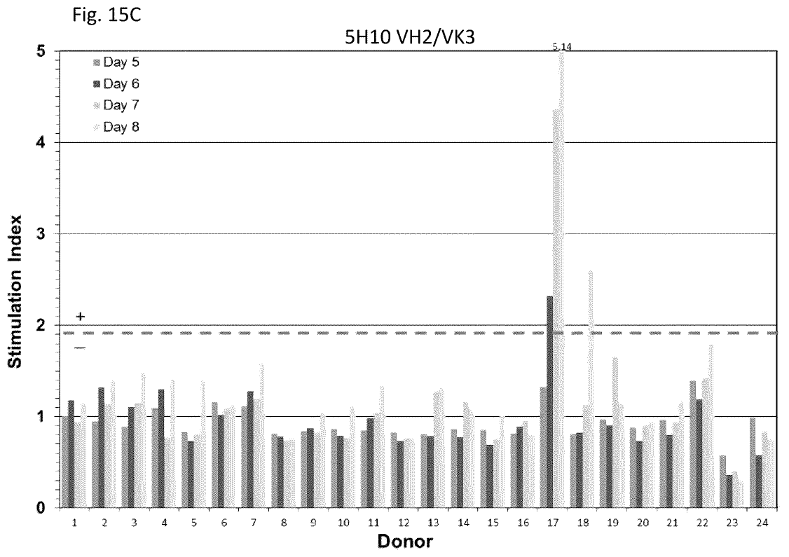

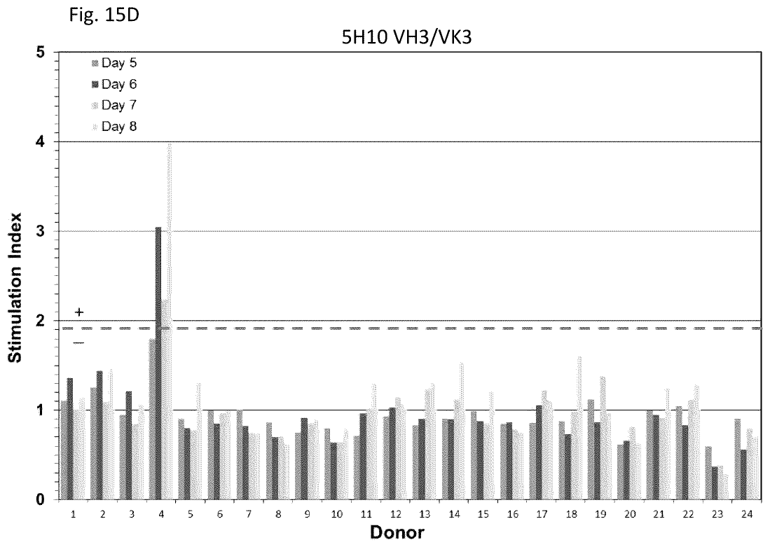

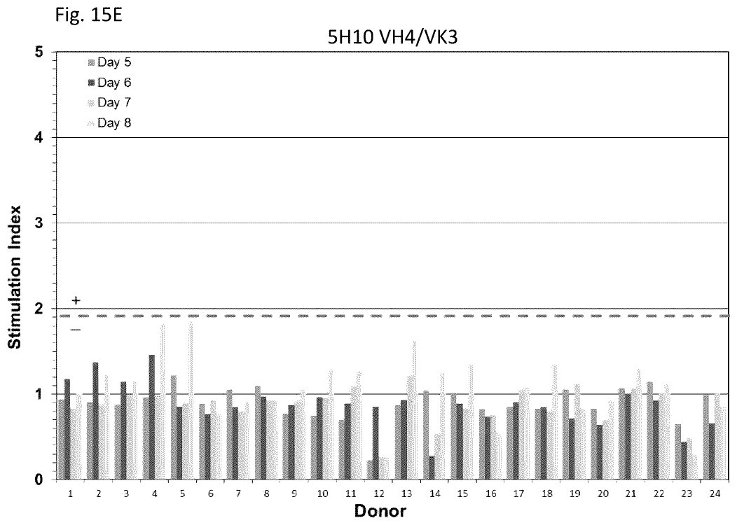

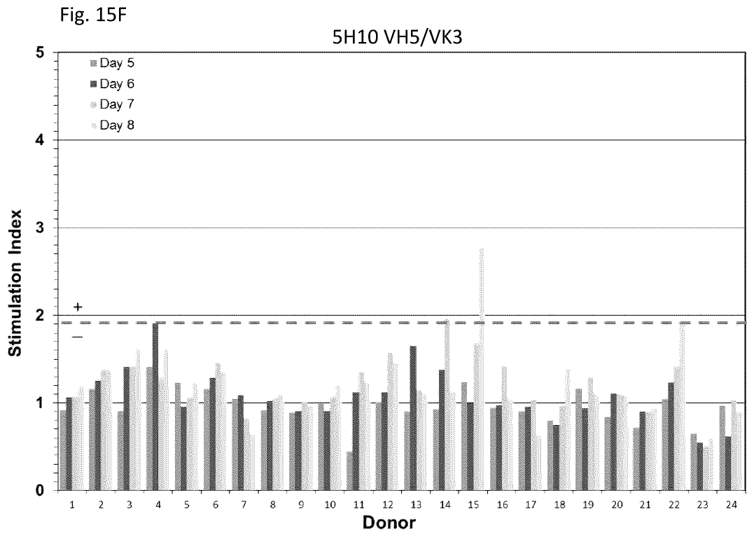

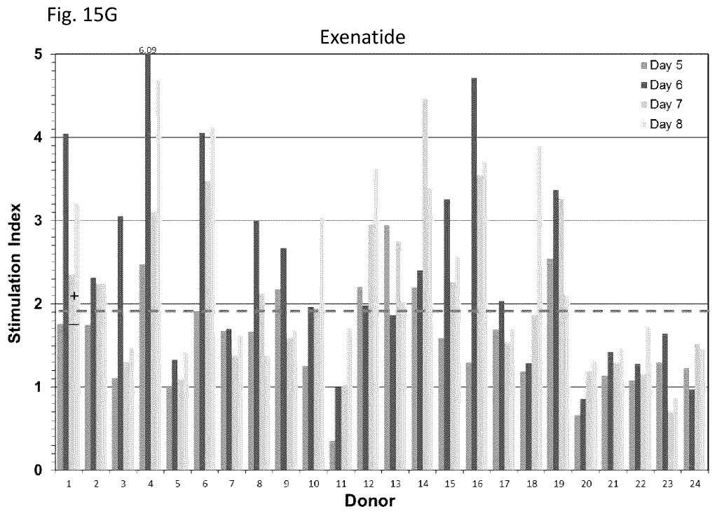

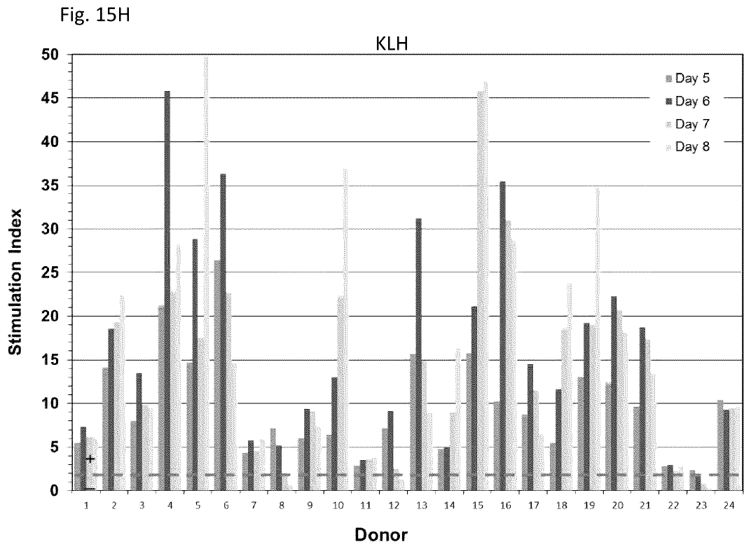

An initial assessment of any cytotoxic effects of the samples on PBMC viability was performed for five donors used in the EPISCREEN™ time course assays. CD8+ T cell depleted PBMC were incubated with the samples and the viability of the cells was determined using a LUNA-FL™ Automated Cell Counter on day 7. The results showed that the mean viabilities of PBMC from five donors treated with anti-SCF mAb were similar to that of cells treated with medium alone, ranging between 83% and 90% (FIG. 14). KLH (Pierce, Life Technologies, UK) was used as a neoantigen. Exenatide (Bydueon, AstraZeneca, UK) was used as a clinical benchmark control. FIG. 15A-H and Table 7 reveal the results obtained the EPISCREEN™ time course T cell proliferation assay of CD4+ T cell responses induced by the samples and controls. Both the clinical benchmark, exenatide, and the neo-antigen, KLH, elicited positive proliferative responses. A low frequency of positive response (SI≥1.90, p<0.05) rates were induced by 5H10 VH1/VK3, 5H10 VH2/VK3, and 5H10 VH3/VK3, ranging from 4% to 8%. Sample 5H10 VH5/VK3 induced higher positive responses in 17% of the donor cohort. 5H10 VH0/VK0 and 5H10 VH4/VK3 did not induce any positive responses. The mean magnitudes of the positive T cell proliferation responses were between 2.14 and 3.61 for all samples (Table 8).

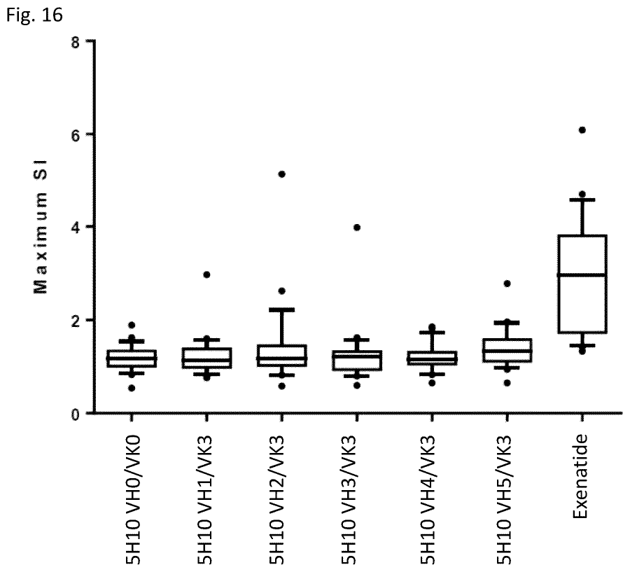

Variance analysis, ANOVA, of the whole proliferation data set (using maximum magnitude of proliferation between days 5-8) was used to determine if there were any statistically significant differences in the maximum magnitude of CD4+ T cell responses to the test conditions compared to each other and the clinical benchmark, exenatide (FIG. 16). The maximum magnitude of T cell proliferative responses to exenatide were statistically higher than the responses to all the samples (Table 9).

TABLE 7
Summary of healthy donor T cell proliferative responses. Positive T cell responses
for proliferation (SI ≥ 1.90, p < 0.05) during the entire time course days 5-8 (“P”). The
frequency of positive responses is shown as a percentage at the bottom of the columns.

| | 5H10 | 5H10 | 5H10 | 5H10 | 5H10 | 5H10 | | |
|---|---|---|---|---|---|---|---|---|
| | VH0/VK0 | VH1/VK3 | VH2/VK3 | VH3/VK3 | VH4/VK3 | VH5/VK3 | Exenatide | KLH |
| Donor 1 | | | | | | | P | P |
| Donor 2 | | | | | | | P | P |
| Donor 3 | | | | | | | P | P |
| Donor 4 | | | | P | | P | P | P |
| Donor 5 | | | | | | | | P |
| Donor 6 | | | | | | | P | P |
| Donor 7 | | | | | | | | P |
| Donor 8 | | | | | | | P | P |
| Donor 9 | | | | | | | P | P |
| Donor 10 | | | | | | | P | P |
| Donor 11 | | | | | | | | P |
| Donor 12 | | | | | | | P | P |
| Donor 13 | | P | | | | | P | P |
| Donor 14 | | | | | | P | P | P |
| Donor 15 | | | | | | P | P | P |
| Donor 16 | | | | | | | P | P |
| Donor 17 | | | P | | | | P | P |
| Donor 18 | | | P | | | | P | P |
| Donor 19 | | | | | | | P | P |
| Donor 20 | | | | | | | | P |
| Donor 21 | | | | | | | | P |
| Donor 22 | | | | | | P | | P |
| Donor 23 | | | | | | | | P |
| Donor 24 | | | | | | | | P |
| Proliferation % | 0 | 4 | 8 | 4 | 0 | 17 | 67 | 100 |

TABLE 8
Summary of the magnitude (±SD) of positive (SI ≥ 1.90,
significant p < 0.05) T cell proliferation responses.
The mean SI was calculated from the average of all positive
donor responses observed during the entire time course (days 5-8).

| Sample | Mean SI | SD | % Response |
|---|---|---|---|
| VH0/VK0 | N/A | N/A | 0 |
| VH1/VK3 | 2.88 | ±0.13 | 4 |
| VH2/VK3 | 3.61 | ±1.36 | 8 |
| VH3/VK3 | 3.09 | ±0.88 | 4 |
| VH4/VK3 | N/A | N/A | 0 |
| VH5/VK3 | 2.14 | ±0.43 | 17 |
| Exenatide | 2.99 | ±0.92 | 67 |
| KLH | 14.95 | ±11.22 | 100 |

N/A indicates not applicable.

TABLE 9
Repeated measures one-way ANOVA (Friedman test) using a Dunn's
post-test pairs comparison. The maximum SI from the proliferation
data from all time points of all donors were analyzed.

| | 5H10 | 5H10 | 5H10 | 5H10 | 5H10 | 5H10 |
|---|---|---|---|---|---|---|
| | VH0/ | VH1/ | VH2/ | VH3/ | VH4/ | VH5/ |
| | VK0 | VK3 | VK3 | VK3 | VK3 | VK3 |
| 5H10 | ns | | | | | |
| VH1/VK3 | | | | | | |
| 5H10 | ns | ns | | | | |
| VH2/VK3 | | | | | | |
| 5H10 | ns | ns | ns | | | |
| VH3/VK3 | | | | | | |
| 5H10 | ns | ns | ns | ns | | |
| VH4/VK3 | | | | | | |
| 5H10 | ns | ns | ns | ns | ns | |
| VH5/VK3 | | | | | | |
| Exenatide | **** | **** | **** | **** | **** | ** |

** p < 0.01,
**** p < 0.0001 and ns = not significant.

In summary, the risk of clinical immunogenicity was determined by measuring ex vivo T cell responses using peripheral blood mononuclear cells (PBMC) isolated from 24 healthy donors representing the European and North American population (based on HLA allotypes) in the EPISCREEN™ time course T cell assay. T cell responses were measured using proliferation assays ([³H]-thymidine uptake). The results showed that four of the lead humanized antibodies had a low potential for clinical immunogenicity.

### Example 8: Evaluation of Murine 5H10 in a Bleomycin Model of Lung Inflammation and Fibrosis

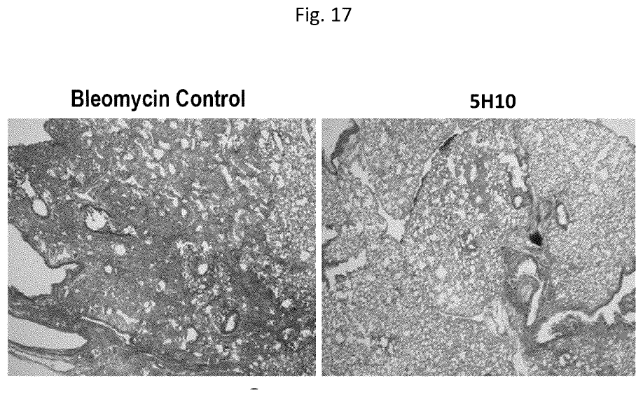

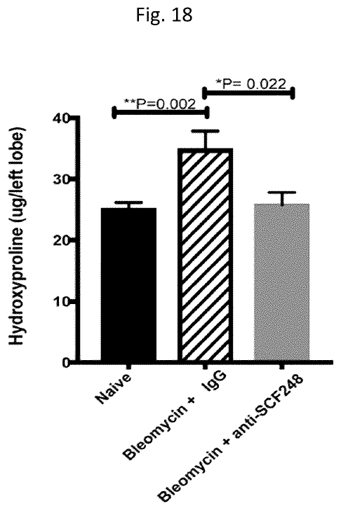

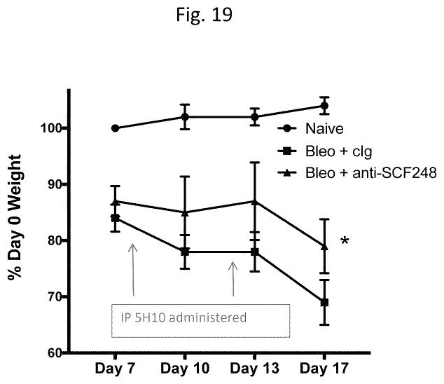

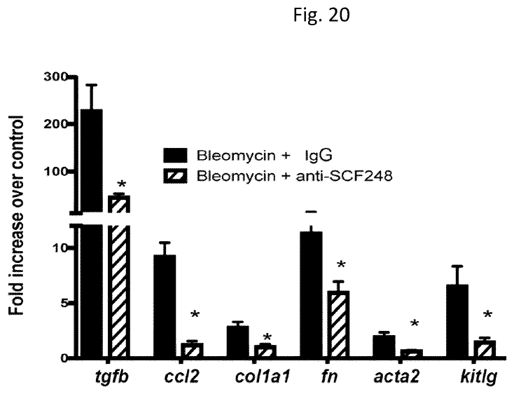

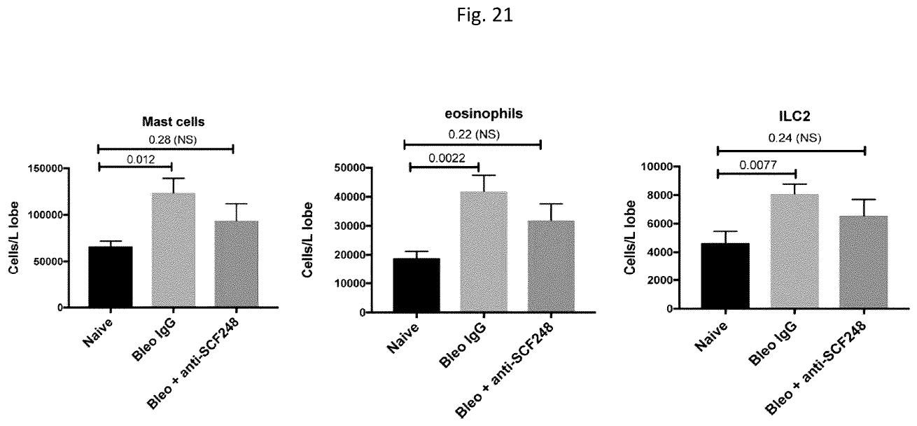

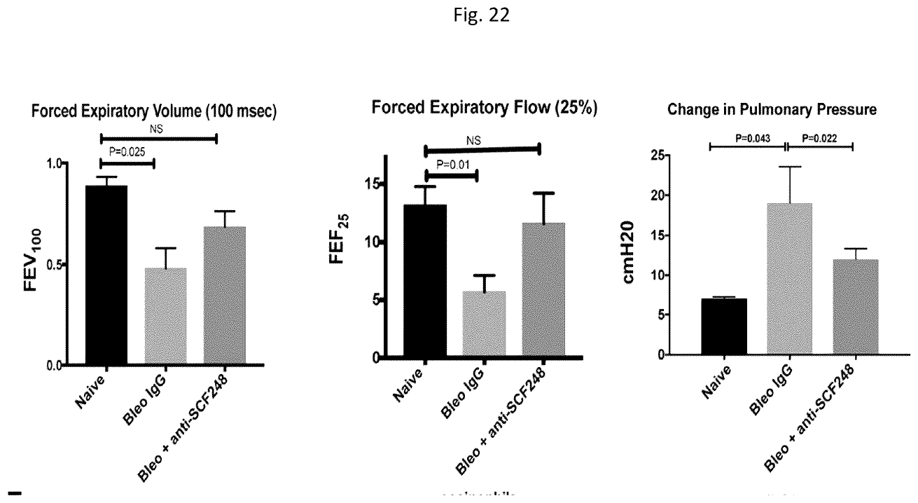

The bleomycin animal model of pulmonary fibrosis was employed to assess in vivo effects of 5H10. Bleomycin is a chemotherapeutic agent that causes pulmonary fibrosis in humans and animals. C57BL6 mice were administered bleomycin intratracheally on Day 1, and on Days 8 and 12 received 5H10 intraperitoneally at 20 mg/kg or isotype-matched control antibody. On Day 17 samples were collected. The 5H10-treated animals had significant improvements in lung histology (FIG. 17), decreases in lung hydroxyproline (a quantitative measure of fibrosis; FIG. 18), maintenance of body weight over time (FIG. 19), decreases in mRNA for inflammatory cytokine and markers of myofibroblast activation (FIG. 20), and in lung mast cells, eosinophils and ILC2 lymphocytes (FIG. 21). Pulmonary function testing was also significantly improved (FIG. 22). Thus, the study demonstrated that 5H10 was effective to reduce fibrosis and inflammation, and improve pulmonary function, in an in vivo model of pulmonary fibrosis.

### Example 9: Evaluation of Humanized 5H10 Antibodies in Chronic Allergic Asthma Model

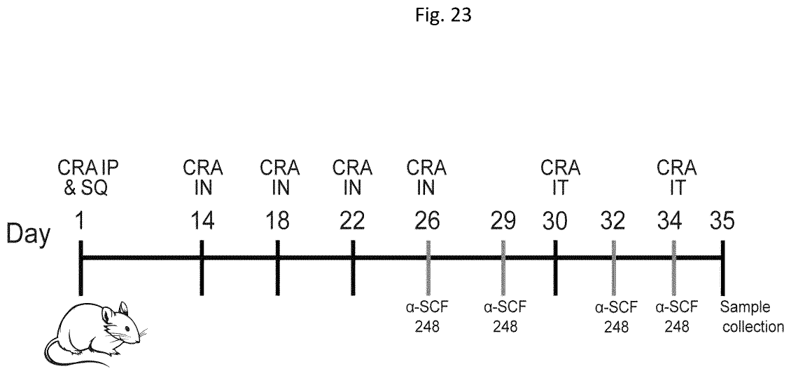

Humanized 5H10 antibodies were tested in an in vivo model of chronic allergic asthma. Mice were sensitized to cockroach antigen (CRA) intraperitoneally and subcutaneously followed by intranasal boosting on days 14, 18, 22 and 26. On days 26, 29, 32, and 34 they were administered indicated humanized 5H10 antibody or PBS control, intraperitoneally at 20 mg/kg or control irrelevant antibody. On days 30 and 34, they also received CRA intratracheally. On Day 35 specimens were collected. A schematic of the study design is provided in FIG. 23.

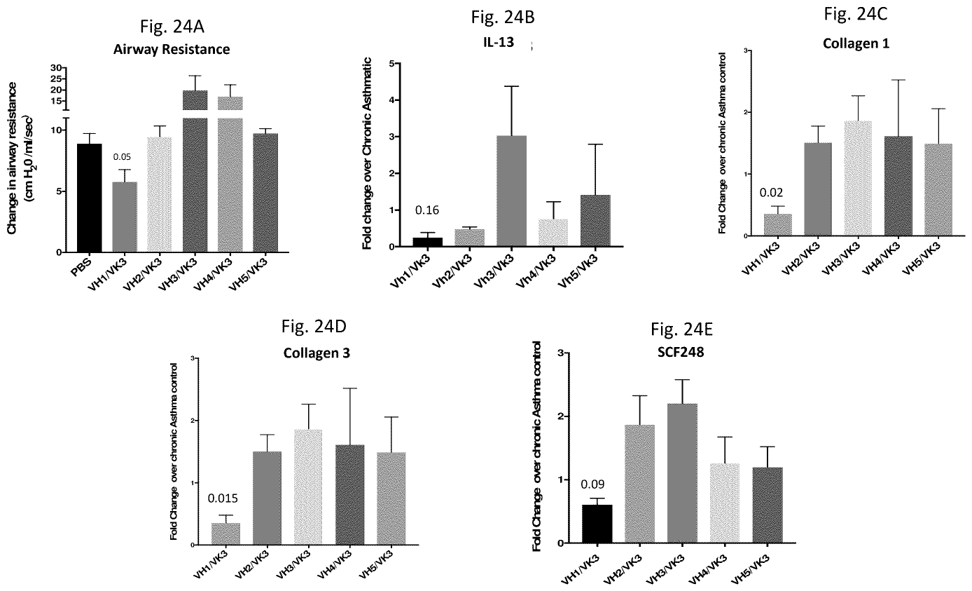

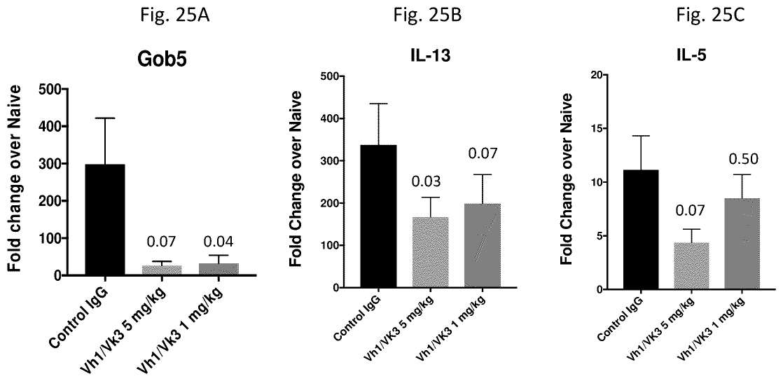

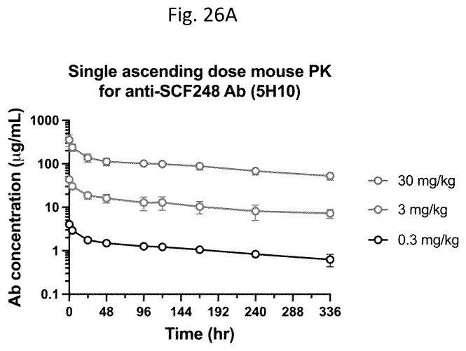

Airway resistance was significantly less in the animals treated with antibody 5H10 VH1/VK3 compared to PBS control as well as to other humanized variant antibodies (FIG. 24A). Further, in animals treated with VH1/VK3 or VH2/VK3, there were significant decreases in lung IL-13 mRNA in relation to the chronic asthma control (i.e., compared to animals administered CRA without any antibody treatment (PBS controls)) (FIG. 24B). Moreover, matrix gene expression was significantly reduced in animals that received VH1/VK3 antibody treatment (Collagen 1 mRNA shown in FIG. 24C; Collagen 3 mRNA shown in FIG. 24D). SCF248 mRNA expression was also significantly reduced in animals treated with VH1/VK3 (FIG. 24E). FIGS. 25A, 25B, and 25C show that VH1/VK3 administration reduced mRNA levels of mucus protein Gob5 and cytokines IL-13 and IL-5 at concentrations of 1 mg/kg and 5 mg/kg. Accordingly, the data showed that the VH1/VK3 antibody blocked cytokine and matrix gene expression in vivo in a model of chronic asthma.

### Example 10: Phase 1a Clinical Trial to Evaluate Safety, Pharmacokinetics, and Pharmacodynamics of the Anti-SCF248 Antibody

A Phase 1a study will enroll 110 healthy volunteers. The primary objective is to obtain safety assessment and obtain accurate pharmacokinetic and pharmacodynamic data for the humanized anti-SCF248 antibody 5H10.

Single and multiple ascending doses of humanized 5H10, or placebo, will be administered, either intravenously or subcutaneously. Six to 8 subjects will make up one group. The starting dose will be based on toxicology studies performed according to good laboratory practices. The baseline pharmacokinetics will be obtained for all development and product lifetime. To assess pharmacodynamics, the number of circulating c-kit+ cells will be assessed, including mast cell progenitor cells and type 2 innate lymphoid (ILC2) cells, in addition to serum inflammatory markers such as SCF165.

### Example 11: a Clinical Study to Evaluate Safety and Efficacy of the Anti-SCF248 Antibody in Patients

A clinical study will enroll patients suffering from an inflammatory disorder such as atopic dermatitis, chronic urticaria, pulmonary fibrosis, and/or others. One object of the study is to establish a dose-response relationship between humanized 5H10 antibody and pharmacodynamic markers in diseased patients e.g. the number of circulating c-kit+ cells, such as mast cell progenitor cells and type 2 innate lymphoid cells (ILC2) cells. Inflammatory biomarkers, such as ADAM8, CCL17, EPX, RNASE3, CCL2, CCL5, tryptase, histamine and SCF165 will also be measured.

A single, ascending dose of 5H10 antibody will be given to each subject group. The starting dose will be based on Phase 1a pharmacodynamic biomarkers. Patients will be treated for one, two, three, or more months. The results of the study will show that humanized 5H10 antibody is effective in stabilizing and/or treating and/or preventing the progression of inflammatory disorders and fibrotic diseases.

### Example 12: Toxicity and Toxicokinetic Study of Human VH1/VK3 Monoclonal Antibody Administered in Rats

Study Objective: This study sought to evaluate the toxicity and determine the toxicokinetics of VH1/VK3 when administered via subcutaneous or intravenous (bolus) injection to rats once weekly for 13 weeks (total of 13 doses) and to assess the reversibility or persistence of any effects after a 12-week recovery phase. VH1/VK3 is a humanized anti-stem cell factor 248 (SCF248) monoclonal IgG4 antibody, also known as a “OpSCF” and comprises a heavy chain amino acid sequence of SEQ ID NO: 42 and a light chain amino acid sequence of SEQ ID NO: 49.

Study design: Male and female rats were assigned to five groups (Groups 1-5), and doses were administered once weekly for a total of 13 doses at a volume of 1.5 mL/kg/dose. The study groups are described in further detail in Table 10 below. Animals were administered 2, 25, or 100 mg/kg/dose subcutaneously (SC) or 100 mg/kg/dose intravenously (IV). Group 1 was administered a vehicle control article (placebo) only. Animals in Groups 1 through 4 were dosed via SC injection in the dorsal region, and animals in Groups 1 and 5 were dosed via IV injection in the lateral tail vein.

TABLE 10
Study groups

| | | | | Dose | Number of | |
|---|---|---|---|---|---|---|
| | | | Dose Level | Concentration | Animals | |
| Group (Dose) | Subgroup | Dose Route | (mg/kg/dose) | (mg/mL) | Males | Females |
| 1 (Control) | 1 (Toxicity) | Subcutaneous | 0 | 0 | 15 | 15 |
| | 2 | Intravenous | 0 | 0 | 3 | 3 |
| | (Toxicokinetic) | | | | | |
| 2 (Low) | 1 (Toxicity) | Subcutaneous | 2 | 1.33 | 10 | 10 |
| | 2 | | 2 | 1.33 | 9 | 9 |
| | (Toxicokinetic) | | | | | |
| 3 | 1 (Toxicity) | Subcutaneous | 25 | 16.67 | 10 | 10 |
| (Intermediate) | 2 | | 25 | 16.67 | 9 | 9 |
| | (Toxicokinetic) | | | | | |
| 4 (High) | 1 (Toxicity) | Subcutaneous | 100 | 66.67 | 15 | 15 |
| | 2 | | 100 | 66.67 | 9 | 9 |
| | (Toxicokinetic) | | | | | |
| 5 (High) | 1 (Toxicity) | Intravenous | 100 | 66.67 | 15 | 15 |
| | 2 | | 100 | 66.67 | 9 | 9 |
| | (Toxicokinetic) | | | | | |

Treatment/Exposure: Study groups were administered a test article (drug product) or a control vehicle article (placebo). The test article comprised VH1/VK3 antibody at a concentration of 104.8 g/L. The vehicle control excluded the active ingredient of the test article and instead comprised 20 mM Histidine, 50 mM Glutamic Acid, 100 mM Arginine, and 0.02% polysorbate 80 at a pH 6.5.

Outcome Measures: This study assessed toxicity based on mortality, clinical observations, body weights, food consumption, ophthalmic observations, and clinical and anatomic pathology. Blood samples were collected for toxicokinetic and anti-drug antibody (ADA) evaluation. Specifically, the following pharmacokinetic parameters were evaluated:
VH1/VK3 mean back-extrapolated concentration at time 0 (C₀)
Maximum observed concentration of VH1/VK3 (Cₘₐₓ)
Time to peak concentration (Tₘₐₓ)
Area under the concentration-time curve (AUC).
   AUC from time 0 to time of last measurable concentration (AUC₀₋ₜ)
   AUC from time 0 to 168 hours (AUC₀₋₁₆₈)
Elimination half-life (t_{1/2})

Results: No adverse test article-related observations were noted for animals administered up to 100 mg/kg/dose IV or SC; thus, the no observed adverse effect level (NOAEL) was 100 mg/kg/dose. This dose level corresponded to mean Cₘₐₓ and AUC₀₋₁₆₈ values of 1320 μg/mL and 192000 h*μg/mL, respectively, via SC injection and 4070 μg/mL and 334000 h*μg/mL, respectively, via IV injection on Day 85 of the dosing phase. Pharmacokinetic results are provided in Table 11 below.

In sum, toxicology testing in rats indicated there were no adverse effects associated with administration of human VH1/VK3 antibody.

TABLE 11
Toxicokinetic parameters for human VH1/VK3 in rat serum following once weekly
subcutaneous and/or intravenous (bolus) injection administration assessed at Day 85

| | | | | | | | SE | | |
|---|---|---|---|---|---|---|---|---|---|
| | | C₀ | Cₘₐₓ | SECₘₐₓ | Tₘₐₓ | AUX₀₋ₜ | AUC₀₋ₜ | AUC₀₋₁₆₈ | |
| Route | Treatment | (μL/mL) | (μL/mL) | (μL/mL) | (h) | (h*μL/mL) | (h*μL/mL) | (h*μL/mL) | t1/2 |
| SC | 2 mg/kg | NA | 54.9 | 1.96 | 72.0 | 54000 | 4500 | 8240 | 339 |
| | VH1/VK3 | | | | | | | | |
| SC | 25 mg/kg | NA | 527 | 27.5 | 72.0 | 575000 | 41300 | 81500 | 339 |
| | VH1/VK3 | | | | | | | | |
| SC | 100 mg/kg | NA | 1320 | 218 | 24.0 | 1190000 | 46600 | 192000 | 331 |
| | VH1/VK3 | | | | | | | | |
| IV | 100 mg/kg | 4170 | 4070 | 143 | 0.250 | 1870000 | 140000 | 334000 | 355 |
| | VH1/VK3 | | | | | | | | |

IV = IV Bolus;
NA = Not Applicable;
NC = Not Calculated;
SC = Subcutaneous.
Note:
Sex Combined

### Example 13: Toxicity and Toxicokinetic Study of Human VH1/VK3 Monoclonal Antibody Administered in Cynomolgus Monkeys

Study Objectives: This study sought to evaluate the toxicity and determine the toxicokinetics of human VH1/VK3 antibody when administered via subcutaneous or intravenous (bolus) injection to cynomolgus monkeys once weekly for 2 weeks (total of 2 doses) or 13 weeks (total of 13 doses) and to assess the reversibility or persistence of any effects after a 12-week recovery phase. VH1/VK3, also referred to as “OpSCF” is a humanized anti-stem cell factor 248 (SCF248) monoclonal antibody and comprises a heavy chain amino acid sequence of SEQ ID NO: 42 and a light chain amino acid sequence of SEQ ID NO: 49.

Study Design: Male and female cynomolgus monkeys were assigned to six groups (Groups 1-6), and doses were administered as indicated in Table 12 below. Animals were dosed once weekly at a volume of 1 mL/kg for 2 weeks via IV (bolus) injection or 13 weeks via IV (bolus) and/or subcutaneous (SC) injection. Group 1 was administered vehicle control article only. Animals in Group 2 were administered 2 total doses during weeks 1 and 2 of the 13 week dosing phase. Animals in Groups 1, 3, 4, 5, and 6 were administered 13 total doses. Three animals per sex per group from Groups 1, 3, 4, 5 and 6 were designated as terminal animals, and two animals per sex per group in Groups 1, 5, and 6 and three animals per sex in Group 2 were designated as recovery animals.

TABLE 12
Study groups

| | | Dose | Dose | Number of | |
|---|---|---|---|---|---|
| | | Level | Concen- | Animals | |
| Group | | (mg/kg/ | tration | | Fe- |
| (Dose) | Dose Route | dose) | (mg/mL) | Males | male |
| 1 (Control) | Subcutaneous, | 0 | 0 | 5 | 5 |
| | Intravenous | | | | |
| 2 (Low; 2 doses) | Intravenous | 1 | 1 | 3 | 3 |
| 3 (Low) | Subcutaneous | 2 | 2 | 3 | 3 |
| 4 (Intermediate) | Subcutaneous | 25 | 25 | 3 | 3 |
| 5 (High) | Subcutaneous | 100 | 100 | 5 | 5 |
| 6 (High) | Intravenous | 100 | 100 | 5 | 5 |

Treatment/Exposure: Study groups were administered a test article (the drug product) or a vehicle control article. The test article comprised VH1/VK3 at a concentration of 104.8 g/L. The vehicle control article comprises 20 mM Histidine, 50 mM Glutamic Acid, 100 mM Arginine, and 0.02% polysorbate 80 at a pH 6.5.

Outcome Measures: Assessment of toxicity was based on mortality, clinical observations, body weights, qualitative food consumption, ophthalmic observations, electrocardiographic (ECG) measurements, and clinical and anatomic pathology. Blood samples were collected for toxicokinetic, anti-drug antibody, and immunotoxicology evaluations. Specifically, the following pharmacokinetic parameters were evaluated:
VH1/VK3 mean back-extrapolated concentration at time 0 (C₀)·
Maximum observed concentration of VH1/VK3 (Cₘₐₓ)
Time to peak concentration (Tₘₐₓ)
Area under the concentration-time curve (AUC)
   AUC from time 0 to time of last measurable concentration (AUC₀₋ₜ)
   AUC from time 0 to 168 hours (AUC₀₋₁₆₈)
Elimination half-life (t_{1/2})

Results: Male and female cynomolgus monkeys were administered vehicle control article or 1, 2, 25, or 100 mg/kg/dose VHI/VK3 via SC injection or 1 and 100 mg/kg/dose VHI/VK3 via IV injection once weekly for 2 or 13 weeks. No adverse test article-related observations were noted for animals administered up to 100 mg/kg/dose VH1/VK3 via IV or SC injection, therefore the NOAEL was determined to be 100 mg/kg/dose. This dose level corresponded to Cₘₐₓ and AUC₀₋₁₆₈ values of 12,500 μg/mL and 1,140,000 h*μg/mL, respectively, in the IV dose group and 3900 μg/mL and 545,000 h*μg/mL, respectively, in the SC dose group on Day 85 of the dosing phase. Pharmacokinetic results for SC and IV injection groups are in Tables 13 and 14, respectively. The SC bioavailability, estimated by comparing AUC₀₋₁₆₈, ranged from 64.8 to 71.3% on Day 1 and 43.8 to 51.2% on Day 85.

TABLE 13
Individual and mean toxicokinetic parameters for 100 mg/kg
VH1/VK3 in cynomolgus monkey serum following once weekly
SC injection administration assessed at Day 85

| | Cₘₐₓ | Tₘₐₓ | AUC₀₋ₜ | AUC₀₋₁₆₈ | |
|---|---|---|---|---|---|
| Animal | (μL/mL) | (h) | (h*μg/mL) | (h*μg/mL) | t_{1/2} |
| P0401 | 4290 | 72.0 | 600000 | 600000 | NC |
| P0402 | 3750 | 6.00 | 541000 | 541000 | NC |
| P0403 | 3060 | 72.0 | 419000 | 419000 | NC |
| P0404 | 5890 | 24.0 | 694000 | 694000 | 251 |
| P0405 | 3460 | 24.0 | 510000 | 510000 | NC |
| P1001 | 3100 | 6.00 | 451000 | 451000 | 399 |
| P1002 | 4150 | 24.0 | 602000 | 602000 | 255 |
| P1003 | 3650 | 48.0 | 485000 | 485000 | 265 |
| P1004 | 4030 | 48.0 | 608000 | 608000 | 373 |
| P1005 | 3590 | 72.0 | 537000 | 537000 | 325 |
| N | 10 | 10 | 10 | 10 | 6 |
| Mean | 3900 | 39.6 | 545000 | 545000 | 311 |
| SD | 810 | 26.4 | 83100 | 83100 | 64.1 |
| CV % | 20.8 | 66.7 | 15.3 | 15.3 | 20.6 |
| Min | 3060 | 6.00 | 419000 | 419000 | 251 |
| Median | 3700 | 36.0 | 539000 | 539000 | 295 |
| Max | 5890 | 72.0 | 694000 | 694000 | 399 |
| Geometric Mean | 3830 | 29.0 | 539000 | 539000 | 306 |
| Geometric CV % | 19.0 | 120.4 | 15.4 | 15.4 | 20.5 |

AUC₀₋₁₆₈ = Area under the concentration-time curve from 0 to 168 hours postdose; AUC₀₋ₜ = Area under the concentration-time curve from 0 to the last measurable concentration; Cₘₐₓ = Maximum observed concentration; CV % = Coefficient of variation; N = Number of animals; NC = Not calculated; SC = Subcutaneous; SD = Standard deviation; t_{1/2} = Half-life; Tₘₐₓ = Time of maximum observed concentration.

TABLE 14
Individual and mean toxicokinetic Parameters for 100 mg/kg
VH1/VK3 in cynomolgus monkey serum following once weekly
IV (Bolus) injection administration assessed at Day 85

| | Cₘₐₓ | C₀ | Tₘₐₓ | AUC₀₋ₜ | AUC₀₋₁₆₈ | |
|---|---|---|---|---|---|---|
| Animal | (μL/mL) | (μL/mL) | (h) | (h*μg/mL) | (h*μg/mL) | t1/2 |
| P0501 | 5420 | 5340 | 0.250 | 471000 | 471000 | 150 |
| P0502 | 6070 | 17500 | 24.0 | 1720000 | 1720000 | NC |
| P0503 | 20300 | 19100 | 0.250 | 1450000 | 1450000 | 246 |
| P0504 | 14500 | 14800 | 4.00 | 1730000 | 1730000 | 169 |
| P0505 | 19900 | 17900 | 0.250 | 1370000 | 1370000 | 121 |
| P1101 | 14100 | 15000 | 4.00 | 1500000 | 1500000 | 219 |
| P1102 | 7870 | 19700 | 4.00 | 1620000 | 1620000 | 62.7 |
| P1103 | 3610 | 3450 | 0.250 | 306000 | 306000 | 118 |
| P1104 | 6310 | 6280 | 0.250 | 547000 | 547000 | 280 |
| P1105 | 5900 | 5610 | 0.250 | 624000 | 624000 | 296 |
| N | 10 | 10 | 10 | 10 | 10 | 9 |
| Mean | 10400 | 12500 | 3.75 | 1140000 | 1140000 | 185 |
| SD | 6270 | 6500 | 7.33 | 574000 | 574000 | 80.0 |
| CV % | 60.2 | 52.1 | 195.5 | 50.6 | 50.6 | 43.3 |
| Min | 3610 | 3450 | 0.250 | 306000 | 306000 | 62.7 |
| Median | 7090 | 14900 | 0.250 | 1410000 | 1410000 | 169 |
| Max | 20300 | 19700 | 24.0 | 1730000 | 1730000 | 296 |
| Geometric | 8830 | 10500 | 0.907 | 967000 | 967000 | 167 |
| Mean | | | | | | |
| Geometric | 66.5 | 73.6 | 444.6 | 72.2 | 72.2 | 53.5 |
| CV % | | | | | | |

AUC₀₋₁₆₈ = Area under the concentration-time curve from 0 to 168 hours postdose;
AUC₀₋ₜ = Area under the concentration-time curve from 0 to the last measurable concentration;
Cₘₐₓ = Maximum observed concentration;
CV % = Coefficient of variation;
N = Number of animals;
NC = Not calculated;
SC = Subcutaneous;
SD = Standard deviation;
t1/2 = Half-life;
Tₘₐₓ = Time of maximum observed concentration.

### Example 14: Safety, Tolerability, and Pharmacokinetics of a Murine 5H10 Antibody Administered in a Bleomycin-Induced Lung Fibrosis Model in a Single Ascending Dose (SAD) Study

Purpose: This study sought to determine the efficacy and safety of a single dose of a murine 5H10 monoclonal antibody administered in the treatment of bleomycin-induced lung fibrosis in mice. The test article is a mouse immunoglobulin (Ig) G4 kappa monoclonal antibody specific for the long splice variant of stem cell factor 248 (SCF248) and comprises a heavy chain amino acid sequence of SEQ ID NO: 7 and a light chain amino acid sequence of SEQ ID NO: 13.

Methods: This study evaluated 5H10 pharmacokinetics (PK) in C57BL/6 mice at three doses including 0.3, 3, and 30 mg/kg. Doses were administered intravenously at baseline and serum antibody concentration was then evaluated over 14 days. The PK data was then analyzed using a naïve pooled fit to a 2-compartment model with linear clearance. Model-based predictions were calculated for each dose using the fit values of CL, V1, V2, and Q.

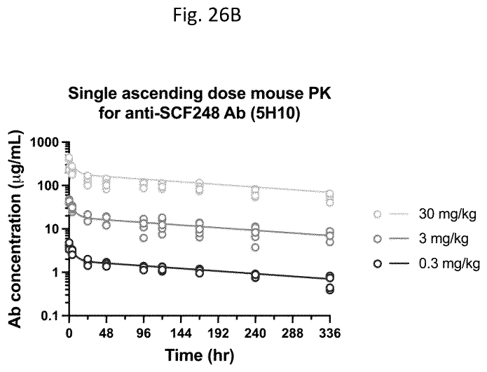

Results: Results are provided in FIGS. 26A-26B. As shown, the PK of 5H10 in C57BL/6 mice was linear following a single ascending IV dose ranging from 0.3-30 mg/kg (FIG. 26A). Moreover, when the PK data was analyzed using a naïve pooled fit to a 2-component model with linear clearance, the 5H10 antibody continued to exhibit linear clearance (FIG. 26B).

### Example 15: Safety, Tolerability, and Pharmacokinetics of a Murine 5H10 Antibody Administered in a Bleomycin-Induced Lung Fibrosis Model in a Multiple Dose Study

Purpose: This study sought to determine the efficacy and safety of multiple doses of a murine 5H10 monoclonal antibody administered in the treatment of bleomycin-induced lung fibrosis in mice. The test article is a mouse immunoglobulin (Ig) G4 kappa monoclonal antibody specific for the long splice variant of stem cell factor 248 (SCF248) and comprises a heavy chain amino acid sequence of SEQ ID NO: 7 and a light chain amino acid sequence of SEQ ID NO: 13.

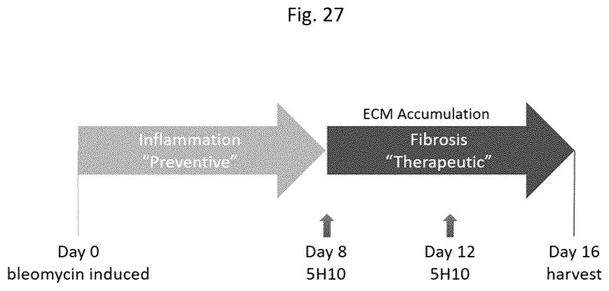

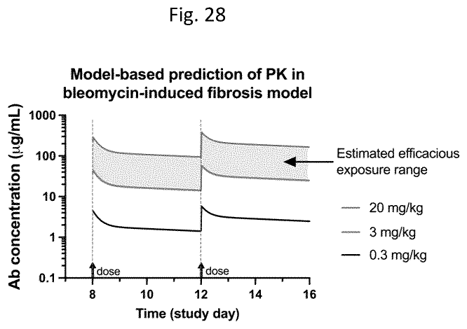

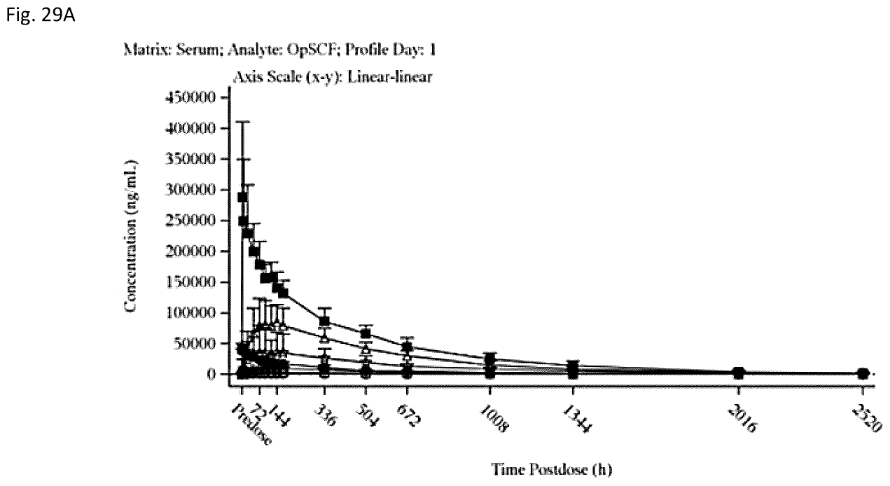

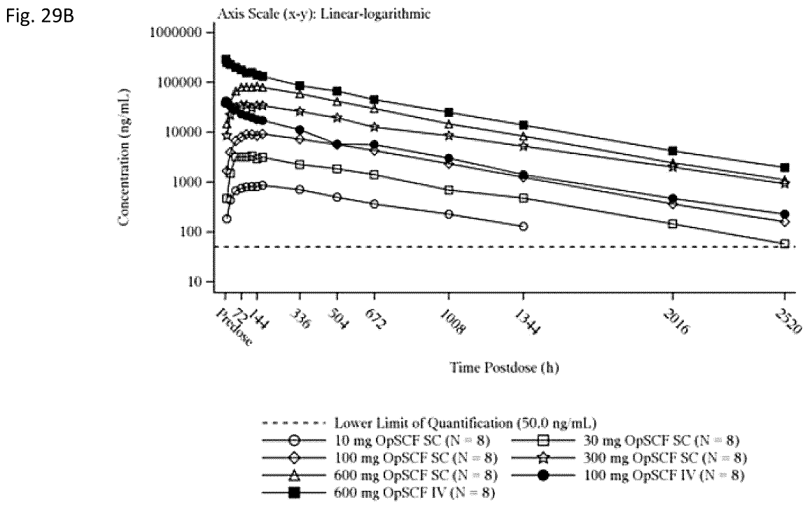

Methods: A study was conducted according to the timeline provided in FIG. 27. C57BL/6 Mice were bleomycin induced at Day 0 and then administered murine 5H10 at doses of 0.3, 3, and 20 mg/kg via intraperitoneal (IP) injection on Days 8 and 12 of the study. Mice were then harvested on Day 16. Model-based prediction of 5H10 antibody concentration was then estimated from Days 8 to 16 of the study. Exposure metrics were derived from model-based predictions following the second dose administration at Day 12 (FIG. 28). The model-based PK analysis estimated an efficacious exposure range, and used these estimations to calculate Cₘₐₓ and AUC from time zero to infinity (AUC_{0-inf}), which is equivalent to AUC from time zero to time at steady state (AUC_{0-t,ss}).

Results: Table 15 shows the predicted Cₘₐₓ and AUC_{0-tau,ss} for 3 mg/kg, 10 mg/kg, and 20 mg/kg doses.

TABLE 15
Exposure metrics derived from model-based PK predictions

| | | |
|---|---|---|
| Dose | Cₘₐₓ | AUC_{0-inf} = AUC_{0-t, ss} |

| | | | | |
|---|---|---|---|---|
| 3 | mg/kg | 58 μg/mL | 6,181 | μg · hr/mL |
| | | (twice a day for | | |
| | | four days dosing) | | |
| 10 | mg/kg | 210 μg/mL | 20,600 | μg · hr/mL |
| | | (once weekly dosing) | | |
| 20 | mg/kg | 388 μg/mL | 41,210 | μg · hr/mL |
| | | (twice a day for | | |
| | | four days dosing) | | |

### Example 16: Phase 1a: Randomized, Double-Blind, Placebo-Controlled Study to Assess the Safety, Tolerability, and Pharmacokinetics of OpSCF after a Single Ascending Dose (SAD) in Healthy Participants

Study Overview: This study was a Phase 1a, randomized, double-blind, placebo-controlled, single ascending intravenous (IV) and subcutaneous (SC) dose first-in-human (FIH) study. Cohorts of healthy adult subjects were assessed for safety, tolerability, immunogenicity, pharmacokinetics (PK), and pharmacodynamics (PD) of OpSCF. OpSCF is a humanized immunoglobulin (Ig) G4 kappa monoclonal antibody specific for the long splice variant of stem cell factor 248 (SCF248) and comprises a heavy chain amino acid sequence of SEQ ID NO: 42 and a light chain amino acid sequence of SEQ ID NO: 49.

Study Objectives: This study sought to assess the primary, secondary and exploratory objectives described below.

### Primary Objective:

To determine the safety and tolerability of a single dose of OpSCF in healthy subjects.

### Secondary Objectives:

To determine the PK parameters of OpSCF following a single dose of OpSCF in healthy subjects.
To determine the immunogenicity of OpSCF in healthy subjects as measured by anti-drug antibodies (ADAs).

### Exploratory Objective:

To assess the PD response after a single dose of OpSCF using PD markers including SCF165, cytokines, mast cell and eosinophil products, and innate lymphoid cell (ILC) precursors in blood.

Study design: Healthy adult subjects were studied in 7 single dose cohorts (Cohorts: 10 mg, 30 mg, 100 mg, 300 mg, and 600 mg for SC, 100 mg and 600 mg for IV) of approximately 10 subjects each who were randomized in an 8:2 ratio to receive a single dose of OpSCF or placebo. Single dosing was performed on Day 1 of the study. The study cohorts are described in further detail below in Table 16. After dosing in the 10 mg IV dose cohort, the safety of dosing each cohort, along with tolerability, laboratory data, and any available PK data, were reviewed by a Dose Escalation Committee (DEC) and escalation proceeded as per dose escalation procedure.

TABLE 16
Study cohorts administered OpSCF in a SAD study

| SAD | Planned Number | Actual Investigational Medicinal |
|---|---|---|
| Cohort # | of Subjects | Product Dose |
| S1 | 10 | 10 mg OpSCF SC/placebo |
| S2 | 10 | 30 mg OpSCF SC/placebo |
| S3 | 10 | 100 mg OpSCF SC/placebo |
| S4 | 10 | 300 mg OpSCF SC/placebo |
| S5 | 10 | 100 mg OpSCF IV/placebo |
| S6 | 10 | 600 mg OpSCF SC/placebo |
| S7 | 10 | 600 mg OpSCF IV/placebo |

Participants: Participants included healthy adults. Seventy subjects were randomized and dosed and 66 subjects (94.3%) completed the study in accordance with the protocol. Three subjects (4.3%) withdrew from the study. One subject was discontinued (1.4%) because of an adverse event (AE). The demographic characteristics of the study population are in Table 17 below.

TABLE 17
Summary of screening demographic data for SAD study

| | | 10 mg | 30 mg | 100 mg | 300 mg | 600 mg | | 100 mg | 600 mg | |
|---|---|---|---|---|---|---|---|---|---|---|
| | Placebo | OpSCF | OpSCF | OpSCF | OpSCF | OpSCF | Placebo | OpSCF | OpSCF | |
| | SC | SC | SC | SC | SC | SC | IV | IV | IV | Overall |
| Demographic | (N = 10) | N = 8) | (N = 8) | (N = 8) | (N = 8) | (N = 8) | (N = 4) | (N = 8) | (N = 8) | (N = 70) |
| Age (years) | 33.3 | 44.0 | 33.9 | 33.8 | 45.3 | 31.0 | 45.0 | 38.6 | 47.5 | 38.6 |
| | (12.23) | (11.29) | (7.81) | (11.39) | (10.35) | (9.49) | (10.36) | (10.60) | (11.45) | (11.72) |
| Sex | | | | | | | | | | |
| Male | 7 | 5 | 1 | 2 | 6 | 3 | 3 | 5 | 6 | 38 |
| | (70.0%) | (62.5%) | (12.5%) | (25.0%) | (75.0%) | (37.5%) | (75.0%) | (62.5%) | (75.0%) | (54.3%) |
| Female | 3 | 3 | 7 | 6 | 2 | 5 | 1 | 3 | 2 | 32 |
| | (30.0%) | (37.5%) | (87.5%) | (75.0%) | (25.0%) | (62.5%) | (25.0%) | (37.5%) | (25.0%) | (45.7%) |
| Race | | | | | | | | | | |
| White | 7 | 8 | 6 | 5 | 6 | 4 | 3 | 4 | 5 | 48 |
| | (70.0%) | (100%) | (75.0%) | (62.5%) | (75.0%) | (50.0%) | (75.0%) | (50.0%) | (62.5%) | (68.6%) |
| Black or | 1 | — | 1 | 3 | 2 | 3 | 1 | 4 | 3 | 18 |
| African | (10.0%) | | (12.5%) | (37.5%) | (25.0%) | (37.5%) | (25.0%) | (50.0%) | (37.5%) | (25.7%) |
| American | | | | | | | | | | |
| Asian | 1 | — | 1 | — | — | — | — | — | — | 2 |
| | (10.0%) | | (12.5%) | | | | | | | (2.9%) |
| Multiple | 1 | — | — | — | — | 1 | — | — | — | 2 |
| | (10.0%) | | | | | (12.5%) | | | | (2.9%) |
| Ethnicity | | | | | | | | | | |
| Hispanic or | 2 | 2 | 1 | 3 | 1 | 1 | — | 1 | 1 | 12 |
| Latino | (20.0%) | (25.0%) | (12.5%) | (37.5%) | (12.5%) | (12.5%) | | (12.5%) | (12.5%) | (17.1%) |
| Not Hispanic | 8 | 6 | 7 | 5 | 7 | 7 | 4 | 7 | 7 | 58 |
| or Latino | (80.0%) | (75.0%) | (87.5%) | (62.5%) | (87.5%) | (87.5%) | (100%) | (87.5%) | (87.5%) | (82.9%) |
| Height (cm) | 175.65 | 171.75 | 161.24 | 166.69 | 173.16 | 167.73 | 174.13 | 167.38 | 173.55 | 170.07 |
| | (10.206) | (9.563) | (6.595) | (10.265) | (13.265) | (9.261) | (14.050) | (8.995) | (7.263) | (109.334) |
| Body Weight | 76.78 | 81.08 | 65.84 | 77.81 | 79.74 | 70.69 | 86.78 | 77.08 | 81.01 | 76.87 |
| (kg) | (11.620) | (13.125) | (8.089) | (9.290) | (17.626) | (7.314) | (15.125) | (14.763) | (12.445) | (12.827) |
| Body Mass | 24.90 | 27.31 | 25.34 | 28.08 | 26.30 | 25.15 | 28.45 | 27.78 | 26.83 | 26.47 |
| Index (kg/m²) | (3.112) | (2.366) | (2.801) | (2.865) | (3.179) | (2.099) | (2.195 | (2.768) | (3.196) | (2.892) |

IV = intravenous;
n = number of subjects with valid observations;
N = number of subjects;
SC = subcutaneous;
SD = standard deviation;
For continuous data, mean (SD) statistics presented; for categorical data, N (%) statistics presented.
Body mass index (kg/m²) = body weight (kg)/height (m)²

Treatment: Each subject received OpSCF or placebo in accordance with the randomization schedule, with a single SC or IV dose on Day 1 for the SAD cohorts.

Clinical endpoints: This study sought to assess the below PK, PD, and safety measures in healthy participants.

PK and PD endpoints were evaluated by the following parameters:
Serial blood sampling of OpSCF conducted after dosing. The following PK parameters of OpSCF were estimated using noncompartmental methods:
   Maximum serum concentration (Cₘₐₓ)
   Minimum serum concentration (Cₘᵢₙ, for the last dose in MAD only)
   Pre-dose trough serum concentration (C_{trough}, for all doses in MAD only)
   Time to Cₘₐₓ (Tₘₐₓ)
   The area under the serum concentration-time curve from time 0 to the last measurable concentration (AUCₜ, for SAD only), from time 0 to infinity (AUC_{inf}, for SAD only), and over a dosing interval (AUCₜₐᵤ, for the first and last dose in MAD only)
   Terminal half-life (t_{1/2})
   Volume of distribution (V and Vₛₛ for IV doses and V/F for SC doses)
   Clearance (CL for IV doses and CL/F for SC doses)
   The absolute bioavailability (F) estimated for 100 mg and 600 mg SC dose levels
   Additional parameters may have been calculated if useful in the interpretation of the data.
For immunogenicity, confirmed positive ADA assay results were reported both as ADA incidence by group and as their titer values.
Serial blood sampling was evaluated for:
   Frequencies of circulating cell populations (c-kit-positive cells including ILC and mast cell precursors; lymphocyte surface marker analysis [TBNK]).
   Levels of soluble proteins (SCF, total IgE, and ILC2, eosinophilic, and mast cell associated proteins).

Safety endpoints were evaluated by:
Monitoring of adverse events (AEs), including determination of serious adverse events (SAEs) and AEs leading to discontinuation; dose-limiting toxicities, and laboratory abnormalities as characterized by type, frequency, timing, severity (grade per National Cancer Institute Common Terminology Criteria for Adverse Events, v5.0 [v4.03 for laboratory abnormalities], seriousness, and relationship to OpSCF.
Monitoring of injection site reactions (ISRs) and infusion-related reactions (IRRs)

### Results:

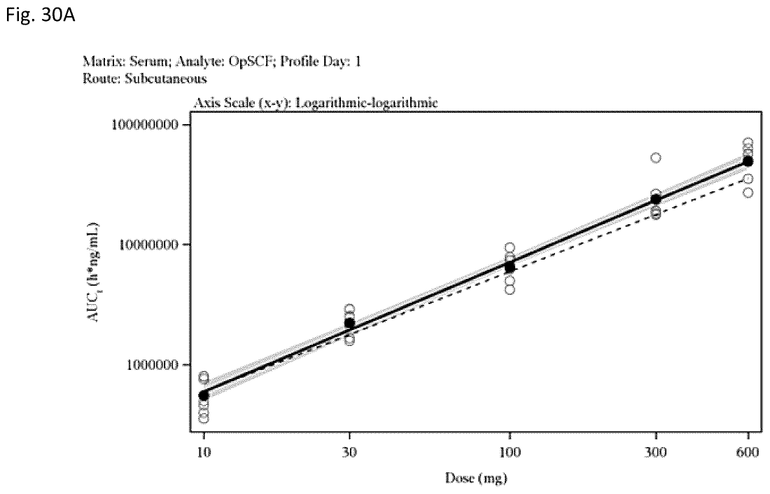

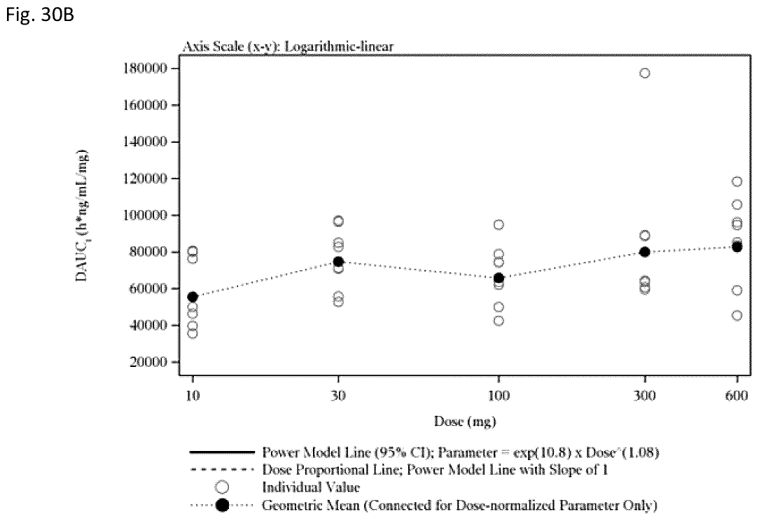

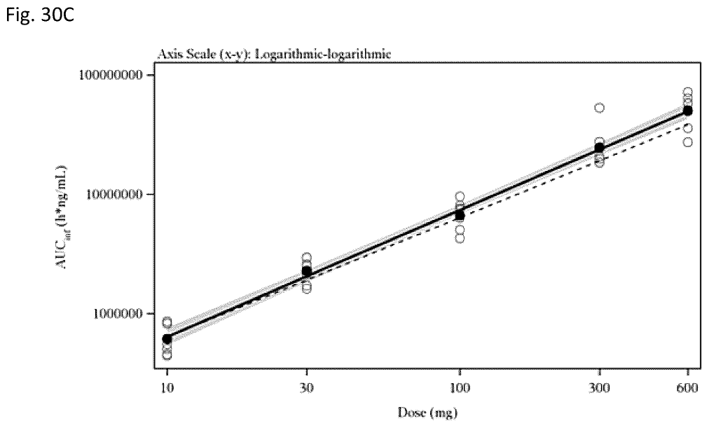

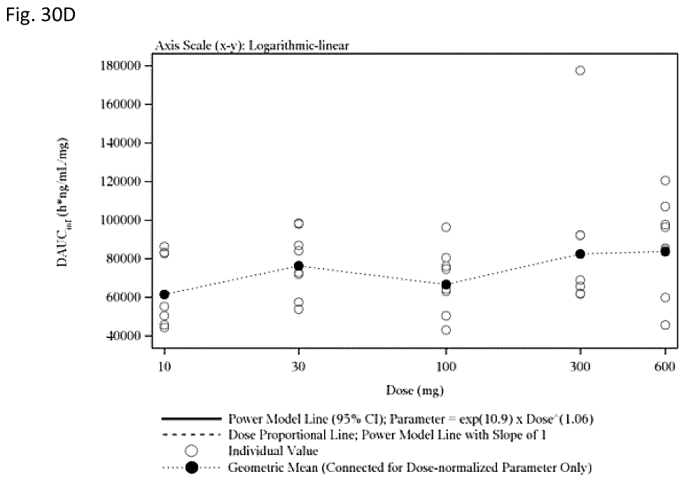

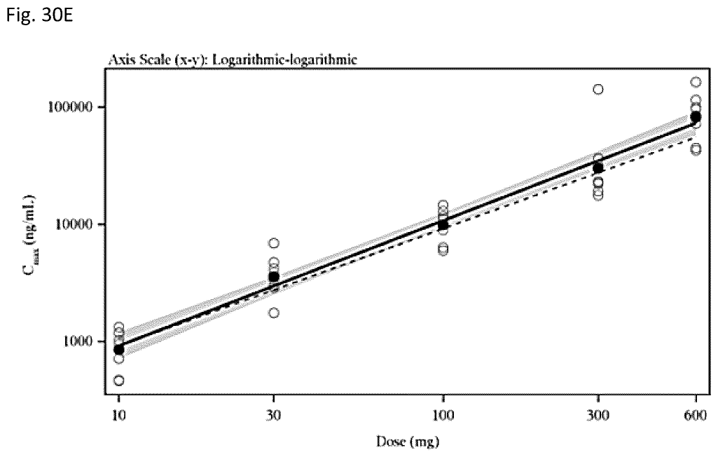

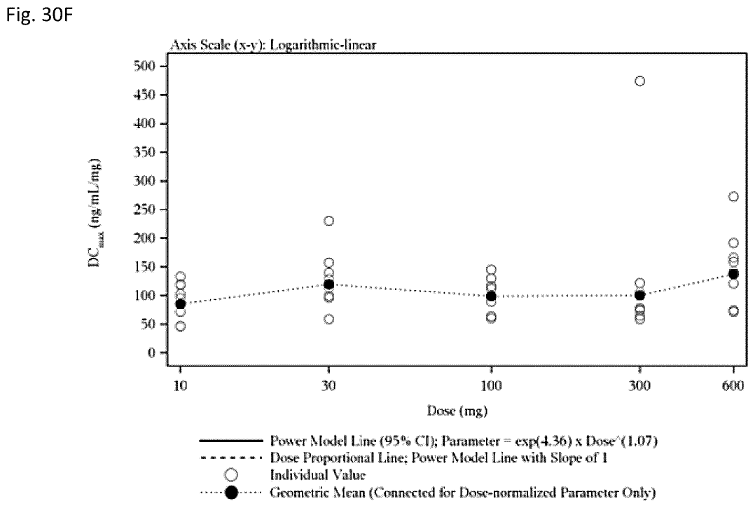

PK and PD: Serum concentrations of OpSCF are presented in FIGS. 29A-29B. The PK parameters of OpSCF are summarized in Table 18. The results of the statistical analysis of dose proportionality for SC doses are presented in Table 19, and in FIG. 30A (AUCₜ), FIG. 30B (DAUCₜ, dose normalized AUCₜ), FIG. 30C (AUC_{inf}), FIG. 30D (DAUC_{inf}, dose normalized AUC_{inf}), FIG. 30E (Cₘₐₓ) and FIG. 30F (DCₘₐₓ, dose normalized Cₘₐₓ).

As shown in FIGS. 29A-29B, the maximal mean serum concentrations of OpSCF were attained immediately after a single IV dose and declined in a monophasic manner. There was a similar geometric mean t_{1/2} at both IV dose levels, with values of 359 and 366 hours (approximately 15 to 15.3 days), respectively, for the 100 and 600 mg doses, and individual subject values ranged from 224 to 535 hours (approximately 9.3 to 22.3 days) across the 2 dose levels.

Over the dose range from 100 mg to 600 mg, the geometric mean AUC_{inf} and Cₘₐₓ after a single IV dose appeared to increase in a manner slightly more than dose-proportional, with an approximately 8-fold increase in geometric mean AUC_{inf} over the 6-fold dose increment (Table 18). After a single SC dose, maximal mean serum concentrations of OpSCF were attained at a similar median Tₘₐₓ at dose levels between 30 and 600 mg, ranging from 132 to 156 hours (5.5 to 6.5 days) postdose. Median Tₘₐₓ was slightly shorter at the 10 mg dose level, at 108 hours (4.5 days) postdose. After reaching Cₘₐₓ, serum concentrations of OpSCF appeared to decline in a monophasic manner. There was no consistent trend between dose level and geometric mean t_{1/2}, with values ranging from 350 to 471 hours (approximately 14.6 to 19.6 days). For individual subjects across all dose levels, the t_{1/2} ranged from 197 to 605 hours.

TABLE 18
Summary of the pharmacokinetic parameters for OpSCF following a single
subcutaneous or intravenous dose for a SAD study

| | 10 mg | 30 mg | 100 mg | 300 mg | 600 mg | 100 mg | 600 mg |
|---|---|---|---|---|---|---|---|
| | OpSCF SC | OpSCF SC | OpSCF SC | OpSCF SC | OpSCF SC | OpSCF IV | OpSCF |
| Parameter | (N = 8) | (N = 8) | (N = 8) | (N = 8) | (N = 8) | (N = 8) | IV (N = 8) |
| AUCₜ (h*ng/ml) | 555000 | 2240000 | 6570000 | 24000000 | 49600000 | 11300000 | 92300000 |
| | (35.9) [7] | (23.4) [8] | (26.3) [8] | (40.7) [7] | (32.5) [8] | (19.9) [7] | (25.0) [7] |
| AUC_{inf} (h*ng/ml) | 615000 | 2290000 | 6660000 | 24800000 | 50200000 | 11400000 | 93300000 |
| | (30.8) [7] | (23.0) [8] | (26.5) [8] | (39.3) [7] | (32.9) [8] | (20.2) [7] | (25.8) [7] |
| % AUCₑₓₜᵣₐₚ (%) | 11.1 | 1.81 | 1.12 | 1.96 | 0.895 | 1.59 | 1.43 |
| | (98.3) [8] | (27.9) [8] | (73.7) [8] | (311.5) [7] | (117.8) [8] | (235.7) [8] | (296.3) [8] |
| Cₘₐₓ (ng/ml) | 854 | 3580 | 9900 | 30000 | 82300 | 42700 | 288000 |
| | (43.8) [8] | (41.8) [8] | (33.2) [8] | (75.8) [8] | (48.1) [8] | (15.8) [8] | (38.6) [8] |
| Tₘₐₓ (h) | 108 (48.0- | 132 (48.0- | 156 (96.0- | 156 (48.0- | 132 (72.0- | 4.00 (0.200- | 4.00 (4.00- |
| | 337) [8] | 168) [8] | 168) [8] | 336) [8] | 168) [8] | 8.00) [8] | 72.0) [8] |
| Tₗₐₛₜ (h) | 2020 (1340- | 2520 (2160- | 2520 (2520- | 2520 (2520- | 2450 (2020- | 2520 (1900- | 2520 (2520- |
| | 2020) [7] | 2540) [8] | 2570) [8] | 2520) [7] | 2520) [8] | 2520) [7] | 2540) [7] |
| T_{1/2} (h) | 471 (14.5) | 423 (10.2) | 375 (13.9) | 459 (32.4) | 350 (26.5) | 359 (38.5) | 366 |
| | [8] | [8] | [8] | [7] | [8] | [8] | (18.9) [8] |
| CL/F (L/h) | 0.0162 (30.8 | 0.01312 | 0.0150 | 0.0121 | 0.0120 | — | — |
| | [7] | (23.0) [8] | (26.5) [8] | (39.3) [7] | (32.9) [8] | | |
| V/F (L) | 11.2 (36.8) | 8.00 (31.6) | 8.12 (24.4) | 8.02 (74.1) | 6.03 (35.0) | — | — |
| | [7] | [8] | [8] | [7] | [8] | | |
| CL (L/h) | — | — | — | — | — | 0.00874 | 0.00643 |
| | | | | | | (20.2) [7] | (25.8) [7] |
| V (L) | — | — | — | — | — | 4.74 (27.5) | 3.54 |
| | | | | | | [7] | (14.4) [7] |
| Vₛₛ (L) | — | — | — | — | — | 4.28 (26.5) | 3.18 |
| | | | | | | [7] | (17.5) [7] |
| DAUCₜ | 55500 | 74800 | 65700 | 79900 (40.7) | 82700 (32.5) | 113000 | 154000 |
| (h*ng/ml/mg) | (35.9) [7] | (23.4) [8] | (26.3) [8] | [7] | [8] | (19.9) [7] | (25.0) [7] |
| DAUC_{inf} | 61500 | 76200 | 66600 | 82500 (39.3) | 83700 (32.9) | 114000 | 156000 |
| (h*ng/ml/mg) | (30.8) [7] | (23.0) [8] | (26.5) [8] | [7] | [8] | (20.2) [7] | (25.8) [7] |
| DCₘₐₓ (ng/mL/mg) | 85.4 (43.8) | 119 (41.8) | 99.0 (33.2) | 99.9 (75.8) | 137 (48.1) | 427 (15.8) | 480 |
| | [8] | [8] | [8] | [8] | [8] | [8] | (38.6) [8] |
| F (%) | — | — | 58.9 | — | 54.4 | — | — |

% AUCₑₓₜᵣₐₚ = percentage of area under the concentration-time curve due to extrapolation from the last quantifiable concentration to infinity;
AUC_{inf} = area under the serum concentration-time curve from time 0 to infinity;
AUCₜ = area under the serum concentration-time curve from time 0 to the time of the last measurable concentration;
CL = clearance;
Cₘₐₓ = maximum serum concentration;
CV = coefficient of variation (%);
F = absolute bioavailability;
IV = intravenous;
n = number of subjects with valid observations;
N = number of subjects;
SC = subcutaneous;
t_{1/2} = terminal half life;
Tₗₐₛₜ = time of the last quantifiable concentration;
Tₘₐₓₓ = time to maximum serum concentration;
V = volume of distribution;
Vₛₛ = volume of distribution at steady state.
Where F is presented as a parameter this has been calculated by dividing the geometric mean of the AUC_{inf} from the SC dose by the corresponding geometric mean of the AUC_{inf} from the IV dose. Presented where the SC and IV doses were administered at the same dose level.
Parmeter starting with ‘D’ letter signifies the corresponding parameter was normalized by dose administered.
For continuous data, mean (SD) [N] statistics presented.

Moreover, over the dose range 10 to 600 mg, geometric mean AUC_{inf} and Cₘₐₓ after a single SC dose generally appeared to increase in a dose-proportional manner. This was confirmed by statistical analysis, with the estimates of the slopes (95% CI) from the regression analysis for AUC_{inf} and Cₘₐₓ being 1.06 (0.998 to 1.13) and 1.07 (0.967 to 1.17), respectively (Table 19). Dose proportionality results are further depicted in FIGS. 30A-30F. The F at the 100 and 600 mg OpSCF dose levels was 58.9% and 54.4%, respectively.

TABLE 19
Statistical analysis of the dose proportionality
of PK Parameters for OpSCF following a single
subcutaneous doses on Day 1 of the SAD study

| | | | Between- | Lack of |
|---|---|---|---|---|
| | | | subject | Fit 2-sides |
| Parameter | Slope (95% CI) | | Geometric CV | P-value |
| AUCₜ (h*ng/mL) | 1.08 | (1.01, 1.15) | 31.9 | 0.4643 |
| AUC_{inf} (h*ng/mL) | 1.06 | (0.998, 1.13) | 30.6 | 0.5340 |
| Cₘₐₓ (ng/mL) | 1.07 | (0.967. 1.17) | 49.7 | 0.3984 |

AUC_{inf} = area under the concentration-time curve from time O extrapolated to infinity;
AUCₜ = area under the concentration-time curve from time Oto the time of the last quantifiable concentration;
CI = confidence interval;
Cₘₐₓ = maximum observed concentration;
CV = coefficient of variation(%);
In = natural log
Model: ln(parameter) = intercept + slope × ln(dose) + random error
Lack of Fit Model: ln(parameter) = intercept + slope × ln(dose) + dose + random error
Dose range studied was 10 to 600 mg

ADA tests were positive for 14 (25.0%) of 56 Op-SCF-treated subjects at 1 or more time points. However, no AEs were reported related to the positive ADA tests reported, and most instances were attributable to biologic variation.

Safety: A total of 21 subjects (30.0%) had at least 1 TEAE (Table 20). There was no apparent difference in the number of subjects reporting TEAEs between dose groups. The majority of TEAEs were Grade 1. The severity of TEAEs did not appear to increase with dose. None of the TEAEs were considered, by the Investigator, to be related to OpSCF administration. There was no apparent difference in relatedness of TEAEs between treatment groups. The majority of TEAEs had recovered/resolved by the end of the study. Overall, there were no deaths or SAEs, and no participants were discontinued due to an AE.

TABLE 20
Summary of Treatment-emergent Adverse Events for SAD study

| | | 10 mg | 30 mg | 100 mg | 300 mg | 600 mg | | 100 mg | 600 mg | |
|---|---|---|---|---|---|---|---|---|---|---|
| | Placebo | OpSCF | OpSCF | OpSCF | OpSCF | OpSCF | Placebo | OpSCF | OpSCF | |
| | SC | SC | SC | SC | SC | SC | IV | IV | IV | Overall |
| Parameter | (N = 10) | (N = 8) | (N = 8) | (N = 8) | (N = 8) | (N = 8) | (N = 4) | (N = 8) | (N = 8) | (N = 70) |
| Overall | 2 | 1 | 3 | 4 | 1 | 4 | 1 | 3 | 2 | 21 |
| | (20.0%) | (12.5%) | (37.5%) | (50.0%) | (12.5%) | (50.0%) | (25.0%) | (37.5%) | (25.0%) | (30.0%) |
| | [4] | [1] | [5] | [5] | [1] | [6] | [3] | [8] | [3] | [36] |
| Serious | — | — | — | — | — | — | — | — | — | — |
| Leading to | — | — | — | — | — | — | — | — | — | — |
| Discontinuation | | | | | | | | | | |
| Leading to Death | — | — | — | — | — | — | — | — | — | — |
| Severity | | | | | | | | | | |
| Grade 1 | 2 | 1 | 3 | 4 | 1 | 3 | 1 | 1 | 2 | 18 |
| | (20.0%) | (12.5%) | (37.5%) | (50.0%) | (12.5%) | (37.5%) | (25.0%) | (12.5%) | (25.0%) | (25.7%) |
| | [3] | [1] | [4] | [5] | [1] | [4] | [3] | [1] | [2] | [24]v |
| Grade 2 | — | — | 1 | — | — | 1 | — | 2 | — | 4 (5.7%) |
| | | | (12.5%) | | | (12.5%) | | (25.0%) | | [10] |
| | | | [1] | | | [2] | | [7] | | |
| Grade 3 | 1 | — | — | — | — | — | — | — | — | 1 (1.4%) |
| | (10.0%) | | | | | | | | | [1] |
| | [1] | | | | | | | | | |
| Grade 4 | — | — | — | — | — | — | — | — | 1 | 1 (1.4%) |
| | | | | | | | | | (12.5%) | [1] |
| | | | | | | | | | [1] | |
| Grade 5 | — | — | — | — | — | — | — | — | — | — |

CTCAE = Common Terminology Criteria for Adverse Events;
IV = intravenous;
nE = number of adverse events;
nS = number of subjects with an adverse event;
N = number of subjects;
SC = subcutaneous;
TEAE = treatment-emergent adverse event;
% = percentage of subjects with an adverse event (nS/Nx 100)
The nS (%) [nE] statistics presented.
Severity grades: 1 = mild; 2 = moderate; 3 = severe; 4 = life-threatening; S = fatal.
Adverse events were assigned severity grade using the CTCAE Version 5.0.
A TEAE was defined as an adverse event that started during or after dosing or started prior to dosing and increased in severity after dosing.
Where a subject experienced multiple TEAEs with the same preferred term for the same treatment, this was counted as 1 TEAE for that treatment under the maximum severity recorded.
For categorical data, N (%) [n of events] statistics presented.

Conclusions: Single SC doses of 10 to 600 mg OpSCF were safe and well tolerated by the healthy subjects in this study. No subjects reported an SAE and no subjects were discontinued because of a TEAE. There were no treatment- or dose-related trends and no clinically significant findings in the clinical laboratory evaluations, vital signs data, 12-lead ECG data, or physical examinations during the study. There were no ISRs or IRRs in this study. Following single SC doses of 10 to 600 mg, OpSCF had a median Tₘₐₓ of between 108 and 156 hours, and a geometric mean t_{1/2} of between 350 and 471 hours. Following single 100-mg and 600-mg IV doses, OpSCF had a median Tₘₐₓ of 4 hours, and a geometric mean t_{1/2} of between 359 and 366 hours. Systemic exposure to OpSCF following single SC doses was dose proportional over the 10 mg to 600 mg dose range. Pharmacodynamic parameters, which included serum flow cytometry and soluble protein parameters, did not change markedly during this study.

### Example 17: Phase 1a: Randomized, Double-Blind, Placebo-Controlled Study to Assess the Safety, Tolerability, and Pharmacokinetics of OpSCF after a Multiple Ascending Dose (MAD) Study in Healthy Subjects

Study Overview: This study was a Phase 1a, randomized, double-blind, placebo-controlled, multiple ascending IV and subcutaneous doses first-in-human (FIH) study. Cohorts of healthy adult subjects were assessed for safety, tolerability, immunogenicity, PK, and PD of OpSCF. OpSCF is a humanized immunoglobulin (Ig) G4 kappa monoclonal antibody specific for the long splice variant of stem cell factor 248 (SCF248) and comprises a heavy chain of SEQ ID NO: 42 and a light chain of SEQ ID NO: 49.

Study Objectives: This study sought to assess the primary, secondary and exploratory objectives described below.

### Primary Objective:

To determine the safety and tolerability of multiple doses of OpSCF in health subjects.

### Secondary Objectives:

To determine the multiple dose PK parameters of OpSCF following multiple doses of OpSCF in healthy subjects.
To determine the immunogenicity of OpSCF in healthy subjects as measured by anti-drug antibodies (ADAs).

### Exploratory Objective:

To assess the PD response after multiple doses of OpSCF using PD markers including SCF165, cytokines, mast cell and eosinophil products, and innate lymphoid cell (ILC) precursors in blood.

Study design: Healthy adult subjects were studied in 3 multiple dose, sequential cohorts (30 mg, 100 mg or 300 mg for IV on day 1 and subcutaneous on Days 8, 15, and 22) of approximately 15 subjects each who were randomized in a 12:3 ratio to receive multiple doses of OpSCF or placebo. The study cohorts are described in further detail below in Table 21. Consented eligible subjects were enrolled into sequential cohorts of increasing doses of OpSCF. Each dose cohort enrolled approximately 15 subjects, and dosing occurred once a week for 4 weeks. All doses were administered in accordance with a randomization schedule on the morning of Days 1, 8, 15, and 22. After dosing in the 30 mg cohort, the safety of dosing each cohort, along with tolerability, laboratory data and any available PK data, were reviewed by the DEC and escalation proceeded as per dose escalation procedures.

TABLE 21
Study cohorts administered OpSCF in a MAD study

| MAD | Planned Number | Actual Investigational Medicinal |
|---|---|---|
| Cohort # | of Subjects | Product Dose |
| M1 | 15 | 30 mg OpSCF IV and SC/placebo^{a} |
| M2 | 15 | 100 mg OpSCF IV and SC/placebo^{a} |
| M3 | 15 | 300 mg OpSCF IV and SC/placebo^{a} |

Abbreviations:
IV = intravenous;
MAD = multiple ascending dose;
SC = subcutaneous
^{a}= IV on Study Day 1, SC on Study Days 8, 15, and 22.

Participants: Participants included healthy adults. Forty-six subjects were randomized and dosed and 38 subjects (82.6%) completed the study in accordance with the protocol. Two subjects (4.3%) discontinued because of an AE, two subjects (4.3%) withdrew from the study, and two subjects (4.3%) were lost to follow-up. Two subjects (4.3%) were discontinued because of a protocol deviation. The demographic characteristics of the study population are in Table 22 below.

TABLE 22
Summary of screening demographic data

| | | | 30 mg | | 100 mg | | 300 mg | | | |
|---|---|---|---|---|---|---|---|---|---|---|
| | Placebo | | OpSCF | | OpSCF | | OpSCF | | Overall | |
| Demographic | (N = 9) | | (N = 13) | | (N = 12) | | (N = 12) | | (N = 46) | |
| Age (years) | 36.3 | (8.25) | 39.2 | (9.75) | 42.6 | (12.99) | 43.0 | (13.53) | 40.5 | (11.41) |
| Sex | | | | | | | | | | |
| Male | 6 | (66.7%) | 8 | (61.5%) | 9 | (75.0%) | 7 | (58.3%) | 30 | (65.2%) |
| Female | 3 | (33.3%) | 5 | (38.5%) | 3 | (25.0%) | 5 | (41.7%) | 16 | (34.8%) |
| Race | | | | | | | | | | |
| White | 3 | (33.3%) | 8 | (61.5%) | 3 | (25.0%) | 9 | (75.0%) | 23 | (50.0%) |
| Black or African American | 5 | (55.6%) | 3 | (23.1%) | 7 | (58.3%) | 1 | (8.3%) | 16 | (34.8%) |
| Asian | 1 | (11.1%) | 1 | (7.7%) | 1 | (8.3%) | 2 | (16.7%) | 5 | (10.9%) |
| Native Hawaiian or Other Pacific | — | | — | | 1 | (8.3%) | — | | 1 | (2.2%) |
| Islander | | | | | | | | | | |
| American Indian or Alaska | — | | 1 | (7.7%) | — | | — | | 1 | (2.2%) |
| Native | | | | | | | | | | |
| Ethnicity | | | | | | | | | | |
| Hispanic or Latino | 2 | (22.2%) | 4 | (30.8%) | 1 | (8.3%) | 3 | (25.0%) | 10 | (21.7%) |
| Not Hispanic or Latino | 7 | (77.8%) | 9 | (69.2%) | 11 | (91.7%) | 9 | (75.0%) | 36 | (78.3%) |
| Height (cm) | 173.51 | (7.516) | 171.91 | (12.623) | 174.62 | (7.615) | 168.82 | (6.830) | 172.12 | (9.115) |
| Body Weight (kg) | 78.93 | (13.163) | 78.18 | (13.669) | 79.54 | (11.824) | 75.89 | (12.497) | 78.09 | (12.449) |
| Body Mass Index (kg/m²) | 26.14 | (3.528) | 26.37 | (3.230) | 26.11 | (3.726) | 26.52 | (3.718) | 26.30 | (3.309) |

V = intravenous;
n = number of subjects with valid observations;
N = number of subjects;
SC = subcutaneous;
SD = standard deviation;
% = percentage of subjects with valid observations (n/N × 100)
For continuous data, mean (SD) statistics presented; for categorical data, n (%) statistics presented.
Body mass index (kg/m2) = body weight (kg)/height (m)2
Dose levels are delivered via IV route on Day 1 and SC route on Days 8, 15, and 22.

Treatment administered: Each subject received OpSCF or placebo in accordance with the randomization schedule, with a single IV dose on Day 1 plus single SC doses administered on Days 8, 15, and 22 for the MAD cohorts.

Clinical endpoints: The same PK, PD, and safety endpoints were assessed as above in Example 16.

### Results:

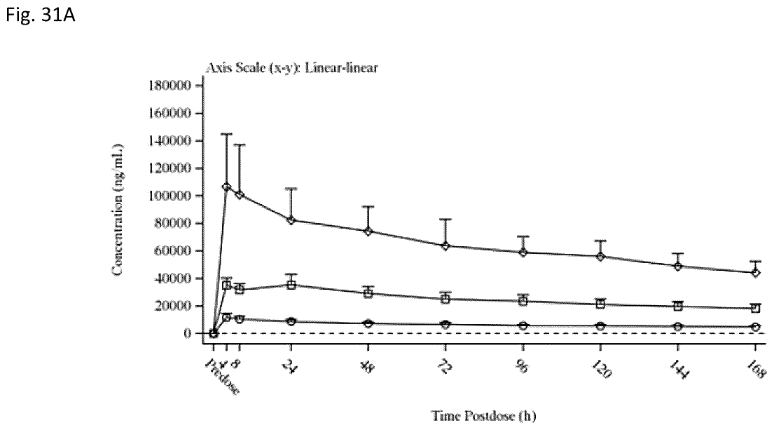

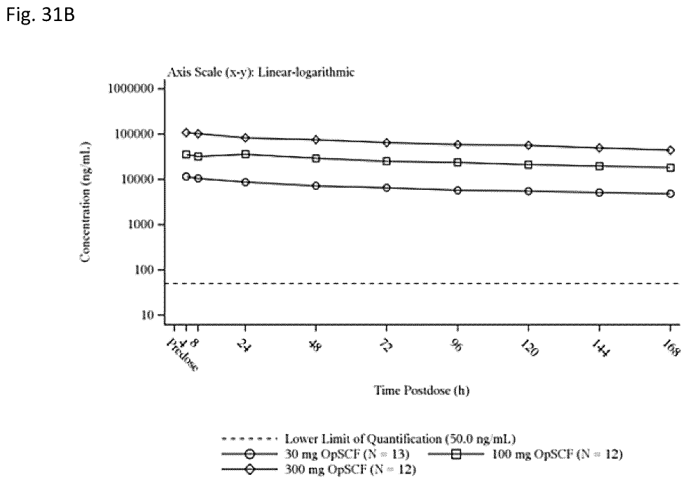

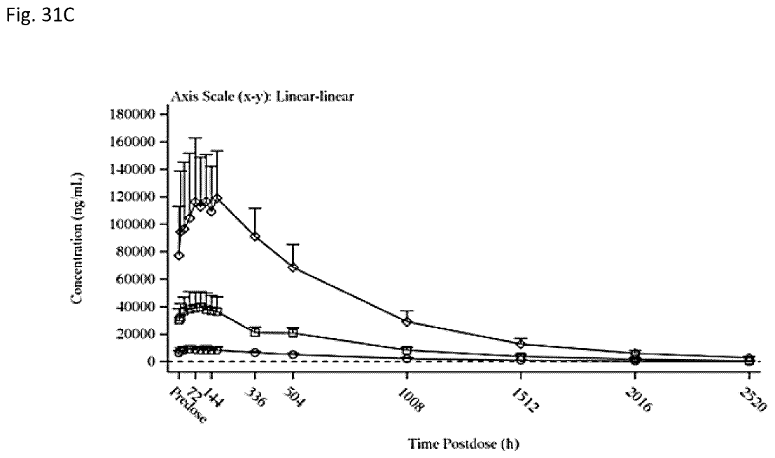

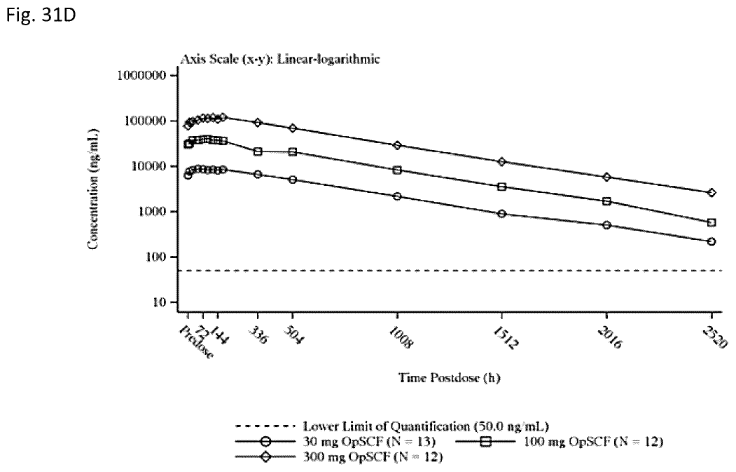

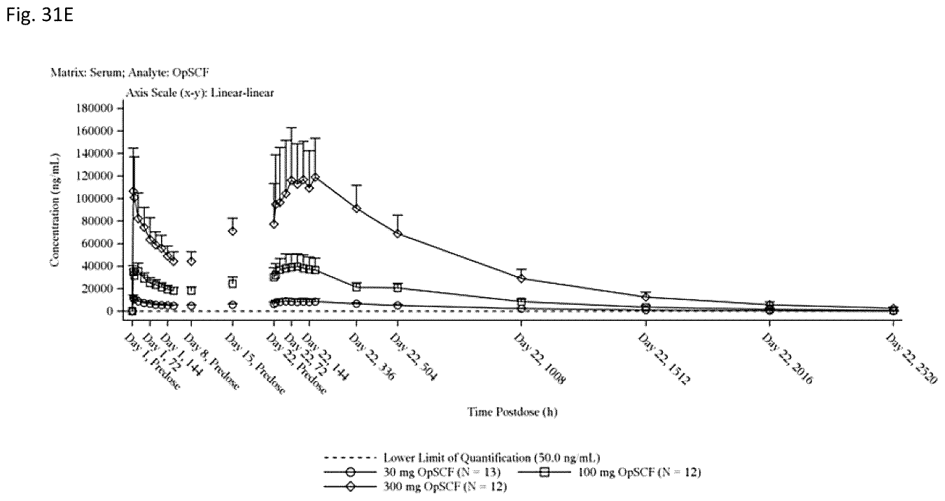

PK and PD: Serum concentrations of OpSCF are presented for Day 1 (FIGS. 31A-31B), Day 22, (FIGS. 31C-31D), and across all days (FIG. 31E). The PK parameters of OpSCF are summarized in Table 23 (Day 1) and Table 24 (Day 22). The results of the statistical analysis of dose proportionality and attainment of steady state for SC doses are presented in Table 25.

In the MAD study, OpSCF was administered by IV on Day 1. As shown in FIGS. 31A-31E, maximal mean serum concentrations of OpSCF were attained immediately after a single IV dose and declined in a monophasic manner.

After a loading IV dose on Day 1 and multiple SC doses on Days 8, 15, and 22, maximal mean serum concentrations of OpSCF on Day 22 were attained at a similar median Tₘₐₓ at dose levels from 30 through 300 mg, ranging from 60 to 120 hours postdose (Tables 23 and 24). After reaching Cₘₐₓ, serum concentrations of OpSCF appeared to decline in a monophasic manner. The geometric mean t_{1/2} on Day 22 was similar across dose levels, with values ranging from 374 to 427 hours. For individual subjects across all dose levels, the t_{1/2} ranged from 219 to 498 hours.

TABLE 23
Summary of the pharmacokinetic parameters for OpSCF following the
first dose in a Multiple Ascending Dose study - Profile Day 1

| Parameter | 30 mg OpSCF (N = 13) | | 100 mg OpSCF (N = 12) | | 300 mg OpSCF (N = 12) | |
|---|---|---|---|---|---|---|
| AUCₜₐᵤ (h*ng/mL) | 1100000 | (15.5) [12] | 4200000 | (17.7) [12] | 10800000 | (23.0) [12] |
| Cₘₐₓ (ng/mL) | 11700 | (22.2) [12] | 37400 | (17.9) [12] | 108000 | (28.8) [ 12] |
| Tₘₐₓ (h) | 4.00 | (0.117-8.00) [12] | 14.0 | (4.00-24.1) [12] | 4.00 | (4.00-96.0) [12] |
| Tₗₐₛₜ (h) | 168 | (168-168) [12] | 168 | (168-168) [12] | 168 | (168-168) [12] |
| DAUCₜₐᵤ (h*ng/mL/mg) | 36500 | (15.5) [12] | 42000 | (17.7) [12] | 36000 | (23.0) [12] |
| DCₘₐₓ (ng/mL/mg) | 390 | (22.2) [12] | 374 | (17.9) [12] | 362 | (28.8) [12] |

AUCₜₐᵤ = area under the serum concentration-time curve over a dosing interval (tau = 168 hours);
Cmax = maximum serum concentration;
CV = coefficient of variation (%);
IV = intravenous;
n = number of subjects with valid observations;
=number of subjects;
SC = subcutaneous;
Tₗₐₛₜ = time of the last quantifiable concentration;
t½ = terminal half life;
Tₘₐₓ = time to maximum serum concentration
Parameter starting with ‘D’ letter signifies the corresponding parameter was normalized by dose administered.
Geometric mean (CV) [n] statistics are presented, except for Tₘₐₓ, and Tₗₐₛₜ, wherein median (minimum-maximum) [n] statistics are presented.
Dose levels were delivered via IV route on Day 1 and SC route on Days 8, 15, and 22.
For continuous data, mean (SD) [N] statistics presented.

TABLE 24
Summary of the Pharmacokinetic Parameters for OpSCF Following Multiple Doses - Profile Day 22

| Parameter | 30 mg OpSCF (N = 13) | | 100 mg OpSCF (N = 12) | | 300 mg OpSCF (N = 12) | |
|---|---|---|---|---|---|---|
| AUCₜₐᵤ (h*ng/mL) | 1350000 | (3 0.1) [11] | 6080000 | (27.1) [10] | 17200000 | (35.6) [12] |
| Cₘₐₓ (ng/mL) | 9370 | (34.6) [11] | 39800 | (27.6) [10] | 120000 | (32.3) [12] |
| Cₘᵢₙ (ng/mL) | 6030 | (29.2) [11] | 28300 | (26.7) [10] | 63300 | (77.0) [12] |
| Tₘₐₓ (h) | 120 | (24.0-168) [11] | 60.0 | (24.0-120) [10] | 108 | (8.00-168) [12] |
| Tₗₐₛₜ (h) | 2520 | (1510-2520) [11] | 2520 | (2500-2520) [8] | 2520 | (1940-3220) [12] |
| t½ | 410 | (24.8) [11] | 374 | (24.6) [8] | 427 | (1 l.6) [12] |
| DAUCₜₐᵤ (h*ng/mL/mg) | 45000 | (30.l) [11] | 60800 | (27.l) [10] | 57300 | (35.6) [12] |
| DCₘₐₓ (ng/mL/mg) | 312 | (34.6) [11] | 393 | (27.6) [10] | 400 | (32.3) r 121 |

AUCₜₐᵤ = area under the serum concentration-time curve over a dosing interval (tau = 168 hours);
Cₘₐₓ = maximum serum concentration;
CV = coefficient of variation (%);
IV = intravenous;
n = number of subjects with valid observations;
=number of subjects;
SC = subcutaneous;
Tₗₐₛₜ = time of the last quantifiable concentration;
t½ = terminal half life;
Tₘₐₓ = time to maximum serum concentration
Parameter starting with ‘D’ letter signifies the corresponding parameter was normalized by dose administered.
Geometric mean (CV) [n] statistics are presented, except for Tₘₐₓ, and Tₗₐₛₜ, wherein median (minimum-maximum) [n] statistics are presented.
Dose levels were delivered via IV route on Day 1 and SC route on Days 8, 15, and 22.
For continuous data, mean (SD) [N] statistics presented.

Over the dose range 30 to 300 mg on Day 22, geometric mean AUCₜₐᵤ and Cₘₐₓ generally appeared to increase in a dose-proportional manner. This was confirmed by statistical analysis, with the estimates of the slopes (95% CI) from the regression analysis for AUCₜₐᵤ and Cₘₐₓ being 1.11 (0.990 to 1.22) and 1.11 (0.993 to 1.22), respectively, on Day 22 (Table 25).

TABLE 25
Statistical Analysis of the Dose Proportionality
of Pharmacokinetic Parameters for OpSCF Following
Multiple Subcutaneous Doses (MAD; Day 22)

| | | Between- | Lack of |
|---|---|---|---|
| | | subject | Fit 2-sided |
| Parameter | Slope (95% CI) | Geometric CV | P-value |
| AUCₜₐᵤ (h*ng/mL) | 1.11 (0.990, 1.22) | 31.4 | 0.1434 |
| Cₘₐₓ (ng/mL) | 1.11 (0.993, 1.22) | 31.8 | 0.3488 |

AUCₜₐᵤ = area wider the serum concentration-time curve over a dosing interval (tau = 168 hours);
Cmax = maximum observed concentration; CV= coefficient of variation(%);
1n = natural log
Model: ln(parameter) = intercept + slope × ln(dose) + random error
Lack of Fit Model: ln(parameter) = intercept + slope × ln(dose) + dose + random error
Dose range studied was 30 to 300 mg

At each dose level, visual examination of the morning trough OpSCF serum concentrations for individual subjects showed that an apparent steady state was achieved by approximately Day 15 due to the IV loading dose (Tables 26, 27, 28). In support of this observation, the 90% CIs for the ratios of geometric least squares means of C_{trough} at each dose level indicated a statistically significant difference between C_{trough} at Days 15 and 22 compared with Day 8, but not between Days 15 and 22.

TABLE 26
Statistical Analysis of Attainment of Steady State
of OpSCF Following Multiple Doses of 30 mg of OpSCF

| Parameter | Day | n | GLSM | Ratio of GLSMs (0% CI) | |
|---|---|---|---|---|---|
| C_{trough} | Day 8 (Reference) | 11 | 4750 | — | |
| (ng/mL) | Day 15 (Test) | 11 | 5470 | 1.15 | (1.06, 1.25) |
| | Day 22 (Test) | 11 | 6100 | 1.28 | (1.01, 1.63) |
| | Day 15 (Reference) | 11 | 5470 | — | |
| | Day 22 (Test) | 11 | 6100 | 1.12 | (0.874, 1.42) |

CI = confidence interval;
Ctrough = trough serum concentration;
CV = coefficient of variation (%);
GLSM = geometric least squares mean;
1n = natural log;
LSM = least square mean;
n = number of subjects with valid observations;
C = not calculated;
SC = subcutaneous
Dose levels are delivered via SC route on Days 8, 15, and 22.
Model: ln(parameter) = treatment + time + time × treatment + random error, with subject fitted as a random effect
An unstructured covariance structure is used to model the correlation between a subject's multiple observations.
The GLSMs, ratios of GLSMs, and corresponding CIs were obtained by taking the exponential of the LSMs, differences in LSMs, and corresponding CIs on the 1n scale.

TABLE 27
Statistical Analysis of Attainment of Steady State of
OpSCF Following Multiple Doses of 100 mg of OpSCF

| Parameter | Day | n | GLSM | Ratio of GLSMs (0% CI) | |
|---|---|---|---|---|---|
| C_{trough} | Day 8 (Reference) | 11 | 18000 | — | |
| (ng/mL) | Day 15 (Test) | 11 | 23700 | 1.32 | (1.21, 1.43) |
| | Day 22 (Test) | 10 | 28700 | 1.59 | (1.24, 2.04) |
| | Day 15 (Reference) | 11 | 23700 | — | |
| | Day 22 (Test) | 10 | 29100 | 1.23 | (0.948, 1.58) |

CI = confidence interval;
Ctrough = trough serum concentration;
CV = coefficient of variation (%);
GLSM = geometric least squares mean;
1n = natural log;
LSM = least square mean;
n = number of subjects with valid observations;
C = not calculated;
SC = subcutaneous
Dose levels are delivered via SC route on Days 8, 15, and 22.
Model: ln(parameter) = treatment + time + time × treatment + random error, with subject fitted as a random effect
An unstructured covariance structure is used to model the correlation between a subject's multiple observations.
The GLSMs, ratios of GLSMs, and corresponding CIs were obtained by taking the exponential of the LSMs, differences in LSMs, and corresponding CIs on the 1n scale.

TABLE 28
Statistical Analysis of Attainment of Steady State of
OpSCF Following Multiple Doses of 300 mg of OpSCF

| Parameter | Day | n | GLSM | Ratio of GLSMs (0% CI) | |
|---|---|---|---|---|---|
| C_{trough} | Day 8 (Reference) | 12 | 43500 | — | |
| (ng/mL) | Day 15 (Test) | 12 | 70300 | 1.62 | (1.49, 1.75) |
| | Day 22 (Test) | 12 | 66500 | 1.53 | (1.22, 1.92) |
| | Day 15 (Reference) | 12 | 70300 | — | |
| | Day 22 (Test) | 12 | 66500 | 0.947 | (0.750, 1.20) |

CI = confidence interval;
Ctrough = trough serum concentration;
CV = coefficient of variation (%);
GLSM = geometric least squares mean;
1n = natural log;
LSM = least square mean;
n = number of subjects with valid observations;
C = not calculated;
SC = subcutaneous
Dose levels are delivered via SC route on Days 8, 15, and 22.
Model: ln(parameter) = treatment + time + time × treatment + random error, with subject fitted as a random effect
An unstructured covariance structure is used to model the correlation between a subject's multiple observations.
The GLSMs, ratios of GLSMs, and corresponding CIs were obtained by taking the exponential of the LSMs, differences in LSMs, and corresponding CIs on the 1n scale.

Given the half-life of 15 to 20 days for OpSCF, steady state is not expected to be achieved within the duration of this study which is shorter than 4 to 5 half-lives. However, the IV loading dose was able to provide an early high exposure that helped reach an apparent steady state much faster.

ADA tests were positive for 9 (16.6%) of 36 Op-SCF-treated subjects at 1 or more time points. However, no AEs were reported related to the positive ADA tests reported, and most instances were attributable to biologic variation.

Safety: A total of 20 subjects (43.5%) had at least 1 TEAE (Table 29). There was no apparent difference in the number of subjects reporting TEAEs between dose groups. The majority of TEAEs were Grade 1. The severity of TEAEs did not appear to increase with dose. Ten treatment-related TEAEs in 8 subjects (17.4%) were reported. One treatment-related TEAE led to discontinuation. All treatment-related TEAEs were Grade 1. There was no apparent difference in relatedness of TEAEs between treatment groups. The majority of TEAEs had recovered/resolved by the end of the study.

TABLE 29
Summary of Treatment-emergent Adverse Events for MAD OpSCF study

| | | | | | |
|---|---|---|---|---|---|
| | Placebo | 30 mg | 100 mg | 300 mg | |
| | SC | OpSCF | OpSCF | OpSCF | Overall |
| Parameter | (N = 9) | (N = 13) | (N = 12) | (N = 12) | (N = 46) |

TEAEs

| Overall | 3 (33.3%) [7] | 7 | (53.8%) [9] | 3 | (25.0%) [5] | 7 | (58.3%) [16] | 20 | (43.5%) [37] |
|---|---|---|---|---|---|---|---|---|---|
| Serious | — | — | | — | | 1 | (8.3%) [2] | 1 | (2.2%) [2] |
| Leading to | — | 1 | (7.7%) [1] | 1 | (8.3%) [1] | — | | 2 | (4.3%) [2] |
| Discontinuation | | | | | | | | | |
| Leading to Death | — | — | | — | | — | | — | |
| Severity | | | | | | | | | |
| Grade 1 | 3 (33.3%) [7] | 5 | (38.5%) [7] | 2 | (16.7%) [4] | 7 | (58.3%) [14] | 17 | (37.0%) [32] |

| | | | | | | | | |
|---|---|---|---|---|---|---|---|---|
| Grade 2 | — | 1 | (7.7%) [1] | 1 | (8.3%) [l] | — | 2 | (4.3%) [2] |

| | | | | | | | |
|---|---|---|---|---|---|---|---|
| Grade 3 | — | — | — | 1 | (8.3%) [2] | 1 | (2.2%) [2] |

| | | | | | | | |
|---|---|---|---|---|---|---|---|
| Grade 4 | — | 1 | (7.7%) [1] | — | — | 1 | (2.2%) [1] |

| | | | | | |
|---|---|---|---|---|---|
| Grade 5 | — | — | — | — | — |

Treatment-related TEAEs

| Overall | 1 (11.1%) [1] | 2 | (15.4%) [3] | l | (8.3%) [2] | 4 | (33.3%) [4] | 8 | (17.4%) [10] |
|---|---|---|---|---|---|---|---|---|---|
| Serious | — | — | | — | | — | | — | |
| Leading to | — | 1 | (7.7%) [l] | — | | — | | l | (2.2%) [l] |
| Discontinuation | | | | | | | | | |
| Leading to Death | — | — | | — | | — | | — | |
| Severity | — | — | | — | | — | | — | |
| Grade 1 | 1 (11.1%) [1] | 2 | (15.4%) [3] | l | (8.3%) [2] | 4 | (33.3%) [4] | 8 | (17.4%) [10] |

| | | | | | |
|---|---|---|---|---|---|
| Grade 2 | — | — | — | — | — |
| Grade 3 | — | — | — | — | — |
| Grade 4 | — | — | — | — | — |
| Grade 5 | — | — | — | — | — |

CTCAE = Common Tenninology Criteria for Adverse Events;
IV = intravenous;
nE = number of adverse events;
nS = number of subjects with an adverse event;
N = number of subjects;
SC = subcutaneous;
TEAE = treatment-emergent adverse event;
% = percentage of subjects with an adverse event (nS/N × 100)
The nS (%) [nE] statistics presented.
Severity grades:
1 = mild;
2 = moderate;
3 = severe;
4 = life-threatening;
5 = fatal
Adverse events were assigned severity grade using the CTCAB Version 5.0.
A TEAE was defined as an adverse event that started during or after dosing, or started prior to dosing and increased in severity after dosing.
Where a subject experienced multiple TEAEs with the same preferred term for the same treatment, this was counted as 1 TEAE for that treatment under the maximum severity recorded.
Dose levels were delivered via IV route on Day 1 and SC route on Days 8, 15, and 22.
For categorical data, N (%) [n of events] statistics presented.

Conclusions: Multiple 30 mg, 100 mg, and 300 mg IV doses on Day 1 and SC doses on Days 8, 15, and 22 of OpSCF were safe and well tolerated by the healthy subjects in this study. One subject had 2 SAEs during the study and 2 subjects were discontinued because of a TEAE. There were no treatment- or dose-related trends and no clinically significant findings in the clinical laboratory evaluations, vital signs data, 12-lead ECG data, or physical examinations during the study. There were no ISRs or IRRs in this study.

Following multiple 30 mg, 100 mg, and 300 mg IV doses on Day 1 and SC doses on Days 8, 15, and 22, OpSCF accumulated in serum, with accumulation ratios of 1.15, 1.28, and 1.12, respectively, for the 30 mg dose, including accumulation ratios of 1.32, 1.59, and 1.23, respectively, for the 100 mg dose and accumulation ratios of 1.62, 1.53, and 0.947, respectively, for the 300 mg dose. The geometric mean t_{1/2} was approximately 374 hours for the 100 mg OpSCF dose and between 410 and 427 hours for the 30 mg and 300 mg doses, respectively, on Day 22. Systemic exposure to OpSCF following single SC doses was dose proportional over the 30 mg to 300 mg dose range. Pharmacodynamic parameters, which included serum flow cytometry and soluble protein parameters, did not change markedly during this study.

### Example 18: a Randomized, Double-Blind, Placebo-Controlled, Phase 2a Study to Evaluate the Efficacy and Safety of Humanized 5H10 Antibody in the Treatment of Adult Subjects with Moderate to Severe Atopic Dermatitis

Study Overview: A Phase 2a Study of Efficacy and Safety of the humanized 5H10 monoclonal antibody (also referred to as “OpSCF”) was performed. OpSCF comprises two heavy chains each with an amino acid sequence of SEQ ID NO: 42 and two light chains, each with an amino acid sequence of SEQ ID NO: 49.

Study Objectives: This study sought to determine the efficacy and safety of OpSCF in the treatment of adults with moderate to severe Atopic Dermatitis (AD) (Eczema). OpSCF was compared to a placebo.

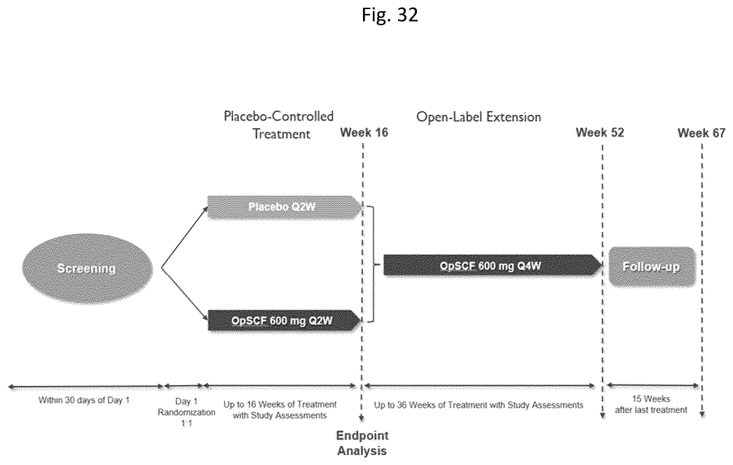

Study design: This Phase 2a study was a randomized, double-blind, placebo-controlled, parallel-group study designated to evaluate the efficacy and safety of OpSCF among 48 individuals from 20 sites with moderate to severe AD. PK, PD and immunogenicity were assessed at weeks 2, 4, 6, 8, 12, 14, 15 and 16. A study timeline is provided in FIG. 32.

OpSCF or placebo was administered at 600 mg every 2 weeks for 14 weeks, and the efficacy was assessed two weeks later. After that, subjects chose to enter an Open Label Extension phase in which all subjects regardless of treatment assignment received 600 mg of OpSCF every 4 weeks for up to 36 weeks. The study was a randomized, placebo-controlled study with quadruple masking (participant, care provider, investigator, outcomes assessor). Doses for each study arm are described in Table 30 below.

TABLE 30
Study design of Phase 2 study

| Participant Group/Arm | Intervention/Treatment |
|---|---|
| Active Comparator: OpSCF | Biological: OpSCF |
| OpSCF, 600 mg, subcutaneously, every two | Monoclonal Antibody |
| weeks × 14 weeks | |
| Placebo Comparator: Placebo | Biological: Placebo |
| Matched placebo, subcutaneously, every two | Formulation buffer without active |
| weeks × 14 weeks | agent |
| Active Comparator: Open Label Extension | Biological: OpSCF |
| Subjects may choose to continue in an Open | Monoclonal Antibody |
| Label Extension (OLE) phase if they complete | Biological: OpSCF (Open Label Extension) |
| the randomized phase. All subjects regardless | Subjects may continue on an Open |
| of treatment assignment in the randomized | Label Extension after completing the |
| phase will receive OpSCF, 600 mg every 4 | 16-week randomized portion of the |
| weeks for up to 36 weeks. | study |

Participants: The inclusion criteria were: (i) subject has clinically confirmed diagnosis of active AD; (ii) subject has at least a 6-month history of AD; (iii) subject is willing to use effective birth control. The exclusion criteria were: (i) Subject is a female who is breastfeeding, pregnant, or who is planning to become pregnant during the study; (ii) subject has any clinically significant medical condition that would put the subject at undue risk or interfere with interpretation of study results; (iii) Subject has used dupilumab within 26 weeks prior to Day 1; and (iv) Subject has used tralokinumab within 12 weeks prior to Day 1. The subjects were from 18 to 75 years old.

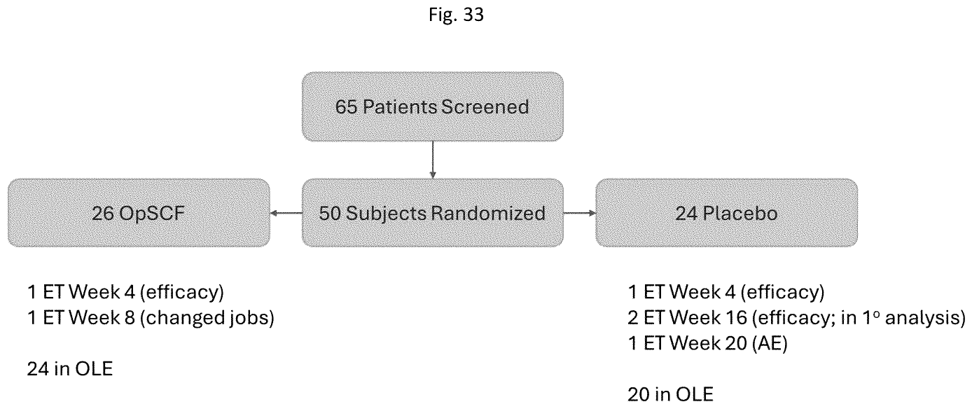

Participants included adults with an Eczema Area and Severity Index (EASI) score ≥16, a validated Investigational Global Assessment scale for AD (vIGA-AD) score ≥3, and an AD related body surface area (BSA) of ≥10% at screening on Day 1. Individuals were excluded for use of a biologic or JAK inhibitor for the treatment of AD and discontinued for lack of efficacy. The participant distribution in each study arm is provided in FIG. 33. The demographic characteristics of the study population are in Table 31 below.

TABLE 31
Summary of screening demographic data

| | OpSCF | Placebo |
|---|---|---|
| n | 26 | 24 |
| Age (years) (average) | 44.1 | 33.8 |
| USA clinic site | 23 | 17 |
| Female | 13 (50%) | 12 (50%) |
| vIGA 3/4 | 18/8 * | 18/6 |
| EASI (average) | 24.2 | 20.0 |
| BSA (average) | 30.9% | 25.3% |

* One subject had an IGA of 4, but was randomized as a 3 (Moderate)

The number of subjects in the placebo and OpSCF groups in the open label extension portion of the study is provided in Table 32 below.

TABLE 32
Number of subjects in open label extension

| | Study week | | | | | | | |
|---|---|---|---|---|---|---|---|---|
| N* | 16 | 20 | 24 | 28 | 32 | 36 | 40 | 44 |
| Placebo | 22 | 22 | 20 | 12 | 6 | 2 | 0 | 0 |
| OpSCF | 23 | 23 | 19 | 12 | 6 | 4 | 4 | 1 |

*N = number of human subjects

Treatment administered: The drug product administered was OpSCF, a monoclonal antibody, and the placebo was the formulation buffer without the active agent.

Clinical endpoints: This study assessed the following primary (Table 33), secondary (Table 34) and exploratory clinical endpoints (Table 35).

TABLE 33
The primary outcome measures were:

| Outcome Measure | Measure Description | Time Frame |
|---|---|---|
| Percent change in | Validated measure of activity of | 16 weeks |
| Eczema Area and | atopic dermatitis. A score of 0 | |
| Severity Index | indicates clear or no eczema, 0.1 | |
| (EASI) score from | to 1.0 indicates almost clear, 1.1 | |
| baseline at Week 16 | to 7 indicates mild disease, 7.1 to | |
| | 21 indicates moderate disease, | |
| | 21.1 to 50 indicates severe | |
| | disease, and greater than 51 | |
| | indicates very severe disease. | |

TABLE 34
The secondary outcome measures were:

| Outcome Measure | Measure Description | Time Frame |
|---|---|---|
| Incidence of AEs and SAEs | Safety | Through study completion, |
| | | approximately 1 year |
| | | including the open label |
| | | extension phase |
| Proportion of subjects achieving | Validated measure of activity of | 16 weeks |
| EASI 50/75/90 | atopic dermatitis. (Described | |
| | above) | |
| Proportion of subjects achieving | Validated Investigator scoring of | 16 weeks |
| at least a 2-grade reduction from | AD activity | |
| baseline to clear (0) or almost | | |
| clear (1) in vIGA-AD | | |
| Change and percent change | Validated scale of subject- | 16 weeks |
| from baseline in weekly average | reported itch. The Peak Pruritus | |
| of the daily Peak Pruritus | Numerical Rating Scale is a single | |
| Numerical Rating Scale (PP- | self-reported item designed to | |
| NRS) | measure peak pruritus, or ‘worst’ | |
| | itch, over the previous 24 hours | |
| | based on the following question: | |
| | ‘On a scale of 0 to 10, with 0 being | |
| | “no itch” and 10 being “worst itch | |
| | imaginable”. | |
| Change and percent change | Body surface area related to AD | 16 weeks |
| from baseline in AD related | | |
| BSA | | |
| Change from baseline in ADCT, | Atopic Dermatitis Control Tool; | 16 weeks |
| POEM and DLQI | Patient Oriented Eczema | |
| | Measure; Dermatology Life | |
| | Quality Index | |

TABLE 35
The other outcome measures were:

| Outcome Measure | Measure Description | Time Frame |
|---|---|---|
| Change from baseline in blood | Blood tests | 16 weeks |
| biomarkers and IgE at Weeks 4, | | |
| 8, 12, and 16 | | |
| Change from baseline in skin | Histopathological | 16 weeks |
| biomarkers collected using skin | examination | |
| biopsies at Weeks 4 and 16 | | |
| (optional for consenting | | |
| subjects) | | |
| Change from baseline in skin | Analysis of proteomics | 16 weeks |
| biomarkers collected using tape | and mRNA levels | |
| strips at Weeks 4 and 16 | | |
| (optional for consenting | | |
| subjects) | | |

### Results:

vIGA-AD

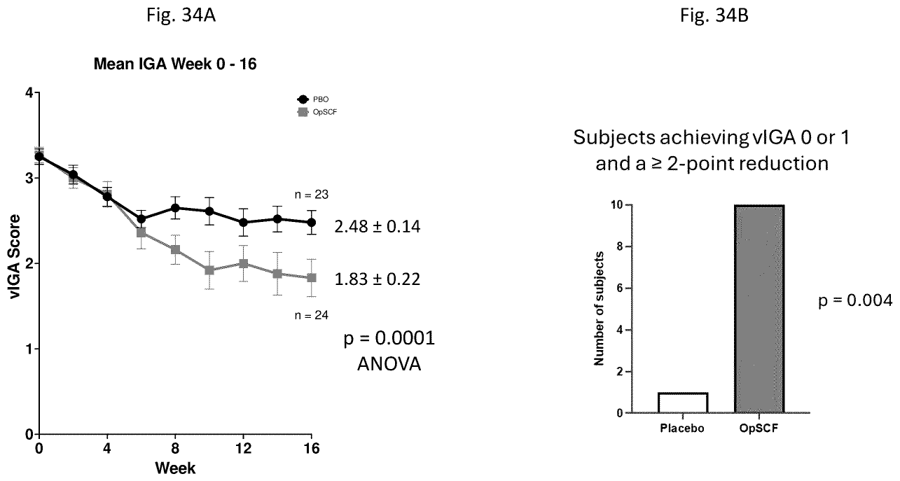

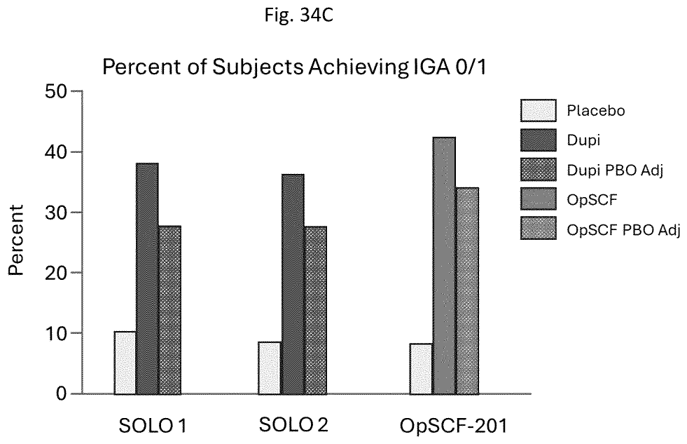

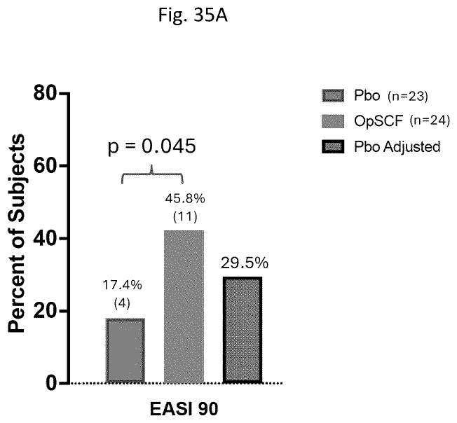

Results are in FIGS. 34A-34C. As shown in FIG. 34A, treatment with OpSCF significantly reduced the vIGA-AD score at Week 16, with the curve separating at Week 6. OpSCF had a significantly higher number of subjects achieve a vIGA-AD of 0 or 1 and a greater than 2-point reduction in comparison to the placebo group (FIG. 34B). No placebo subjects achieved a vIGA-AD of 0 (clear). When OpSCF was further compared to dupilumab, an approved monoclonal antibody drug, OpSCF had an equivalent or higher percent of subjects achieve an vIGA-AD of 0 or 1 (FIG. 34C). The placebo-adjusted treatment effect of OpSCF was also the same or higher as dupilumab. In sum, OpSCF significantly improved vIGA-AD and was comparable to or better than dupilumab for improving vIGA-AD.

### EASI

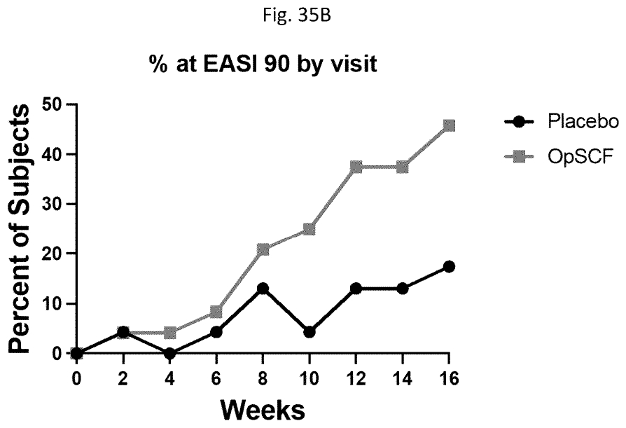

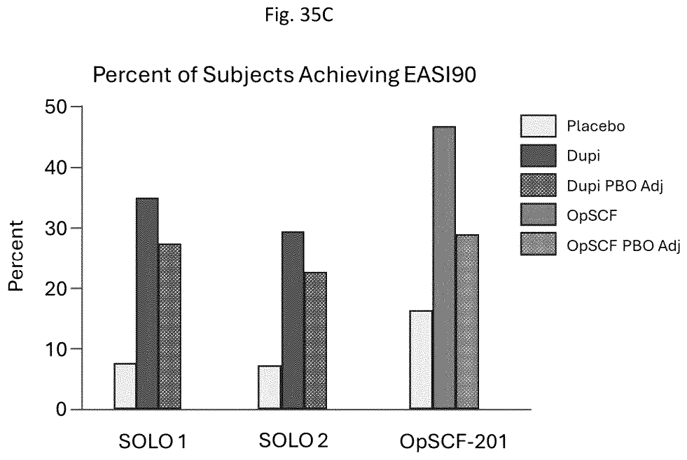

Results are in FIGS. 35A-35C. As shown in FIG. 35A, OpSCF outperformed placebo across all EASI endpoints, including on a placebo-adjusted basis. Specifically, greater than 40% of OpSCF-treated subjects achieved IGA 0/1 and EASI 75, and 8 of those 10 achieved EASI 90, compared to one placebo-treated subject. While placebo response muted the adjusted performance against weaker endpoints, OpSCF significantly outperformed placebo in EASI 90 (FIG. 35B). Moreover, when compared to dupilumab, the observed EASI 90 response of OpSCF was higher than dupilumab (FIG. 35C). The placebo-adjusted treatment effect of OpSCF was also the same or higher than dupilumab, despite a higher placebo rate.

### BSA

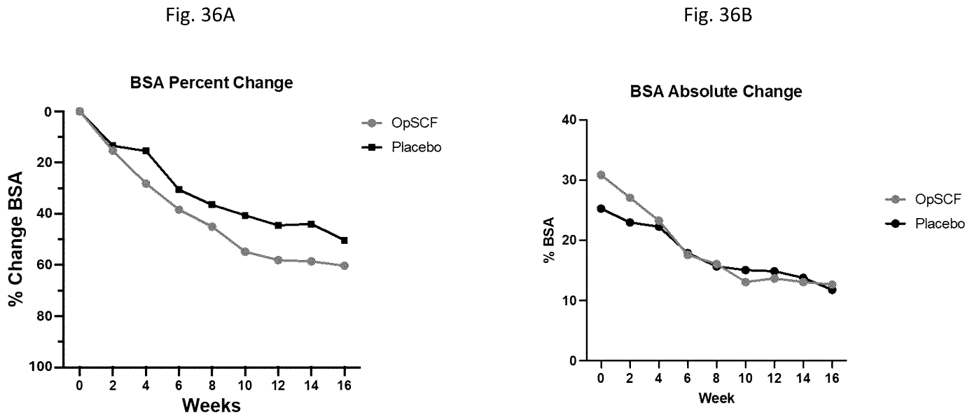

Results are in FIGS. 36A-36B. As shown, the OpSCF arm had a higher mean BSA at baseline versus placebo (31% vs 25%, respectively). Moreover, the OpSCF group mean BSA improved faster and to a greater degree than placebo consistently over time.

### Safety and Tolerability

Safety tolerability results are in Table 36 below. Overall, OpSCF was well tolerated. There were no SAEs, ADAs, or injection site reactions (ISRs). All reported AEs were mild to moderate, and no conjunctivitis, blepharitis or zoster was observed.

TABLE 36
Summary of Safety and tolerability data

| | OpSCF 600 mg | | Placebo | |
|---|---|---|---|---|
| | (N = 26) | | (N = 24) | |
| | n (%) [E] | | n (%) [E] | |
| Subjects with at least one | 16 | (61.5) [37] | 16 | (66.7) [46] |
| AE | | | | |
| Subjects with at least one | 15 | (57.7) [33] | 16 | (66.7) [45] |
| TEAE | | | | |
| Subjects with at least one | | | | |
| TEAE by greatest | | | | |
| relationship to study drug | | | | |
| No reasonable possibility | 11 | (42.3) [20] | 14 | (58.3) [21] |
| Reasonable possibility | 4 | (15.4) [9] | 2 | (8.3)[19] |
| Subjects with at least one | | | | |
| TEAE by worst severity | | | | |
| Mild | 7 | (26.9) [10] | 8 | (33.3) [34] |
| Moderate | 8 | (30.8) [11] | 8 | (33.3) [10] |

| | | |
|---|---|---|
| Severe | 0 | 0 |
| Life threatening | 0 | 0 |
| Death | 0 | 0 |

| | | | | |
|---|---|---|---|---|
| Subjects with at least one | 1 | (3.8) [1] | 1 | (4.2) [19] |

| injection site TEAE | | | |
|---|---|---|---|
| Subjects with at least one | 0 | 0 | |
| Serious TEAE | | | |
| Subjects with at least one | 0 | 2 | (8.3) [ 2] |
| TEAE leading to | | | |
| discontinuation from study | | | |
| treatment | | | |

### PK & PD

A summary of serum concentration (C_{trough}) of OpSCF by study visit is provided in Table 37 below.

TABLE 37
Summary of serum concentration of OpSCF by study visit

| | Study Visit (Week) | | | | | | | | |
|---|---|---|---|---|---|---|---|---|---|
| | | | | | | | 14 | | |
| | | | | | | | (Day | | |
| | 2 | 4 | 6 | 8 | 12 | 14 | 102) | 15 | 16 |
| N | 26 | 26 | 25 | 25 | 23 | 24 | 23 | 22 | 24 |
| Mean | 40.2 | 63.6 | 72 | 79 | 85.7 | 82.4 | 114 | 111 | 80.6 |
| (mg/L) | | | | | | | | | |
| SD | 14.3 | 26.9 | 32.1 | 36.5 | 46.4 | 39.9 | 56.9 | 53.1 | 34.5 |
| (mg/L) | | | | | | | | | |
| CV % | 35.5 | 42.3 | 44.6 | 46.2 | 54.1 | 48.4 | 50.0 | 47.8 | 42.8 |

### Open Label Extension

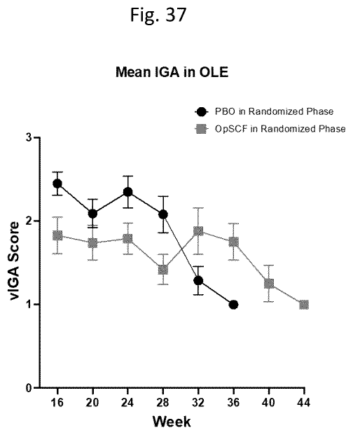

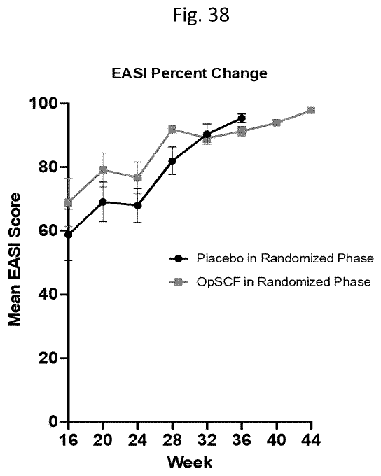

As shown in FIG. 37, the open label extension study confirmed strong vIGA-AD response with OpSCF dosing every four weeks in comparison to placebo. Moreover, as seen in FIG. 38 the subjects in the open label extension study showed improved EASI scores overtime. Specifically, higher EASI scores were maintained, and lower EASI scores were improved.

Conclusions: In sum, the Phase 2a clinical trial showed that stem cell factor was relevant in disease. OpSCF treatment effect was significant, being both internally consistent across measures and externally validated with comparators. Moreover, OpSCF was competitive with dupilumab in both observed and placebo adjusted responses. The open label extension confirmed strong activity, given that at half the dosing frequency OpSCF improved responses of placebo non responders, and demonstrated durability of response in all responders.

### INCORPORATION BY REFERENCE

Publications, patents and patent applications cited herein are specifically incorporated by reference in their entireties. While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

1. A method for treating atopic dermatitis in a human patient with atopic dermatitis, comprising administering to the human patient a therapeutically effective dose of a full length anti-stem cell factor 248 (anti-SCF248) antibody, wherein the full length anti-SCF248 antibody comprises two heavy chains each having the amino acid sequence of SEQ ID NO: 42 and two light chains each having the amino acid sequence of SEQ ID NO: 49; wherein the therapeutically effective dose is from 10 mg to 600 mg regardless of the human patient's body weight, and wherein treating is reducing or eliminating itching or a skin rash in the human patient.

2-105. (canceled)
